SEQUENCE LISTING (94 sequences)

SEQ ID NO: 1 - DNA, 1054 bp, Choristoneura fumiferana
cctgagtgcgtagtacccgagactcagtgcgccatgaagcggaaagagaagaaagcacagaaggagaaggacaaactgcctgtcagcacgacgacggtggacgaccacatgccgcccattatgcagtgtgaacctccacctcctgaagcagcaaggattcacgaagtggtcccaaggtttctctccgacaagctgttggagacaaaccggcagaaaaacatcccccagttgacagccaaccagcagttccttatcgccaggctcatctggtaccaggacgggtacgagcagccttctgatgaagatttgaagaggattacgcagacgtggcagcaagcggacgatgaaaacgaagagtctgacactcccttccgccagatcacagagatgactatcctcacggtccaacttatcgtggagttcgcgaagggattgccagggttcgccaagatctcgcagcctgatcaaattacgctgcttaaggcttgctcaagtgaggtaatgatgctccgagtcgcgcgacgatacgatgcggcctcagacagtgttctgttcgcgaacaaccaagcgtacactcgcgacaactaccgcaaggctggcatggcctacgtcatcgaggatctactgcacttctgccggtgcatgtactctatggcgttggacaacatccattacgcgctgctcacggctgtcgtcatcttttctgaccggccagggttggagcagccgcaactggtggaagaaatccagcggtactacctgaatacgctccgcatctatatcctgaaccagctgagcgggtcggcgcgttcgtccgtcatatacggcaagatcctctcaatcctctctgagctacgcacgctcggcatgcaaaactccaacatgtgcatctccctcaagctcaagaacagaaagctgccgcctttcctcgaggagatctgggatgtggcggacatgtcgcacacccaaccgccgcctatcctcgagtcccccacgaatctctagcccctgcgcgcacgcatcgccgatgccgcgtccggccgcgctgctctga

SEQ ID NO: 2 - DNA, 1288 bp, Choristoneura fumiferana
aagggccctgcgccccgtcagcaagaggaactgtgtctggtatgcggggacagagcctccggataccactacaatgcgctcacgtgtgaagggtgtaaagggttcttcagacggagtgttaccaaaaatgcggtttatatttgtaaattcggtcacgcttgcgaaatggacatgtacatgcgacggaaatgccaggagtgccgcctgaagaagtgcttagctgtaggcatgaggcctgagtgcgtagtacccgagactcagtgcgccatgaagcggaaagagaagaaagcacagaaggagaaggacaaactgcctgtcagcacgacgacggtggacgaccacatgccgcccattatgcagtgtgaacctccacctcctgaagcagcaaggattcacgaagtggtcccaaggtttctctccgacaagctgttggagacaaaccggcagaaaaacatcccccagttgacagccaaccagcagttccttatcgccaggctcatctggtaccaggacgggtacgagcagccttctgatgaagatttgaagaggattacgcagacgtggcagcaagcggacgatgaaaacgaagagtctgacactcccttccgccagatcacagagatgactatcctcacggtccaacttatcgtggagttcgcgaagggattgccagggttcgccaagatctcgcagcctgatcaaattacgctgcttaaggcttgctcaagtgaggtaatgatgctccgagtcgcgcgacgatacgatgcggcctcagacagtgttctgttcgcgaacaaccaagcgtacactcgcgacaactaccgcaaggctggcatggcctacgtcatcgaggatctactgcacttctgccggtgcatgtactctatggcgttggacaacatccattacgcgctgctcacggctgtcgtcatcttttctgaccggccagggttggagcagccgcaactggtggaagaaatccagcggtactacctgaatacgctccgcatctatatcctgaaccagctgagcgggtcggcgcgttcgtccgtcatatacggcaagatcctctcaatcctctctgagctacgcacgctcggcatgcaaaactccaacatgtgcatctccctcaagctcaagaacagaaagctgccgcctttcctcgaggagatctgggatgtggcggacatgtcgcacacccaaccgccgcctatcctcgagtcccccacgaatctctagcccctgcgcgcacgcatcgccgatgccgcgtccggccgcgctgctctga

SEQ ID NO: 3 - DNA, 1650 bp, Drosophila melanogaster
cggccggaatgcgtcgtcccggagaaccaatgtgcgatgaagcggcgcgaaaagaaggcccagaaggagaaggacaaaatgaccacttcgccgagctctcagcatggcggcaatggcagcttggcctctggtggcggccaagactttgttaagaaggagattcttgaccttatgacatgcgagccgccccagcatgccactattccgctactacctgatgaaatattggccaagtgtcaagcgcgcaatataccttccttaacgtacaatcagttggccgttatatacaagttaatttggtaccaggatggctatgagcagccatctgaagaggatctcaggcgtataatgagtcaacccgatgagaacgagagccaaacggacgtcagctttcggcatataaccgagataaccatactcacggtccagttgattgttgagtttgctaaaggtctaccagcgtttacaaagataccccaggaggaccagatcacgttactaaaggcctgctcgtcggaggtgatgatgctgcgtatggcacgacgctatgaccacagctcggactcaatattcttcgcgaataatagatcatatacgcgggattcttacaaaatggccggaatggctgataacattgaagacctgctgcatttctgccgccaaatgttctcgatgaaggtggacaacgtcgaatacgcgcttctcactgccattgtgatcttctcggaccggccgggcctggagaaggcccaactagtcgaagcgatccagagctactacatcgacacgctacgcatttatatactcaaccgccactgcggcgactcaatgagcctcgtcttctacgcaaagctgctctcgatcctcaccgagctgcgtacgctgggcaaccagaacgccgagatgtgtttctcactaaagctcaaaaaccgcaaactgcccaagttcctcgaggagatctgggacgttcatgccatcccgccatcggtccagtcgcaccttcagattacccaggaggagaacgagcgtctcgagcgggctgagcgtatgcgggcatcggttgggggcgccattaccgccggcattgattgcgactctgcctccacttcggcggcggcagccgcggcccagcatcagcctcagcctcagccccagccccaaccctcctccctgacccagaacgattcccagcaccagacacagccgcagctacaacctcagctaccacctcagctgcaaggtcaactgcaaccccagctccaaccacagcttcagacgcaactccagccacagattcaaccacagccacagctccttcccgtctccgctcccgtgcccgcctccgtaaccgcacctggttccttgtccgcggtcagtacgagcagcgaatacatgggcggaagtgcggccataggacccatcacgccggcaaccaccagcagtatcacggctgccgttaccgctagctccaccacatcagcggtaccgatgggcaacggagttggagtcggtgttggggtgggcggcaacgtcagcatgtatgcgaacgcccagacggcgatggccttgatgggtgtagccctgcattcgcaccaagagcagcttatcgggggagtggcggttaagtcggagcactcgacgactgcatag

SEQ ID NO: 4 - DNA, 894 bp, Tenebrio molitor
aggccggaatgtgtggtaccggaagtacagtgtgctgttaagagaaaagagaagaaagcccaaaaggaaaaagataaaccaaacagcactactaacggctcaccagacgtcatcaaaattgaaccagaattgtcagattcagaaaaaacattgactaacggacgcaataggatatcaccagagcaagaggagctcatactcatacatcgattggtttatttccaaaacgaatatgaacatccgtctgaagaagacgttaaacggattatcaatcagccgatagatggtgaagatcagtgtgagatacggtttaggcataccacggaaattacgatcctgactgtgcagctgatcgtggagtttgccaagcggttaccaggcttcgataagctcctgcaggaagatcaaattgctctcttgaaggcatgttcaagcgaagtgatgatgttcaggatggcccgacgttacgacgtccagtcggattccatcctcttcgtaaacaaccagccttatccgagggacagttacaatttggccggtatgggggaaaccatcgaagatctcttgcatttttgcagaactatgtactccatgaaggtggataatgccgaatatgctttactaacagccatcgttattttctcagagcgaccgtcgttgatagaaggctggaaggtggagaagatccaagaaatctatttagaggcattgcgggcgtacgtcgacaaccgaagaagcccaagccggggcacaatattcgcgaaactcctgtcagtactaactgaattgcggacgttaggcaaccaaaattcagagatgtgcatctcgttgaaattgaaaaacaaaaagttaccgccgttcctggacgaaatctgggacgtcgacttaaaagca

SEQ ID NO: 5 - DNA, 948 bp, Amblyomma americanum
cggccggaatgtgtggtgccggagtaccagtgtgccatcaagcgggagtctaagaagcaccagaaggaccggccaaacagcacaacgcgggaaagtccctcggcgctgatggcgccatcttctgtgggtggcgtgagccccaccagccagcccatgggtggcggaggcagctccctgggcagcagcaatcacgaggaggataagaagccagtggtgctcagcccaggagtcaagcccctctcttcatctcaggaggacctcatcaacaagctagtctactaccagcaggagtttgagtcgccttctgaggaagacatgaagaaaaccacgcccttccccctgggagacagtgaggaagacaaccagcggcgattccagcacattactgagatcaccatcctgacagtgcagctcattgtggagttctccaagcgggtccctggctttgacacgctggcacgagaagaccagattactttgctgaaggcctgctccagtgaagtgatgatgctgagaggtgcccggaaatatgatgtgaagacagattctatagtgtttgccaataaccagccgtacacgagggacaactaccgcagtgccagtgtgggggactctgcagatgccctgttccgcttctgccgcaagatgtgtcagctgagagtagacaacgctgaatacgcactcctgacggccattgtaattttctctgaacggccatcactggtggacccgcacaaggtggagcgcatccaggagtactacattgagaccctgcgcatgtactccgagaaccaccggcccccaggcaagaactactttgcccggctgctgtccatcttgacagagctgcgcaccttgggcaacatgaacgccgaaatgtgcttctcgctcaaggtgcagaacaagaagctgccaccgttcctggctgagatttgggacatccaagag

SEQ ID NO: 6 - protein, 334 aa, Choristoneura fumiferana
PECVVPETQCAMKRKEKKAQKEKDKLPVSTTTVDDHMPPIMQCEPPPPEAARIHEVVPRFLSDKLLETNRQKNIPQLTANQQFLIARLIWYQDGYEQPSDEDLKRITQTWQQADDENEESDTPFRQITEMTILTVQLIVEFAKGLPGFAKISQPDQITLLKACSSEVMMLRVARRYDAASDSVLFANNQAYTRDNYRKAGMAYVIEDLLHFCRCMYSMALDNIHYALLTAVVIFSDRPGLEQPQLVEEIQRYYLNTLRIYILNQLSGSARSSVIYGKILSILSELRTLGMQNSNMCISLKLKNRKLPPFLEEIWDVADMSHTQPPPILESPTNL

SEQ ID NO: 7 - protein, 549 aa, Drosophila melanogaster
RPECVVPENQCAMKRREKKAQKEKDKMTTSPSSQHGGNGSLASGGGQDFVKKEILDLMTCEPPQHATIPLLPDEILAKCQARNIPSLTYNQLAVIYKLIWYQDGYEQPSEEDLRRIMSQPDENESQTDVSFRHITEITILTVQLIVEFAKGLPAFTKIPQEDQITLLKACSSEVMMLRMARRYDHSSDSIFFANNRSYTRDSYKMAGMADNIEDLLHFCRQMFSMKVDNVEYALLTAIVIFSDRPGLEKAQLVEAIQSYYIDTLRIYILNRHCGDSMSLVFYAKLLSILTELRTLGNQNAEMCFSLKLKNRKLPKFLEEIWDVHAIPPSVQSHLQITQEENERLERAERMRASVGGAITAGIDCDSASTSAAAAAAQHQPQPQPQPQPSSLTQNDSQHQTQPQLQPQLPPQLQGQLQPQLQPQLQTQLQPQIQPQPQLLPVSAPVPASVTAPGSLSAVSTSSEYMGGSAAIGPITPATTSSITAAVTASSTTSAVPMGNGVGVGVGVGGNVSMYANAQTAMALMGVALHSHQEQLIGGVAVKSEHSTTA

SEQ ID NO: 8 - protein, 401 aa, Choristoneura fumiferana
CLVCGDRASGYHYNALTCEGCKGFFRRSVTKNAVYICKFGHACEMDMYMRRKCQECRLKKCLAVGMRPECVVPETQCAMKRKEKKAQKEKDKLPVSTTTVDDHMPPIMQCEPPPPEAARIHEVVPRFLSDKLLETNRQKNIPQLTANQQFLIARLIWYQDGYEQPSDEDLKRITQTWQQADDENEESDTPFRQITEMTILTVQLIVEFAKGLPGFAKISQPDQITLLKACSSEVMMLRVARRYDAASDSVLFANNQAYTRDNYRKAGMAYVIEDLLHFCRCMYSMALDNIHYALLTAVVIFSDRPGLEQPQLVEEIQRYYLNTLRIYILNQLSGSARSSVIYGKILSILSELRTLGMQNSNMCISLKLKNRKLPPFLEEIWDVADMSHTQPPPILESPTNL

SEQ ID NO: 9 - protein, 298 aa, Tenebrio molitor
RPECVVPEVQCAVKRKEKKAQKEKDKPNSTTNGSPDVIKIEPELSDSEKTLTNGRNRISPEQEELILIHRLVYFQNEYEHPSEEDVKRIINQPIDGEDQCEIRFRHTTEITILTVQLIVEFAKRLPGFDKLLQEDQIALLKACSSEVMMFRMARRYDVQSDSILFVNNQPYPRDSYNLAGMGETIEDLLHFCRTMYSMKVDNAEYALLTAIVIFSERPSLIEGWKVEKIQEIYLEALRAYVDNRRSPSRGTIFAKLLSVLTELRTLGNQNSEMCISLKLKNKKLPPFLDEIWDVDLKA

SEQ ID NO: 10 - protein, 316 aa, Amblyomma americanum
RPECVVPEYQCAIKRESKKHQKDRPNSTTRESPSALMAPSSVGGVSPTSQPMGGGGSSLGSSNHEEDKKPVVLSPGVKPLSSSQEDLINKLVYYQQEFESPSEEDMKKTTPFPLGDSEEDNQRRFQHITEITILTVQLIVEFSKRVPGFDTLAREDQITLLKACSSEVMMLRGARKYDVKTDSIVFANNQPYTRDNYRSASVGDSADALFRFCRKMCQLRVDNAEYALLTAIVIFSERPSLVDPHKVERIQEYYIETLRMYSENHRPPGKNYFARLLSILTELRTLGNMNAEMCFSLKVQNKKLPPFLAEIWDIQE

SEQ ID NO: 11 - DNA, 711 bp, Artificial Sequence
  Description of Artificial Sequence Synthetic Chimeric RXR ligand binding domain
gccaacgaggacatgcctgtagagaagattctggaagccgagcttgctgtcgagcccaagactgagacatacgtggaggcaaacatggggctgaaccccagctcaccaaatgaccctgttaccaacatctgtcaagcagcagacaagcagctcttcactcttgtggagtgggccaagaggatcccacacttttctgagctgcccctagacgaccaggtcatcctgctacgggcaggctggaacgagctgctgatcgcctccttctcccaccgctccatagctgtgaaagatgggattctcctggccaccggcctgcacgtacaccggaacagcgctcacagtgctggggtgggcgccatctttgacagggtgctaacagagctggtgtctaagatgcgtgacatgcagatggacaagactgaacttggctgcttgcgatctgttattcttttcaatccagaggtgaggggtttgaaatccgcccaggaagttgaacttctacgtgaaaaagtatatgccgctttggaagaatatactagaacaacacatcccgatgaaccaggaagatttgcaaaacttttgcttcgtctgccttctttacgttccataggccttaagtgtttggagcatttgtttttctttcgccttattggagatgttccaattgatacgttcctgatggagatgcttgaatcaccttctgattcataa

SEQ ID NO: 12 - DNA, 720 bp, Homo sapiens
gcccccgaggagatgcctgtggacaggatcctggaggcagagcttgctgtggaacagaagagtgaccagggcgttgagggtcctgggggaaccgggggtagcggcagcagcccaaatgaccctgtgactaacatctgtcaggcagctgacaaacagctattcacgcttgttgagtgggcgaagaggatcccacacttttcctccttgcctctggatgatcaggtcatattgctgcgggcaggctggaatgaactcctcattgcctccttttcacaccgatccattgatgttcgagatggcatcctccttgccacaggtcttcacgtgcaccgcaactcagcccattcagcaggagtaggagccatctttgatcgggtgctgacagagctagtgtccaaaatgcgtgacatgaggatggacaagacagagcttggctgcctgagggcaatcattctgtttaatccagatgccaagggcctctccaaccctagtgaggtggaggtcctgcgggagaaagtgtatgcatcactggagacctactgcaaacagaagtaccctgagcagcagggacggtttgccaagctgctgctacgtcttcctgccctccggtccattggccttaagtgtctagagcatctgtttttcttcaagctcattggtgacacccccatcgacaccttcctcatggagatgcttgaggctccccatcaactggcctga

SEQ ID NO: 13 - DNA, 635 bp, Locusta migratoria
tgcatacagacatgcctgttgaacgcatacttgaagctgaaaaacgagtggagtgcaaagcagaaaaccaagtggaatatgagctggtggagtgggctaaacacatcccgcacttcacatccctacctctggaggaccaggttctcctcctcagagcaggttggaatgaactgctaattgcagcattttcacatcgatctgtagatgttaaagatggcatagtacttgccactggtctcacagtgcatcgaaattctgcccatcaagctggagtcggcacaatatttgacagagttttgacagaactggtagcaaagatgagagaaatgaaaatggataaaactgaacttggctgcttgcgatctgttattcttttcaatccagaggtgaggggtttgaaatccgcccaggaagttgaacttctacgtgaaaaagtatatgccgctttggaagaatatactagaacaacacatcccgatgaaccaggaagatttgcaaaacttttgcttcgtctgccttctttacgttccataggccttaagtgtttggagcatttgtttttctttcgccttattggagatgttccaattgatacgttcctgatggagatgcttgaatcaccttctgattcataa

SEQ ID NO: 14 - protein, 236 aa, Artificial Sequence
  Description of Artificial Sequence Synthetic Chimeric RXR ligand binding domain
ANEDMPVEKILEAELAVEPKTETYVEANMGLNPSSPNDPVTNICQAADKQLFTLVEWAKRIPHFSELPLDDQVILLRAGWNELLIASFSHRSIAVKDGILLATGLHVHRNSAHSAGVGAIFDRVLTELVSKMRDMQMDKTELGCLRSVILFNPEVRGLKSAQEVELLREKVYAALEEYTRTTHPDEPGRFAKLLLRLPSLRSIGLKCLEHLFFFRLIGDVPIDTFLMEMLESPSDS

SEQ ID NO: 15 - protein, 239 aa, Homo sapiens
APEEMPVDRILEAELAVEQKSDQGVEGPGGTGGSGSSPNDPVTNICQAADKQLFTLVEWAKRIPHFSSLPLDDQVILLRAGWNELLIASFSHRSIDVRDGILLATGLHVHRNSAHSAGVGAIFDRVLTELVSKMRDMRMDKTELGCLRAIILFNPDAKGLSNPSEVEVLREKVYASLETYCKQKYPEQQGRFAKLLLRLPALRSIGLKCLEHLFFFKLIGDTPIDTFLMEMLEAPHQLA

SEQ ID NO: 16 - protein, 210 aa, Locusta migratoria
HTDMPVERILEAEKRVECKAENQVEYELVEWAKHIPHFTSLPLEDQVLLLRAGWNELLIAAFSHRSVDVKDGIVLATGLTVHRNSAHQAGVGTIFDRVLTELVAKMREMKMDKTELGCLRSVILFNPEVRGLKSAQEVELLREKVYAALEEYTRTTHPDEPGRFAKLLLRLPSLRSIGLKCLEHLFFFRLIGDVPIDTFLMEMLESPSDS

SEQ ID NO: 17 - protein, 240 aa, Choristoneura fumiferana
LTANQQFLIARLIWYQDGYEQPSDEDLKRITQTWQQADDENEESDTPFRQITEMTILTVQLIVEFAKGLPGFAKISQPDQITLLKACSSEVMMLRVARRYDAASDSVLFANNQAYTRDNYRKAGMAYVIEDLLHFCRCMYSMALDNIHYALLTAVVIFSDRPGLEQPQLVEEIQRYYLNTLRIYILNQLSGSARSSVIYGKILSILSELRTLGMQNSNMCISLKLKNRKLPPFLEEIWDV

SEQ ID NO: 18 - protein, 237 aa, Drosophila melanogaster
LTYNQLAVIYKLIWYQDGYEQPSEEDLRRIMSQPDENESQTDVSFRHITEITILTVQLIVEFAKGLPAFTKIPQEDQITLLKACSSEVMMLRMARRYDHSSDSIFFANNRSYTRDSYKMAGMADNIEDLLHFCRQMFSMKVDNVEYALLTAIVIFSDRPGLEKAQLVEAIQSYYIDTLRIYILNRHCGDSMSLVFYAKLLSILTELRTLGNQNAEMCFSLKLKNRKLPKFLEEIWDV

SEQ ID NO: 19 - protein, 240 aa, Amblyomma americanum
PGVKPLSSSQEDLINKLVYYQQEFESPSEEDMKKTTPFPLGDSEEDNQRRFQHITEITILTVQLIVEFSKRVPGFDTLAREDQITLLKACSSEVMMLRGARKYDVKTDSIVFANNQPYTRDNYRSASVGDSADALFRFCRKMCQLRVDNAEYALLTAIVIFSERPSLVDPHKVERIQEYYIETLRMYSENHRPPGKNYFARLLSILTELRTLGNMNAEMCFSLKVQNKKLPPFLAEIWDI

SEQ ID NO: 20 - DNA, 1586 bp, Bamecia argentifoli
gaattcgcggccgctcgcaaacttccgtacctctcaccccctcgccaggaccccccgccaaccagttcaccgtcatctcctccaatggatactcatcccccatgtcttcgggcagctacgacccttatagtcccaccaatggaagaatagggaaagaagagctttcgccggcgaatagtctgaacgggtacaacgtggatagctgcgatgcgtcgcggaagaagaagggaggaacgggtcggcagcaggaggagctgtgtctcgtctgcggggaccgcgcctccggctaccactacaacgccctcacctgcgaaggctgcaagggcttcttccgtcggagcatcaccaagaatgccgtctaccagtgtaaatatggaaataattgtgaaattgacatgtacatgaggcgaaaatgccaagagtgtcgtctcaagaagtgtctcagcgttggcatgaggccagaatgtgtagttcccgaattccagtgtgctgtgaagcgaaaagagaaaaaagcgcaaaaggacaaagataaacctaactcaacgacgagttgttctccagatggaatcaaacaagagatagatcctcaaaggctggatacagattcgcagctattgtctgtaaatggagttaaacccattactccagagcaagaagagctcatccataggctagtttattttcaaaatgaatatgaacatccatccccagaggatatcaaaaggatagttaatgctgcaccagaagaagaaaatgtagctgaagaaaggtttaggcatattacagaaattacaattctcactgtacagttaattgtggaattttctaagcgattacctggttttgacaaactaattcgtgaagatcaaatagctttattaaaggcatgtagtagtgaagtaatgatgtttagaatggcaaggaggtatgatgctgaaacagattcgatattgtttgcaactaaccagccgtatacgagagaatcatacactgtagctggcatgggtgatactgtggaggatctgctccgattttgtcgacatatgtgtgccatgaaagtcgataacgcagaatatgctcttctcactgccattgtaattttttcagaacgaccatctctaagtgaaggctggaaggttgagaagattcaagaaatttacatagaagcattaaaagcatatgttgaaaatcgaaggaaaccatatgcaacaaccatttttgctaagttactatctgttttaactgaactacgaacattagggaatatgaattcagaaacatgcttctcattgaagctgaagaatagaaaggtgccatccttcctcgaggagatttgggatgttgtttcataaacagtcttacctcaattccatgttacttttcatatttgatttatctcagcaggtggctcagtacttatcctcacattactgagctcacggtatgctcatacaattataacttgtaatatcatatcggtgatgacaaatttgttacaatattctttgttaccttaacacaatgttgatctcataatgatgtatgaatttttctgtttttgcaaaaaaaaaagcggccgcgaattc

SEQ ID NO: 21 - DNA, 1109 bp, Nephotetix cincticeps
caggaggagctctgcctgttgtgcggagaccgagcgtcgggataccactacaacgctctcacctgcgaaggatgcaagggcttctttcggaggagtatcaccaaaaacgcagtgtaccagtccaaatacggcaccaattgtgaaatagacatgtatatgcggcgcaagtgccaggagtgccgactcaagaagtgcctcagtgtagggatgaggccagaatgtgtagtacctgagtatcaatgtgccgtaaaaaggaaagagaaaaaagctcaaaaggacaaagataaacctgtctcttcaaccaatggctcgcctgaaatgagaatagaccaggacaaccgttgtgtggtgttgcagagtgaagacaacaggtacaactcgagtacgcccagtttcggagtcaaacccctcagtccagaacaagaggagctcatccacaggctcgtctacttccagaacgagtacgaacaccctgccgaggaggatctcaagcggatcgagaacctcccctgtgacgacgatgacccgtgtgatgttcgctacaaacacattacggagatcacaatactcacagtccagctcatcgtggagtttgcgaaaaaactgcctggtttcgacaaactactgagagaggaccagatcgtgttgctcaaggcgtgttcgagcgaggtgatgatgctgcggatggcgcggaggtacgacgtccagacagactcgatcctgttcgccaacaaccagccgtacacgcgagagtcgtacacgatggcaggcgtgggggaagtcatcgaagatctgctgcggttcggccgactcatgtgctccatgaaggtggacaatgccgagtatgctctgctcacggccatcgtcatcttctccgagcggccgaacctggcggaaggatggaaggttgagaagatccaggagatctacctggaggcgctcaagtcctacgtggacaaccgagtgaaacctcgcagtccgaccatcttcgccaaactgctctccgttctcaccgagctgcgaacactcggcaaccagaactccgagatgtgcttctcgttaaactacgcaaccgcaaacatgccaccgttcctcgaagaaatctggga

SEQ ID NO: 22 - DNA, 735 bp, Choristoneura fumiferana
taccaggacgggtacgagcagccttctgatgaagatttgaagaggattacgcagacgtggcagcaagcggacgatgaaaacgaagagtctgacactcccttccgccagatcacagagatgactatcctcacggtccaacttatcgtggagttcgcgaagggattgccagggttcgccaagatctcgcagcctgatcaaattacgctgcttaaggcttgctcaagtgaggtaatgatgctccgagtcgcgcgacgatacgatgcggcctcagacagtgttctgttcgcgaacaaccaagcgtacactcgcgacaactaccgcaaggctggcatggcctacgtcatcgaggatctactgcacttctgccggtgcatgtactctatggcgttggacaacatccattacgcgctgctcacggctgtcgtcatcttttctgaccggccagggttggagcagccgcaactggtggaagaaatccagcggtactacctgaatacgctccgcatctatatcctgaaccagctgagcgggtcggcgcgttcgtccgtcatatacggcaagatcctctcaatcctctctgagctacgcacgctcggcatgcaaaactccaacatgtgcatctccctcaagctcaagaacagaaagctgccgcctttcctcgaggagatctgggatgtggcggacatgtcgcacacccaaccgccgcctatcctcgagtcccccacgaatctctag

SEQ ID NO: 23 - DNA, 1338 bp, Drosophila melanogaster
tatgagcagccatctgaagaggatctcaggcgtataatgagtcaacccgatgagaacgagagccaaacggacgtcagctttcggcatataaccgagataaccatactcacggtccagttgattgttgagtttgctaaaggtctaccagcgtttacaaagataccccaggaggaccagatcacgttactaaaggcctgctcgtcggaggtgatgatgctgcgtatggcacgacgctatgaccacagctcggactcaatattcttcgcgaataatagatcatatacgcgggattcttacaaaatggccggaatggctgataacattgaagacctgctgcatttctgccgccaaatgttctcgatgaaggtggacaacgtcgaatacgcgcttctcactgccattgtgatcttctcggaccggccgggcctggagaaggcccaactagtcgaagcgatccagagctactacatcgacacgctacgcatttatatactcaaccgccactgcggcgactcaatgagcctcgtcttctacgcaaagctgctctcgatcctcaccgagctgcgtacgctgggcaaccagaacgccgagatgtgtttctcactaaagctcaaaaaccgcaaactgcccaagttcctcgaggagatctgggacgttcatgccatcccgccatcggtccagtcgcaccttcagattacccaggaggagaacgagcgtctcgagcgggctgagcgtatgcgggcatcggttgggggcgccattaccgccggcattgattgcgactctgcctccacttcggcggcggcagccgcggcccagcatcagcctcagcctcagccccagccccaaccctcctccctgacccagaacgattcccagcaccagacacagccgcagctacaacctcagctaccacctcagctgcaaggtcaactgcaaccccagctccaaccacagcttcagacgcaactccagccacagattcaaccacagccacagctccttcccgtctccgctcccgtgcccgcctccgtaaccgcacctggttccttgtccgcggtcagtacgagcagcgaatacatgggcggaagtgcggccataggacccatcacgccggcaaccaccagcagtatcacggctgccgttaccgctagctccaccacatcagcggtaccgatgggcaacggagttggagtcggtgttggggtgggcggcaacgtcagcatgtatgcgaacgcccagacggcgatggccttgatgggtgtagccctgcattcgcaccaagagcagcttatcgggggagtggcggttaagtcggagcactcgacgactgcatag

SEQ ID NO: 24 - DNA, 960 bp, Choristoneura fumiferana
cctgagtgcgtagtacccgagactcagtgcgccatgaagcggaaagagaagaaagcacagaaggagaaggacaaactgcctgtcagcacgacgacggtggacgaccacatgccgcccattatgcagtgtgaacctccacctcctgaagcagcaaggattcacgaagtggtcccaaggtttctctccgacaagctgttggagacaaaccggcagaaaaacatcccccagttgacagccaaccagcagttccttatcgccaggctcatctggtaccaggacgggtacgagcagccttctgatgaagatttgaagaggattacgcagacgtggcagcaagcggacgatgaaaacgaagagtctgacactcccttccgccagatcacagagatgactatcctcacggtccaacttatcgtggagttcgcgaagggattgccagggttcgccaagatctcgcagcctgatcaaattacgctgcttaaggcttgctcaagtgaggtaatgatgctccgagtcgcgcgacgatacgatgcggcctcagacagtgttctgttcgcgaacaaccaagcgtacactcgcgacaactaccgcaaggctggcatggcctacgtcatcgaggatctactgcacttctgccggtgcatgtactctatggcgttggacaacatccattacgcgctgctcacggctgtcgtcatcttttctgaccggccagggttggagcagccgcaactggtggaagaaatccagcggtactacctgaatacgctccgcatctatatcctgaaccagctgagcgggtcggcgcgttcgtccgtcatatacggcaagatcctctcaatcctctctgagctacgcacgctcggcatgcaaaactccaacatgtgcatctccctcaagctcaagaacagaaagctgccgcctttcctcgaggagatctgggatgtggcggacatgtcg

SEQ ID NO: 25 - DNA, 969 bp, Drosophila melanogaster
cggccggaatgcgtcgtcccggagaaccaatgtgcgatgaagcggcgcgaaaagaaggcccagaaggagaaggacaaaatgaccacttcgccgagctctcagcatggcggcaatggcagcttggcctctggtggcggccaagactttgttaagaaggagattcttgaccttatgacatgcgagccgccccagcatgccactattccgctactacctgatgaaatattggccaagtgtcaagcgcgcaatataccttccttaacgtacaatcagttggccgttatatacaagttaatttggtaccaggatggctatgagcagccatctgaagaggatctcaggcgtataatgagtcaacccgatgagaacgagagccaaacggacgtcagctttcggcatataaccgagataaccatactcacggtccagttgattgttgagtttgctaaaggtctaccagcgtttacaaagataccccaggaggaccagatcacgttactaaaggcctgctcgtcggaggtgatgatgctgcgtatggcacgacgctatgaccacagctcggactcaatattcttcgcgaataatagatcatatacgcgggattcttacaaaatggccggaatggctgataacattgaagacctgctgcatttctgccgccaaatgttctcgatgaaggtggacaacgtcgaatacgcgcttctcactgccattgtgatcttctcggaccggccgggcctggagaaggcccaactagtcgaagcgatccagagctactacatcgacacgctacgcatttatatactcaaccgccactgcggcgactcaatgagcctcgtcttctacgcaaagctgctctcgatcctcaccgagctgcgtacgctgggcaaccagaacgccgagatgtgtttctcactaaagctcaaaaaccgcaaactgcccaagttcctcgaggagatctgggacgtt

SEQ ID NO: 26 - protein, 244 aa, Choristoneura fumiferana
YQDGYEQPSDEDLKRITQTWQQADDENEESDTPFRQITEMTILTVQLIVEFAKGLPGFAKISQPDQITLLKACSSEVMMLRVARRYDAASDSVLFANNQAYTRDNYRKAGMAYVIEDLLHFCRCMYSMALDNIHYALLTAVVIFSDRPGLEQPQLVEEIQRYYLNTLRIYILNQLSGSARSSVIYGKILSILSELRTLGMQNSNMCISLKLKNRKLPPFLEEIWDVADMSHTQPPPILESPTNL

SEQ ID NO: 27 - protein, 445 aa, Drosophila melanogaster
YEQPSEEDLRRIMSQPDENESQTDVSFRHITEITILTVQLIVEFAKGLPAFTKIPQEDQITLLKACSSEVMMLRMARRYDHSSDSIFFANNRSYTRDSYKMAGMADNIEDLLHFCRQMFSMKVDNVEYALLTAIVIFSDRPGLEKAQLVEAIQSYYIDTLRIYILNRHCGDSMSLVFYAKLLSILTELRTLGNQNAEMCFSLKLKNRKLPKFLEEIWDVHAIPPSVQSHLQITQEENERLERAERMRASVGGAITAGIDCDSASTSAAAAAAQHQPQPQPQPQPSSLTQNDSQHQTQPQLQPQLPPQLQGQLQPQLQPQLQTQLQPQIQPQPQLLPVSAPVPASVTAPGSLSAVSTSSEYMGGSAAIGPITPATTSSITAAVTASSTTSAVPMGNGVGVGVGVGGNVSMYANAQTAMALMGVALHSHQEQLIGGVAVKSEHSTTA

SEQ ID NO: 28 - protein, 320 aa, Choristoneura fumiferana
PECVVPETQCAMKRKEKKAQKEKDKLPVSTTTVDDHMPPIMQCEPPPPEAARIHEVVPRFLSDKLLETNRQKNIPQLTANQQFLIARLIWYQDGYEQPSDEDLKRITQTWQQADDENEESDTPFRQITEMTILTVQLIVEFAKGLPGFAKISQPDQITLLKACSSEVMMLRVARRYDAASDSVLFANNQAYTRDNYRKAGMAYVIEDLLHFCRCMYSMALDNIHYALLTAVVIFSDRPGLEQPQLVEEIQRYYLNTLRIYILNQLSGSARSSVIYGKILSILSELRTLGMQNSNMCISLKLKNRKLPPFLEEIWDVADMS

SEQ ID NO: 29 - protein, 323 aa, Drosophila melanogaster
RPECVVPENQCAMKRREKKAQKEKDKMTTSPSSQHGGNGSLASGGGQDFVKKEILDLMTCEPPQHATIPLLPDEILAKCQARNIPSLTYNQLAVIYKLIWYQDGYEQPSEEDLRRIMSQPDENESQTDVSFRHITEITILTVQLIVEFAKGLPAFTKIPQEDQITLLKACSSEVMMLRMARRYDHSSDSIFFANNRSYTRDSYKMAGMADNIEDLLHFCRQMFSMKVDNVEYALLTAIVIFSDRPGLEKAQLVEAIQSYYIDTLRIYILNRHCGDSMSLVFYAKLLSILTELRTLGNQNAEMCFSLKLKNRKLPKFLEEIWDV

SEQ ID NO: 30 - DNA, 987 bp, Artificial Sequence
  Description of Artificial Sequence Synthetic polynucleotide
tgtgctatctgtggggaccgctcctcaggcaaacactatggggtatacagttgtgagggctgcaagggcttcttcaagaggacagtacgcaaagacctgacctacacctgccgagacaacaaggactgcctgatcgacaagagacagcggaaccggtgtcagtactgccgctaccagaagtgcctggccatgggcatgaagcgggaagctgtgcaggaggagcggcagcggggcaaggaccggaatgagaacgaggtggagtccaccagcagtgccaacgaggacatgcctgtagagaagattctggaagccgagcttgctgtcgagcccaagactgagacatacgtggaggcaaacatggggctgaaccccagctcaccaaatgaccctgttaccaacatctgtcaagcagcagacaagcagctcttcactcttgtggagtgggccaagaggatcccacacttttctgagctgcccctagacgaccaggtcatcctgctacgggcaggctggaacgagctgctgatcgcctccttctcccaccgctccatagctgtgaaagatgggattctcctggccaccggcctgcacgtacaccggaacagcgctcacagtgctggggtgggcgccatctttgacagggtgctaacagagctggtgtctaagatgcgtgacatgcagatggacaagacggagctgggctgcctgcgagccattgtcctgttcaaccctgactctaaggggctctcaaaccctgctgaggtggaggcgttgagggagaaggtgtatgcgtcactagaagcgtactgcaaacacaagtaccctgagcagccgggcaggtttgccaagctgctgctccgcctgcctgcactgcgttccatcgggctcaagtgcctggagcacctgttcttcttcaagctcatcggggacacgcccatcgacaccttcctcatggagatgctggaggcaccacatcaagccacctag

SEQ ID NO: 31 - DNA, 789 bp, Artificial Sequence
  Description of Artificial Sequence Synthetic polynucleotide
aagcgggaagctgtgcaggaggagcggcagcggggcaaggaccggaatgagaacgaggtggagtccaccagcagtgccaacgaggacatgcctgtagagaagattctggaagccgagcttgctgtcgagcccaagactgagacatacgtggaggcaaacatggggctgaaccccagctcaccaaatgaccctgttaccaacatctgtcaagcagcagacaagcagctcttcactcttgtggagtgggccaagaggatcccacacttttctgagctgcccctagacgaccaggtcatcctgctacgggcaggctggaacgagctgctgatcgcctccttctcccaccgctccatagctgtgaaagatgggattctcctggccaccggcctgcacgtacaccggaacagcgctcacagtgctggggtgggcgccatctttgacagggtgctaacagagctggtgtctaagatgcgtgacatgcagatggacaagacggagctgggctgcctgcgagccattgtcctgttcaaccctgactctaaggggctctcaaaccctgctgaggtggaggcgttgagggagaaggtgtatgcgtcactagaagcgtactgcaaacacaagtaccctgagcagccgggcaggtttgccaagctgctgctccgcctgcctgcactgcgttccatcgggctcaagtgcctggagcacctgttcttcttcaagctcatcggggacacgcccatcgacaccttcctcatggagatgctggaggcaccacatcaagccacctag

SEQ ID NO: 32 - DNA, 714 bp, Artificial Sequence
  Description of Artificial Sequence Synthetic polynucleotide
gccaacgaggacatgcctgtagagaagattctggaagccgagcttgctgtcgagcccaagactgagacatacgtggaggcaaacatggggctgaaccccagctcaccaaatgaccctgttaccaacatctgtcaagcagcagacaagcagctcttcactcttgtggagtgggccaagaggatcccacacttttctgagctgcccctagacgaccaggtcatcctgctacgggcaggctggaacgagctgctgatcgcctccttctcccaccgctccatagctgtgaaagatgggattctcctggccaccggcctgcacgtacaccggaacagcgctcacagtgctggggtgggcgccatctttgacagggtgctaacagagctggtgtctaagatgcgtgacatgcagatggacaagacggagctgggctgcctgcgagccattgtcctgttcaaccctgactctaaggggctctcaaaccctgctgaggtggaggcgttgagggagaaggtgtatgcgtcactagaagcgtactgcaaacacaagtaccctgagcagccgggcaggtttgccaagctgctgctccgcctgcctgcactgcgttccatcgggctcaagtgcctggagcacctgttcttcttcaagctcatcggggacacgcccatcgacaccttcctcatggagatgctggaggcaccacatcaagccacctag

SEQ ID NO: 33 - DNA, 536 bp, Artificial Sequence
  Description of Artificial Sequence Synthetic polynucleotide
ggatcccacacttttctgagctgcccctagacgaccaggtcatcctgctacgggcaggctggaacgagctgctgatcgcctccttctcccaccgctccatagctgtgaaagatgggattctcctggccaccggcctgcacgtacaccggaacagcgctcacagtgctggggtgggcgccatctttgacagggtgctaacagagctggtgtctaagatgcgtgacatgcagatggacaagacggagctgggctgcctgcgagccattgtcctgttcaaccctgactctaaggggctctcaaaccctgctgaggtggaggcgttgagggagaaggtgtatgcgtcactagaagcgtactgcaaacacaagtaccctgagcagccgggcaggtttgccaagctgctgctccgcctgcctgcactgcgttccatcgggctcaagtgcctggagcacctgttcttcttcaagctcatcggggacacgcccatcgacaccttcctcatggagatgctggaggcaccacatcaagccacctag

SEQ ID NO: 34 - DNA, 672 bp, Artificial Sequence
  Description of Artificial Sequence Synthetic polynucleotide
gccaacgaggacatgcctgtagagaagattctggaagccgagcttgctgtcgagcccaagactgagacatacgtggaggcaaacatggggctgaaccccagctcaccaaatgaccctgttaccaacatctgtcaagcagcagacaagcagctcttcactcttgtggagtgggccaagaggatcccacacttttctgagctgcccctagacgaccaggtcatcctgctacgggcaggctggaacgagctgctgatcgcctccttctcccaccgctccatagctgtgaaagatgggattctcctggccaccggcctgcacgtacaccggaacagcgctcacagtgctggggtgggcgccatctttgacagggtgctaacagagctggtgtctaagatgcgtgacatgcagatggacaagacggagctgggctgcctgcgagccattgtcctgttcaaccctgactctaaggggctctcaaaccctgctgaggtggaggcgttgagggagaaggtgtatgcgtcactagaagcgtactgcaaacacaagtaccctgagcagccgggcaggtttgccaagctgctgctccgcctgcctgcactgcgttccatcgggctcaagtgcctggagcacctgttcttcttcaagctcatcggggacacgcccatcgacacc

SEQ ID NO: 35 - DNA, 1123 bp, Artificial Sequence
  Description of Artificial Sequence Synthetic Novel polynucleotide sequence
tgcgccatctgcggggaccgctcctcaggcaagcactatggagtgtacagctgcgaggggtgcaagggcttcttcaagcggacggtgcgcaaggacctgacctacacctgccgcgacaacaaggactgcctgattgacaagcggcagcggaaccggtgccagtactgccgctaccagaagtgcctggccatgggcatgaagcgggaagccgtgcaggaggagcggcagcgtggcaaggaccggaacgagaatgaggtggagtcgaccagcagcgccaacgaggacatgccggtggagaggatcctggaggctgagctggccgtggagcccaagaccgagacctacgtggaggcaaacatggggctgaaccccagctcgccgaacgaccctgtcaccaacatttgccaagcagccgacaaacagcttttcaccctggtggagtgggccaagcggatcccacacttctcagagctgcccctggacgaccaggtcatcctgctgcgggcaggctggaatgagctgctcatcgcctccttctcccaccgctccatcgccgtgaaggacgggatcctcctggccaccgggctgcacgtccaccggaacagcgcccacagcgcaggggtgggcgccatctttgacagggtgctgacggagcttgtgtccaagatgcgggacatgcagatggacaagacggagctgggctgcctgcgcgccatcgtcctctttaaccctgactccaaggggctctcgaacccggccgaggtggaggcgctgagggagaaggtctatgcgtccttggaggcctactgcaagcacaagtacccagagcagccgggaaggttcgctaagctcttgctccgcctgccggctctgcgctccatcgggctcaaatgcctggaacatctcttcttcttcaagctcatcggggacacacccattgacaccttccttatggagatgctggaggcgccgcaccaaatgacttaggcctgcgggcccatcctttgtgcccacccgttctggccaccctgcctggacgccagctgttcttctcagcctgagccctgtccctgcccttctctgcctggcctgtttggactttggggcacagcctgtcactgct

SEQ ID NO: 36 - DNA, 925 bp, Artificial Sequence
  Description of Artificial Sequence Synthetic Novel polynucleotide sequence
aagcgggaagccgtgcaggaggagcggcagcgtggcaaggaccggaacgagaatgaggtggagtcgaccagcagcgccaacgaggacatgccggtggagaggatcctggaggctgagctggccgtggagcccaagaccgagacctacgtggaggcaaacatggggctgaaccccagctcgccgaacgaccctgtcaccaacatttgccaagcagccgacaaacagcttttcaccctggtggagtgggccaagcggatcccacacttctcagagctgcccctggacgaccaggtcatcctgctgcgggcaggctggaatgagctgctcatcgcctccttctcccaccgctccatcgccgtgaaggacgggatcctcctggccaccgggctgcacgtccaccggaacagcgcccacagcgcaggggtgggcgccatctttgacagggtgctgacggagcttgtgtccaagatgcgggacatgcagatggacaagacggagctgggctgcctgcgcgccatcgtcctctttaaccctgactccaaggggctctcgaacccggccgaggtggaggcgctgagggagaaggtctatgcgtccttggaggcctactgcaagcacaagtacccagagcagccgggaaggttcgctaagctcttgctccgcctgccggctctgcgctccatcgggctcaaatgcctggaacatctcttcttcttcaagctcatcggggacacacccattgacaccttccttatggagatgctggaggcgccgcaccaaatgacttaggcctgcgggcccatcctttgtgcccacccgttctggccaccctgcctggacgccagctgttcttctcagcctgagccctgtccctgcccttctctgcctggcctgtttggactttggggcacagcctgtcactgct

SEQ ID NO: 37 - DNA, 850 bp, Artificial Sequence
  Description of Artificial Sequence Synthetic Novel polynucleotide sequence
gccaacgaggacatgccggtggagaggatcctggaggctgagctggccgtggagcccaagaccgagacctacgtggaggcaaacatggggctgaaccccagctcgccgaacgaccctgtcaccaacatttgccaagcagccgacaaacagcttttcaccctggtggagtgggccaagcggatcccacacttctcagagctgcccctggacgaccaggtcatcctgctgcgggcaggctggaatgagctgctcatcgcctccttctcccaccgctccatcgccgtgaaggacgggatcctcctggccaccgggctgcacgtccaccggaacagcgcccacagcgcaggggtgggcgccatctttgacagggtgctgacggagcttgtgtccaagatgcgggacatgcagatggacaagacggagctgggctgcctgcgcgccatcgtcctctttaaccctgactccaaggggctctcgaacccggccgaggtggaggcgctgagggagaaggtctatgcgtccttggaggcctactgcaagcacaagtacccagagcagccgggaaggttcgctaagctcttgctccgcctgccggctctgcgctccatcgggctcaaatgcctggaacatctcttcttcttcaagctcatcggggacacacccattgacaccttccttatggagatgctggaggcgccgcaccaaatgacttaggcctgcgggcccatcctttgtgcccacccgttctggccaccctgcctggacgccagctgttcttctcagcctgagccctgtccctgcccttctctgcctggcctgtttggactttggggcacagcctgtcactgct

SEQ ID NO: 38 - DNA, 670 bp, Artificial Sequence
  Description of Artificial Sequence Synthetic polynucleotide
atcccacacttctcagagctgcccctggacgaccaggtcatcctgctgcgggcaggctggaatgagctgctcatcgcctccttctcccaccgctccatcgccgtgaaggacgggatcctcctggccaccgggctgcacgtccaccggaacagcgcccacagcgcaggggtgggcgccatctttgacagggtgctgacggagcttgtgtccaagatgcgggacatgcagatggacaagacggagctgggctgcctgcgcgccatcgtcctctttaaccctgactccaaggggctctcgaacccggccgaggtggaggcgctgagggagaaggtctatgcgtccttggaggcctactgcaagcacaagtacccagagcagccgggaaggttcgctaagctcttgctccgcctgccggctctgcgctccatcgggctcaaatgcctggaacatctcttcttcttcaagctcatcggggacacacccattgacaccttccttatggagatgctggaggcgccgcaccaaatgacttaggcctgcgggcccatcctttgtgcccacccgttctggccaccctgcctggacgccagctgttcttctcagcctgagccctgtccctgcccttctctgcctggcctgtttggactttggggcacagcctgtcactgct

SEQ ID NO: 39 - DNA, 672 bp, Artificial Sequence
  Description of Artificial Sequence Synthetic polynucleotide
gccaacgaggacatgccggtggagaggatcctggaggctgagctggccgtggagcccaagaccgagacctacgtggaggcaaacatggggctgaaccccagctcgccgaacgaccctgtcaccaacatttgccaagcagccgacaaacagcttttcaccctggtggagtgggccaagcggatcccacacttctcagagctgcccctggacgaccaggtcatcctgctgcgggcaggctggaatgagctgctcatcgcctccttctcccaccgctccatcgccgtgaaggacgggatcctcctggccaccgggctgcacgtccaccggaacagcgcccacagcgcaggggtgggcgccatctttgacagggtgctgacggagcttgtgtccaagatgcgggacatgcagatggacaagacggagctgggctgcctgcgcgccatcgtcctctttaaccctgactccaaggggctctcgaacccggccgaggtggaggcgctgagggagaaggtctatgcgtccttggaggcctactgcaagcacaagtacccagagcagccgggaaggttcgctaagctcttgctccgcctgccggctctgcgctccatcgggctcaaatgcctggaacatctcttcttcttcaagctcatcggggacacacccattgacacc

SEQ ID NO: 40 - protein, 328 aa, Artificial Sequence
  Description of Artificial Sequence Synthetic polypeptide
CAICGDRSSGKHYGVYSCEGCKGFFKRTVRKDLTYTCRDNKDCLIDKRQRNRCQYCRYQKCLAMGMKREAVQEERQRGKDRNENEVESTSSANEDMPVEKILEAELAVEPKTETYVEANMGLNPSSPNDPVTNICQAADKQLFTLVEWAKRIPHFSELPLDDQVILLRAGWNELLIASFSHRSIAVKDGILLATGLHVHRNSAHSAGVGAIFDRVLTELVSKMRDMQMDKTELGCLRAIVLFNPDSKGLSNPAEVEALREKVYASLEAYCKHKYPEQPGRFAKLLLRLPALRSIGLKCLEHLFFFKLIGDTPIDTFLMEMLEAPHQAT

SEQ ID NO: 41 - protein, 262 aa, Artificial Sequence
  Description of Artificial Sequence Synthetic polypeptide
KREAVQEERQRGKDRNENEVESTSSANEDMPVEKILEAELAVEPKTETYVEANMGLNPSSPNDPVTNICQAADKQLFTLVEWAKRIPHFSELPLDDQVILLRAGWNELLIASFSHRSIAVKDGILLATGLHVHRNSAHSAGVGAIFDRVLTELVSKMRDMQMDKTELGCLRAIVLFNPDSKGLSNPAEVEALREKVYASLEAYCKHKYPEQPGRFAKLLLRLPALRSIGLKCLEHLFFFKLIGDTPIDTFLMEMLEAPHQAT

SEQ ID NO: 42 - protein, 237 aa, Artificial Sequence
  Description of Artificial Sequence Synthetic polypeptide
ANEDMPVEKILEAELAVEPKTETYVEANMGLNPSSPNDPVTNICQAADKQLFTLVEWAKRIPHFSELPLDDQVILLRAGWNELLIASFSHRSIAVKDGILLATGLHVHRNSAHSAGVGAIFDRVLTELVSKMRDMQMDKTELGCLRAIVLFNPDSKGLSNPAEVEALREKVYASLEAYCKHKYPEQPGRFAKLLLRLPALRSIGLKCLEHLFFFKLIGDTPIDTFLMEMLEAPHQAT

SEQ ID NO: 43 - protein, 177 aa, Artificial Sequence
  Description of Artificial Sequence Synthetic polypeptide
IPHFSELPLDDQVILLRAGWNELLIASFSHRSIAVKDGILLATGLHVHRNSAHSAGVGAIFDRVLTELVSKMRDMQMDKTELGCLRAIVLFNPDSKGLSNPAEVEALREKVYASLEAYCKHKYPEQPGRFAKLLLRLPALRSIGLKCLEHLFFFKLIGDTPIDTFLMEMLEAPHQAT

SEQ ID NO: 44 - protein, 224 aa, Artificial Sequence
  Description of Artificial Sequence Synthetic polypeptide
ANEDMPVEKILEAELAVEPKTETYVEANMGLNPSSPNDPVTNICQAADKQLFTLVEWAKRIPHFSELPLDDQVILLRAGWNELLIASFSHRSIAVKDGILLATGLHVHRNSAHSAGVGAIFDRVLTELVSKMRDMQMDKTELGCLRAIVLFNPDSKGLSNPAEVEALREKVYASLEAYCKHKYPEQPGRFAKLLLRLPALRSIGLKCLEHLFFFKLIGDTPIDT

SEQ ID NO: 45 - protein, 328 aa, Artificial Sequence
  Description of Artificial Sequence Synthetic polypeptide
CAICGDRSSGKHYGVYSCEGCKGFFKRTVRKDLTYTCRDNKDCLIDKRQRNRCQYCRYQKCLAMGMKREAVQEERQRGKDRNENEVESTSSANEDMPVERILEAELAVEPKTETYVEANMGLNPSSPNDPVTNICQAADKQLFTLVEWAKRIPHFSELPLDDQVILLRAGWNELLIASFSHRSIAVKDGILLATGLHVHRNSAHSAGVGAIFDRVLTELVSKMRDMQMDKTELGCLRAIVLFNPDSKGLSNPAEVEALREKVYASLEAYCKHKYPEQPGRFAKLLLRLPALRSIGLKCLEHLFFFKLIGDTPIDTFLMEMLEAPHQMT

SEQ ID NO: 46 - protein, 262 aa, Artificial Sequence
  Description of Artificial Sequence Synthetic polypeptide
KREAVQEERQRGKDRNENEVESTSSANEDMPVERILEAELAVEPKTETYVEANMGLNPSSPNDPVTNICQAADKQLFTLVEWAKRIPHFSELPLDDQVILLRAGWNELLIASFSHRSIAVKDGILLATGLHVHRNSAHSAGVGAIFDRVLTELVSKMRDMQMDKTELGCLRAIVLFNPDSKGLSNPAEVEALREKVYASLEAYCKHKYPEQPGRFAKLLLRLPALRSIGLKCLEHLFFFKLIGDTPIDTFLMEMLEAPHQMT

SEQ ID NO: 47 - protein, 237 aa, Artificial Sequence
  Description of Artificial Sequence Synthetic polypeptide
ANEDMPVERILEAELAVEPKTETYVEANMGLNPSSPNDPVTNICQAADKQLFTLVEWAKRIPHFSELPLDDQVILLRAGWNELLIASFSHRSIAVKDGILLATGLHVHRNSAHSAGVGAIFDRVLTELVSKMRDMQMDKTELGCLRAIVLFNPDSKGLSNPAEVEALREKVYASLEAYCKHKYPEQPGRFAKLLLRLPALRSIGLKCLEHLFFFKLIGDTPIDTFLMEMLEAPHQMT

SEQ ID NO: 48 - protein, 177 aa, Artificial Sequence
  Description of Artificial Sequence Synthetic polypeptide
IPHFSELPLDDQVILLRAGWNELLIASFSHRSIAVKDGILLATGLHVHRNSAHSAGVGAIFDRVLTELVSKMRDMQMDKTELGCLRAIVLFNPDSKGLSNPAEVEALREKVYASLEAYCKHKYPEQPGRFAKLLLRLPALRSIGLKCLEHLFFFKLIGDTPIDTFLMEMLEAPHQMT

SEQ ID NO: 49 - protein, 224 aa, Artificial Sequence
  Description of Artificial Sequence Synthetic polypeptide
ANEDMPVERILEAELAVEPKTETYVEANMGLNPSSPNDPVTNICQAADKQLFTLVEWAKRIPHFSELPLDDQVILLRAGWNELLIASFSHRSIAVKDGILLATGLHVHRNSAHSAGVGAIFDRVLTELVSKMRDMQMDKTELGCLRAIVLFNPDSKGLSNPAEVEALREKVYASLEAYCKHKYPEQPGRFAKLLLRLPALRSIGLKCLEHLFFFKLIGDTPIDT

SEQ ID NO: 50 - DNA, 635 bp, Locusta migratoria; identical to SEQ ID NO: 13

SEQ ID NO: 51 - DNA, 687 bp, Amblyomma americanum
cctcctgagatgcctctggagcgcatactggaggcagagctgcgggttgagtcacagacggggaccctctcggaaagcgcacagcagcaggatccagtgagcagcatctgccaagctgcagaccgacagctgcaccagctagttcaatgggccaagcacattccacattttgaagagcttccccttgaggaccgcatggtgttgctcaaggctggctggaacgagctgctcattgctgctttctcccaccgttctgttgacgtgcgtgatggcattgtgctcgctacaggtcttgtggtgcagcggcatagtgctcatggggctggcgttggggccatatttgatagggttctcactgaactggtagcaaagatgcgtgagatgaagatggaccgcactgagcttggatgcctgcttgctgtggtactttttaatcctgaggccaaggggctgcggacctgcccaagtggaggccctgagggagaaagtgtatctgccttggaagagcactgccggcagcagtacccagaccagcctgggcgctttgccaagctgctgctgcggttgccagctctgcgcagtattggcctcaagtgcctcgaacatctctttttcttcaagctcatcggggacacgcccatcgacaactttcttctttccatgctggaggccccctctgacccctaa

SEQ ID NO: 52 - DNA, 693 bp, Amblyomma americanum
tctccggacatgccactcgaacgcattctcgaagccgagatgcgcgtcgagcagccggcaccgtccgttttggcgcagacggccgcatcgggccgcgaccccgtcaacagcatgtgccaggctgccccgccacttcacgagctcgtacagtgggcccggcgaattccgcacttcgaagagcttcccatcgaggatcgcaccgcgctgctcaaagccggctggaacgaactgcttattgccgccttttcgcaccgttctgtggcggtgcgcgacggcatcgttctggccaccgggctggtggtgcagcggcacagcgcacacggcgcaggcgttggcgacatcttcgaccgcgtactagccgagctggtggccaagatgcgcgacatgaagatggacaaaacggagctcggctgcctgcgcgccgtggtgctcttcaatccagacgccaagggtctccgaaacgccaccagagtagaggcgctccgcgagaaggtgtatgcggcgctggaggagcactgccgtcggcaccacccggaccaaccgggtcgcttcggcaagctgctgctgcggctgcctgccttgcgcagcatcgggctcaaatgcctcgagcatctgttcttcttcaagctcatcggagacactcccatagacagcttcctgctcaacatgctggaggcaccggcagacccctag

SEQ ID NO: 53 - DNA, 801 bp, Celuca pugilator
tcagacatgccaattgccagcatacgggaggcagagctcagcgtggatcccatagatgagcagccgctggaccaaggggtgaggcttcaggttccactcgcacctcctgatagtgaaaagtgtagctttactttaccttttcatcccgtcagtgaagtatcctgtgctaaccctctgcaggatgtggtgagcaacatatgccaggcagctgacagacatctggtgcagctggtggagtgggccaagcacatcccacacttcacagaccttcccatagaggaccaagtggtattactcaaagccgggtggaacgagttgcttattgcctcattctcacaccgtagcatgggcgtggaggatggcatcgtgctggccacagggctcgtgatccacagaagtagtgctcaccaggctggagtgggtgccatatttgatcgtgtcctctctgagctggtggccaagatgaaggagatgaagattgacaagacagagctgggctgccttcgctccatcgtcctgttcaacccagatgccaaaggactaaactgcgtcaatgatgtggagatcttgcgtgagaaggtgtatgctgccctggaggagtacacacgaaccacttaccctgatgaacctggacgctttgccaagttgcttctgcgacttcctgcactcaggtctataggcctgaagtgtcttgagtacctcttcctgtttaagctgattggagacactcccctggacagctacttgatgaagatgctcgtagacaacccaaatacaagcgtcactccccccaccagctag

SEQ ID NO: 54 - DNA, 690 bp, Tenebrio molitor
gccgagatgcccctcgacaggataatcgaggcggagaaacggatagaatgcacacccgctggtggctctggtggtgtcggagagcaacacgacggggtgaacaacatctgtcaagccactaacaagcagctgttccaactggtgcaatgggctaagctcatacctcactttacctcgttgccgatgtcggaccaggtgcttttattgagggcaggatggaatgaattgctcatcgccgcattctcgcacagatctatacaggcgcaggatgccatcgttctagccacggggttgacagttaacaaaacgtcggcgcacgccgtgggcgtgggcaacatctacgaccgcgtcctctccgagctggtgaacaagatgaaagagatgaagatggacaagacggagctgggctgcttgagagccatcatcctctacaaccccacgtgtcgcggcatcaagtccgtgcaggaagtggagatgctgcgtgagaaaatttacggcgtgctggaagagtacaccaggaccacccacccgaacgagcccggcaggttcgccaaactgcttctgcgcctcccggccctcaggtccatcgggttgaaatgttccgaacacctctttttcttcaagctgatcggtgatgttccaatagacacgttcctgatggagatgctggagtctccggcggacgcttag

SEQ ID NO: 55 - DNA, 681 bp, Apis mellifera
cattcggacatgccgatcgagcgtatcctggaggccgagaagagagtcgaatgtaagatggagcaacagggaaattacgagaatgcagtgtcgcacatttgcaacgccacgaacaaacagctgttccagctggtagcatgggcgaaacacatcccgcattttacctcgttgccactggaggatcaggtacttctgctcagggccggttggaacgagttgctgatagcctccttttcccaccgttccatcgacgtgaaggacggtatcgtgctggcgacggggatcaccgtgcatcggaactcggcgcagcaggccggcgtgggcacgatattcgaccgtgtcctctcggagcttgtctcgaaaatgcgtgaaatgaagatggacaggacagagcttggctgtctcagatctataatactcttcaatcccgaggttcgaggactgaaatccatccaggaagtgaccctgctccgtgagaagatctacggcgccctggagggttattgccgcgtagcttggcccgacgacgctggaagattcgcgaaattacttctacgcctgcccgccatccgctcgatcggattaaagtgcctcgagtacctgttcttcttcaaaatgatcggtgacgtaccgatcgacgattttctcgtggagatgttagaatcgcgatcagatccttag

SEQ ID NO: 56 - protein, 210 aa, Locusta migratoria; identical to SEQ ID NO: 16

SEQ ID NO: 57 - protein, 228 aa, Amblyomma americanum
PPEMPLERILEAELRVESQTGTLSESAQQQDPVSSICQAADRQLHQLVQWAKHIPHFEELPLEDRMVLLKAGWNELLIAAFSHRSVDVRDGIVLATGLVVQRHSAHGAGVGAIFDRVLTELVAKMREMKMDRTELGCLLAVVLFNPEAKGLRTCPSGGPEGESVSALEEHCRQQYPDQPGRFAKLLLRLPALRSIGLKCLEHLFFFKLIGDTPIDNFLLSMLEAPSDP

SEQ ID NO: 58 - protein, 230 aa, Amblyomma americanum
SPDMPLERILEAEMRVEQPAPSVLAQTAASGRDPVNSMCQAAPPLHELVQWARRIPHFEELPIEDRTALLKAGWNELLIAAFSHRSVAVRDGIVLATGLVVQRHSAHGAGVGDIFDRVLAELVAKMRDMKMDKTELGCLRAVVLFNPDAKGLRNATRVEALREKVYAALEEHCRRHHPDQPGRFGKLLLRLPALRSIGLKCLEHLFFFKLIGDTPIDSFLLNMLEAPADP

SEQ ID NO: 59 - protein, 266 aa, Celuca pugilator
SDMPIASIREAELSVDPIDEQPLDQGVRLQVPLAPPDSEKCSFTLPFHPVSEVSCANPLQDVVSNICQAADRHLVQLVEWAKHIPHFTDLPIEDQVVLLKAGWNELLIASFSHRSMGVEDGIVLATGLVIHRSSAHQAGVGAIFDRVLSELVAKMKEMKIDKTELGCLRSIVLFNPDAKGLNCVNDVEILREKVYAALEEYTRTTYPDEPGRFAKLLLRLPALRSIGLKCLEYLFLFKLIGDTPLDSYLMKMLVDNPNTSVTPPTS

SEQ ID NO: 60 - protein, 229 aa, Tenebrio molitor
AEMPLDRIIEAEKRIECTPAGGSGGVGEQHDGVNNICQATNKQLFQLVQWAKLIPHFTSLPMSDQVLLLRAGWNELLIAAFSHRSIQAQDAIVLATGLTVNKTSAHAVGVGNIYDRVLSELVNKMKEMKMDKTELGCLRAIILYNPTCRGIKSVQEVEMLREKIYGVLEEYTRTTHPNEPGRFAKLLLRLPALRSIGLKCSEHLFFFKLIGDVPIDTFLMEMLESPADA

SEQ ID NO: 61 - protein, 226 aa, Apis mellifera
HSDMPIERILEAEKRVECKMEQQGNYENAVSHICNATNKQLFQLVAWAKHIPHFTSLPLEDQVLLLRAGWNELLIASFSHRSIDVKDGIVLATGITVHRNSAQQAGVGTIFDRVLSELVSKMREMKMDRTELGCLRSIILFNPEVRGLKSIQEVTLLREKIYGALEGYCRVAWPDDAGRFAKLLLRLPAIRSIGLKCLEYLFFFKMIGDVPIDDFLVEMLESRSDP

SEQ ID NO: 62 - DNA, 714 bp, Mus musculus; identical to SEQ ID NO: 32

SEQ ID NO: 63 - DNA, 720 bp, Mus musculus
gcccctgaggagatgcctgtggacaggatcctggaggcagagcttgctgtggagcagaagagtgaccaaggcgttgagggtcctggggccaccgggggtggtggcagcagcccaaatgacccagtgactaacatctgccaggcagctgacaaacagctgttcacactcgttgagtgggcaaagaggatcccgcacttctcctccctacctctggacgatcaggtcatactgctgcgggcaggctggaacgagctcctcattgcgtccttctcccatcggtccattgatgtccgagatggcatcctcctggccacgggtcttcatgtgcacagaaactcagcccattccgcaggcgtgggagccatctttgatcgggtgctgacagagctagtgtccaaaatgcgtgacatgaggatggacaagacagagcttggctgcctgcgggcaatcatcatgtttaatccagacgccaagggcctctccaaccctggagaggtggagatccttcgggagaaggtgtacgcctcactggagacctattgcaagcagaagtaccctgagcagcagggccggtttgccaagctgctgttacgtcttcctgccctccgctccatcggcctcaagtgtctggagcacctgttcttcttcaagctcattggcgacacccccattgacaccttcctcatggagatgcttgaggctccccaccagctagcctga

SEQ ID NO: 64 - DNA, 705 bp, Mus musculus
agccacgaagacatgcccgtggagaggattctagaagccgaacttgctgtggaaccaaagacagaatcctacggtgacatgaacgtggagaactcaacaaatgaccctgttaccaacatatgccatgctgcagataagcaacttttcaccctcgttgagtgggccaaacgcatcccccacttctcagatctcaccttggaggaccaggtcattctactccgggcagggtggaatgaactgctcattgcctccttctcccaccgctcggtttccgtccaggatggcatcctgctggccacgggcctccacgtgcacaggagcagcgctcacagccggggagtcggctccatcttcgacagagtccttacagagttggtgtccaagatgaaagacatgcagatggataagtcagagctggggtgcctacgggccatcgtgctgtttaacccagatgccaagggtttatccaacccctctgaggtggagactcttcgagagaaggtttatgccaccctggaggcctataccaagcagaagtatccggaacagccaggcaggtttgccaagcttctgctgcgtctccctgctctgcgctccatcggcttgaaatgcctggaacacctcttcttcttcaagctcattggagacactcccatcgacagcttcctcatggagatgttggagaccccactgcagatcacctga

SEQ ID NO: 65 - DNA, 850 bp, Homo sapiens; identical to SEQ ID NO: 37

SEQ ID NO: 66 - DNA, 720 bp, Homo sapiens; identical to SEQ ID NO: 12

SEQ ID NO: 67 - DNA, 705 bp, Homo sapiens
ggtcatgaagacatgcctgtggagaggattctagaagctgaacttgctgttgaaccaaagacagaatcctatggtgacatgaatatggagaactcgacaaatgaccctgttaccaacatatgtcatgctgctgacaagcagcttttcaccctcgttgaatgggccaagcgtattccccacttctctgacctcaccttggaggaccaggtcattttgcttcgggcagggtggaatgaattgctgattgcctctttctcccaccgctcagtttccgtgcaggatggcatccttctggccacgggtttacatgtccaccggagcagtgcccacagtgctggggtcggctccatctttgacagagttctaactgagctggtttccaaaatgaaagacatgcagatggacaagtcggaactgggatgcctgcgagccattgtactctttaacccagatgccaagggcctgtccaacccctctgaggtggagactctgcgagagaaggtttatgccacccttgaggcctacaccaagcagaagtatccggaacagccaggcaggtttgccaagctgctgctgcgcctcccagctctgcgttccattggcttgaaatgcctggagcacctcttcttcttcaagctcatcggggacacccccattgacaccttcctcatggagatgttggagaccccgctgcagatcacctga

SEQ ID NO: 68 - protein, 237 aa, Mus musculus; identical to SEQ ID NO: 42

SEQ ID NO: 69 - protein, 239 aa, Mus musculus
APEEMPVDRILEAELAVEQKSDQGVEGPGATGGGGSSPNDPVTNICQAADKQLFTLVEWAKRIPHFSSLPLDDQVILLRAGWNELLIASFSHRSIDVRDGILLATGLHVHRNSAHSAGVGAIFDRVLTELVSKMRDMRMDKTELGCLRAIIMFNPDAKGLSNPGEVEILREKVYASLETYCKQKYPEQQGRFAKLLLRLPALRSIGLKCLEHLFFFKLIGDTPIDTFLMEMLEAPHQLA

SEQ ID NO: 70 - protein, 234 aa, Mus musculus
SHEDMPVERILEAELAVEPKTESYGDMNVENSTNDPVTNICHAADKQLFTLVEWAKRIPHFSDLTLEDQVILLRAGWNELLIASFSHRSVSVQDGILLATGLHVHRSSAHSRGVGSIFDRVLTELVSKMKDMQMDKSELGCLRAIVLFNPDAKGLSNPSEVETLREKVYATLEAYTKQKYPEQPGRFAKLLLRLPALRSIGLKCLEHLFFFKLIGDTPIDSFLMEMLETPLQIT

SEQ ID NO: 71 - protein, 237 aa, Homo sapiens; identical to SEQ ID NO: 47

SEQ ID NO: 72 - protein, 239 aa, Homo sapiens; identical to SEQ ID NO: 15

SEQ ID NO: 73 - protein, 234 aa, Homo sapiens
GHEDMPVERILEAELAVEPKTESYGDMNMENSTNDPVTNICHAADKQLFTLVEWAKRIPHFSDLTLEDQVILLRAGWNELLIASFSHRSVSVQDGILLATGLHVHRSSAHSAGVGSIFDRVLTELVSKMKDMQMDKSELGCLRAIVLFNPDAKGLSNPSEVETLREKVYATLEAYTKQKYPEQPGRFAKLLLRLPALRSIGLKCLEHLFFFKLIGDTPIDTFLMEMLETPLQIT

SEQ ID NO: 74 - DNA, 516 bp, Locusta migratoria
atccctacctctggaggaccaggttctcctcctcagagcaggttggaatgaactgctaattgcagcattttcacatcgatctgtagatgttaaagatggcatagtacttgccactggtctcacagtgcatcgaaattctgcccatcaagctggagtcggcacaatatttgacagagttttgacagaactggtagcaaagatgagagaaatgaaaatggataaaactgaacttggctgcttgcgatctgttattcttttcaatccagaggtgaggggtttgaaatccgcccaggaagttgaacttctacgtgaaaaagtatatgccgctttggaagaatatactagaacaacacatcccgatgaaccaggaagatttgcaaaacttttgcttcgtctgccttctttacgttccataggccttaagtgtttggagcatttgttttctttcgccttattggagatgttccaattgatacgttcctgatggagatgcttgaatcaccttctgattcataa

SEQ ID NO: 75 - DNA, 528 bp, Amblyomma americanum
attccacattttgaagagcttccccttgaggaccgcatggtgttgctcaaggctggctggaacgagctgctcattgctgctttctcccaccgttctgttgacgtgcgtgatggcattgtgctcgctacaggtcttgtggtgcagcggcatagtgctcatggggctggcgttggggccatatttgatagggttctcactgaactggtagcaaagatgcgtgagatgaagatggaccgcactgagcttggatgcctgcttgctgtggtactttttaatcctgaggccaaggggctgcggacctgcccaagtggaggccctgagggagaaagtgtatctgccttggaagagcactgccggcagcagtacccagaccagcctgggcgctttgccaagctgctgctgcggttgccagctctgcgcagtattggcctcaagtgcctcgaacatctctttttcttcaagctcatcggggacacgcccatcgacaactttcttctttccatgctggaggccccctctgacccctaa

SEQ ID NO: 76 - DNA, 531 bp, Amblyomma americanum
attccgcacttcgaagagcttcccatcgaggatcgcaccgcgctgctcaaagccggctggaacgaactgcttattgccgccttttcgcaccgttctgtggcggtgcgcgacggcatcgttctggccaccgggctggtggtgcagcggcacagcgcacacggcgcaggcgttggcgacatcttcgaccgcgtactagccgagctggtggccaagatgcgcgacatgaagatggacaaaacggagctcggctgcctgcgcgccgtggtgctcttcaatccagacgccaagggtctccgaaacgccaccagagtagaggcgctccgcgagaaggtgtatgcggcgctggaggagcactgccgtcggcaccacccggaccaaccgggtcgcttcggcaagctgctgctgcggctgcctgccttgcgcagcatcgggctcaaatgcctcgagcatctgttcttcttcaagctcatcggagacactcccatagacagcttcctgctcaacatgctggaggcaccggcagacccctag

SEQ ID NO: 77 - DNA, 552 bp, Celuca pugilator
atcccacacttcacagaccttcccatagaggaccaagtggtattactcaaagccgggtggaacgagttgcttattgcctcattctcacaccgtagcatgggcgtggaggatggcatcgtgctggccacagggctcgtgatccacagaagtagtgctcaccaggctggagtgggtgccatatttgatcgtgtcctctctgagctggtggccaagatgaaggagatgaagattgacaagacagagctgggctgccttcgctccatcgtcctgttcaacccagatgccaaaggactaaactgcgtcaatgatgtggagatcttgcgtgagaaggtgtatgctgccctggaggagtacacacgaaccacttaccctgatgaacctggacgctttgccaagttgcttctgcgacttcctgcactcaggtctataggcctgaagtgtcttgagtacctcttcctgtttaagctgattggagacactcccctggacagctacttgatgaagatgctcgtagacaacccaaatacaagcgtcactccccccaccagctag

SEQ ID NO: 78 - DNA, 531 bp, Tenebrio molitor
atacctcactttacctcgttgccgatgtcggaccaggtgcttttattgagggcaggatggaatgaattgctcatcgccgcattctcgcacagatctatacaggcgcaggatgccatcgttctagccacggggttgacagttaacaaaacgtcggcgcacgccgtgggcgtgggcaacatctacgaccgcgtcctctccgagctggtgaacaagatgaaagagatgaagatggacaagacggagctgggctgcttgagagccatcatcctctacaaccccacgtgtcgcggcatcaagtccgtgcaggaagtggagatgctgcgtgagaaaatttacggcgtgctggaagagtacaccaggaccacccacccgaacgagcccggcaggttcgccaaactgcttctgcgcctcccggccctcaggtccatcgggttgaaatgttccgaacacctctttttcttcaagctgatcggtgatgttccaatagacacgttcctgatggagatgctggagtctccggcggacgcttag

SEQ ID NO: 79 - DNA, 531 bp, Apis mellifera
atcccgcattttacctcgttgccactggaggatcaggtacttctgctcagggccggttggaacgagttgctgatagcctccttttcccaccgttccatcgacgtgaaggacggtatcgtgctggcgacggggatcaccgtgcatcggaactcggcgcagcaggccggcgtgggcacgatattcgaccgtgtcctctcggagcttgtctcgaaaatgcgtgaaatgaagatggacaggacagagcttggctgtctcagatctataatactcttcaatcccgaggttcgaggactgaaatccatccaggaagtgaccctgctccgtgagaagatctacggcgccctggagggttattgccgcgtagcttggcccgacgacgctggaagattcgcgaaattacttctacgcctgcccgccatccgctcgatcggattaaagtgcctcgagtacctgttcttcttcaaaatgatcggtgacgtaccgatcgacgattttctcgtggagatgttagaatcgcgatcagatccttag

SEQ ID NO: 80 - protein, 176 aa, Locusta migratoria
IPHFTSLPLEDQVLLLRAGWNELLIAAFSHRSVDVKDGIVLATGLTVHRNSAHQAGVGTIFDRVLTELVAKMREMKMDKTELGCLRSVILFNPEVRGLKSAQEVELLREKVYAALEEYTRTTHPDEPGRFAKLLLRLPSLRSIGLKCLEHLFFFRLIGDVPIDTFLMEMLESPSDS

SEQ ID NO: 81 - protein, 175 aa, Amblyomma americanum
IPHFEELPLEDRMVLLKAGWNELLIAAFSHRSVDVRDGIVLATGLVVQRHSAHGAGVGAIFDRVLTELVAKMREMKMDRTELGCLLAVVLFNPEAKGLRTCPSGGPEGESVSALEEHCRQQYPDQPGRFAKLLLRLPALRSIGLKCLEHLFFFKLIGDTPIDNFLLSMLEAPSDP

SEQ ID NO: 82 - protein, 176 aa, Amblyomma americanum
IPHFEELPIEDRTALLKAGWNELLIAAFSHRSVAVRDGIVLATGLVVQRHSAHGAGVGDIFDRVLAELVAKMRDMKMDKTELGCLRAVVLFNPDAKGLRNATRVEALREKVYAALEEHCRRHHPDQPGRFGKLLLRLPALRSIGLKCLEHLFFFKLIGDTPIDSFLLNMLEAPADP

SEQ ID NO: 83 - protein, 183 aa, Celuca pugilator
IPHFTDLPIEDQVVLLKAGWNELLIASFSHRSMGVEDGIVLATGLVIHRSSAHQAGVGAIFDRVLSELVAKMKEMKIDKTELGCLRSIVLFNPDAKGLNCVNDVEILREKVYAALEEYTRTTYPDEPGRFAKLLLRLPALRSIGLKCLEYLFLFKLIGDTPLDSYLMKMLVDNPNTSVTPPTS

SEQ ID NO: 84 - protein, 176 aa, Tenebrio molitor
IPHFTSLPMSDQVLLLRAGWNELLIAAFSHRSIQAQDAIVLATGLTVNKTSAHAVGVGNIYDRVLSELVNKMKEMKMDKTELGCLRAIILYNPTCRGIKSVQEVEMLREKIYGVLEEYTRTTHPNEPGRFAKLLLRLPALRSIGLKCSEHLFFFKLIGDVPIDTFLMEMLESPADA

SEQ ID NO: 85 - protein, 176 aa, Apis mellifera
IPHFTSLPLEDQVLLLRAGWNELLIASFSHRSIDVKDGIVLATGITVHRNSAQQAGVGTIFDRVLSELVSKMREMKMDRTELGCLRSIILFNPEVRGLKSIQEVTLLREKIYGALEGYCRVAWPDDAGRFAKLLLRLPAIRSIGLKCLEYLFFFKMIGDVPIDDFLVEMLESRSDP

SEQ ID NO: 86 - protein, 259 aa, Choristoneura fumiferana
LTANQQFLIARLIWYQDGYEQPSDEDLKRITQTWQQADDENEESDTPFRQITEMTILTVQLIVEFAKGLPGFAKISQPDQITLLKACSSEVMMLRVARRYDAASDSVLFANNQAYTRDNYRKAGMAYVIEDLLHFCRCMYSMALDNIHYALLTAVVIFSDRPGLEQPQLVEEIQRYYLNTLRIYILNQLSGSARSSVIYGKILSILSELRTLGMQNSNMCISLKLKNRKLPPFLEEIWDVADMSHTQPPPILESPTNLG

SEQ ID NO: 87 - protein, 674 aa, Artificial Sequence
  Description of Artificial Sequence Synthetic polypeptide
MDYKDDDDKEMPVDRILEAELAVEQKSDQGVEGPGGTGGSGSSPNDPVTNICQAADKQLFTLVEWAKRIPHFSSLPLDDQVILLRAGWNELLIASFSHRSIDVRDGILLATGLHVHRNSAHSAGVGAIFDRVLTELVSKMRDMRMDKTELGCLRAIILFNPEVRGLKSAQEVELLREKVYAALEEYTRTTHPDEPGRFAKLLLRLPSLRSIGLKCLEHLFFFRLIGDVPIDTFLMEMLESPSDSQISYASRGGGSSGGGEDAKNIKKGPAPFYPLEDGTAGEQLHKAMKRYALVPGTIAFTDAHIEVNITYAEYFEMSVRLAEAMKRYGLNTNHRIVVCSENSLQFFMPVLGALFIGVAVAPANDIYNERELLNSMNISQPTVVFVSKKGLQKILNVQKKLPIIQKIIIMDSKTDYQGFQSMYTFVTSHLPPGFNEYDFVPESFDRDKTIALIMNSSGSTGLPKGVALPHRTACVRFSHARDPIFGNQIIPDTAILSVVPFHHGFGMFTTLGYLICGFRVVLMYRFEEELFLRSLQDYKIQSALLVPTLFSFFAKSTLIDKYDLSNLHEIASGGAPLSKEVGEAVAKRFHLPGIRQGYGLTETTSAILITPEGDDKPGAVGKVVPFFEAKVVDLDTGKTLGVNQRGELCVRGPMIMSGYVNNPEATNALIDKDG

SEQ ID NO: 88 - protein, 463 aa, Artificial Sequence
  Description of Artificial Sequence Synthetic polypeptide
QVAPAELESILLQHPNIFDAGVAGLPDDDAGELPAAVVVLEHGKTMTEKEIVDYVASQVTTAKKLRGGVVFVDEVPKGLTGKLDARKIREILIKAKKGGKSKLGGGSSGGGQISYASRGRPECVVPETQCAMKRKEKKAQKEKDKLPVSTTTVDDHMPPIMQCEPPPPEAARIHEVVPRFLSDKLLVTNRQKNIPQLTANQQFLIARLIWYQDGYEQPSDEDLKRITQTWQQADDENEESDTPFRQITEMTILTVQLIVEFAKGLPGFAKISQPDQITLLKACSSEVMMLRVARRYDAASDSILFANNQAYTRDNYRKAGMAEVIEDLLHFCRCMYSMALDNIHYALLTAVVIFSDRPGLEQPQLVEEIQRYYLNTLRIYILNQLSGSARSSVIYGKILSILSELRTLGMQNSNMCISLKLKNRKLPPFLEEIWDVADMSHTQPPPILESPTNLYPYDVPDYA

SEQ ID NO: 89 - protein, 675 aa, Artificial Sequence
  Description of Artificial Sequence Synthetic polypeptide
WYQDGYEQPSDEDLKRITQTWQQADDENEESDTPFRQITEMTILTVQLIVEFAKGLPGFAKISQPDQITLLKACSSEVMMLRVARRYDAASDSILFANNQAYTRDNYRKAGMAEVIEDLLHFCRCMYSMALDNIHYALLTAVVIFSDRPGLEQPQLVEEIQRYYLNTLRIYILNQLSGSARSSVIYGKILSILSELRTLGMQNSNMCISLKLKNRKLPPFLEEIWDVADMSHTQPPPILESPTNLQISYASRGGGSSGGGEDAKNIKKGPAPFYPLEDGTAGEQLHKAMKRYALVPGTIAFTDAHIEVNITYAEYFEMSVRLAEAMKRYGLNTNHRIVVCSENSLQFFMPVLGALFIGVAVAPANDIYNERELLNSMNISQPTVVFVSKKGLQKILNVQKKLPIIQKIIIMDSKTDYQGFQSMYTFVTSHLPPGFNEYDFVPESFDRDKTIALIMNSSGSTGLPKGVALPHRTACVRFSHARDPIFGNQIIPDTAILSVVPFHHGFGMFTTLGYLICGFRVVLMYRFEEELFLRSLQDYKIQSALLVPTLFSFFAKSTLIDKYDLSNLHEIASGGAPLSKEVGEAVAKRFHLPGIRQGYGLTETTSAILITPEGDDKPGAVGKVVPFFEAKVVDLDTGKTLGVNQRGELCVRGPMIMSGYVNNPEATNALIDKDG

SEQ ID NO: 90 - protein, 412 aa, Artificial Sequence
  Description of Artificial Sequence Synthetic polypeptide
MSGYVNNPEATNALIDKDGWLHSGDIAYWDEDEHFFIVDRLKSLIKYKGYQVAPAELESILLQHPNIFDAGVAGLPDDDAGELPAAVVVLEHGKTMTEKEIVDYVASQVTTAKKLRGGVVFVDEVPKGLTGKLDARKIREILIKAKKGGKSKLGGGSSGGGQISYASRGEMPVDRILEAELAVEQKSDQGVEGPGGTGGSGSSPNDPVTNICQAADKQLFTLVEWAKRIPHFSSLPLDDQVILLRAGWNELLIASFSHRSIDVRDGILLATGLHVHRNSAHSAGVGAIFDRVLTELVSKMRDMRMDKTELGCLRAIILFNPEVRGLKSAQEVELLREKVYAALEEYTRTTHPDEPGRFAKLLLRLPSLRSIGLKCLEHLFFFRLIGDVPIDTFLMEMLESPSDSDYKDDDDK

SEQ ID NO: 91 - protein, 1189 aa, Artificial Sequence
  Description of Artificial Sequence Synthetic polypeptide
MYPYDVPDYASQWYELQQLDSKFLEQVHQLYDDSFPMEIRQYLAQWLEKQDWEHAANDVSFATIRFHDLLSQLDDQYSRFSLENNFLLQHNIRKSKRNLQDNFQEDPIQMSMIIYSCLKEERKILENAQRFNQAQSGNIQSTVMLDKQKELDSKVRNVKDKVMCIEHEIKSLEDLQDEYDFKCKTLQNREHETNGVAKSDQKQEQLLLKKMYLMLDNKRKEVVHKIIELLNVTELTQNALINDELVEWKRRQQSACIGGPPNACLDQLQNWFTIVAESLQQVRQQLKKLEELEQKYTYEHDPITKNKQVLWDRTFSLFQQLIQSSFVVERQPCMPTHPQRPLVLKTGVQFTVKLRLLVKLQELNYNLKVKVLFDKDVNERNTVKGFRKFNILGTHTKVMNMEESTNGSLAAEFRHLQLKEQKNAGTRTNEGPLIVTEELHSLSFETQLCQPGLVIDLETTSLPVVVISNVSQLPSGWASILWYNMLVAEPRNLSFFLTPPCARWAQLSEVLSWQFSSVTKRGLNVDQLNMLGEKLLGPNASPDGLIPWTRFCKENINDKNFPFWLWIESILELIKKHLLPLWNDGCIMGFISKERERALLKDQQPGTFLLRFSESSREGAITFTWVERSQNGGEPDFHAVEPYTKKELSAVTFPDIIRNYKVMAAENIPENPLKYLYPNIDKDHAFGKYYSRPKEAPEPMELDGPKGTGYIKTELISVSEVHPSRLQTTDNLLPMSPEEFDEVSRIVGSVEFDSMMNTVQISYASRGGGSSGGGEDAKNIKKGPAPFYPLEDGTAGEQLHKAMKRYALVPGTIAFTDAHIEVNITYAEYFEMSVRLAEAMKRYGLNTNHRIVVCSENSLQFFMPVLGALFIGVAVAPANDIYNERELLNSMNISQPTVVFVSKKGLQKILNVQKKLPIIQKIIIMDSKTDYQGFQSMYTFVTSHLPPGFNEYDFVPESFDRDKTIALIMNSSGSTGLPKGVALPHRTACVRFSHARDPIFGNQIIPDTAILSVVPFHHGFGMFTTLGYLICGFRVVLMYRFEEELFLRSLQDYKIQSALLVPTLFSFFAKSTLIDKYDLSNLHEIASGGAPLSKEVGEAVAKRFHLPGIRQGYGLTETTSAILITPEGDDKPGAVGKVVPFFEAKVVDLDTGKTLGVNQRGELCVRGPMIMSGYVNNPEATNALIDKDG

SEQ ID NO: 92 - protein, 926 aa, Artificial Sequence
  Description of Artificial Sequence Synthetic polypeptide
MSGYVNNPEATNALIDKDGWLHSGDIAYWDEDEHFFIVDRLKSLIKYKGYQVAPAELESILLQHPNIFDAGVAGLPDDDAGELPAAVVVLEHGKTMTEKEIVDYVASQVTTAKKLRGGVVFVDEVPKGLTGKLDARKIREILIKAKKGGKSKLGGGSSGGGQISYASRGSQWYELQQLDSKFLEQVHQLYDDSFPMEIRQYLAQWLEKQDWEHAANDVSFATIRFHDLLSQLDDQYSRFSLENNFLLQHNIRKSKRNLQDNFQEDPIQMSMIIYSCLKEERKILENAQRFNQAQSGNIQSTVMLDKQKELDSKVRNVKDKVMCIEHEIKSLEDLQDEYDFKCKTLQNREHETNGVAKSDQKQEQLLLKKMYLMLDNKRKEVVHKIIELLNVTELTQNALINDELVEWKRRQQSACIGGPPNACLDQLQNWFTIVAESLQQVRQQLKKLEELEQKYTYEHDPITKNKQVLWDRTFSLFQQLIQSSFVVERQPCMPTHPQRPLVLKTGVQFTVKLRLLVKLQELNYNLKVKVLFDKDVNERNTVKGFRKFNILGTHTKVMNMEESTNGSLAAEFRHLQLKEQKNAGTRTNEGPLIVTEELHSLSFETQLCQPGLVIDLETTSLPVVVISNVSQLPSGWASILWYNMLVAEPRNLSFFLTPPCARWAQLSEVLSWQFSSVTKRGLNVDQLNMLGEKLLGPNASPDGLIPWTRFCKENINDKNFPFWLWIESILELIKKHLLPLWNDGCIMGFISKERERALLKDQQPGTFLLRFSESSREGAITFTWVERSQNGGEPDFHAVEPYTKKELSAVTFPDIIRNYKVMAAENIPENPLKYLYPNIDKDHAFGKYYSRPKEAPEPMELDGPKGTGYIKTELISVSEVHPSRLQTTDNLLPMSPEEFDEVSRIVGSVEFDSMMNTVDYKDDDDK

SEQ ID NO: 93 - protein, 335 aa, Artificial Sequence
  Description of Artificial Sequence Synthetic polypeptide
RPECVVPETQCAMKRKEKKAQKEKDKLPVSTTTVDDHMPPIMQCEPPPPEAARIHEVVPRFLSDKLLVTNRQKNIPQLTANQQFLIARLIWYQDGYEQPSDEDLKRITQTWQQADDENEESDTPFRQITEMTILTVQLIVEFAKGLPGFAKISQPDQITLLKACSSEVMMLRVARRYDAASDSILFANNQAYTRDNYRKAGMAEVIEDLLHFCRCMYSMALDNIHYALLTAVVIFSDRPGLEQPQLVEEIQRYYLNTLRIYILNQLSGSARSSVIYGKILSILSELRTLGMQNSNMCISLKLKNRKLPPFLEEIWDVADMSHTQPPPILESPTNL

SEQ ID NO: 94 - protein, 235 aa, Artificial Sequence
  Description of Artificial Sequence Synthetic polypeptide
EMPVDRILEAELAVEQKSDQGVEGPGGTGGSGSSPNDPVTNICQAADKQLFTLVEWAKRIPHFSSLPLDDQVILLRAGWNELLIASFSHRSIDVRDGILLATGLHVHRNSAHSAGVGAIFDRVLTELVSKMRDMRMDKTELGCLRAIILFNPEVRGLKSAQEVELLREKVYAALEEYTRTTHPDEPGRFAKLLLRLPSLRSIGLKCLEHLFFFRLIGDVPIDTFLMEMLESPSDS

(19) United States
(12) Patent Application Publication
(10) Pub. No.: US 2018/0348231 A1
(43) Pub. Date: Dec. 6, 2018

(54) LIGAND INDUCIBLE POLYPEPTIDE COUPLER SYSTEM

(71) Applicant: Intrexon Corporation, Blacksburg, VA (US)

(72) Inventors: Daniel Paul BEDNARIK, Germantown, MD (US); Charles REED, Germantown, MD (US); Vinodh KURELLA, Rockville, MD (US)

(73) Assignee: Intrexon Corporation, Blacksburg, VA (US)

(21) Appl. No.: 15/562,290

(22) PCT Filed: Mar. 29, 2016
(86) PCT No.: PCT/US2016/024690

§ 371 Date: Sep. 27, 2017

Related U.S. Application Data
(60) Provisional application No. 62/140,380, filed on Mar. 30, 2015.

(51) Int. Cl. G01N 33/68; C07K 14/435; C07K 14/705

(52) U.S. Cl. CPC G01N 33/6845; C07K 14/70567; C07K 14/43563

(57) ABSTRACT

The invention relates to a novel ligand inducible polypeptide coupling system and methods of modulating cell signal transduction pathways and other intracellular and extracellular protein-protein interactions.

### SEQUENCE LISTING

The instant application contains a Sequence Listing which has been submitted electronically in ASCII format and is hereby incorporated by reference in its entirety. Said ASCII copy, created on Mar. 24, 2016, is named 0100-0013WO1_SL.txt and is 192,837 bytes in size.

### FIELD OF THE INVENTION

The field of the invention is cell and molecular biology. Specifically, the field of the invention is cell signal transduction and methods of genetically engineering or modifying the same. More specifically, the invention relates to a novel nuclear receptor-based ligand inducible polypeptide coupler and methods of modulating protein-protein interactions within a host cell.

### BACKGROUND OF THE INVENTION

In the field of genetic engineering and medicine, precise control and modulation of cellular signaling pathways is a valuable and sought after tool for studying, manipulating, and controlling development and other physiological processes (e.g., pathological conditions). Signaling pathways are known to regulate a wide array of cellular processes and functions, including proliferation, differentiation, and apoptosis. Signaling pathways can be regulated through a number of mechanisms such as post-translational modifications (e.g., phosphorylation, ubiquitination, etc.) and protein-protein interactions. One common mechanism for activating or regulating a signaling pathway is through the formation of multi-protein complexes (e.g., dimers, trimers, and oligomers) via protein-protein interactions. Such complexes can include multiple copies of the same protein (homo-complex) or copies of distinct proteins (hetero-complex). The induction of the protein-protein interaction and formation of the complex is in some cases triggered by binding of a ligand to one or more of the member proteins (e.g., a receptor molecule). While numerous such cell signaling pathways have been discovered and characterized, there remains a need to be able to target and manipulate such pathways in a rapid, efficient, and reliable manner using pharmaceutically acceptable and available activating ligands.

In contrast to the relative scarcity of modulation systems for cell signaling pathways, methods for regulating gene expression through induction of protein-protein interactions between transcritption factors have been developed and employed. In order for gene expression to be triggered, such that it produces the RNA necessary as the first step in protein synthesis, a transcriptional activator must be brought into proximity of a promoter that controls gene transcription. Typically, the transcriptional activator itself is associated with a protein that has at least one DNA binding domain that binds to DNA binding sites present in the promoter regions of genes. Thus, for gene expression to occur, a protein comprising a DNA binding domain and an activation domain located at an appropriate distance from the DNA binding domain must be brought into the correct position in the promoter region of the gene.

One method for inducing protein-protein interactions relies on immunosuppressive molecules such as FK506, rapamycin and cyclosporine A, which can bind to immunophilins, FKBP12, cyclophilin, etc. A general strategy has been devised to bring together any two proteins by placing FK506 on each of the two proteins or by placing FK506 on one and cyclosporine A on another one. A synthetic homodimer of FK506 (FK1012) or a compound resulting from fusion of FK506-cyclosporine (FKCsA) can then be used to induce dimerization of these molecules (Spencer et al., 1993, Science 262: 1019-24; Belshaw et al., 1996 Proc Natl Acad Sci USA 93: 4604-7). A Gal4 DNA binding domain fused to FKBP12 and a VP16 activator domain fused to cyclophilin, and FKCsA compound were used to show heterodimerization and activation of a reporter gene under the control of a promoter containing Gal4 binding sites. Unfortunately, this system includes immunosuppressants which can have unwanted side effects and therefore, limits its use for various mammalian applications.

Higher eukaryotic transcription activation systems such as steroid hormone receptor systems have also been employed to regulate gene expression. Steroid hormone receptors are members of the nuclear receptor superfamily and are found in vertebrate and invertebrate cells. Unfortunately, use of steroidal compounds that activate the receptors for the regulation of gene expression, particularly in plants and mammals, is limited due to their involvement in many other natural biological pathways in such organisms. In order to overcome such difficulties, an alternative system has been developed using insect ecdysone receptors (EcR).

Growth, molting, and development in insects are regulated by the ecdysone steroid hormone (molting hormone) and the juvenile hormones (Dhadialla, et al., 1998, Annu. Rev. Entomol. 43: 545-569). The molecular target for ecdysone in insects consists of at least ecdysone receptor (EcR) and ultraspiracle protein (USP). EcR is a member of the nuclear steroid receptor super family that is characterized by signature DNA and ligand binding domains, and an activation domain (Koelle et al. 1991, Cell, 67:59-77). EcR receptors are responsive to a number of steroidal compounds such as ponasterone A and muristerone A. Non-steroidal compounds with ecdysteroid agonist activity have also been described, including the commercially available insecticides tebufenozide and methoxyfenozide that (see International Patent Application No. PCT/EP96/00686 and U.S. Pat. No. 5,530,028, each of which is incorporated by reference herein in its entirety). Both analogs have exceptional safety profiles in other organisms.

The insect ecdysone receptor (EcR) heterodimerizes with Ultraspiracle (USP), the insect homologue of the mammalian retinoid X receptor (RXR), binds ecdysteroids through its ligand binding domain, and also binds ecdysone receptor response elements to activate transcription of ecdysone responsive genes (Riddiford et al., 2000).

EcR has five modular domains, A/B (transactivation), C (DNA binding, heterodimerization)), D (Hinge, heterodimerization), E (ligand binding, heterodimerization and transactivation) and F (transactivation) domains. Some of these domains such as A/B, C and E retain their function when they are fused to other proteins. EcR is a member of the nuclear receptor superfamily and classified into subfamily 1, group H (referred to herein as “Group H nuclear receptors”). The members of each group share 40-60% amino acid identity in the E (ligand binding) domain (Laudet et al., A Unified Nomenclature System for the Nuclear Receptor Subfamily, 1999; Cell 97: 161-163). In addition to the ecdysone receptor, other members of this nuclear receptor subfamily 1, group H, include: ubiquitous receptor (UR), Orphan receptor 1 (OR-1), steroid hormone nuclear receptor 1 (NER-1), RXR interacting protein-15 (RIP-15), liver x receptor β(LXRβ), steroid hormone receptor like protein (RLD-1), liver×receptor (LXR), liver×receptor α (LXRα), farnesoid×receptor (FXR), receptor interacting protein 14 (RIP-14), and farnesol receptor (HRR-1).

In mammalian cells, it has been demonstrated that insect ecdysone receptor (EcR) can heterodimerize with mammalian retinoid X receptor (RXR) and can be used to regulate expression of target genes in a ligand dependent manner. The use of such expression system components, however, has not been contemplated, demonstrated, or applied for regulating protein-protein interaction or for use, for example, in regulating, controlling, inducing or inhibiting extracellular and intracellular signal transduction pathways and protein-protein associations.

While other gene expression systems have been developed, a need remains for systems that allow precise modulation of cell signaling pathways, in both plants and animals, via regulation of protein-protein interactions.

Various publications are cited herein, the disclosures of which are incorporated by reference herein in their entireties.

### SUMMARY OF THE INVENTION

In some embodiments, the invention comprises two polypeptides comprising a first non-naturally occurring polypeptide comprising a fragment or domain of a nuclear receptor protein and a second non-naturally occurring polypeptide comprising a different fragment or domain of a nuclear receptor protein, wherein the first polypeptide is capable of binding an activating ligand, wherein the second polypeptide is capable of associating with the first polypeptide in the presence of the activating ligand, wherein each of the first and second polypeptides further comprise heterologous amino acids or polypeptide sequences such that activating ligand induced association of the first and second polypeptides results in an activated functional, biological or cell signal transduction condition.

In certain embodiments of the invention, one or both nuclear receptor protein fragments or domains comprise an arthropod nuclear receptor amino acid sequence.

In some embodiments of the invention, one or both nuclear receptor protein fragments or domains comprise a Group H nuclear receptor amino acid sequence.

In certain embodiments of the invention, the nuclear receptor amino acid sequence of the first polypeptide comprises an ecdysone receptor (EcR) ligand binding domain, polypeptide fragment, or substitution mutant thereof.

In some embodiments of the invention, the second polypeptide nuclear receptor protein fragment or domain comprises a mammalian nuclear receptor amino acid sequence.

In certain embodiments of the invention, the mammalian nuclear receptor protein fragment or domain comprises a RXR nuclear receptor polypeptide fragment, or substitution mutant thereof.

In some embodiments of the invention, the second polypeptide nuclear receptor protein fragment or domain comprises a chimera of invertebrate and mammalian nuclear receptor amino acid sequences, or substitution mutants thereof.

In certain embodiments of the invention, the second polypeptide nuclear receptor protein fragment or domain comprises a chimera of invertebrate USP (RXR homologue) and mammalian RXR nuclear receptor amino acid sequences, or substitution mutants thereof.

In some embodiments, the invention comprises a ligand inducible polypeptide coupling (LIPC) system comprising: a)A first non-naturally occurring polypeptide comprising a fragment or domain of an arthropod nuclear receptor protein, and b) A second non-naturally occurring polypeptide comprising a fragment or domain of an arthropod and/or mammalian nuclear receptor protein, wherein the first and second polypeptides comprise additional heterologous sequences capable of producing an activated functional, biological or cell signal transduction condition following contact with an activating ligand.

In some embodiments of the invention, one or both nuclear receptor protein fragments or domains of the LIPC comprise a Group H nuclear receptor amino acid sequence.

In certain embodiments of the invention, the first polypeptide of the LIPC comprises an ecdysone receptor (EcR) ligand binding domain, polypeptide fragment, or substitution mutant thereof.

In some embodiments of the invention, the second polypeptide of the LIPC comprises a mammalian nuclear receptor amino acid sequence.

In certain embodiments of the invention, the second polypeptide of the LIPC comprises a RXR nuclear receptor polypeptide fragment, or substitution mutant thereof.

In some embodiments of the invention, the second polypeptide of the LIPC comprises a chimera of invertebrate and mammalian nuclear receptor amino acid sequences, or substitution mutants thereof.

In certain embodiments of the invention, the second polypeptide of the LIPC comprises a chimera of invertebrate USP (RXR homologue) and mammalian RXR nuclear receptor amino acid sequences, or substitution mutants thereof.

In some embodiments of the invention, the nuclear receptor protein fragments of the first and second polypeptides of the invention, including of the LIPC, are derived from an ecdysone receptor polypeptide selected from the group consisting of a spruce budworm Choristoneura fumiferana EcR (“CfEcR”) LBD, a beetle Tenebrio molitor EcR (“TmEcR”) LBD, a Manduca sexta EcR (“MsEcR”) LBD, a Heliothies virescens EcR (“HvEcR”) LBD, a midge Chironomus tentans EcR (“CfEcR”) LBD, a silk moth Bombyx mori EcR (“BmEcR”) LBD, a fruit fly Drosophila melanogaster EcR (“DmEcR”) LBD, a mosquito Aedes aegypti EcR (“AaEcR”) LBD, a blowfly Lucilia capitata EcR (“LcEcR”) LBD, a blowfly Lucilia cuprina EcR (“LucEcR”) LBD, a Mediterranean fruit fly Ceratitis capitata EcR (“CcEcR”) LBD, a locust Locusta migratoria EcR (“LmEcR”) LBD, an aphid Myzus persicae EcR (“MpEcR”) LBD, a fiddler crab Celuca pugilator EcR (“CpEcR”) LBD, a whitefly Bamecia argentifoli EcR (BaEcR) LBD, a leafhopper Nephotetix cincticeps EcR (NcEcR) LBD, and an ixodid tick Amblyomma americanum EcR (“AmaEcR”) LBD.

In certain embodiments of the invention, the nuclear receptor protein fragments of the first and second polypeptides of the invention, including of the LIPC, are derived from are derived from an ecdysone receptor polypeptide encoded by a polynucleotide comprising a nucleic acid sequence of SEQ ID NO: 1 (CfEcR-DEF), SEQ ID NO: 2 (CfEcR-CDEF), SEQ ID NO: 3 (DmEcR-DEF), SEQ ID NO: 4 (TmEcR-DEF) SEQ ID NO: 5 (AmaEcR-DEF), or a polynucleotide encoding a functional variant that is substantially identical thereto.

In certain embodiments of the invention, at least one of the ecdysone receptor polypeptides comprises a polypeptide sequence of SEQ ID NO: 6 (CfEcR-DEF), SEQ ID NO: 7 (DmEcR-DEF), SEQ ID NO: 8 (CfEcR-CDEF), SEQ ID NO: 9 (TmEcR-DEF), SEQ ID NO: 10 (AmaEcR-DEF), or a polypeptide sequence substantially identical thereto.

In certain embodiments of the invention, the ecdysone receptor polypeptide sequence comprises about or at least 1, 2, 3, 4, 5, 6, 7, 8, 9, 10, 11, 12, 13, 14, 15, 16, 17, 18, 19, 20, 21, 22, 23, 24, or substitution mutations relative to the corresponding wild-type ecdysone receptor polypeptide.

In certain embodiments of the invention, the ecdysone receptor polypeptide is encoded by a polynucleotide comprising a codon mutation that results in a substitution of an amino acid residue, wherein the amino acid residue is at a position equivalent to or analogous to a) amino acid residue 20, 21, 48, 51, 52, 55, 58, 59, 61, 62, 92, 93, 95, 96, 107, 109, 110, 120, 123, 125, 175, 218, 219, 223, 230, 234, or 238 of SEQ ID NO: 17, b) amino acid residues 95 and 110 of SEQ ID NO: 17, c) amino acid residues 218 and 219 of SEQ ID NO: 17, d) amino acid residues 107 and 175 of SEQ ID NO: 17, e) amino acid residues 127 and 175 of SEQ ID NO: 17, f) amino acid residues 107 and 127 of SEQ ID NO: 17, g) amino acid residues 107, 127 and 175 of SEQ ID NO: 17, h) amino acid residues 52, 107 and 175 of SEQ ID NO: 17, i) amino acid residues 96, 107 and 175 of SEQ ID NO: 17, j) amino acid residues 107, 110 and 175 of SEQ ID NO: 17, k) amino acid residue 107, 121, 213, or 217 of SEQ ID NO: 18, or 1) amino acid residue 91 or 105 of SEQ ID NO: 19.

In certain embodiments of the invention, the substitution mutation the ecdysone receptor polypeptide is selected from the group consisting of a) E20A, Q21A, F48A, I51A, T52A, T52V, T52I, T52L, T55A, T58A, V59A, L61A, I62A, M92A, M93A, R95A, V96A, V96T, V96D, V96M, V107I, F109A, A110P, A110S, A110M, A110L, Y120A, A123F, M125A, R175E, M218A, C219A, L223A, L230A, L234A, W238A, R95A/A110P, M218A/C219A, V107/IR175E, Y127E/R175E, V107/IY127E, V107/IY127E/R175E, T52V/V107/IR175E, V96A/V107I/R175E, T52A/V107I/R175E, V96T/V107/IR175E, or V107I/A110P/R175E substitution mutation of SEQ ID NO: 17, b) A107P, G121R, G121L, N213A, C217A, or C217S substitution mutation of SEQ ID NO: 18, and c) G91A or A105P substitution mutation of SEQ ID NO: 19.

In some embodiments of the invention, the retinoid X receptor polypeptide comprises a polypeptide selected from the group consisting of a vertebrate retinoid X receptor polypeptide, an invertebrate retinoid X receptor polypeptide (USP), and a chimeric retinoid X polypeptide comprising polypeptide fragments from a vertebrate and invertebrate RXR.

In certain embodiments of the invention, the chimeric retinoid X receptor polypeptide comprises at least two different retinoid X receptor polypeptide fragments selected from the group consisting of a vertebrate species retinoid X receptor polypeptide fragment, an invertebrate species retinoid X receptor polypeptide fragment, and a non-Dipteran/non-Lepidopteran invertebrate species retinoid X receptor polypeptide fragment.

In some embodiments of the invention, the chimeric retinoid X receptor polypeptide comprises a retinoid X receptor polypeptide comprising at least one retinoid X receptor polypeptide fragment selected from the group consisting of an EF-domain helix 1, an EF-domain helix 2, an EF-domain helix 3, an EF-domain helix 4, an EF-domain helix 5, an EF-domain helix 6, an EF-domain helix 7, an EF-domain helix 8, an EF-domain helix 9, an EF-domain helix 10, an EF-domain helix 11, an EF-domain helix 12, an F-domain, and an EF-domain β-pleated sheet, wherein the retinoid X receptor polypeptide fragment is from a different species retinoid X receptor polypeptide or a different isoform retinoid X receptor polypeptide than the second retinoid X receptor polypeptide fragment.

In certain embodiments of the invention, the chimeric retinoid X receptor polypeptide is encoded by a polynucleotide comprising a nucleic acid sequence of a) SEQ ID NO: 11, b) nucleotides 1-348 of SEQ ID NO: 12 and nucleotides 268-630 of SEQ ID NO: 13, c) nucleotides 1-408 of SEQ ID NO: 12 and nucleotides 337-630 of SEQ ID NO: 13, d) nucleotides 1465 of SEQ ID NO: 12 and nucleotides 403-630 of SEQ ID NO: 13, e) nucleotides 1-555 of SEQ ID NO: 12 and nucleotides 490-630 of SEQ ID NO: 13, f) nucleotides 1-624 of SEQ ID NO: 12 and nucleotides 547-630 of SEQ ID NO: 13, g) nucleotides 1-645 of SEQ ID NO: 12 and nucleotides 601-630 of SEQ ID NO: 13, and h) nucleotides 1-717 of SEQ ID NO: 12, nucleotides 613-630 of SEQ ID NO: 13, or a polynucleotide encoding a functional variant that is substantially identical thereto.

In some embodiments of the invention, the chimeric retinoid X polypeptide comprises a polypeptide sequence of a) SEQ ID NO: 14, b) amino acids 1-116 of SEQ ID NO: 15 and amino acids 90-210 of SEQ ID NO: 16, c) amino acids 1-136 of SEQ ID NO: 15 and amino acids 113-210 of SEQ ID NO: 16, d) amino acids 1-155 of SEQ ID NO: 15 and amino acids 135-210 of SEQ ID NO: 16, e) amino acids 1-185 of SEQ ID NO: 15 and amino acids 164-210 of SEQ ID NO: 16, f) amino acids 1-208 of SEQ ID NO: 15 and amino acids 183-210 of SEQ ID NO: 16, g) amino acids 1-215 of SEQ ID NO: 15 and amino acids 201-210 of SEQ ID NO: 16, and h) amino acids 1-239 of SEQ ID NO: 15, amino acids 205-210 of SEQ ID NO: 16, or a polypeptide sequence substantially identical thereto.

In certain embodiments of the invention, one or both additional heterologous sequences of the first and second polypeptides or the LIPC system comprise a transmembrane domain.

In certain embodiments of the invention, at least one of the transmembrane domains of the first and second polypeptides or the LIPC system is a single-pass type I transmembrane.

In certain embodiments of the invention, LIPC components are fused to heterologous polypeptides which result in or produce cell death, or anergy, upon ligand-induced dimerization; such systems may be referred to as “suicide” or “kill” switches.

In some embodiments, the invention comprises an isolated polynucleotide comprising a polynucleotide sequence that encodes the first or second polypeptides described herein.

In certain embodiments, the invention comprises, a first polynucleotide comprising a nucleotide sequence encoding the first polypeptide and a second polynucleotide comprising a nucleotide sequence encoding a second polypeptide described herein.

In some embodiments, the invention comprises a vector comprising any one of the polynucleotides above. In certain embodiments, the invention comprises a vector comprising both of the first and second polynucleotides described herein. In some embodiments, the vector of the invention is an expression vector.

In certain embodiments, the invention comprises a host cell comprising any one of the vectors above. In some embodiments, the host cell is a mammalian T-cell. In certain embodiments, the host cell is a human T-cell.

In some embodiments, the invention comprises a method of inducing cell signal transduction comprising introducing the first and second polypeptides, the LIPC system, the polynucleotides, and/or any of the vectors described herein and contacting the host cell with an activating ligand.

In certain embodiments of the invention, the activating ligand of the first and second polypeptides, the LIPC system, the polynucleotides, the vector, and/or the method described herein is:
a) a compound of the formula:

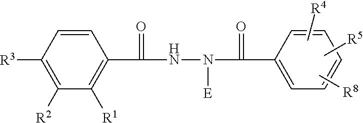

wherein:

E is a (C₄-C₆)alkyl containing a tertiary carbon or a cyano(C₃-C5)alkyl containing a tertiary carbon; R¹ is H, Me, Et, i-Pr, F, formyl, CF₃, CHF₂, CHCl₂, CH₂F, CH₂Cl, CH₂OH, CH₂OMe, CH₂CN, CN, C≡CH, 1-propynyl, 2-propynyl, vinyl, OH, OMe, OEt, cyclopropyl, CF₂CF₃, CH═CHCN, allyl, azido, SCN, or SCHF₂;

R² is H, Me, Et, n-Pr, i-Pr, formyl, CF₃, CHF₂, CHCl₂, CH₂F, CH₂Cl, CH₂OH, CH₂OMe, CH₂CN, CN, C≡CH, 1-propynyl, 2-propynyl, vinyl, Ac, F, Cl, OH, OMe, OEt, O-n-Pr, OAc, NMe₂, NEt₂, SMe, SEt, SOCF₃, OCF₂CF₂H, COEt, cyclopropyl, CF₂CF₃, CH═CHCN, allyl, azido, OCF₃, OCHF₂, O-i-Pr, SCN, SCHF₂, SOMe, NH—CN, or joined with R³ and the phenyl carbons to which R² and R³ are attached to form an ethylenedioxy, a dihydrofuryl ring with the oxygen adjacent to a phenyl carbon, or a dihydropyryl ring with the oxygen adjacent to a phenyl carbon;

R³ is H, Et, or joined with R² and the phenyl carbons to which R² and R³ are attached to form an ethylenedioxy, a dihydrofuryl ring with the oxygen adjacent to a phenyl carbon, or a dihydropyryl ring with the oxygen adjacent to a phenyl carbon;

R⁴, R⁵, and R⁶ are independently H, Me, Et, F, Cl, Br, formyl, CF₃, CHF₂, CHCl₂, CH₂F, CH₂Cl, CH₂OH, CN, C≡CH, 1-propynyl, 2-propynyl, vinyl, OMe, OEt, SMe, or Set; or
b) an ecdysone, 20-hydroxyecdysone, ponasterone A , muristerone A, an oxysterol, a 22(R) hydroxycholesterol, 24(S) hydroxycholesterol, 25-epoxycholesterol, T0901317, 5-alpha-6-alpha-epoxycholesterol-3-sulfate, 7-ketocholesterol-3-sulfate, farnesol, a bile acid, a 1,1-biphosphonate ester, or a Juvenile hormone III.

In certain embodiments of the invention, the activating ligand of the first and second polypeptides, the LIPC system, the polynucleotides, the vector, and/or the method described herein is a compound of the formula:

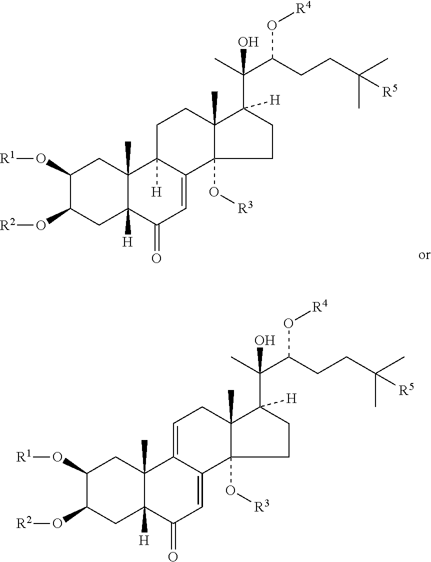

wherein R¹, R², R³, and R⁴ are: a) H, (C₁-C₆)alkyl; (C₁-C₆)haloalkyl; (C₁-C₆)cyanoalkyl; (C₁-C₆)hydroxyalkyl; (C₁-C₄)alkoxy(C₁-C₆)alkyl; (C₂-C₆)alkenyl optionally substituted with halo, cyano, hydroxyl, or (C₁-C₄)alkyl; (C₂-C₆)alkynyl optionally substituted with halo, cyano, hydroxyl, or (C₁-C₄)alkyl; (C₃-C₅)cycloalkyl optionally substituted with halo, cyano, hydroxyl, or (C₁-C₄)alkyl; or b) unsubstituted or substituted benzyl wherein the substituents are independently 1 to 5 H, halo, nitro, cyano, hydroxyl, (C₁-C₆)alkyl, or (Ci-C₆)alkoxy; and

R⁵ is H; OH; F; Cl; or (C₁-C₆)alkoxy;

provided that: when R¹, R², R³, and R⁴ are isopropyl, then R⁵ is not hydroxyl;

when R⁵ is H, hydroxyl, methoxy, or fluoro, then at least one of R¹, R², R³, and R⁴ is not H;

when only one of R¹, R², R³, and R⁴ is methyl, and R⁵ is H or hydroxyl, then the remainder of R¹, R², R³, and R⁴ are not H;

when both R⁴ and one of R¹, R², and R³ are methyl, then R⁵ is neither H nor hydroxyl;

when R¹, R², R³, and R⁴ are all methyl, then R⁵ is not hydroxyl;

when R¹, R², and R³ are all H and R⁵ is hydroxyl, then R⁴ is not ethyl, n-propyl, n-butyl, allyl, or benzyl.

In certain embodiments of the invention, the activating ligand of the first and second polypeptides, the LIPC system, the polynucleotides, the vector, and/or the method described herein is a compound of the formula:

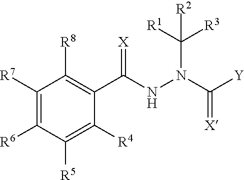

wherein X and X′ are independently 0 or S;
Y is:

(a) substituted or unsubstituted phenyl wherein the substitutents are independently 1-5H, (C₁-C₄)alkyl, (C₁-C₄)alkoxy, (C₂-C₄)alkenyl, halo (F, Cl, Br, I), (C₁-C₄)haloalkyl, hydroxy, amino, cyano, or nitro; or

(b) substituted or unsubstituted 2-pyridyl, 3-pyridyl, or 4-pyridyl, wherein the substitutents are independently 1-4H, (C₁-C₄)alkyl, (C₁-C₄)alkoxy, (C₂-C₄)alkenyl, halo (F, Cl, Br, I), (C₁-C₄)haloalkyl, hydroxy, amino, cyano, or nitro;
R¹ and R² are independently: H; cyano; cyano-substituted or unsubstituted (C₁-C₇) branched or straight-chain alkyl; cyano-substituted or unsubstituted (C₂-C₇) branched or straight-chain alkenyl; cyano-substituted or unsubstituted (C₃-C₇) branched or straight-chain alkenylalkyl; or together the valences of R¹ and R² form a (C₁-C₇) cyano-substituted or unsubstituted alkylidene group (R^{a}R^{b}C═) wherein the sum of non-substituent carbons in R^{a} and R^{b} is 0-6;
R³ is H, methyl, ethyl, n-propyl, isopropyl, or cyano;
R⁴, R⁷, and R⁸ are independently: H, (C₁-C₄)alkyl, (C₁-C₄)alkoxy, (C₂-C₄)alkenyl, halo (F, Cl, Br, I), (C₁-C₄)haloalkyl, hydroxy, amino, cyano, or nitro; and
R⁵ and R⁶ are independently: H, (C₁-C₄)alkyl, (C₂-C₄)alkenyl, (C₃-C₄)alkenylalkyl, halo (F, Cl, Br, I), C₁-C₄ haloalkyl, (C₁-C₄)alkoxy, hydroxy, amino, cyano, nitro, or together as a linkage of the type (—OCHR⁹CHR¹⁰O—) form a ring with the phenyl carbons to which they are attached;
wherein R⁹ and R¹⁰ are independently: H, halo, (C₁-C₃)alkyl, (C₂-C₃)alkenyl, (C₁-C₃)alkoxy(C₁-C₃)alkyl, benzoyloxy(C₁-C₃)alkyl, hydroxy(C₁-C₃)alkyl, halo(C₁-C₃)alkyl, formyl, formyl(C₁-C₃)alkyl, cyano, cyano(C₁-C₃)alkyl, carboxy, carboxy(C₁-C₃)alkyl, (C₁-C₃)alkoxycarbonyl(C₁-C₃)alkyl, (C₁-C₃)alkylcarbonyl(C₁-C₃)alkyl, (C₁-C₃)alkanoyloxy(C₁-C₃)alkyl, amino(C₁-C₃)alkyl, (C₁-C₃)alkylamino(C₁-C₃)alkyl (—(CH₂)ₙR³R³), oximo (—CH═NOH), oximo(C₁-C₃)alkyl, (C₁-C₃)alkoximo (—C═NOR^{d}), alkoximo(C₁-C₃)alkyl, (C₁-C₃)carboxamido (—C(O)NR^{e}R^{f}), (C₁-C₃)carboxamido(C₁-C₃)alkyl, (C₁-C₃)semicarbazido (—C═NNHC(O)NR^{e}R^{f}), semicarbazido(C₁-C₃)alkyl, aminocarbonyloxy (—OC(O)NHR^{g}), aminocarbonyloxy(C₁-C₃)alkyl, pentafluorophenyloxycarbonyl, pentafluorophenyloxycarbonyl(C₁-C₃)alkyl, p-toluenesulfonyl oxy(C₁-C₃)alkyl, arylsulfonyl oxy(C₁-C₃)alkyl, (C₁-C₃)thio(C₁-C₃)alkyl, (C₁-C₃)alkylsulfoxido(C₁-C₃)alkyl, (C₁-C₃)alkylsulfonyl(C₁-C₃)alkyl, or (C₁-C₅)trisubstituted-siloxy(C₁-C₃)alkyl (—(CH₂)ₙSiOR^{d}R^{e}R^{g}); wherein n=1-3, R^{c} and R^{d} represent straight or branched hydrocarbon chains of the indicated length, R^{e}, R^{f} represent H or straight or branched hydrocarbon chains of the indicated length, R^{g} represents (C₁-C₃)alkyl or aryl optionally substituted with halo or (C₁-C₃)alkyl, and R^{c}, R^{d}, R^{e}, R^{f}, and R^{g} are independent of one another;
provided that

i) when R⁹ and R¹⁰ are both H, or

ii) when either R⁹ or R¹⁰ are halo, (C₁-C₃)alkyl, (C₁-C₃)alkoxy(C₁-C₃)alkyl, or benzoyloxy(C₁-C₃)alkyl, or

iii) when R⁵ and R⁶ do not together form a linkage of the type (—OCHR⁹CHR¹⁰O—),

then the number of carbon atoms, excluding those of cyano substitution, for either or both of groups R¹ or R² is greater than 4, and the number of carbon atoms, excluding those of cyano substitution, for the sum of groups R¹, R², and R³ is 10, 11, or 12.

### BRIEF DESCRIPTION OF THE DRAWINGS

A more complete understanding of the present invention may be obtained by reference to the accompanying drawings, when considered in conjunction with the subsequent detailed description. The embodiments illustrated in the drawings are intended only to exemplify the invention and should not be construed as limiting the invention to the illustrated embodiments. Additional embodiments and configurations can provide further useful embodiments.

FIG. 1: A schematic illustration demonstrating the configuration and mode of operation of an exemplary transcriptional switch using EcR and RXR components

FIG. 2: A schematic of the concept of the ligand inducible polypeptide coupler (LIPC) components. In the presence of activating ligand, the EcR and RXR components associate, resulting in association of the fused components (e.g., signaling molecules, signaling domains, complementary protein fragments, and protein subunits).

FIG. 3: A schematic demonstrating a ligand inducible polypeptide coupler (LIPC) system where intracellular EcR and RXR components are fused to extracellular components (e.g., signaling molecules or domains) via a transmembrane domain. In the presence of ligand, the EcR and RXR components associate, resulting in association of the extracellular fused components.

FIG. 4A and 4B: A schematic demonstrating a ligand inducible polypeptide coupler (LIPC) system where extracellular EcR and RXR components are fused to intracellular components (e.g., signaling molecules or domains) via a transmembrane domain (FIG. 4A). In the presence of ligand, the EcR and RXR components associate, resulting in association of the intracellular fused components. A schematic demonstrating a ligand inducible polypeptide coupler (LIPC) system where intracellular EcR and RXR components are tethered to the membrane and are fused to intracellular components (e.g., signaling molecules or domains) (FIG. 4B). In the presence of ligand, the EcR and RXR components associate, resulting in association of the intracellular fused components.

FIG. 5: A schematic demonstrating a ligand inducible polypeptide coupler (LIPC) system where the EcR or RXR component is tethered to the membrane while the other complimentary component is free in the cytoplasm. In the presence of ligand, the membrane-tethered EcR or RXR component associates with the cytosolic EcR or RXR component, resulting in association of the fused components (e.g., signaling molecules or domains).

FIG. 6: A schematic illustration of the split luciferase (fLuc) ligand inducible polypeptide coupler (LIPC) system. Only in the presence of ligand do the EcR and RXR components associate, driving association of the split fLuc and subsequent activity.

FIG. 7: Data demonstrating that the ligand inducible polypeptide coupler (LIPC) described herein drives split fLuc signal only in the presence of activating ligand.

FIG. 8: A schematic of exemplary constructs used in the construction of the ligand inducible polypeptide coupler (LIPC) system as described herein.

FIG. 9: A ligand dose response curve for R×R Nluc+Cluc_EcR and EcR_Nluc+Cluc_R×R using Veledimex ligand.

FIG. 10: A ligand dose response curve for R×R Nluc+Cluc_EcR and EcR_Nluc+Cluc_R×R using Veledimex ligand.

FIG. 11: EcR dimerization induction via Veledimex ligand.

FIG. 12: EcR dimerization induction via Veledimex ligand.

### DETAILED DESCRIPTION OF THE INVENTION

The invention provided herein uses components of EcR-RXR transcriptional switch systems (see e.g., PCT Publication Nos. WO 2001/070816, WO 2002/066612, WO 2002/066613, WO 2002/066614, WO 2002/066615, WO 2003/027266, WO 2003/027289, and WO 2005/108617 each of which is hereby incorporated herein by reference its entirety) which can be expressed in, or by, a host cell to control, regulate or modulate association of fused protein components. One role of protein-protein interactions is to initiate cell signal transduction processes, such as by activating cytoplasmic and/or extracellular signaling domains or restoring functionality to a fragmented or split protein via receptor-ligand binding interactions. Thus, this naturally occurring system can be artificially modulated by driving the association of two inactive signaling domains via induced formation of a “bridge” between an EcR and an RXR component (in the presence of an EcR ligand) wherein the latter components have been incorporated with (i.e., fused to) the signaling domain polypeptides.

In certains embodiments, described herein are systems and methods relating to selective activation of cellular signaling domains via ligand-induced polypeptide coupling. The systems and methods provide a ligand induced polylpeptide coupling system which allows for induction (e.g., modulation, control, regulation) of protein-protein interactions and (“on demand”) activation of signaling domains, or inactivation/inhibition of signaling domains.

Accordingly, disclosed herein are systems and methods that use protein components of a gene transcriptional switch system (expressed in a host cel) for inducing physical association with one another (via an activating ligand) to form a complex (i.e., induce protein-protein interactions) of other associated proteins or domains. Ligand induced protein association can, for example, initiate functions such as activating cytoplasmic and/or extracellular signaling domains in the presence of activating ligand. Thus, in the presence of activating ligand, two signaling domains that are normally inactive can be activated by bringing them together via a “bridge” between the EcR and USP/RXR components.

The use of the word “a” or “an” when used in conjunction with the term “comprising” in the claims and/or the specification may mean “one,” but it is also consistent with the meaning of “one or more,” “at least one,” and “one or more than one.”

The use of the term “for example” and its corresponding abbreviation “e.g.” (whether italicized or not) means that the specific terms cited are representative examples only (that is, specimens, samples, illustrations, models, etc) and embodiments of the invention are not intended to be limited to the specific examples referenced or cited unless explicitly stated otherwise.

The forward slash character (“/”), when used herein in reference to gene or polypeptide components (unless indicated otherwise) is an abbreviation for the words “and/or”. For example, unless specified otherwise, the term “USP/RXR” indicates a polypeptide that can have a mixture of components of both USP and RXR polypeptides or fragments thereof (e.g., a chimeric polypeptide), or USP polypeptide components or fragements thereof (e.g., domains) only, or RXR components or fragements thereof (e.g., domains) only.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps. It is contemplated that any embodiment discussed in this specification can be implemented with respect to any method, system, host cell, expression vector, or composition of the invention. Furthermore, systems, host cells, expression vectors, and/or compositions of the invention can be used to achieve methods of the invention.

“Synthetic” as used herein refers to compounds formed through a chemical process by human agency, as opposed to those of natural origin.

By “isolated” is meant the removal of a nucleic acid, peptide, or polypeptide from its natural environment. By “purified” is meant that a given nucleic acid, whether one that has been removed from nature (including genomic DNA and mRNA) or synthesized (including cDNA) and/or amplified under laboratory conditions, peptide, or polypeptide has been increased in purity, wherein “purity” is a relative term, not “absolute purity.” It is to be understood, however, that nucleic acids, peptides, and polypeptides may be formulated with diluents or adjuvants and still for practical purposes be isolated. For example, nucleic acids typically are mixed with an acceptable carrier or diluent when used for introduction into cells.

A “nucleic acid” is a polymeric compound comprised of covalently linked subunits called nucleotides. Nucleic acid includes polyribonucleic acid (RNA) and polydeoxyribonucleic acid (DNA), both of which may be single-stranded or double-stranded. DNA includes but is not limited to cDNA, genomic DNA, plasmids DNA, synthetic DNA, and semi-synthetic DNA. DNA may be linear, circular, or supercoiled.

A “nucleic acid molecule” refers to the phosphate ester polymeric form of ribonucleosides (adenosine, guanosine, uridine or cytidine; “RNA molecules”) or deoxyribonucleosides (deoxyadenosine, deoxyguanosine, deoxythymidine, or deoxycytidine; “DNA molecules”), or any phosphoester analogs thereof, such as phosphorothioates and thioesters, in either single stranded form, or a double-stranded helix. Double stranded DNA-DNA, DNA-RNA and RNA-RNA helices are possible. The term nucleic acid molecule, and in particular DNA or RNA molecule, refers only to the primary and secondary structure of the molecule, and does not limit it to any particular tertiary forms. Thus, this term includes double-stranded DNA found, inter alia, in circular or linear DNA molecules (e.g., restriction fragments), plasmids, and chromosomes. In discussing the structure of particular double-stranded DNA molecules, 5′ sequences may be described herein according to the normal convention of indicating only the sequence in the 5′ to 3′ direction along the non-transcribed strand of DNA, i.e., the strand having a sequence complementary to the mRNA. A “recombinant DNA molecule” is a DNA molecule that has undergone a molecular biological manipulation.

The term “fragment” will be understood to mean, in reference to polynucleotides, a nucleotide sequence of reduced length relative to the reference nucleic acid and comprising, over the common portion, a nucleotide sequence identical to the reference nucleic acid. Such a nucleic acid fragment, according to the invention may be, where appropriate, included in a larger polynucleotide of which it is a constituent. Such fragments comprise, or alternatively consist of, oligonucleotides ranging in length from at least 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 105, 110, 120, 125, 130, 135, 140, 145, 150, 200, 300, 400, 500, 600, 700, 800, 900, 1000, 1250, 1500, 2000, 2500, 3000, 3500, 4000, 4500, 5000 or 6000 consecutive nucleotides of a nucleic acid according to the invention. In certain embodiments, such fragments may comprise, or alternatively consist of, oligonucleotides of any integer in length ranging, for example, from 6 to 6,000 nucleotides. In certain embodiments such fragments may be any integer in length which is evenly divisible by 3 (e.g., such that the the polynucleotide encodes a full or partial polypeptide open reading frame). In certain embodiments such partial polypeptide fragments may be any integer in length (e.g., such that the polynucleotide may be used as a PCR primer or other hybridizable fragment or for use in generating synthetic or restriction fragment length polynucleotides.)

As used herein, an “isolated nucleic acid fragment” is a polymer of RNA or DNA that is single- or double-stranded, optionally containing synthetic, non-natural or altered nucleotide bases. An isolated nucleic acid fragment in the form of a polymer of DNA may be comprised of one or more segments of cDNA, genomic DNA or synthetic DNA.

A “gene” refers to an assembly of nucleotides that encode a polypeptide, and includes cDNA and genomic DNA nucleic acids. “Gene” also refers to a nucleic acid fragment that expresses a specific protein or polypeptide, including regulatory sequences preceding (5′ non-coding sequences) and following (3′ non-coding sequences) the coding sequence. “Native gene” refers to a gene as found in nature with its own regulatory sequences. “Chimeric gene” refers to any gene that is not a native gene, comprising regulatory and/or coding sequences that are not found together in nature. Accordingly, a chimeric gene may comprise regulatory sequences and coding sequences that are derived from different sources, or regulatory sequences and coding sequences derived from the same source, but arranged in a manner different than that found in nature. A chimeric gene may comprise coding sequences derived from different sources and/or regulatory sequences derived from different sources. “Endogenous gene” refers to a native gene in its natural location in the genome of an organism. A “foreign” gene or “heterologous” gene refers to a gene not normally found in a host organism or cell, but that is introduced into the host organism or cell by gene transfer. Foreign genes can comprise, without limitation, native genes inserted into a non-native organism and chimeric genes. A “transgene” is a foreign or heterologous gene that has been introduced into the genome of a host organism or cell. “Heterologous” DNA refers to DNA not naturally located a the cell, or in a chromosomal site of a cell's genome. In some embodiments, heterologous DNA includes a gene foreign to the cell.

“Polynucleotide” or “oligonucleotide” as used herein refers to a polymeric form of nucleotides of any length, either ribonucleotides or deoxyribonucleotides. This term refers only to the primary structure of the molecule. Thus, this term includes double and single stranded DNA, triplex DNA, as well as double and single stranded RNA. It also includes modified, for example, by methylation and/or by capping, and unmodified forms of the polynucleotide. The term is also meant to include molecules that include non-naturally occurring or synthetic nucleotides as well as nucleotide analogs. In certain embodiments, an oligonucleotide is hybridizable to a genomic DNA molecule, a cDNA molecule, a plasmid DNA or an mRNA molecule. Oligonucleotides can be labeled (e.g., with ³²P-nucleotides or nucleotides to which a label, such as biotin, has been covalently conjugated). In some embodiments, a labeled oligonucleotide can be used as a probe to detect the presence of a nucleic acid. Oligonucleotides (one or both of which may be labeled) can be used as PCR primers, either for cloning full length or a fragment of a nucleic acid, or to detect the presence of a nucleic acid. An oligonucleotide can also be used to form a triple helix with a DNA molecule. In certain embodiments, oligonucleotides are prepared synthetically, for example, on a nucleic acid synthesizer. Accordingly, oligonucleotides can be prepared with non-naturally occurring phosphoester analog bonds, such as thioester bonds, etc.

Nucleic acids and/or nucleic acid sequences are “homologous” when they are derived, naturally or artificially, from a common ancestral nucleic acid or nucleic acid sequence. Proteins and/or protein sequences are homologous when their encoding DNAs are derived, naturally or artificially, from a common ancestral nucleic acid or nucleic acid sequence. The homologous molecules can be termed homologs. For example, any naturally occurring proteins, as described herein, can be modified by any available mutagenesis method. When expressed, this mutagenized nucleic acid encodes a polypeptide that is homologous to the protein encoded by the original nucleic acid. Homology is generally inferred from sequence identity between two or more nucleic acids or proteins (or sequences thereof). The precise percentage of identity between sequences that is useful in establishing homology varies with the nucleic acid and protein at issue, but as little as 25% sequence identity is routinely used to establish homology. Higher levels of sequence identity, e.g., 30%, 40%, 50%, 60%, 70%, 80%, 90%, 95% or 99% or more can also be used to establish homology. Methods for determining sequence identity percentages (e.g., BLASTP and BLASTN using default parameters) are described herein and are generally available.

A DNA “coding sequence” is a double-stranded DNA sequence that is transcribed and translated into a polypeptide in a cell in vitro or in vivo when placed under the control of appropriate regulatory sequences. “Suitable regulatory sequences” refer to nucleotide sequences located upstream (5′ non-coding sequences), within, or downstream (3′ non-coding sequences) of a coding sequence, and which influence the transcription, RNA processing or stability, or translation of the associated coding sequence. Regulatory sequences may include promoters, translation leader sequences, introns, polyadenylation recognition sequences, RNA processing site, effector binding site and stem-loop structure. The boundaries of the coding sequence are determined by a start codon at the 5′ (amino) terminus and a translation stop codon at the 3′ (carboxyl) terminus. A coding sequence can include, but is not limited to, prokaryotic sequences, cDNA from mRNA, genomic DNA sequences, and synthetic DNA sequences. If the coding sequence is intended for expression in a eukaryotic cell, a polyadenylation signal and transcription termination sequence will usually be located 3′ to the coding sequence.

“Open reading frame,” abbreviated ORF, means a length of nucleic acid sequence, either DNA, cDNA or RNA, that comprises a translation start signal or initiation codon, such as an ATG or AUG, and a termination codon, and can be potentially translated into a polypeptide sequence.

“Homologous recombination” refers to the insertion of a foreign DNA sequence into another DNA molecule (e.g., insertion of a vector in a chromosome). In some embodiments, the vector targets a specific chromosomal site for homologous recombination. For specific homologous recombination, the vector will contain sufficiently long regions of homology to sequences of the chromosome to allow complementary binding and incorporation of the vector into the chromosome. Longer regions of homology, and greater degrees of sequence similarity, may increase the efficiency of homologous recombination.

A “vector” or “expression vector” is any modality for the cloning of and/or transfer of a nucleic acid into a host cell. A vector may be a replicon to which another DNA segment may be attached so as to bring about the replication of the attached segment. A “replicon” is any genetic element (e.g., plasmid, phage, cosmid, chromosome, virus) that functions as an autonomous unit of DNA replication in a cell. The term “vector” includes both viral and nonviral means for introducing the nucleic acid into a cell in vitro, ex vivo or in vivo.

The term “plasmid” refers to an extra chromosomal element often carrying a gene that is not part of the central metabolism of the cell, and may be in the form of circular double-stranded DNA molecules. Such elements may be autonomously replicating sequences, genome integrating sequences, phage or nucleotide sequences, linear, circular, or supercoiled, of a single- or double-stranded DNA or RNA, derived from any source, in which a number of nucleotide sequences have been joined or recombined into a unique construction which is capable of introducing a promoter fragment and DNA sequence for a selected gene product along with appropriate 3′ untranslated sequence into a cell.

Vectors may be introduced into the desired host cells by methods known in the art, e.g., transfection, electroporation, microinjection, transduction, cell fusion, DEAE dextran, calcium phosphate precipitation, lipofection (lysosome fusion), use of a gene gun, or a DNA vector transporter (see, e.g., Wu et al., 1992, J. Biol. Chem. 267: 963-967; Wu and Wu, 1988, J. Biol. Chem. 263: 14621-14624; and Hartmut et al., Canadian Patent Application No. 2,012,311, filed Mar. 15, 1990, each of which is incorporated by reference here in its entirety).

It is also possible to introduce a vector in vivo as a naked DNA plasmid (see, e.g., U.S. Pat. Nos. 5,693,622, 5,589,466 and 5,580,859, each of which is incorporated by reference herein in its entirety). Receptor-mediated DNA delivery approaches can also be used (see, e.g., Curel et al., 1992, Hum. Gene Ther 3: 147-154; and Wu and Wu, 1987, J. Biol. Chem 262: 4429-4432, each of which is incorporated by reference herein in its entirety).

The term “transfection” means the uptake of exogenous or heterologous RNA or DNA by a cell. A cell has been “transfected” by exogenous or heterologous RNA or DNA when such RNA or DNA has been introduced inside the cell. A cell has been “transformed” by exogenous or heterologous RNA or DNA when the transfected RNA or DNA effects a phenotypic change. The transforming RNA or DNA can be integrated (covalently linked) into chromosomal DNA making up the genome of the cell.

“Transformation” refers to the transfer of a nucleic acid fragment into the genome of a host organism, resulting in genetically stable inheritance. Host organisms containing the transformed nucleic acid fragments are referred to as “transgenic” or “recombinant” or “transformed” organisms.

The term “selectable marker” means an identifying factor, usually an antibiotic or chemical resistance gene, that is able to be selected for based upon the marker gene's effect, i.e., resistance to an antibiotic, resistance to a herbicide, colorimetric markers, enzymes, fluorescent markers, and the like, wherein the effect is used to track the inheritance of a nucleic acid of interest and/or to identify a cell or organism that has inherited the nucleic acid of interest. Examples of selectable marker genes known and used in the art include, but are not limited to: genes providing resistance to ampicillin, streptomycin, gentamycin, kanamycin, hygromycin, bialaphos herbicide, sulfonamide, and the like; and genes that are used as phenotypic markers, for example, anthocyanin regulatory genes, isopentanyl transferase gene, and the like.

The term “reporter gene” means a nucleic acid encoding an identifying factor that is able to be identified based upon the reporter gene's effect, wherein the effect is used to track the inheritance of a nucleic acid of interest, to identify a cell or organism that has inherited the nucleic acid of interest, and/or to measure gene expression induction or transcription. Examples of reporter genes known and used in the art include, but are not limited to: luciferase (Luc), green fluorescent protein (GFP), chloramphenicol acetyltransferase (CAT), β-galactosidase (LacZ), β-glucuronidase (Gus), and the like. Selectable marker genes may also be considered reporter genes.

“Operably linked” as used herein refers to refers to the physical and/or functional linkage of a DNA segment to another DNA segment in such a way as to allow the segments to function in their intended manners. A DNA sequence encoding a gene product is operably linked to a regulatory sequence when it is linked to the regulatory sequence, such as, for example, promoters, enhancers and/or silencers, in a manner which allows modulation of transcription of the DNA sequence, directly or indirectly. For example, a DNA sequence is operably linked to a promoter when it is ligated to the promoter downstream with respect to the transcription initiation site of the promoter, in the correct reading frame with respect to the transcription initiation site and allows transcription elongation to proceed through the DNA sequence. An enhancer or silencer is operably linked to a DNA sequence coding for a gene product when it is ligated to the DNA sequence in such a manner as to increase or decrease, respectively, the transcription of the DNA sequence. Enhancers and silencers may be located upstream, downstream or embedded within the coding regions of the DNA sequence. A DNA for a signal sequence is operably linked to DNA coding for a polypeptide if the signal sequence is expressed as a preprotein that participates in the secretion of the polypeptide. The terms “cassette,” “expression cassette,” and “gene expression cassette” refer to a segment of DNA that can be inserted into a nucleic acid or polynucleotide (e.g., specific restriction sites or by homologous recombination). The segment of DNA may comprise a polynucleotide that encodes a polypeptide of interest, and the cassette and restriction sites may be designed to ensure insertion of the cassette in the proper reading frame for transcription and translation. “Transformation cassette” refers to a vector comprising a polynucleotide that encodes a polypeptide of interest and having elements in addition to the polynucleotide that facilitate transformation of a particular host cell. Cassettes, expression cassettes, gene expression cassettes and transformation cassettes of the invention may also comprise elements that allow for enhanced expression of a polynucleotide encoding a polypeptide of interest in a host cell. These elements may include, but are not limited to: a promoter, a minimal promoter, an enhancer, a response element, a terminator sequence, a polyadenylation sequence, and the like. “Regulatory region” means a nucleic acid sequence that regulates the expression of a second nucleic acid sequence. A regulatory region may include sequences which are naturally responsible for expressing a particular nucleic acid (a homologous region) or may include sequences of a different origin that are responsible for expressing different proteins or even synthetic proteins (a heterologous region). In particular, the sequences can be sequences of prokaryotic, eukaryotic, or viral genes or derived sequences that stimulate or repress transcription of a gene in a specific or non-specific manner and in an inducible or non-inducible manner. Regulatory regions include origins of replication, RNA splice sites, promoters, enhancers, transcriptional termination sequences, and signal sequences which direct the polypeptide into the secretory pathways of the target cell. A regulatory region from a “heterologous source” is a regulatory region that is not naturally associated with the expressed nucleic acid. Included among the heterologous regulatory regions are regulatory regions from a different species, regulatory regions from a different gene, hybrid regulatory sequences, and regulatory sequences which do not occur in nature.

“Peptide” is used herein to refer to a compound containing two or more amino acid residues linked in a chain. A “polypeptide” is a polymeric compound comprised of covalently linked amino acid residues. Amino acids have the following general structure:

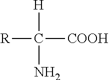

Amino acids are classified into seven groups on the basis of the side chain R: (1) aliphatic side chains, (2) side chains containing a hydroxylic (OH) group, (3) side chains containing sulfur atoms, (4) side chains containing an acidic or amide group, (5) side chains containing a basic group, (6) side chains containing an aromatic ring, and (7) proline, an imino acid in which the side chain is fused to the amino group.

A “protein” comprises a polypeptide. An “isolated polypeptide” or “isolated protein” is a polypeptide or protein that is substantially free of those compounds that are normally associated therewith in its natural state (e.g., other proteins or polypeptides, nucleic acids, carbohydrates, lipids). “Isolated” is not meant to exclude artificial or synthetic mixtures with other compounds, or the presence of impurities which do not interfere with biological activity, and which may be present, for example, due to incomplete purification, addition of stabilizers, or compounding into a pharmaceutically acceptable preparation.

A “substitution mutant polypeptide” or a “substitution mutant” as used herein means a polypeptide comprising a substitution or substitutions (or consisting of a substitution or substitutions) of about or at least 1, 2, 3, 4, 5, 6, 7, 8, 9, 10, 11, 12, 13, 14, 15, 16, 17, 18, 19, 20, 21, 22, 23, 24, 25, or more wild-type or naturally occurring amino acid with a different amino acid relative to the wild-type or naturally occurring polypeptide. A substitution mutant polypeptide may comprising only one (1) amino acid substitution compared to the wild-type or naturally occurring polypeptide may be referred to as a “point mutant” or a “single point mutant” polypeptide.

When a substitution mutant polypeptide includes, or consists of, a substitution of one (1) or more wild-type or naturally occurring amino acids, this substitution may comprise, or consist of, either an equivalent number of wild-type or naturally occurring amino acids deleted for the substitution, i.e., two wild-type or naturally occurring amino acids replaced with two non-wild-type or non-naturally occurring amino acids, or a non-equivalent number of wild-type amino acids deleted for the substitution, e.g., two wild-type amino acids replaced with one non-wild-type amino acid (a substitution+deletion mutation), or two wild-type amino acids replaced with three non-wild-type amino acids (a substitution+insertion mutation). Substitution mutants may be described using an abbreviated nomenclature system to indicate the amino acid residue and number replaced within the reference polypeptide sequence and the new substituted amino acid residue. For example, a substitution mutant in which the twentieth (20^{th}) amino acid residue of a polypeptide is substituted may be abbreviated as “x20z,” wherein “x” is the parent, normally occurring or naturally occurring amino acid to be replaced, “20” is the amino acid residue position or number referenced within the polypeptide, and “z” is the newly substituted amino acid. Therefore, a substitution mutant abbreviated interchangeably as “E20A” or “Glu20Ala” indicates that the substitution mutant comprises an alanine residue (typically abbreviated in the art as “A” or “Ala”) in place of a glutamic acid (typically abbreviated in the art as “E” or “Glu”) at position 20 of the polypeptide.

“Fragment,” when used in relation to a polypeptide, as used herein means a polypeptide whose amino acid sequence is shorter than that of a reference polypeptide and which comprises, or consists of, over the entire portion of the reference polypeptide, an identical amino acid sequence (unless explicitly stated otherwise, e.g., “a fragment 95% identical to . . . ”). Such fragments may, where appropriate, be included in a larger polypeptide of which they are a part. Such fragments of a polypeptide according to the invention may comprise, or alternatively consist of, a polymer ranging in length from at least 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 105, 110, 120, 125, 130, 135, 140, 145, 150, 200, 300, 400, 500, 600, 700, 800, 900, 1000, 1250, 1500, 2000, 2500, 3000, 3500, 4000, 4500, or 5000 amino acid residues. In certain embodiments, such fragments may comprise, or alternatively consist of, amino acid polymers (i.e., peptides, polypeptides) of any integer in length ranging, for example, from 4 to 5,000 residues.

“Truncate” or “truncated,” when used in relation to a polypeptide, is a polypeptide fragment whose amino acid sequence is shorter (at either the N-terminus, C-terminus, or both N- and C- termini) compared to that of a reference polypeptide (e.g., such as may result from a deletion or enzymatic processing of amino acid residues).

A “variant” of a polypeptide or protein is any analogue, fragment, truncation, derivative, or mutant which is derived from, or differing from, a similar polypeptide or protein but which retains at least one biological property of the original, or reference, polypeptide or protein. Different variants of the polypeptide or protein may exist in nature. These variants may be naturally occurring allelic variations characterized by differences in the nucleotide sequences of the structural gene coding for the protein, or may involve differential splicing or post-translational modification, or variants may be artificially (e.g., genetically, synthetically, recombinantly) engineered. The skilled artisan can produce variants having single or multiple amino acid substitutions, deletions, additions, or replacements. These variants may include, inter alfa: (a) variants in which one or more amino acid residues are substituted with conservative or non-conservative amino acids, (b) variants in which one or more amino acids are added to the polypeptide or protein, (c) variants in which one or more of the amino acids includes a substituent group, and/or (d) variants in which the polypeptide or protein is fused with another polypeptide. The techniques for obtaining these variants, including genetic (suppressions, deletions, mutations, etc.), chemical, and enzymatic techniques, are known to persons having ordinary skill in the art. A “functional variant” or “functional fragment” of a protein disclosed herein retains at least a portion of the function of a reference protein. For example, a “functional variant” or “functional fragment” of a protein can retain at least about 10%, about 20%, about 30%, about 40%, about 50%, about 60%, about 70%, about 80%, about 90%, or about 100% of the biological activity or function of the reference protein to which it is compared. In addition, a “functional variant” or “functional fragment” of a protein can, for example, comprise, or consist of, the amino acid sequence of the reference protein with at least 1, 2, 3, 4, 5, 6, 7, 8, 9, 10, 11, 12, 13, 14, 15, 16, 17, 18, 19, or 20 conservative amino acid substitutions per every 100 consecutive amino acid residues. The phrase “conservative amino acid substitution” or “conservative mutation” refers to the replacement of one amino acid by another amino acid with a common property (e.g., hydrophobicity, hydrophilicity, ionic charge, basic, acidic, polar, non-polar, etc). A functional way to define common properties between individual amino acids is to analyze the normalized frequencies of amino acid changes between corresponding proteins of homologous organisms (Schulz, G. E. and Schirmer, R. H., Principles of Protein Structure, Springer-Verlag, New York (1979), which is incorporated by reference herein in its entirety). According to such analyses, groups of amino acids may be defined where amino acids within a group exchange preferentially with each other, and therefore resemble each other most in their impact on the overall protein structure (Schulz, G. E. and Schirmer, R. H., supra). Examples of conservative mutations include amino acid substitutions of amino acids within the sub-groups above, for example, lysine for arginine and vice versa such that a positive charge may be maintained; glutamic acid for aspartic acid and vice versa such that a negative charge may be maintained; serine for threonine such that a free —OH can be maintained; and glutamine for asparagine such that a free —NH₂ can be maintained. In some instances, it may be preferable for the conservative amino acid substitution to not interfere with, or inhibit the biological activity of, the functional variant. In some instances the conservative amino acid substitution may enhance the biological activity of the functional variant, such that the biological activity of the functional variant is increased as compared to the parent molecule. In other instances, it may be desirable for the conservative substitution to interfere with, eliminate, or reduce at least one or more biological activities.

Alternatively or additionally, functional variants can comprise, or consist of, the amino acid sequence of the reference protein with at least one non-conservative amino acid substitution. “Non-conservative mutations” involve amino acid substitutions between different groups (i.e., wherein the original and substituted AA have a different chemical property, such as differences in properties relating to hydrophobicity, hydrophilicity, ionic charge, polar, non-polar, acidic, basic properties, etc.). A few examples of non-conservative substitutions would be, lysine (basic) for tryptophan (non-polar) or for glutamic acid (acidic), aspartic acid (acidic) for tyrosine (polar) or for histidine (basic), or phenylalanine (non-polar) for arginine (basic) or for serine (polar), etc. In some instances, it may be preferable for the non-conservative amino acid substitution to not interfere with, or inhibit the biological activity of, the functional variant. In some instances the non-conservative amino acid substitution may enhance the biological activity of the functional variant, such that the biological activity of the functional variant is increased as compared to the parent molecule. In other instances, it may be desirable for the non-conservative substitution to interfere with, eliminate, or reduce at least one or more biological activities.

A “heterologous protein” refers to a protein not naturally produced in the cell. A “mature protein” refers to a post-translationally processed polypeptide, i.e., one from which any pre- or propeptides present in the primary translation product have been removed. “Precursor” protein refers to the primary product of translation of mRNA, i.e., with pre- and propeptides still present. Pre- and propeptides may be but are not limited to signal peptides or intracellular localization signals.

The term “signal peptide” refers to an amino terminal polypeptide preceding the secreted mature protein. The signal peptide is cleaved from and is therefore not present in the mature protein. Signal peptides have the function of directing and translocating secreted proteins across cell membranes. Signal peptide is also referred to as signal protein.

A “signal sequence” is included at the beginning of the coding sequence of a protein to be expressed on the surface of a cell. This sequence encodes a signal peptide, N-terminal to the mature polypeptide, that directs the host cell to translocate the polypeptide. The term “translocation signal sequence” may also be used to refer to this type of signal sequence. Translocation signal sequences can be found associated with a variety of proteins native to eukaryotes and prokaryotes, and are often functional in both types of organisms.

The term “homology” refers to the percent of identity between two polynucleotide or two polypeptidemolecules. The correspondence between the sequence of one molecule to another can be determined by techniques known to the art. For example, homology can be determined by a direct comparison of the sequence information between two polypeptide molecules by aligning the sequence information and using readily available computer programs. Alternatively, homology can be determined by hybridization of polynucleotides under conditions that form stable duplexes between homologous regions, followed by digestion with single-stranded-specific nuclease(s) and size determination of the digested fragments.

Accordingly, the term “sequence similarity” in all its grammatical forms refers to the degree of identity, homology, or correspondence between nucleic acid or amino acid sequences of proteins that may or may not share a common evolutionary origin (see Reeck et al., 1987, Cell 50:667, which is incorporated by reference herein in its entirety). In certain embodiments, two DNA sequences are “substantially homologous” or “substantially similar” when at least about 50%, at least about 75%, at least about 80%, at least about 85%, at least about 90%, at least about 95% at least about 97%, at least about 98%, at least about 99%, of the nucleotides match over the defined length of the DNA or amino acid sequences. Sequences that are substantially homologous can be identified by comparing the sequences using standard software available in sequence data banks, or in a Southern hybridization experiment under, for example, stringent conditions as understood by those of ordinary skill in the art. For example, stringent hybridization conditions may comprise, or alternatively consist of, hybridization of either target, “probe”, or detection-reagent DNA to filter bound DNA in 6x sodium chloride/sodium citrate (SSC) at about 45 degrees Celsius, followed by one or more washes in 0.2x SSC, 0.1% SDS at about 50-65 degrees Celsius), followed by one or more washes in 0.1x SSC, 0.2% SDS at about 68 degrees Celsius; or, under other stringent hybridization conditions which are known to those of skill in the art (see, for example, Ausubel, F. M. et al., eds., 1989 Current Protocols in Molecular Biology, Green publishing associates, Inc., and John Wiley & Sons Inc., New York, at pages 6.3.1-6.3.6 and 2.10.3). Polynucleotides encoding such polypeptides are also encompassed by the invention.

The terms “identical” or “sequence identity” in the context of two nucleic acid sequences or amino acid sequences of polypeptides refers to the residues in the two sequences which are the same when aligned for maximum correspondence over a specified comparison window. A “comparison window”, as used herein, refers to a segment of at least about 10, at least about 20, at least about 50, at least about 100, at least about 200, at least about 300, at least about 500, or at least about 1000 residues in which a sequence may be compared to a reference sequence of the same number of contiguous positions after the two sequences are aligned optimally. Methods of alignment of sequences for comparison are well-known in the art. Optimal alignment of sequences for comparison may be conducted by the local homology algorithm of Smith and Waterman (1981) Adv. Appl. Math. 2:482, incorporated by reference herein in its entirety; by the alignment algorithm of Needleman and Wunsch (1970) J. Mol. Biol. 48:443, incorporated by reference herein in its entirety; by the search for similarity method of Pearson and Lipman (1988) Proc. Nat. Acad. Sci U.S.A. 85:2444, incorporated by reference herein in its entirety; by computerized implementations of these algorithms (including, but not limited to CLUSTAL in the PC/Gene program by Intelligentics, Mountain View Calif., GAP, BESTFIT, BLAST, FASTA, and TFASTA in the Wisconsin Genetics Software Package, Genetics Computer Group (GCG), 575 Science Dr., Madison, Wis., U.S.A.); the CLUSTAL program is well described by Higgins and Sharp (1988) Gene 73:237-244 and Higgins and Sharp (1989) CABIOS 5:151-153; Corpet et al. (1988) Nucleic Acids Res. 16:10881-10890; Huang et al. (1992) Computer Applications in the Biosciences 8:155-165; and Pearson et al. (1994) Methods in Molecular Biology 24:307-331, each of which is incorporated by reference herein in its entirety. In addition to computer software-based alignments, alignments may also be performed by manual inspection and manual alignment.

In one class of embodiments, polypeptides are 70%, at least 70%, 75%, at least 75%, 80%, at least 80%, 85%, at least 85%, 90%, at least 90%, 95%, at least 95%, 97%, at least 97%, 98%, at least 98%, 99%, or at least 99% or 100% identical to a reference polypeptide, or a fragment thereof (e.g., as measured by BLASTP or CLUSTAL, or other alignment software) using default parameters. Similarly, nucleic acids can also be described with reference to a starting nucleic acid, e.g., they can be 50%, at least 50%, 60%, at least 60%, 70%, at least 70%, 75%, at least 75%, 80%, at least 80%, 85%, at least 85%, 90%, at least 90%, 95%, at least 95%, 97%, at least 97%, 98%, at least 98%, 99%, at least 99%, or 100% identical to a reference nucleic acid or a fragment thereof (e.g., as measured by BLASTN or CLUSTAL, or other alignment software using default parameters). When one molecule is said to have a certain percentage of sequence identity with a larger molecule, it means that when the two molecules are optimally aligned, said percentage of residues in the smaller molecule finds a match residue in the larger molecule in accordance with the order by which the two molecules are optimally aligned, and the “%” (percent) identity is calculated in accord with the length of the smaller molecule.

The term “substantially identical” as applied to nucleic acid or amino acid sequences means that a nucleic acid or amino acid sequence comprises, or consists of, a sequence that has 70%, at least 70%, 75%, at least 75%, 80%, at least 80%, 85%, at least 85%, 90%, at least 90%, 95%, at least 95%, 97%, at least 97%, 98%, at least 98%, 99%, or at least 99% or 100%, compared to a reference sequence. As indicated above, sequence identity may be calculated, for example, using programs well-known and routinely used by those of ordinary skill in the art. For example, the BLASTN program (for nucleotide sequences) uses as defaults a word length (W) of 11, an expectation (E) of 10, M=5, N=−4, and a comparison of both strands. For amino acid sequences, the BLASTP program uses as defaults a word length (W) of 3, an expectation (E) of 10, and the BLOSUM62 scoring matrix (see Henikoff & Henikoff, Proc. Natl. Acad. Sci. USA 89:10915 (1992), incorporated by reference herein in its entirety). Percentage of sequence identity is determined by comparing two optimally aligned sequences over a comparison window, wherein the portion of the polynucleotide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison and multiplying the result by 100 to yield the percentage of sequence identity. Preferably, the substantial identity exists over a region of the sequences that is at least about 10, at least about 20, at least about 50, at least about 100, at least about 200, at least about 300, at least about 500, or at least about 1000 residues in length. In a most preferred embodiment, the sequences are substantially identical over the entire length of the coding region.

Proteins disclosed herein (including functional portions and functional variants thereof) may comprise synthetic amino acids in place of one or more naturally-occurring amino acids. Such synthetic amino acids are known in the art, and include, for example but not limited to, aminocyclohexane carboxylic acid, norleucine, α-amino n-decanoic acid, homoserine, S-acetylaminomethyl-cysteine, trans-3- and trans-4-hydroxyproline, 4-aminophenylalanine, 4-nitrophenylalanine, 4-chlorophenylalanine, 4-carboxyphenylalanine, β-phenylserine β-hydroxyphenylalanine, phenylglycine, α-naphthylalanine, cyclohexylalanine, cyclohexylglycine, indoline-2-carboxylic acid, 1,2,3,4-tetrahydroisoquinoline-3-carboxylic acid, aminomalonic acid, aminomalonic acid monoamide, N′-benzyl-N′-methyl-lysine, N′,N′-dibenzyl-lysine, 6-hydroxylysine, ornithine, α-aminocyclopentane carboxylic acid, α-aminocyclohexane carboxylic acid, α-aminocycloheptane carboxylic acid, α-(2-amino-2-norbornane)-carboxylic acid, α,γ-diaminobutyric acid, α, β-diaminopropionic acid, homophenylalanine, and α-tert-butylglycine.

The term “substantially purified” refers to a nucleic acid sequence, polypeptide, protein or other compound which is essentially free, i.e., is more than about 50% free of, more than about 70% free of, more than about 90% free of, the polynucleotides, proteins, polypeptides and other molecules that the nucleic acid, polypeptide, protein or other compound is naturally associated with.

“Synthetic genes” can be assembled from oligonucleotide building blocks that are chemically synthesized using procedures known to those or ordinary skill in the art. These building blocks are ligated and annealed to form gene segments that are then enzymatically assembled to construct the entire gene. “Chemically synthesized,” as related to a sequence of DNA, means that the component nucleotides were assembled in vitro. Manual chemical synthesis of DNA may be accomplished using well-established procedures.The skilled artisan appreciates the likelihood of enhanced gene expression if codon usage is biased towards those codons favored by the host cell or organism in which it is expressed. Determination of preferred codons can be based on a survey of genes derived from the host cell where sequence information is available.

The term “hybrid,” when used in reference to a polypeptide, nucleotide, or fragment thereof, as used herein refers to a polypeptide, polynucleotide, or fragment thereof, whose amino acid and/or nucleotide sequence is not found in nature. For example, a fusion protein of two heterologous proteins or polypeptides or a cDNA encoding a fusion polypeptide.

“Ligand Inducible Polypeptide Coupler” and “Ligand Inducible Polypeptide Couplers” is used interchangeably herein with “LIPC” and “LIPCs”, irrespectively, that is, “LIPC” can mean “Coupler” (singular) or “Couplers” plural) As such, LIPC refers to a system and polypeptide components of that system for bringing together (“coupling”; i.e., oligomerizing, dimerizing) polypeptides, in a small molecule ligand-dependent manner via incorporation of nuclear receptor polypeptide components into fusion proteins (e.g., use of Group H nuclear receptor and EcR receptor polypeptide components (e.g. EcR polypeptide fragments or domains); including EcR ligand binding polypeptides and nuclear receptor USP and/or RXR nuclear receptor polypeptide components (e.g. polypeptide fragments or domain thereof) as described herein.

Administration of an activating ligand and configuration of LIPC components can be used to regulate the timing and location of dimerization and polypeptide coupling activation. LIPC relies upon protein factors encoded by genes which are not native to the host, and which are encoded by heterologous sequences. A LIPC that is used to control the spatial and temporal association of polypeptide components in a host system can be derived from a foreign source such as bacteria, yeast, plants, insects, or viruses. Thus, the LIPC nuclear receptor polypeptide components confer utility in the host by providing a mechanism to control the association (e.g., dimerization, oligomerization) of polypeptides or proteins with which LIPC components are “fused” (i.e., engineered to be fusion proteins).

“Genetic switches,” also referred to as “gene switches” or “transcriptional switches,” are used for controlling gene expression and are artificially designed for the deliberate regulation of transgenes. Gene switches typically encode a trans-activator or trans-inhibitor whose activity can be regulated and a trans-activator-responsive or trans-inhibitor-susceptible promoter for controlling a gene of interest. These factors may be ligand-responsive, chimeric proteins containing a DNA-binding domain, a ligand-binding domain and a transcriptional activation domain or inhibition domain, respectively. These include for example, antibiotic responsive switches based on tetracycline-sensory trans-activators and trans-inhibitors, mammalian or insect steroid receptor-derived trans-activators, and rapamycin-induced trans-activators. Other genetic switches make use of endogenous transcription factors that can be deliberately activated by physical cues or signals, and whose transient activation is tolerated by the host cell. Examples of systems of this kind include gene switches that make use of transcription factors which can be activated by heat or ionizing radiation for example. See e.g., Auslander, S. and Fussenegger, M. (2012). Trends in Biotechnology (electronic release) pp. 1-14; Vilaboa N, Boellmann F, Voellmy R (2011) Gene Switches for Deliberate Regulation of Transgene Expression: Recent Advances in System Development and Uses. J Genet Syndr Gene Ther 2:107, each of which is incorporated by reference herein in its entirety.

In one embodiment, the genetic switch includes the following components: 1) Co-Activation Partner (CAP) and a Ligand-inducible Transcription Factor (LTF) which form unstable and unproductive heterodimers in the absence of Activator Ligand; 2) Activator Ligand: a molecule (e.g., an ecdysone analog or other a non-steroid small molecule); and 3) an Inducible Promoter, (e.g., a customizable promoter which binds the LTF). In one embodiment, the genetic switch allows for the expression of transduced genes only when the small molecule activator ligand combines with the switch components (CAP and LTF) thereby activating gene transcription from an inducible promoter, and ultimately resulting in expression of desired proteins. The timing, location, and concentration of genetic switch can be regulated in a dose dependent manner with the activator ligand. In certain embodiments components of the EcR-based genetic switch developed by Applicant (for example, as referenced under the trademark) RHEOSWITCH®)are used as component parts to generate ligand inducible polypeptide couplers (LIPCs) of the present invention (see for example, PCT Publication Nos. WO 2001/070816, WO 2002/066612, WO 2002/066613, WO 2002/066614, WO 2002/066615, WO 2003/027266, WO 2003/027289, and WO 2005/108617 each of which is hereby incorporated by reference herein in its entirety).

In the present invention, components of EcR-based “genetic switches” are employed to create “ligand inducible polypeptide couplers” described, and envisaged by, the disclosure herein. “Ecdysone receptor” and “EcR” are used interchangeably herein and refer to members of the Arthropod superfamily of nuclear receptors, classified into subfamily 1, group H (referred to herein as “Group H nuclear receptors”). The members of each group share 40-60% amino acid identity in the E (ligand binding) domain (Laudet et al., A Unified Nomenclature System for the Nuclear Receptor Subfamily, 1999; Cell 97: 161-163, which is incorporated by reference herein in its entirety). In addition to the ecdysone receptor, other members of this nuclear receptor subfamily 1, group H include: ubiquitous receptor (UR), Orphan receptor 1 (OR-1), steroid hormone nuclear receptor 1 (NER-1), RXR interacting protein-15 (RIP-15), liver x receptor β (LXRβ), steroid hormone receptor like protein (RLD-1), liver x receptor (LXR), liver x receptor α(LXRα), farnesoid x receptor (FXR), receptor interacting protein 14 (RIP-14), and farnesol receptor (HRR-1). EcR proteins are characterized by signature DNA and ligand binding domains (LBD), and an activation domain (Koelle et al. 1991, Cell, 67:59-77, which is incorporated by reference herein in its entirety). EcR receptors are responsive to a number of steroidal and non-steroidal compounds, i.e., activating ligands.

“Retinoid X receptor” and “RXR” are used interchangeably herein and refer to a member of the nuclear hormone receptor family, in particular the steroid and thyroid hormone receptor superfamily. Vertebrate RXR includes at least three distinct genes (RXR alpha, beta and gamma), which give rise to a large number of protein products through differential promoter usage and alternative splicing. Invertebrate homologs of RXR (e.g., the ultraspiracle (USP) protein) are found in a wide range of species and are envisaged for use in the present invention.

“Activating ligand” as used herein refers to a compound that is capable of binding to a member of the nuclear steroid receptor super family (e.g., EcR and RXR) and activating the member by inducing association (e.g., dimerization, oligomerization, or protein-protein interaction) of the nuclear receptor components. Exemplary activating ligands for the present invention are provided below.

The term “inactive” or “inactivated,” when referencing inactive polypeptides, domains, signaling molecules, protein or polypeptide fragments, or protein subunits of polypeptides, as used herein means a protein or polypeptide that is not presently generating all or substantially all of one or more of its inherent biological functions or activities. In some embodiments, an inactive or inactivated protein or polypeptide becomes activated through association with another protein or polypeptide, i.e., protein-protein interaction. Such activation can occur, for example, through oligomerization induced by the binding of a first nuclear receptor ligand binding protein fragment to a second nuclear receptor protein fragment, wherein the first and second nuclear receptor fragments are part of two separate, larger, first and second heterologous polypeptides, wherein the first and second heterologous polypeptides change from a biologically inactive to a biologically active state upon ligand induced oligomerization.

“T cell” or “T lymphocyte” as used herein is a type of lymphocyte that plays a central role in cell-mediated immunity. They may be distinguished from other lymphocytes, such as B cells and natural killer cells (NK cells), by the presence of a T-cell receptor (TCR) on the cell surface.

“Antibody” as used herein refers to monoclonal or polyclonal antibodies. The term “monoclonal antibodies,” as used herein, refers to antibodies that bind to the same epitope (for example, such as antibodies that are produced by a single clone of B-cells). In contrast, “polyclonal antibodies” refer to a population of antibodies that bind to different epitopes of the same antigen (for example, such as antibodies that are produced by a heterogenous mixture of different B-cells). Ligand Inducible Polypeptide Coupler (LIPC) of the Invention

Described herein is a ligand inducible polypeptide coupler (LIPC) thatutilizes the ability of a pair of interacting nuclear receptor proteins (by engineering the LIPC (i.e., nuclear receptor) components to generate fusion proteins) to bring together separate proteins or domains and induce their association (e.g., dimerization, oligomerization) of otherwise separate proteins or domains (e.g., separated, biologically inactive polypeptide monomers, such as receptor tyrosine kinase polypeptides (RTKs) which typically require dimerization to form an active signaling complex). In certain embodiments, the switch system of the presnt invention is an ecdysone receptor (EcR)-based system. The ecdysone receptor-based ligand inducible polypeptide couplermay be either heterodimeric or homodimeric with respect to the “parent” non-nuclear receptor (LIPC) polypeptide components or domains. On the other hand, it is understood that a functional nuclear receptor (e.g., EcR complex) generally refers to a heterodimeric protein complex containing two or more members of the steroid receptor family. For example, an ecdysone receptor protein obtained from various insects, and an ultraspiracle (USP) protein or vertebrate homolog of USP, retinoid X receptor (RXR) protein (see, e.g., Yao, et al. (1993) Nature 366, 476-479 and Yao, et al., (1992) Cell 71, 63-72, each of which is incorporated by reference herein in its entirety).

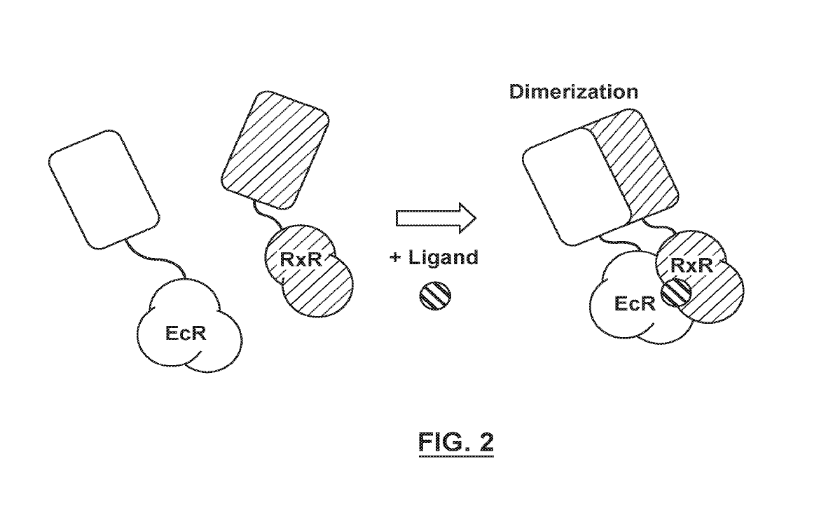

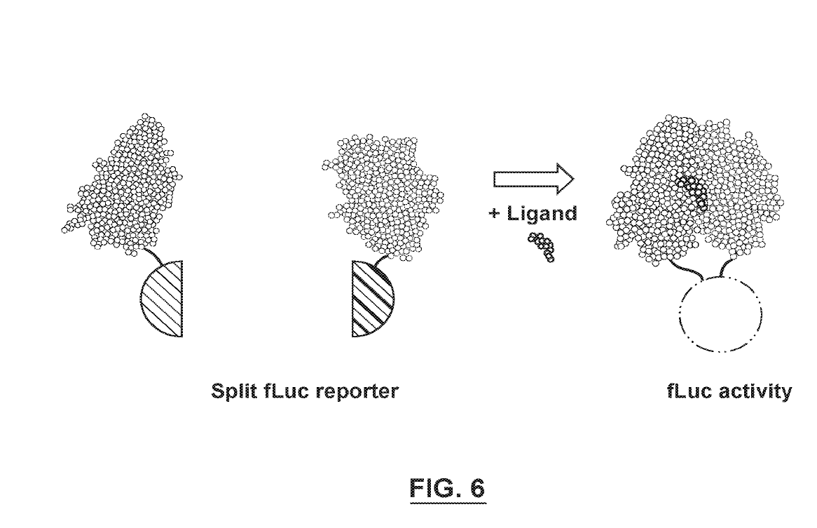

The present invention can include two or more expression cassettes; e.g., encoding EcR and USP/RXR components fused to separate polypeptides or domains (e.g., signaling molecules, signaling domains, complementary protein fragments, protein subunits, and natural or engineered partial or truncated proteins). In the presence of activating ligand, the interaction of EcR-containing polypeptides with the USP/RXR-containing polypeptides brings the attached (fusion) proteins or domains in close proximity allowing for their association (protein-protein interaction), see e.g., FIGS. 2-6.

The ecdysone receptor complex typically includes proteins which are members of the nuclear receptor superfamily wherein all members are generally characterized by the presence of an amino-terminal transactivation domain, a DNA binding domain (“DBD”), and a ligand binding domain (“LBD”) separated from the DBD by a hinge region. Members of the nuclear receptor superfamily are also characterized by the presence of four or five domains: A/B, C, D, E, and in some members F (see, e.g., US patent 4,981,784 and Evans, Science 240:889-895(1988), each of which is incorporated by reference herein in its entirety). The “A/B” domain corresponds to the transactivation domain, “C” corresponds to the DNA binding domain, “D” corresponds to the hinge region, and “E” corresponds to the ligand binding domain. Some members of the family may also have another transactivation domain on the carboxy-terminal side of the LBD corresponding to “F.”

These domains may be either native (i.e., naturally-occurring), modified, or chimeras (i.e., heterologous fusion proteins) of domains from different nuclear receptor proteins. Because the domains of EcR, USP, and RXR are modular in nature, the LBD, DBD, and transactivation domains may be interchanged.

Within certain embodiments, a dipteran (fruit fly Drosophila melanogaster) or a lepidopteran (spruce bud worm Choristoneura fumiferana) ultraspiracle protein (USP) is utilized as part of an LIPC system. In certain embodiments, a vertebrate or mammalian retinoid X receptor (RXR) (see, e.g., International Publ. No. WO/2001/070816, which is incorporated by reference herein in its entirety) is utilized as part of an LIPC system. In certain embodiments, the ultraspiracle protein of Locusta migratoria (“LmUSP”) and the RXR homolog 1 and RXR homolog 2 of the ixodid tick Amblyomma americanum (“AmaRXR1” and “AmaRXR2,” respectively) and their non-Dipteran, non-Lepidopteran homologs including, but not limited to: fiddler crab Celuca pugilator RXR homolog (“CpRXR”), beetle Tenebrio molitor RXR homolog (“TmRXR”), honeybee Apis mellifera RXR homolog (“AmRXR”), and an aphid Myzus persicae RXR homolog (“MpRXR”), all of which are referred to herein collectively as invertebrate RXRs (and which can function similar to vertebrate retinoid X receptor (RXR)) are utilized as part of an LIPC system.

EcR Components

The present invention provides for ecdysone receptor (EcR) polypeptide components, e.g., EcR ligand binding domains (LBD), to be employed in a ligand inducible polypeptide coupler system described herein. Exemplary EcR components that can be used in the invention are described, for example, in International PCT Publ. Nos. WO 2001/070816, WO 2002/066612, WO 2002/066613, WO 2002/066614, WO 2002/066615, WO 2003/027266, WO 2003/027289, WO 2005/108617, and WO 2009/114201each of which is incorporated by reference herein in its entirety.

In certain embodiments, the LIPC EcR component is an EcR ligand binding domain (LBD), or a related steroid/thyroid hormone nuclear receptor family member LBD, analog, combination, modification, or fragement thereof. In some embodiments, the LIPC LBD is from a truncated EcR polypeptide or EcR LBD. A truncation or substitution mutation thereof may be made by any method used in the art, including but not limited to restriction endonuclease digestion/deletion, PCR-mediated oligonucleotide-directed deletion, chemical mutagenesis, DNA strand breakage, and the like.

The LIPC EcR polypeptide component may be an invertebrate EcR, for example, selected from the class Arthropod. In some embodiments, the LIPC EcR polypeptide component (or fragments thereof) is selected from the group consisting of a Lepidopteran EcR, a Dipteran EcR, an Orthopteran EcR, a Homopteran EcR and a Hemipteran EcR. In particular embodiments, the EcR is a from spruce budwonn Choristoneura fumiferana EcR (“CfEcR”), a beetle Tenebrio molitor EcR (“TmEcR”), a Manduca sexta EcR (“MsEcR”), a Heliothies virescens EcR (“HvEcR”), a midge Chironomus tentans EcR (“CfEcR”), a silk moth Bombyx mori EcR (“BmEcR”), a fruit fly Drosophila melanogaster EcR (“DmEcR”), a mosquito Aedes aegypti EcR (“AaEcR”), a blowfly Lucilia capitata EcR (“LcEcR”), a blowfly Lucilia cuprina EcR (“LucEcR”), a Mediterranean fruit fly Ceratitis capitata EcR (“CcEcR”), a locust Locusta migratoria EcR (“LmEcR”), an aphid Myzus persicae EcR (“MpEcR”), a fiddler crab Celuca pugilator EcR (“CpEcR”), an ixodid tic Amblyomma americanurn EcR (“AmaEcR”), a whitefly Bamecia argentifoli EcR (“BaEcR”, SEQ ID NO: 20) or a leafhopper Nephotetix cincticeps EcR (“NcEcR”, SEQ ID NO: 21). In one embodiment, the LIPC LBD (or fragment thereof) is from spruce budworm (Choristoneura fumiferana) EcR (“CfEcR”) or fruit fly Drosophila melanogaster EcR (“DmEcR”).

In certain embodiments, the LIPC LBD is from a truncated EcR polypeptide. In some embodiments, the LIPC EcR polypeptide truncation results in a deletion of at least 1, 2, 3, 4, 5, 6, 7, 8, 9, 10, 15, 20, 25, 30, 35, 40, 45, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, 200, 205, 210, 215, 220, 225, 230, 235, 240, 245, 250, 255, 260, or 265 amino acids. Preferably, an LIPC EcR polypeptide truncation results in a deletion of at least a partial polypeptide domain. More preferably, the LIPC EcR polypeptide truncation results in a deletion of at least an entire polypeptide domain. In a certain embodiments, the LIPC EcR polypeptide truncation results in a deletion of at least an AB-domain, a C-domain, a D-domain, an F-domain, an A/B/C-domains, an A/B/¹/₂-C-domains, an A/B/C/D-domains, an A/B/C/D/F-domains, an A/B/F-domains, an A/B/C/F-domains, a partial E domain, or a partial F domain. A combination of several complete and/or partial domain deletions may also be performed.

In some embodiments, an LIPC ecdysone receptor polypeptide component, or fragment thereof, is encoded by a polynucleotide comprising a nucleic acid sequence of SEQ ID NO: 22 (CfEcR-EF), SEQ ID NO: 23 (DmEcR-EF), SEQ ID NO: 24 (CfEcR-DE), or SEQ ID NO: 25 (DmEcR-DE), or a fragment thereof.

In some embodiments, an LIPC ecdysone receptor polypeptide component, or fragment thereof, is encoded by a polynucleotide comprising a nucleic acid sequence of SEQ ID NO: 1 (CfEcR-DEF), SEQ ID NO: 2 (CfEcR-CDEF), SEQ ID NO: 3 (DmEcR-DEF), SEQ ID NO: 4 (TmEcR-DEF) or SEQ ID NO: 5 (AmaEcR-DEF), or a fragment thereof.

In certain embodiments, an LIPC ecdysone receptor polypeptide component comprises an amino acid sequence of SEQ ID NO: 26 (CfEcR-EF), SEQ ID NO: 27 (DmEcR-EF), SEQ ID NO: 28 (CfEcR-DE), or SEQ ID NO: 29 (DmEcR-DE), or a fragment thereof. In some embodiments, an LIPC ecdysone receptor polypeptide component comprises an amino acid sequence of SEQ ID NO: 6 (CfEcR-DEF), SEQ ID NO: 8 (CfEcR-CDEF), SEQ ID NO: 7 (DmEcR-DEF), SEQ ID NO: 9 (TmEcR-DEF), or SEQ ID NO: 10 (AmaEcR-DEF), or a fragment thereof.

In addition, amino acid residues that are involved in ligand binding to Group H nuclear receptor ligand binding domains (e.g., EcR ligand binding domains) that affect the ligand sensitivity and magnitude of gene expression induction in an ecdysone receptor-based inducible gene expression (“gene switch”) system have been identified (see, e.g., International Publ. No. WO 02/066612, which is incorporated by reference herein in its entirety). These substitution mutant nuclear receptor polypeptides and their use in a LIPC system can provide improved ligand-induced (“activated”) polypeptide coupling in host cells and organisms in which regulation (modulation, control) of ligand sensitivity and magnitude of ligand induced oligomerization may be selected as desired, depending upon the application. As described further below, Group H nuclear receptors which comprise substitution mutations (referred to herein as “substitution mutants”) can be employed in ligand inducible polypeptide couplers (LIPC) of the present invention.

LIPC ecdysone receptor (EcR) polypeptide components (including EcR ligand binding domains (LBD)) used in the present invention may be from an invertebrate EcR, e.g., selected from the class Arthropod EcR. In certain embodiments, the LIPC EcR polypeptide component is selected from the group consisting of a Lepidopteran EcR, a Dipteran EcR, an Orthopteran EcR, a Homopteran EcR and a Hemipteran EcR. In certain embodiments, the EcR ligand binding domain for use in the present invention is from a spruce budworm Choristoneura fumiferana EcR (“CfEcR”), a beetle Tenebrio molitor EcR (“TmEcR”), a Manduca sexta EcR (“MsEcR”), a Heliothies virescens EcR (“HvEcR”), a midge Chironomus tentans EcR (“CtEcR”), a silk moth Bombyx mori EcR (“BmEcR”), a squinting bush brown Bicyclus anynana EcR (“BanEcR”), a buckeye Junonia coenia EcR (“JcEcR”), a fruit fly Drosophila melanogaster EcR (“DmEcR”), a mosquito Aedes aegypti EcR (“AaEcR”), a blowfly Lucilia capitata (“LcEcR”), a blowfly Lucilia cuprina EcR (“LucEcR”), a blowfly Caliphora vicinia EcR (“CvEcR”), a Mediterranean fruit fly Ceratitis capitata EcR (“CcEcR”), a locust Locusta migratoria EcR (“LmEcR”), an aphid Myzus persicae EcR (“MpEcR”), a fiddler crab Celuca pugilator EcR (“CpEcR”), an ixodid tick Amblyomma americanum EcR (“AmaEcR”), a whitefly Bamecia argentifoli EcR or a leafhopper Nephotetix cincticeps EcR. In some embodiments, the LIPC polypeptide component is from a CfEcR, a DmEcR, or an AmaEcR.

In certain embodiments, the LIPC Group H nuclear receptor polypeptide component is encoded by a polynucleotide comprising, or consisting of, a codon mutation that results in a substitution of a) amino acid residue 20, 21, 48, 51, 52, 55, 58, 59, 61, 62, 92, 93, 95, 96, 107, 109, 110, 120, 123, 125, 175, 218, 219, 223, 230, 234, or 238 of SEQ ID NO: 17, b) amino acid residues 95 and 110 of SEQ ID NO: 17, c) amino acid residues 218 and 219 of SEQ ID NO: 17, d) amino acid residues 107 and 175 of SEQ ID NO: 17, e) amino acid residues 127 and 175 of SEQ ID NO: 17, f) amino acid residues 107 and 127 of SEQ ID NO: 17, g) amino acid residues 107, 127 and 175 of SEQ ID NO: 17, h) amino acid residues 52, 107 and 175 of SEQ ID NO: 17, i) amino acid residues 96, 107, and 175 of SEQ ID NO: 17, j) amino acid residues 107, 110, and 175 of SEQ ID NO: 17, k) amino acid residue 107, 121, 213, or 217 of SEQ ID NO: 18, or 1) amino acid residue 91 or 105 of SEQ ID NO: 19. In certain embodiments, the Group H nuclear receptor ligand binding domain is from an ecdysone receptor. In certain embodiments, an LIPC EcR polypeptide component comprising a substitution mutation can comprise, or consist of, a substitution of about or at least 1, 2, 3, 4, 5, 6, 7, 8, 9, 10, 11, 12, 13, 14, 15, 16, 17, 18, 19, 20, 21, 22, 23, 24, 25, or more wild-type or naturally occurring amino acid with a different amino acid relative to the wild-type or naturally occurring EcR receptor ligand binding domain polypeptide.

In another embodiment, the LIPC Group H nuclear receptor ligand polypeptide component is encoded by a polynucleotide comprising, or consisting of, a codon mutation that results in a substitution of a) an alanine residue at a position equivalent or analogous to amino acid residue 20, 21, 48, 51, 55, 58, 59, 61, 62, 92, 93, 95, 109, 120, 125, 218, 219, 223, 230, 234, or 238 of SEQ ID NO: 17, b) an alanine, valine, isoleucine, or leucine residue at a position equivalent or analogous to amino acid residue 52 of SEQ ID NO: 17, c) an alanine, threonine, aspartic acid, or methionine residue at a position equivalent or analogous to amino acid residue 96 of SEQ ID NO: 17, d) a proline, serine, methionine, or leucine residue at a position equivalent or analogous to amino acid residue 110 of SEQ ID NO: 17, e) a phenylalanine residue at a position equivalent or analogous to amino acid residue 123 of SEQ ID NO: 17, f) an alanine residue at a position equivalent or analogous to amino acid residue 95 of SEQ ID NO: 17 and a proline residue at a position equivalent or analogous to amino acid residue 110 of SEQ ID NO: 17, g) an alanine residue at a position equivalent or analogous to amino acid residues 218 and 219 of SEQ ID NO: 17, h) an isoleucine residue at a position equivalent or analogous to amino acid residue 107 of SEQ ID NO: 17, i) an glutamine residue at a position equivalent or analogous to amino acid residues 175 of SEQ ID NO: 17, j) an isoleucine residue at a position equivalent or analogous to amino acid residue 107 of SEQ ID NO: 17 and a glutamine residue at a position equivalent or analogous to amino acid residue 175 of SEQ ID NO: 17, k) a glutamine residue at a position equivalent or analogous to amino acid residues 127 and 175 of SEQ ID NO: 17, 1) an isoleucine residue at a position equivalent or analogous to amino acid residue 107 of SEQ ID NO: 17 and a glutamine residue at a position equivalent or analogous to amino acid residue 127 of SEQ ID NO: 17, m) an isoleucine residue at a position equivalent or analogous to amino acid residue 107 of SEQ ID NO: 17 and a glutamine residue at a position equivalent or analogous to amino acid residues 127 and 175 of SEQ ID NO: 17, n) a valine residue at a position equivalent or analogous to amino acid residue of SEQ ID NO: 17, an isoleucine residue at a position equivalent or analogous to amino acid residue 107 of SEQ ID NO: 17 and a glutamine residue at a position equivalent or analogous to amino acid residue 175 of SEQ ID NO: 17, o) an alanine residue at a position equivalent or analogous to amino acid residue 96 of SEQ ID NO: 17, an isoleucine residue at a position equivalent or analogous to amino acid residue 107 of SEQ ID NO: 17 and a glutamine residue at a position equivalent or analogous to amino acid residue of SEQ ID NO: 17, p) an alanine residue at a position equivalent or analogous to amino acid residue 52 of SEQ ID NO: 17, an isoleucine residue at a position equivalent or analogous to amino acid residue 107 of SEQ ID NO: 17, and a glutamine residue at a position equivalent or analogous to amino acid residue 175 of SEQ ID NO: 17, q) a threonine residue at a position equivalent or analogous to amino acid residue 96 of SEQ ID NO: 17, an isoleucine residue at a position equivalent or analogous to amino acid residue 107 of SEQ ID NO: 17, and a glutamine residue at a position equivalent or analogous to amino acid residue 175 of SEQ ID NO: 17, r) an isoleucine residue at a position equivalent or analogous to amino acid residue 107 of SEQ ID NO: 17, a proline residue at a position equivalent or analogous to amino acid 110 of SEQ ID NO: 17, and a glutamine residue at a position equivalent or analogous to amino acid 175 of SEQ ID NO: 17, s) a proline at a position equivalent or analogous to amino acid residue 107 of 25 SEQ ID NO: 18, t) an arginine or a leucine at a position equivalent or analogous to amino acid residue 121 of SEQ ID NO: 18, u) an alanine at a position equivalent or analogous to amino acid residue 213 of SEQ ID NO: 18, v) an alanine or a serine at a position equivalent or analogous to amino acid residue 217 of SEQ ID NO: 18, w) an alanine at a position equivalent or analogous to amino acid residue 91 of SEQ ID NO: 19, or x) a proline at a position equivalent or analogous to amino acid residue 105 of SEQ ID NO: 19. In certain embodiments, the LIPC Group H nuclear receptor polypeptide component is from an ecdysone receptor.

In another embodiment, the LIPC Group H nuclear receptor polypeptide component having a substitution mutation is an ecdysone receptor ligand binding domain comprising, or consisting of, a substitution mutation encoded by a polynucleotide comprising, or consisting of, a codon mutation that results in a substitution mutation selected from the group consisting of a) E20A, Q21A, F48A, I51A, T52A, T52V, T52I, T52L, T55A, T58A, V59A, L61 A, I62A, M92A, M93A, R95A, V96A, V96T, V96D, V96M, V107I, F109A, A110P, A110S, A110M, A110L, Y120A, A123F, M125A, R175E, M218A, C219A, L223A, L230A, L234A, W238A, R95A/A110P, M218A/C219A, V107I/R175E, Y127E/R175E, V107I/Y127E, V107I/Y127E/R175E, T52V/V107I/R175E, V96A/V107I/R175E, T52A/V107I/R175E, V96T/V107I/R175E or V107I/A110P/R175E substitution mutation of SEQ ID NO: 17, b) A107P, G121R, G121L, N213A, C217A, or C217S substitution mutation of SEQ ID NO: 18, and c) G91A or A105P substitution mutation of SEQ ID NO: 19.

In other embodiments, the LIPC Group H nuclear receptor polypeptide component having a substitution mutation is an ecdysone receptor ligand binding domain polypeptide comprising, or consisting of, a substitution mutation encoded by a polynucleotide that hybridizes to a polynucleotide comprising a codon mutation that results in a substitution mutation selected from the group consisting of a) T58A, A110P, A110L, A110S, or A110M of SEQ ID NO: 17, b) A107P of SEQ ID NO: 18, and c) A105P of SEQ ID NO: 19 under hybridization conditions comprising a hybridization step in less than 500 mM salt and at least 37 degrees Celsius, and a washing step in 2XSSPE at least 63 degrees Celsius. In certain embodiments, the hybridization conditions comprise less than 200 mM salt and at least 37 degrees Celsius for the hybridization step. In another embodiment, the hybridization conditions comprise 2XSSPE and 63 degrees Celsius for both the hybridization and washing steps. In another embodiment, the ecdysone receptor ligand binding domain lacks or exhibits reduced steroid binding activity, such as 20-hydroxyecdysone binding activity, ponasterone A binding activity, or muristerone A binding activity.

In another embodiment, the LIPC Group H nuclear receptor polypeptide component has a substitution mutation at a position equivalent or analogous to a) amino acid residue 20, 21, 48, 51, 52, 55, 58, 59, 61, 62, 92, 93, 95, 96, 107, 109, 110, 120, 123, 125, 175, 218, 219, 223, 230, 234, or 238 of SEQ ID NO: 17, b) amino acid residues 95 and 110 of SEQ ID NO: 17, c) amino acid residues 218 and 219 of SEQ ID NO: 17, d) amino acid residues 107 and 175 of SEQ ID NO: 17, e) amino acid residues 127 and 175 of SEQ ID NO: 17, f) amino acid residues 107 and 127 of SEQ ID NO: 17, g) amino acid residues 107, 127 and 175 of SEQ ID NO: 17, h) amino acid residues 52, 107 and 175 of SEQ ID NO: 17, i) amino acid residues 96, 107 and 175 of SEQ ID NO: 17, j) amino acid residues 107, 110, and 175 of SEQ ID NO: 17, k) amino acid residue 107, 121, 213, or 217 of SEQ ID NO: 18, or 1) amino acid residue 91 or 105 of SEQ ID NO: 19. In certain embodiments, the LIPC Group H nuclear receptor polypeptide component is from an ecdysone receptor.

In some embodiments, the LIPC Group H nuclear receptor polypeptide component has a substitution of a) an alanine residue at a position equivalent or analogous to amino acid residue 20, 21, 48, 51, 55, 58, 59, 61, 62, 92, 93, 95, 109, 120, 125, 218, 219, 223, 230, 234, or 238 of SEQ ID NO: 17, b) an alanine, valine, isoleucine, or leucine residue at a position equivalent or analogous to amino acid residue 52 of SEQ ID NO: 17, c) an alanine, threonine, aspartic acid, or methionine residue at a position equivalent or analogous to amino acid residue 96 of SEQ ID NO: 17, d) a proline, serine, methionine, or leucine residue at a position equivalent or analogous to amino acid residue 110 of SEQ ID NO: 17, e) a phenylalanine residue at a position equivalent or analogous to amino acid residue 123 of SEQ ID NO: 17, f) an alanine residue at a position equivalent or analogous to amino acid residue 95 of SEQ ID NO: 17 and a proline residue at a position equivalent or analogous to amino acid residue 110 of SEQ ID NO: 17, g) an alanine residue at a position equivalent or analogous to amino acid residues 218 and 219 of SEQ ID NO: 17, h) an isoleucine residue at a position equivalent or analogous to amino acid residue 107 of SEQ ID NO: 17, 1) a glutamine residue at a position equivalent or analogous to amino acid residues 175 of SEQ ID NO: 17, j) an isoleucine residue at a position equivalent or analogous to amino acid residue 107 of SEQ ID NO: 17 and a glutamine residue at a position equivalent or analogous to amino acid residue 175 of SEQ ID NO: 17, k) a glutamine residue at a position equivalent or analogous to amino acid residues 127 and 175 of SEQ ID NO: 17, 1) an isoleucine residue at a position equivalent or analogous to amino acid residue 107 of SEQ ID NO: 17 and a glutamine residue at a position equivalent or analogous to amino acid residue 127 of SEQ ID NO: 17, m) an isoleucine residue at a position equivalent or analogous to amino acid residue 107 of SEQ ID NO: 17 and a glutamine residue at a position equivalent or analogous to amino acid residues 127 and 175 of SEQ ID NO: 17, n) a valine residue at a position equivalent or analogous to amino acid residue 52 of SEQ ID NO: 17, an isoleucine residue at a position equivalent or analogous to amino acid residue 107 of SEQ ID NO: 17 and a glutamine residue at a position equivalent or analogous to amino acid residue 175 of SEQ ID NO: 17, o) an alanine residue at a position equivalent or analogous to amino acid residue 96 of SEQ ID NO: 17, an isoleucine residue at a position equivalent or analogous to amino acid residue 107 of SEQ ID NO: 17 and a glutamine residue at a position equivalent or analogous to amino acid residue 175 of SEQ ID NO: 17, p) an alanine residue at a position equivalent or analogous to amino acid residue 52 of SEQ ID NO: 17, an isoleucine residue at a position equivalent or analogous to amino acid residue 107 of SEQ ID NO: 17, and a glutamine residue at a position equivalent or analogous to amino acid residue 175 of SEQ ID NO: 17, q) a threonine residue at a position equivalent or analogous to amino acid residue 96 of SEQ ID NO: 17, an isoleucine residue at a position equivalent or analogous to amino acid residue 107 of SEQ ID NO: 17, and a glutamine residue at a position equivalent or analogous to amino acid residue 175 of SEQ ID NO. 17, r) an isoleucine residue at a position equivalent or analogous to amino acid residue 107 of SEQ ID NO: 17, a proline residue at a position equivalent or analogous to amino acid 110 of SEQ ID NO: 17, and a glutamine residue at a position equivalent or analogous to amino acid 175 of SEQ ID NO: 17, s) a proline at a position equivalent or analogous to amino acid residue 107 of SEQ ID NO: 18, t) an arginine or a leucine at a position equivalent or analogous to amino acid residue 121 of SEQ ID NO: 18, u) an alanine at a position equivalent or analogous to amino acid residue 213 of SEQ ID NO: 18, v) an alanine or a serine at a position equivalent or analogous to amino acid residue 217 of SEQ ID NO: 18, w) an alanine at a position equivalent or analogous to amino acid residue 91 of SEQ ID NO: 19, or x) a proline at a position equivalent or analogous to amino acid residue 105 of SEQ ID NO: 19. In certain embodiments, the LIPC Group H nuclear receptor polypeptide component is from an ecdysone receptor.

In another embodiment, an LIPC Group H nuclear receptor polypeptide component having a substitution mutation is an ecdysone receptor ligand binding domain polypeptide composing a substitution mutation, wherein the substitution mutation is selected from the group consisting of a) E20A, Q21A, F48A, I51A, T52A, T52V, T52I, T52L, T55A, T58A, V59A, L61A, I62A, M92A, M93A, R95A, V96A, V96T, V96D, V96M, V107L F109A, A110P, A110S, A110M, A110L, Y120A, A123F, M125A, R175E, M218A, C219A, L223A, L230A, L234A, W238A, R95A/A110P, M218A C219A, V107I/R175E, Y127E/R175E, V107I/Y127E, V107I/Y127E/R175E, T52V/V107I/R175E, V96A/V107I/R175E, T52A/V107I/R175E, V96T/V107I/R175E, or V107I/A110P/R175E substitution mutation of SEQ ID NO: 17, b) A107P, G121R, G121L, N213A, C217A, or C217S substitution mutation of SEQ ID NO: 18, and c) G91A or A105P substitution mutation of SEQ ID NO: 19. In certain embodiments an EcR polypeptide component (amino acid sequence) used in an LIPC protein of the invention comprises, or alternatively consists of, one or more substitution mutations selected from the group consisting of substitutions indicated in Table 1.

TABLE 1
EcR polypeptide substitution mutations that can be used in the LIPC system.

| Reference PCT | EcR Domain Single Amino Acid | EcR Domain Combination |
|---|---|---|
| Publication | Substitutions | Substitution Mutations |
| WO 2002/066612 | In SEQ ID NO: 1 of WO 2002/066612 | In SEQ ID NO: 1 of WO 2002/066612 |
| (PCT/US2002/005090) | (provided herein as SEQ ID NO: 17): | (provided herein as SEQ ID NO: 17): |
| “NOVEL | E20X or A | T52X + V107X + R175X |
| SUBSTITUTION | Q21X or A | T52A + V107I + R175E |
| MUTANT | F48X or A, L, W, Y, K, R, N | T52V + V107I + R175E |
| RECEPTORS AND | I51X or A, M, N, L | T52V + A110P |
| THEIR USE IN A | T52X or A, V, I, L, M, E, | R95X + A110X |
| NUCLEAR | P, R, W, G, Q | R95A + A110P |
| RECEPTOR-BASED | M54W or T | V96X + V107X + R175X |
| INDUCIBLE GENE | T55X or A | V96A + V107I + R175E |
| EXPRESSION | T58X or A | V96T + V107I + R175E |
| SYSTEM”, | V59X or A | V96T + 119F |
| which is hereby | L61X or A | V107X + A110X + R175X |
| incorporated by | I62X or A | V107X + Y127X |
| reference herein in its | M92X or A, L, E | V107X + Y127X + R175X |
| entirety. | M93X or A | V107X + R175X |
| | R95X or A, H, M, W | V107I + A110P + Y127E |
| | V96X or A, T, D, M, S, E | V107I + A110P + Y127E |
| | V107X or I | V107I + A110P + R175E |
| | F109X or A, W, P, N, M | V107I + Y127E |
| | A110X or P, S, M, L, E, N, W | V107I + Y127E + L152V |
| | N119F | V107I + Y127E + R175E |
| | Y120X or A, W, M | V107I + R175E |
| | A123X or F | A110P + V128F |
| | M125X or A, P, R, E, L, | Y127X + R175X |
| | C, W, G, I, N, S, V | Y127E + R175E |
| | V128F | N218X + M219X |
| | L132M or N, V, E | |
| | R175X or E | |
| | N218X | |
| | M219X | |
| | L223X or A, K, R, Y | |
| | L230X or A | |
| | L234X or A, M, I, R, W | |
| | W238X or A, P, E, Y, M, L | |
| INX00068-WO | In SEQ ID NO: 1 of WO 2005/108617 | In SEQ ID NO: 1 of WO 2005/108617 |
| WO 2005/108617 | (provided herein as SEQ ID NO: 86): | (provided herein as SEQ ID NO: 86): |
| (PCT/US2005/015089) | F48X or N, R, Y, W, L, K | T52X + A110X |
| “MUTANT | I51X or M, N, L | T52X + V107X + Y127X |
| RECEPTORS AND | T52X or L, P, M, R, W, G, | T52V + A110P |
| THEIR USE IN A | Q, E, V | T52V + V107I + Y127E |
| NUCLEAR | M54X or W, T | V96X + N119X |
| RECEPTOR-BASED | M92X or L, E | V96T + N119F |
| INDUCIBLE GENE | R95X or H, M, W | V107X + A110X + Y127X |
| EXPRESSION | V96X or L, S, E, W, T | V107I + A110P + Y127E |
| SYSTEM” | V107I | V107X + Y127X + 259X* |
| Which is hereby | F109X or W, P, L, M, N | V107I + Y127E + 259G* |
| incorporated by | A110X or E, W, N, P | A110X + V128X |
| reference herein in its | N119X or F | A110P + V128F |
| entirety. | Y120X or W, M | |
| | M125X or E, P, L, C, W, | |
| | G, I, N, S, V, R | |
| | V128X or F | |
| | L132X or M, N, E, V | |
| | M219X or A, K, W, Y | |
| | L223X or K, R, Y | |
| | L234X or M, R, W, I | |
| | W238X or P, E, L, M, Y | |

RXR Components

The present invention provides for particular RXR components, including RXR ligand binding domains (LBD), to be employed in ligand inducible polypeptide couplers (LIPCs) described herein. Exemplary RXR components that can be used in the present invention include, for example, those described in International PCT Publ. Nos.: WO 2001/070816; WO 2002/066612; WO 2002/066613; WO 2002/066614; WO 2002/066615; WO 2003/027266; WO 2003/027289; WO 2005/108617 and, WO 2009/114201, each of which is incorporated by reference herein in its entirety.

In certain embodiments, the LIPC RXR component is a mouse Mus musculus RXR (MmRXR) or a human Homo sapiens RXR (HsRXR). The LIPC RXR component may be an RXR_{α}, RXR_{β}, or RXR_{γ}isoform, or fragment thereof.

In some embodiments, the RXR LIPC component is a truncated RXR. The LIPC RXR polypeptide truncation can comprise, or consist of, a deletion of at least 1, 2, 3, 4, 5, 10, 15, 20, 25, 30, 35, 40, 45, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, 200, 205, 210, 215, 220, 225, 230, 235, 240, 245, 250, 255, 260, or 265 amino acids. In certain embodiments, the LIPC RXR polypeptide truncation comprises, or consists of, a deletion of at least a partial polypeptide domain. In some embodiments, the LIPC RXR polypeptide truncation comprises, or consists of, a deletion of at least an entire polypeptide domain. In a specific embodiment, the LIPC RXR polypeptide truncation comprises, or consists of, a deletion of at least an AB-domain deletion, a C-domain deletion, a D-domain deletion, an E-domain deletion, an F-domain deletion, an A/B/C-domains deletion, an A/B/1/2-C-domains deletion, an A B/C/D-domains deletion, an A/B/C D/F-domains deletion, an A/B/F-domains, and an A/B/C/F-domains deletion. A combination of several complete and/or partial domain deletions may also be performed.

In certain embodiments, the LIPC RXR polypeptide component is encoded by a polynucleotide comprising, or consisting of, a nucleic acid sequence selected from the group consisting of SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, and SEQ ID NO: 39, or a fragment thereof.

In another embodiment, the LIPC RXR component comprises or consists of a polypeptide sequence selected from the group consisting of SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, and SEQ ID NO: 49, or a fragment thereof.

In certain embodiments, LIPC of the invention include a chimeric RXR polypeptide comprising at least two polypeptide fragments selected from the group consisting of: 1) a vertebrate species RXR polypeptide fragment; 2) an invertebrate species RXR polypeptide fragment; and, 3) a non-Dipteran/non-Lepidopteran invertebrate species RXR polypeptide fragment. An LIPC chimeric RXR polypeptide component of the invention may comprise or consist of two different animal species RXR polypeptide fragments, or when the animal species is the same, the two or more polypeptide fragments may be from two or more different isoforms of the animal species RXR polypeptide fragment.

In some embodiments, the vertebrate species LIPC RXR polypeptide fragment comprises or consists of a mouse Mus musculus RXR (MmRXR) or a human Homo sapiens RXR (HsRXR), or fragment thereof. The LIPC RXR polypeptide component may comprise or consist of an RXR_{α}, RXR_{β}, or RXR_{γ}isoform, or fragment thereof.

In some embodiments, the vertebrate species LIPC RXR polypeptide fragment is from a vertebrate species RXR encoded by a polynucleotide comprising, or consisting of, a nucleic acid sequence selected from the group consisting of SEQ ID NO: 62, SEQ ID NO: 63, SEQ ID NO: 64, SEQ ID NO: 65, SEQ ID NO: 66, and SEQ ID NO: 67, or fragment thereof. In another embodiment, the vertebrate species LIPC RXR polypeptide fragment is from a vertebrate species RXR comprising, or consisting of, an amino acid sequence selected from the group consisting of SEQ ID NO: 68, SEQ ID NO: 69, SEQ ID NO: 70, SEQ ID NO: 71, SEQ ID NO: 72, and SEQ ID NO: 73, or fragment thereof.

In another embodiment, a LIPC invertebrate species RXR polypeptide fragment is from a locust Locusta migratoria ultraspiracle polypeptide (LmUSP), an ixodid tick Amblyomma americanum RXR homolog 1 (AmaRXR1), a ixodid tick Amblyomma americanum RXR homolog 2 (AmaRXR2), a fiddler crab Celuca pugilator RXR homolog (CpRXR), a beetle Tenebrio molitor RXR homolog (TmRXR), a honeybee Apis mellifera RXR homolog (AmRXR), and an aphid Myzus persicae RXR homolog (MpRXR).

In certain embodiments, a LIPC invertebrate species RXR polypeptide fragment is from a invertebrate species RXR polypeptide encoded by a polynucleotide comprising or consisting of a nucleic acid sequence of SEQ ID NO: 50, SEQ ID NO: 51, SEQ ID NO: 52, SEQ ID NO: 53, SEQ ID NO: 54, or SEQ ID NO: 55, or fragment thereof. In another embodiment, a LIPC invertebrate species RXR polypeptide fragment is from a invertebrate species RXR polypeptide comprising or consisting of an amino acid sequence of SEQ ID NO: 56, SEQ ID NO: 57, SEQ ID NO: 58, SEQ ID NO: 59, SEQ ID NO: 60, or SEQ ID NO: 61, or fragment thereof.

In certain embodiments, a LIPC invertebrate species RXR polypeptide fragment is from a non-Dipteran/non-Lepidopteran invertebrate species RXR homolog.

In some embodiments, a LIPC chimeric RXR component comprises or consists of at least one vertebrate species RXR polypeptide fragment and one invertebrate species RXR polypeptide fragment.

In another embodiment, a LIPC chimeric RXR component comprises or consists of at least one vertebrate species RXR polypeptide fragment and one non-Dipteran/non-Lepidopteran invertebrate species RXR homolog polypeptide fragment.

In another embodiment, a LIPC chimeric RXR component comprises or consists of at least one invertebrate species RXR polypeptide fragment and one non-Dipteran/non-Lepidopteran invertebrate species RXR homolog polypeptide fragment.

In another embodiment, a LIPC chimeric RXR component comprises or consists of at least one vertebrate species RXR polypeptide fragment and one different vertebrate species RXR polypeptide fragment.

In another embodiment, a LIPC chimeric RXR component comprises or consists of at least one invertebrate species RXR polypeptide fragment and one different invertebrate species RXR polypeptide fragment.

In another embodiment, a LIPC chimeric RXR component comprises or consists of at least one non-Dipteran/non-Lepidopteran invertebrate species RXR polypeptide fragment and one different non-Dipteran non-Lepidopteran invertebrate species RXR polypeptide fragment.

In certain embodiments, a LIPC chimeric RXR component has an RXR region comprising at least one polypeptide fragment selected from the group consisting of an EF-domain helix 1, an EF-domain helix 2, an EF-domain helix 3, an EF-domain helix 4, an EF-domain helix 5, an EF-domain helix 6, an EF-domain helix 7, an EF-domain helix 8, and EF-domain helix 9, an EF-domain helix 10, an EF-domain helix 11, an EF-domain helix 12, an F-domain, and/or an EF-domain β-pleated sheet, wherein at least one of two or more domains are from different species RXR (e.g., a human RXR polypeptide fragment and a murine RXR polypeptide fragment).

In another embodiment, a first polypeptide fragment of a LIPC chimeric RXR component component comprises or consists of helices 1-6, helices 1-7, helices 1-8, helices 1-9, helices 1-10, helices 1-11, or helices 1-12 of a first species RXR, and a second polypeptide fragment of the chimeric LIPC RXR component comprises or consists of helices 7-12, helices 8-12, helices 9-12, helices 10-12, helices 11-12, helix 12, or F domain of a second species RXR, respectively.

In another embodiment, a first polypeptide fragment of a LIPC chimeric RXR component comprises or consists of helices 1-6 of a first species RXR, and a second polypeptide fragment of the LIPC chimeric RXR component comprises helices 7-12 of a second species RXR.

In another embodiment, a first polypeptide fragment of a LIPC chimeric RXR component comprises or consists of helices 1-7 of a first species RXR, and a second polypeptide fragment of the LIPC chimeric RXR component comprises or consists of helices 8-12 of a second species RXR.

In another embodiment, a first polypeptide fragment of a LIPC chimeric RXR component comprises or consists of helices 1-8 of a first species RXR, and a second polypeptide fragment of the LIPC chimeric RXR component comprises or consists of helices 9-12 of a second species RXR.

In another embodiment, a first polypeptide fragment of a LIPC chimeric RXR component comprises or consists of helices 1-9 of a first species RXR, and a second polypeptide fragment of the LIPC chimeric RXR component comprises or consists of helices 10-12 of a second species RXR.

In another embodiment, a first polypeptide fragment of a LIPC chimeric RXR component comprises or consists of helices 1-10 of a first species RXR, and a second polypeptide fragment of the LIPC chimeric RXR component comprises or consists of helices 11-12 of a second species RXR.

In another embodiment, a first polypeptide fragment of a LIPC chimeric RXR component comprises or consists of helices 1-11 of a first species RXR, and a second polypeptide fragment of the LIPC chimeric RXR component comprises or consists of helix 12 of a second species RXR.

In another embodiment, a first polypeptide fragment of a LIPC chimeric RXR component comprises or consists of helices 1-12 of a first species RXR, and a second polypeptide fragment of the LIPC chimeric RXR component comprises or consists of an F domain of a second species RXR.

In another embodiment, a LIPC RXR component comprises or consists of a truncated chimeric RXR. A chimeric RXR truncation can comprise a deletion of at least 1, 2, 3, 4, 5, 6, 8, 10, 13, 14, 15, 16, 17, 18, 19, 20, 21, 22, 25, 26, 30, 35, 40, 45, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, 200, 205, 210, 215, 220, 225, 230, 235, or 240 amino acids. In certain embodiments, a chimeric RXR truncation results in a deletion of at least a partial polypeptide domain. In other embodiments, a chimeric RXR truncation results in a deletion of at least an entire polypeptide domain. In another embodiment, a chimeric RXR truncation results in a deletion of at least a partial E-domain, a complete E-domain, a partial F-domain, a complete F-domain, an EF-domain helix 1, an EF-domain helix 2, an EF-domain helix 3, an EF-domain helix 4, an EF-domain helix 5, an EF-domain helix 6, an EF-domain helix 7, an EF-domain helix 8, and EF-domain helix 9, an EF-domain helix 10, an EF-domain helix 11, an EF-domain helix 12, and/or an EF-domain f3-pleated sheet. A combination of several partial and or complete domain deletions may also be performed.

In certain embodiments, a LIPC truncated chimeric RXRcomponent is encoded by a polynucleotide comprising or consisting of a nucleic acid sequence of SEQ ID NO: 74, SEQ ID NO: 75, SEQ ID NO: 76, SEQ ID NO: 77, SEQ ID NO: 78, or SEQ ID NO: 79, or fragments thereof. In another embodiment, a LIPC truncated chimeric RXR component comprises or consists of a nucleic acid sequence of SEQ ID NO: 80, SEQ ID NO: 81, SEQ ID NO: 82, SEQ ID NO: 83, SEQ ID NO: 84, or SEQ ID NO: 85, or fragment thereof.

In another embodiment, a LIPC chimeric RXR component is encoded by a polynucleotide comprising or consisting of a nucleic acid sequence of a) SEQ ID NO: 11, b) nucleotides 1-348 of SEQ BD NO: 12 and nucleotides 268-630 of SEQ ID NO: 13, c) nucleotides 1-408 of SEQ ID NO: 12 and nucleotides 337-630 of SEQ ID NO: 13, d) nucleotides 1-465 of SEQ ID NO: 12 and nucleotides 403-630 of SEQ ID NO: 13, e) nucleotides 1-555 of SEQ ID NO: 12 and nucleotides 490-630 of SEQ ID NO: 13, f) nucleotides 1-624 of SEQ ID NO: 12 and nucleotides 547-630 of SEQ ID NO: 13, g) nucleotides 1-645 of SEQ ID NO: 12 and nucleotides 601-630 of SEQ ID NO: 13, and h) nucleotides 1-717 of SEQ ID NO: 12 and/or nucleotides 613-630 of SEQ ID NO: 13, or a fragment thereof.

In another preferred embodiment, a LIPC chimeric RXR component comprises of consists of an amino acid sequence of a) SEQ ID NO: 14, b) amino acids 1-116 of SEQ ID NO: 15 and amino acids 90-210 of SEQ ID NO: 16, c) amino acids 1-136 of SEQ ID NO: 15 and amino acids 113-210 of SEQ ID NO: 16, d) amino acids 1-155 of SEQ ID NO: 15 and amino acids 135-210 of SEQ ID NO: 16, e) amino acids 1-185 of SEQ ID NO: 15 and amino acids 164-210 of SEQ ID NO: 16, f) amino acids 1-208 of SEQ ID NO: 15 and amino acids 183-210 of SEQ ID NO: 16, g) amino acids 1-215 of SEQ ID NO: 15 and amino acids 201-210 of SEQ ID NO: 16, and/or h) amino acids 1-239 of SEQ ID NO: 15 or amino acids 205-210 of SEQ ID NO: 16, or a fragment thereof.

EcR and/or RXR Polypeptide Components

In certain embodiments, EcR and/or USP/RXR polypeptides used in a LIPC of the invention comprise, or consist of, at least one or more EcR and/or RXR substitution mutants selected from the group consisting of substitution mutants described in any one or more of International PCT Publ. Nos. WO 2001/070816, WO 2002/066612, WO 2002/066613, WO 2002/066614, WO 2002/066615, WO 2003/027266, WO 2003/027289, and WO 2005/108617, each of which is incorporated by reference herein in its entirety.

Gene Expression Cassettes of the Present Invention

One embodiment of the invention includes a ligand inducible polypeptide coupler (LIPC) system comprising: a) a first expression cassette that is capable of being expressed in a host cell comprising a polynucleotide that encodes a first fusion protein (polypeptide) comprising i) a nuclear receptor polypeptide or fragment thereof and ii) a first inactive signaling domain; and b) a second expression cassette that is capable of being expressed in the host cell comprising a polynucleotide sequence that encodes a second, separate, fusion protein (polypeptide) comprising i) a second nuclear receptor polypeptide or fragment thereof and ii) a second inactive signaling domain; wherein the first and second inactive signaling domains are activated upon association of the two fusion proteins with one another.

Another embodiment of the invention includes a ligand inducible polypeptide coupler (LIPC) system comprising: a) a first expression cassette that is capable of being expressed in a host cell comprising a polynucleotide that encodes a first fusion protein (polypeptide) comprising i) an arthropod nuclear receptor polypeptide or fragment thereof; and ii) a first inactive signaling domain; and b) a second expression cassette that is capable of being expressed in the host cell comprising a polynucleotide sequence that encodes a second, separate, fusion protein (polypeptide) comprising i) a second, non-arthropod nuclear receptor polypeptide or fragment thereof; and ii) a second inactive signaling domain; wherein the first and second inactive signaling domains are activated upon association of the two fusion proteins with one another. In another embodiment the non-arthropod nuclear receptor comprises a non-dipteran/non-lepidopteran nuclear receptor polypeptide or fragment thereof. In another embodiment the non-arthropod nuclear receptor comprises a mammalian nuclear receptor polypeptide or fragment thereof. In another embodiment the non-arthropod nuclear receptor comprises a human nuclear receptor polypeptide or fragment thereof. In another embodiment the non-arthropod nuclear receptor comprises a murine nuclear receptor polypeptide or fragment thereof. In another embodiment the non-arthropod nuclear receptor comprises a chimeric nuclear receptor polypeptide or fragments thereof, wherin the chimera comprises polypeptide components from two or more different species.

One embodiment of the invention includes a ligand inducible polypeptide coupler (LIPC) system comprising: a) a first expression cassette that is capable of being expressed in a host cell comprising a polynucleotide that encodes a first fusion protein (polypeptide) comprising i) an ecdysone receptor (EcR) polypeptide or fragment thereof and ii) a first inactive signaling domain; and b) a second expression cassette that is capable of being expressed in the host cell comprising a polynucleotide sequence that encodes a second, separate, fusion protein (polypeptide) comprising i) a retinoid X receptor polypeptide or fragment thereof and ii) a second inactive signaling domain; wherein the first and second inactive signaling domains are activated upon association of the two fusion proteins with one another.

Ligands, optionally, for use in invention as described below, when combined with an EcR ligand binding domain and a RXR ligand binding domain, as described herein, provide the means for external temporal regulation (activation or withdrawal of activation; i.e., via cessation of administration, or contact with, ligand) of the signaling domain(s). Binding of ligand to the LIPC EcR and RXR polypeptide components enables protein-protein interaction of LIPC-fusion proteins, and in certain embodiments activation, of the signaling domains. In some embodiments, one or more of the LIPC domains is varied producing a hybrid LIPC. In certain embodiments, hybrid genes and the resulting hybrid proteins are optimized in the chosen host cell or organism for desired activity and complementary binding of the ligand.

### Inactive Signaling Domains

Embodiments of the invention include ligand inducible polypeptide coupler systems that allow for tailored (e.g., dose-regulated, inducible) activation of inactive domains (e.g., signaling molecules, signaling domains, complementary protein fragments, protein subunits, and natural or engineered partial or truncated proteins) through protein-protein interactin or association.

In certain embodiments, a signaling protein and/or polypeptide domain whose activity is to be modulated is a homologous protein or fragment thereof with respect to the host cell. In other embodiments, the signaling protein and/or polypeptide domain whose activity is to be modulated is a heterologous protein or fragment thereof with respect to the host cell.

Embodiments of the invention include compostions and uses of signaling proteins and polypeptide domains encoding polypeptides or signaling domains involved in a disease, a disorder, a dysfunction, a genetic defect, targets for drug discovery, and proteomics analyses and applications, etc.

Numerous cell signaling polypeptides and domains (e.g., signaling proteins) that require association (e.g., dimerization or oligomerization) or protein-protein interaction for activation have been identified in a wide-range of organisms and can be used in the present invention. Many of these signaling molecules participate in signaling pathways that are conserved throughout a large number of organisms.

For example, many cell surface receptors anchored in the membrane with a single transmembrane domain are primarily activated by endogenous (i.e., naturally occurring) ligand-induced dimerization or oligomerization. Generally, these molecules do not associate on their own, but are brought together (or in close proximity to their binding partner) through interactions with an endogenous extracellular ligand. In contrast to endogenous naturally occurring cell signal protein activation, the present invention provides for a small-molecule, ligand inducible polypeptide coupler system to modulate (i.e., turn on, turn off, increase or decrease) activity, i.e., dimerization or oligomerization, of cell signaling proteins and domains via “on demand” administration (or withdrawal of administration) of a small molecule nuclear receptor activating ligand. For a review of various molecules and pathways that utilize protein dimerization or oligomerization for activation, see, e.g., Klemm, et al. Annu. Rev. Immunol. 16:569-92 (1998), which is incorporated by reference herein in its entirety.

In certain embodiments the following signaling molecules and/or domains from cell surface receptors, intracellular signaling proteins, and their associated pathway members are envisaged for use with the invention as the first and/or second inactive signaling domain, signaling molecule, complementary protein fragment, protein subunit, or natural or engineered partial or truncated protein of the invention:

Receptor tyrosine kinase (RTK) receptors and their associated pathway members, including RTK class I (EGF receptor family) (ErbB family), RTK class II (Insulin receptor family), RTK class III (PDGF receptor family), RTK class IV (FGF receptor family), RTK class V (VEGF receptors family), RTK class VI (HGF receptor family), RTK class VII (Trk receptor family), RTK class VIII (Eph receptor family), RTK class IX (AXL receptor family), RTK class X (LTK receptor family), RTK class XI (TIE receptor family), RTK class XII (ROR receptor family), RTK class XIII (DDR receptor family), RTK class XIV (RET receptor family), RTK class XV (KLG receptor family), RTK class XVI (RYK receptor family), and RTK class XVII (MuSK receptor family).

Cytokine receptors and their associated pathway members, including type I cytokine receptor (e.g., Type I interleukin receptors, Erythropoietin receptor, GM-CSF receptor, G-CSF receptor, growth hormone receptor, prolactin receptor, Oncostatin M receptor, and Leukemia inhibitory factor receptor), type II cytokine receptor (e.g., Type II interleukin receptors, interferon-alpha/beta receptor, and interferon-gamma receptor), members of the immunoglobulin superfamily (e.g., Interleukin-1 receptor, CSF1, C-kit receptor, and Interleukin-18 receptor). Tumor necrosis factor receptor family (e.g., CD27, CD30, CD40, CD120, and Lymphotoxin beta receptor). Chemokine receptors (e.g., Interleukin-8 receptor, CCR1, CXCR4, MCAF receptor, and NAP-2 receptor). TGF beta receptors (e.g., TGF beta receptor 1 and TGF beta receptor 2). Antigen receptor signaling receptors (e.g., B cell and T cell antigen receptors).

Additional signaling proteins and/or domains that are envisaged to be used with the present invention include, but are not limited to, firefly luciferase (fLuc), Signal Transducer and Activator of Transcription (STAT) proteins, NF-κB proteins, antibodies (including antibody fragments), transcription factors, nuclear receptors, including nuclear hormone receptors, 14-3-3 proteins, G-protein coupled receptors, G proteins, kinesin, triosephosphateisomerase (TIM), alcohol dehydrogenase, Factor XI, Factor XIII, Toll-like receptors, fibrinogen, Bcl-2 family members, Smad family members, and the like.

In certain embodiments, the inactive signaling domain of the invention have a transmembrane domain. In some embodiments the transmembrane domain is a single-pass transmembrane domain. In certain embodiments, the single-pass transmembrane domain is a single-pass type I transmembrane domain. In other embodiments, the transmembrane domain is a multi-pass transmembrane domain. In certain embodiments, the transmembrane domain(s) have a hydrophilic alpha helix motif.

### Activating Ligands

Acceptable activating ligands that can be used with the invention are any that modulate protein-protein interaction of the signaling domains of the switch system wherein the presence of the ligand results in activation of the inactive signaling domains. Such ligands include those disclosed in International PCT Publ. Nos. WO 2002/066612, WO 2002/066614, WO 2003/105849, WO 2004/072254, WO 2004/005478, WO 2004/078924, WO 2005/017126, WO 2008/153801, WO 2009/114201, WO 2013/036758, WO 2014/144380 and in U.S. Pat. Nos. 6,258,603 and 8,748,125, each of which is incorporated by reference herein in its entirety.

Exemplary ligands include, but are not limited to, ponasterone, muristerone A, 9-cis-retinoic acid, synthetic analogs of retinoic acid, N,N′-diacylhydrazines such as those disclosed in U.S. Pat. Nos. 6,013,836, 5,117,057, 5,530,028 and 537,872, each of which is incorporated by reference herein in its entirety; dibenzoylalkyl cyanohydrazines such as those disclosed in European Application No. 461809, which is incorporated by reference herein in its entirety; N-alkyl-N,N′-diaroylhydrazines such as those disclosed in U.S. Pat. No. 5,225,443 which is incorporated by reference herein in its entirety; N-acyl-N-alkylcarbonylhydrazines such as those disclosed in European Application No. 234994 which is incorporated by reference herein in its entirety; N-aroyl-N-alkyl-N′-aroylhydrazines such as those described in U. S. Pat. No. 4,985,461, which is incorporated by reference herein in its entirety, and other similar materials including 3,5-di-tert-butyl-4-hydroxy-N-isobutyl-benzamide, 8-0-acetylharpagide, and the like.

In certain embodiments, the ligand for use in the methods of the present invention is a compound of the formula:

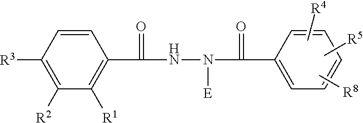

wherein E is a (C₄-C₆)alkyl containing a tertiary carbon or a cyano(C₃-C5)alkyl containing a tertiary carbon; R¹ is H, Me, Et, i-Pr, F, formyl, CF₃, CHF₂, CHCl₂, CH₂F, CH₂Cl, CH₂OH, CH₂OMe, CH₂CN, CN, C≡CH, 1-propynyl, 2-propynyl, vinyl, OH, OMe, OEt, cyclopropyl, CF₂CF₃, CH═CHCN, allyl, azido, SCN, or SCHF₂;

R² is H, Me, Et, n-Pr, i-Pr, formyl, CF₃, CHF₂, CHCl₂, CH₂F, CH₂Cl, CH₂OH, CH₂OMe, CH₂CN, CN, C≡CH, 1-propynyl, 2-propynyl, vinyl, Ac, F, Cl, OH, OMe, OEt, O-n-Pr, OAc, NMe₂, NEt₂, SMe, SEt, SOCF₃, OCF₂CF₂H, COEt, cyclopropyl, CF₂CF₃, CH═CHCN, allyl, azido, OCF₃, OCHF₂, O-i-Pr, SCN, SCHF₂, SOMe, NH—CN, or joined with R³ and the phenyl carbons to which R² and R³ are attached to form an ethylenedioxy, a dihydrofuryl ring with the oxygen adjacent to a phenyl carbon, or a dihydropyryl ring with the oxygen adjacent to a phenyl carbon;

R³ is H, Et, or joined with R² and the phenyl carbons to which R² and R³ are attached to form an ethylenedioxy, a dihydrofuryl ring with the oxygen adjacent to a phenyl carbon, or a dihydropyryl ring with the oxygen adjacent to a phenyl carbon; R⁴, R⁵, and R⁶ are independently H, Me, Et, F, Cl, Br, formyl, CF₃, CHF₂, CHCl₂, CH₂F, CH₂Cl, CH₂OH, CN, C≡CH, 1-propynyl, 2-propynyl, vinyl, OMe, OEt, SMe, or Set

In some embodiments, the ligand for use with the methods of the present invention is a compound of the formula:

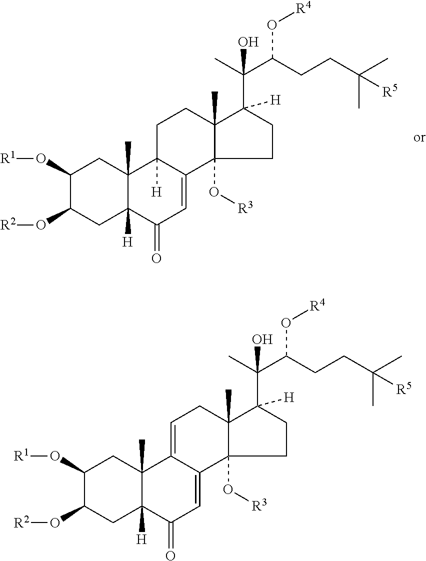

wherein R¹, R², R³, and R⁴ are:

a) H, (C₁-C₆)alkyl; (C₁-C₆)haloalkyl; (C₁-C₆)cyanoalkyl; (C₁-C₆)hydroxyalkyl; (C₁-C₄)alkoxy(C₁-C₆)alkyl; (C₂-C₆)alkenyl optionally substituted with halo, cyano, hydroxyl, or (C₁-C₄)alkyl; (C₂-C₆)alkynyl optionally substituted with halo, cyano, hydroxyl, or (C₁-C₄)alkyl; (C₃-C₅)cycloalkyl optionally substituted with halo, cyano, hydroxyl, or (C₁-C₄)alkyl; oxiranyl optionally substituted with halo, cyano, or (C₁-C₄)alkyl; or

b) unsubstituted or substituted benzyl wherein the substituents are independently 1 to 5 H, halo, nitro, cyano, hydroxyl, (C₁-C₆)alkyl, or (C₁-C₆)alkoxy; and R⁵ is H; OH; F; Cl; or (C₁-C₆)alkoxy.

In some embodiments, when R¹, R², R³, and R⁴ are H, then R⁵ is not H or hydroxy.

In certain embodiments, at least one of R¹, R², R³, and R⁴ is not H. In another embodiment, at least two of R¹, R², R³, and R⁴ are not H. In another embodiment, at least three R¹, R², R³, and R⁴ are not H. In another embodiment, each of R¹, R², R³, and R⁴ are not H.

In some embodiments, when R¹, R², R³, and R⁴ are H, then R⁵ is not methoxy, when R¹, R², R³, and R⁴ are isopropyl, then R⁵ is not hydroxy, and when R¹, R², and R³ are H and R⁵ is hydroxy, then R⁴ is not methyl or ethyl.

In specific embodiments, R¹, R², R³, and R⁴ are: a) H, (C₁-C₆)alkyl; (C₁-C₆)haloalkyl; (C₁-C₆)cyanoalkyl; (C₁-C₆)hydroxyalkyl; (C₁-C₄)alkoxy(C₁-C₆)alkyl; (C₂-C₆)alkenyl; (C₂-C₆)alkynyl; oxiranyl optionally substituted with halo, cyano, or (C₁-C₄)alkyl; or b) unsubstituted or substituted benzyl wherein the substituents are independently 1 to 5 H, halo, cyano, or (C₁-C₆)alkyl; and R⁵ is H, OH, F, Cl, or (C₁-C₆)alkoxy.

In other specific embodiments, R¹, R², R³, and R⁴ are H, (C₁-C₆)alkyl; (C₂-C₆)alkenyl; (C₂-C₆)alkynyl; 2′-ethyloxiranyl, or benzyl; and R⁵ is H; OH; or F.

In specific embodiments, when R¹, R², R³, and R⁴ are isopropyl, then R⁵ is not hydroxyl; when R⁵ is H, hydroxyl, methoxy, or fluoro, then at least one of R¹, R², R³, and R⁴ is not H; when only one of R¹, R², R³, and R⁴ is methyl, and R⁵ is H or hydroxyl, then the remainder of R¹, R², R³, and R⁴ are not H; when both R⁴ and one of R¹, R², and R³ are methyl, then R⁵ is neither H nor hydroxyl; when R¹, R², R³, and R⁴ are all methyl, then R⁵ is not hydroxyl; and when R¹, R², and R³ are all H and R⁵ is hydroxyl, then R⁴ is not ethyl, n-propyl, n-butyl, allyl, or benzyl.

Certain embodiments of the invention include the use of the following steroidal ligands: 20-hydroxyecdysone, 2-methyl ether; 20-hydroxyecdysone, 3-methyl ether; 20-hydroxyecdysone, 14-methyl ether; 20-hydroxyecdysone, 2,22-dimethyl ether; 20-hydroxyecdysone, 3,22-dimethyl ether; 20-hydroxyecdysone, 14,22-dimethyl ether; 20-hydroxyecdysone, 22,25-dimethyl ether; 20-hydroxyecdysone, 2,3,14,22-tetramethyl ether; 20-hydroxyecdysone, 22-H-propyl ether; 20-hydroxyecdysone, 22-n-butyl ether; 20-hydroxyecdysone, 22-allyl ether; 20-hydroxyecdysone, 22-benzyl ether; 20-hydroxyecdysone, 22-(28R,S)-2′-ethyloxiranyl ether; ponasterone A, 2-methyl ether; ponasterone A, 14-methyl ether; ponasterone A, 22-methyl ether; ponasterone A, 2,22-dimethyl ether; ponasterone A, 3,22-dimethyl ether; ponasterone A, 14,22-dimethyl ether; dacryhainansterone, 22-methyl ether.

Additional embodiments of the invention include the use of the following steroidal ligands: 25,26-didehydroponasterone A, (iso-stachysterone C (Δ25(26))), shidasterone (stachysterone D), stachysterone C, 22-deoxy-20-hydroxyecdysone (taxisterone), ponasterone A, polyporusterone B, 22-dehydro-20-hydroxyecdysone, ponasterone A 22-methyl ether, 20-hydroxyecdysone, pterosterone, (25R)-inokosterone, (25S)-inokosterone, pinnatasterone, 25-fluoroponasterone A, 24(28)-dehydromakisterone A, 24-epi-makisterone A, makisterone A, 20-hydroxyecdysone-22-methyl ether, 20-hydroxyecdysone-25-methyl ether, abutasterone, 22,23-di-epi-geradiasterone, 20,26-dihydroxyecdysone (podecdysone C), 24-epi-abutasterone, geradiasterone, 29-norcyasterone, ajugasterone B, 24(28)[Z]-dehydroamarasterone B, amarasterone A, makisterone C, rapisterone C, 20-hydroxyecdysone-22,25-dimethyl ether, 20-hydroxyecdysone-22-ethyl ether, carthamosterone, 24(25)-dehydroprecyasterone, leuzeasterone, cyasterone, 20-hydroxyecdysone-22-allyl ether, 24(28)[Z]-dehydro-29-hydroxymakisterone C, 20-hydroxyecdysone-22-acetate, viticosterone E (20-hydroxyecdysone 25-acetate), 20-hydroxyecdysone-22-n-propyl ether, 24-hydroxycyasterone, 20-hydroxyecdysone-22-n-butyl ether, ponasterone A 22-hemi succinate, 22-acetoacetyl-20-hydroxyecdysone, 20-hydroxyecdysone-22-benzyl ether, canescensterone, 20-hydroxyecdysone-22-hemisuccinate, inokosterone-26-hemisuccinate, 20-hydroxyecdysone-22-benzoate, 20-hydroxyecdysone-22-β-D-glucopyranoside, 20-hydroxyecdysone-25-β-D-glucopyranoside, sileneoside A (20-hydroxyecdysone-22α-galactoside), 3-deoxy-1β,20-dihydroxyecdysone (3-deoxyintegri sterone A), 2-deoxyintegristerone A, 1-epi-integristerone A, integristerone A, sileneoside C (integristerone A 22α-galactoside), 2,22-dideoxy-20-hydroxyecdysone, 2-deoxy-20-hydroxyecdysone, 2-deoxy-20-hydroxyecdysone-3-acetate, 2-deoxy-20,26-dihydroxyecdysone, 2-deoxy-20-hydroxyecdysone-22-acetate, 2-deoxy-20-hydroxyecdysone-3,22-diacetate, 2-deoxy-20-hydroxyecdysone-22-benzoate, ponasterone A 2-hemi succinate, 20-hydroxyecdysone-2-methyl ether, 20-hydroxyecdysone-2-acetate, 20-hydroxyecdysone-2-hemisuccinate, 20-hydroxyecdysone-2-β-D-glucopyranoside, 2-dansyl-20-hydroxyecdysone, 20-hydroxyecdysone-2,22-dimethyl ether, ponasterone A 3B-D-xylopyranoside (limnantheoside B), 20-hydroxyecdysone-3-methyl ether, 20-hydroxyecdysone-3-acetate, 20-hydroxyecdysone-3β-D-xylopyranoside (limnantheoside A), 20-hydToxyecdysone-3-β-D-glucopyranoside, sileneoside D (20-hydroxyecdysone-3α-galactoside), 20-hydroxyecdysone 3β-D-glucopyranosyl-[1-3]-β-D-xylopyranoside (limnantheoside C), 20-hydroxyecdysone-3,22-dimethyl ether, cyasterone-3-acetate, 2-dehydro-3-epi-20-hydroxyecdysone, 3-epi-20-hydroxecdysone (coronatasterone), rapisterone D, 3-dehydro-20-hydroxyecdysone, 5β-hydroxy-25,26-didehydroponasterone A, 5β-hydroxystachysterone C, 25-deoxypolypodine B, polypodine B, 25-fluoropolypodine B, 5β-hydroxyabutasterone, 26-hydroxypolypodine B, 29-norsengosterone, sengosterone, 6β-hydroxy-20-hydroxyecdysone, 6α-hydroxy-20-hydroxyecdysone, 20-hydroxyecdysone-6-oxime, ponasterone A 6-carboxymethyloxime, 20-hydroxyecdysone-6-carboxymethyloxime, ajugasterone C, rapisterone B, muristerone A, atrotosterone B, atrotosterone A, turkesterone-2-acetate, punisterone (rhapontisterone), turkesterone, atrotosterone C, 25-hydroxyatrotosterone B, 25-hydroxyatrotosterone A, paxillosterone, rurkesterone-2,22-diacetate, turkesterone-22-acetate, turkesterone-11α-acetate, turkesterone-2, 11α-diacetate, turkesterone-11α-propionate, turkesterone-11α-butanoate, turkesterone-11α-hexanoate, turkesterone-11α-decanoate, turkesterone-11α-laurate, turkesterone-11α-myristate, turkesterone-11α-arachidate, 22-dehydro-12β-hydroxynorsengosterone, 22-dehydro-12β-hydroxycyasterone, 22-dehydro-12β-hydroxysengosterone, 14-deoxy(14α-H)-20-hydroxyecdysone, 20-hydroxyecdysone-14-methyl ether, 14α-perhydroxy-20-hydroxyecdysone, 20-hydroxyecdysone 14,22-dimethyl ether, 20-hydroxyecdysone-2,3,14,22-tetramethyl ether, (20S)-22-deoxy-20,21-dihydroxyecdysone, 22,25-dideoxyecdysone, (22S)-20-(2,2′-dimethylfuranyl)ecdysone, (22R)-20-(2,2′-dimethylfuranyl)ecdysone, 22-deoxyecdysone, 25-deoxyecdysone, 22-dehydroecdysone, ecdysone, 22-epi-ecdysone, 24-methylecdysone (20-deoxymakisterone A), ecdysone-22-hemisuccinate, 25-deoxyecdysone-22-β-D-glucopyranoside, ecdysone-22-myristate, 22-dehydro-20-iso-ecdysone, 20-iso-ecdysone, 20-iso-22-epi-ecdysone, 2-deoxyecdysone, sileneoside E (2-deoxyecdysone 3β-glucoside; blechnoside A), 2-deoxyecdysone-22-acetate, 2-deoxyecdysone-3,22-diacetate, 2-deoxyecdysone-22-3-D-glucopyranoside, 2-deoxyecdysone glucopyranoside, 2-deoxy-21-hydroxyecdysone, 3-epi-22-iso-ecdysone, 3-dehydro-2-deoxyecdysone (silenosterone), 3-dehydroecdysone, 3-dehydro-2-deoxyecdysone-22-acetate, ecdysone-6-carboxymethyloxime, ecdysone-2,3-acetonide, 14-epi-20-hydroxyecdysone-2,3-acetonide, 20-hydroxyecdysone-2,3-acetonide, 20-hydroxyecdysone-20,22-acetonide, 14-epi-20-hydroxyecdysone-2,3,20,22-diacetonide, paxillosterone-20,22-p-hydroxybenzylidene acetal, poststerone, (20S)-dihydropoststerone, (20S)dihydropoststerone, poststerone-20-dansylhydrazine, (20S)-dihydropoststerone-2,3,20-tribenzoate, (20R)-dihydropoststerone-2,3,20-tribenzoate, (20R)dihydropoststerone-2,3-acetonide, (20S)dihydropoststerone-2,3-acetonide, (5α-H)-dihydrorubrosterone, 2,14,22,25-tetradeoxy-5 α-ecdysone, 5 α-ketodiol, bombycosterol, 2α, 3 α,22S,25-tetrahydroxy-5α-cholestan-6-one, (5α-H)-2-deoxy-21-hydroxyecdysone, castasterone, 24-epi-castasterone, (5αα-H)-2-deoxyintegri sterone A, (5α-H)-22-deoxyintegristerone A, (5α-H)-20-hydroxyecdysone, 24,25-didehydrodacryhaninansterone, 25,26-didehydrodacryhainansterone, 5-deoxykaladasterone (dacryhainansterone), (14α-H)-14-deoxy-25-hydroxydacryhainansterone, 25-hydroxydacryhainansterone, rubrosterone, (5β-H)-dihydrorubrosterone, dihydrorubrosterone-17β-acetate, sidisterone, 20-hydroxyecdysone-2,3,22-triacetate, 14-deoxy(14β-H)-20-hydroxyecdysone, 14-epi-20-hydroxyecdysone, 9β,20-dihydroxyecdysone, malacosterone, 2-deoxypolypodine B-3-β-D-glucopyranoside, ajugalactone, cheilanthone B, 2β3β,6α-trihydroxy-5β-cholestane, 2β,3β,6β-trihydroxy-5β-cholestane, 14-dehydroshidasterone, stachysterone B, 2β,3β,9α,20R,22R,25-hexahydroxy-5β(3-cholest-7, 14-dien-6-one, kaladasterone, (14β-H)-14-deoxy-25-hydroxydacryhainansterone, 4-dehydro-20-hydroxyecdysone, 14-methyl-12-en-shidasterone, 14-methyl-12-en-15,20-dihydroxyecdysone, podecdysone B, 2β,3 β,20R,22R-tetrahydroxy-25-fluoro-5β-cholest-8,14-dien-6-one (25-fluoropodecdysone B), calonysterone, 14-deoxy-14,18-cyclo-20-hydroxyecdysone, 9α,14α-epoxy-20-hydroxyecdysone, 9βα, 14 β-epoxy-20-hydroxyecdysone, 9α,14α-epoxy-20-hydroxyecdysone 2,3,20,22-diacetonide, 28-homobrassinolide, iso-homobrassinolide.

In some embodiments, the ligand for use with the methods of the present invention is a compound of the general formula:

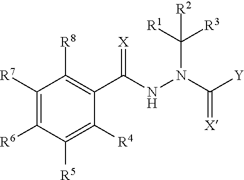

wherein X and X′ are independently O or S;

Y is:

(a) substituted or unsubstituted phenyl wherein the substitutents are independently 1-5H, (C₁-C₄)alkyl, (C₁-C₄)alkoxy, (C₂-C₄)alkenyl, halo (F, Cl, Br, I), (C₁-C₄)haloalkyl, hydroxy, amino, cyano, or nitro; or

(b) substituted or unsubstituted 2-pyridyl, 3-pyridyl, or 4-pyridyl, wherein the substitutents are independently 1-4H, (C₁-C₄)alkyl, (C₁-C₄)alkoxy, (C₂-C₄)alkenyl, halo (F, Cl, Br, I), (C₁-C₄)haloalkyl, hydroxy, amino, cyano, or nitro;

R¹ and R² are independently: H; cyano; cyano-substituted or unsubstituted (C₁-C₇) branched or straight-chain alkyl; cyano-substituted or unsubstituted (C₂-C₇) branched or straight-chain alkenyl; cyano-substituted or unsubstituted (C₃-C₇) branched or straight-chain alkenylalkyl; or together the valences of R¹ and R² form a (C₁-C₇)cyano-substituted or unsubstituted alkylidene group (R^{a}R^{b}C═) wherein the sum of non-substituent carbons in R^{a} and R^{b} is 0-6;

R³ is H, methyl, ethyl, n-propyl, isopropyl, or cyano;

R⁴, R⁷, and R⁸ are independently: H, (C₁-C₄)alkyl, (C₁-C₄)alkoxy, (C₂-C₄)alkenyl, halo (F, Cl, Br, I), (C₁-C₄)haloalkyl, hydroxy, amino, cyano, or nitro; and

R⁵ and R⁶ are independently: H, (C₁-C₄)alkyl, (C₂-C₄)alkenyl, (C₃-C₄)alkenylalkyl, halo (F, Cl, Br, I), C₁-C₄ haloalkyl, (C₁-C₄)alkoxy, hydroxy, amino, cyano, nitro, or together as a linkage of the type (—OCHR⁹CHR¹⁰O—) form a ring with the phenyl carbons to which they are attached; wherein R⁹ and R¹⁰ are independently: H, halo, (C₁-C₃)alkyl, (C₂-C₃)alkenyl, (C₁-C₃)alkoxy(C₁-C₃)alkyl, benzoyloxy(C₁-C₃)alkyl, hydroxy(C₁-C₃)alkyl, halo(C₁-C₃)alkyl, formyl, formyl(C₁-C₃)alkyl, cyano, cyano(C₁-C₃)alkyl, carboxy, carboxy(C₁-C₃)alkyl, (C₁-C₃)alkoxycarbonyl(C₁-C₃)alkyl, (C₁-C₃)alkylcarbonyl(C₁-C₃)alkyl, (C₁-C₃)alkanoyloxy(C₁-C₃)alkyl, amino(C₁-C₃)alkyl, (C₁-C₃)alkylamino(C₁-C₃)alkyl (—(CH₂)ₙR^{c}R^{c}), oximo (—CH═NOH), oximo(C₁-C₃)alkyl, (C₁-C₃)alkoximo (—C═NOR^{d}), alkoximo(C₁-C₃)alkyl, (C₁-C₃)carboxamido (—C(O)NR^{e}R^{f}), (C₁-C₃)carboxamido(C₁-C₃)alkyl, (C₁-C₃)semicarbazido (—C═NNHC(O)NR^{e}R^{f}), semicarbazido(C₁-C₃)alkyl, aminocarbonyloxy (—OC(O)NHR^{g}), aminocarbonyloxy(C₁-C₃)alkyl, pentafluorophenyloxycarbonyl, pentafluorophenyloxycarbonyl(C₁-C₃)alkyl, p-toluenesulfonyloxy(C₁-C₃)alkyl, arylsulfonyloxy(C₁-C₃)alkyl, (C₁-C₃)thio(C₁-C₃)alkyl, (C₁-C₃)alkylsulfoxido(C₁-C₃)alkyl, (C₁-C₃)alkylsulfonyl(C₁-C₃)alkyl, or (C₁-C₅)trisubstituted-siloxy(C₁-C₃)alkyl (—(CH₂)ₙSiOR^{d}R^{e}R^{g}); wherein n=1-3, R^{c} and R^{d} represent straight or branched hydrocarbon chains of the indicated length, R^{e}, R^{f} represent H or straight or branched hydrocarbon chains of the indicated length, R^{g} represents (C₁-C₃)alkyl or aryl optionally substituted with halo or (C₁-C₃)alkyl, and R^{c}, R^{d}, R^{e}, R^{f}, and R^{g} are independent of one another;

provided that

i) when R⁹ and R¹⁰ are both H, or

ii) when either R⁹ or R¹⁰ are halo, (C₁-C₃)alkyl, (C₁-C₃)alkoxy(C₁-C₃)alkyl, or benzoyloxy(C₁-C₃)alkyl, or

iii) when R⁵ and R⁶ do not together form a linkage of the type (—OCHR⁹CHR¹⁰O—),

then the number of carbon atoms, excluding those of cyano substitution, for either or both of groups R¹ or R² is greater than 4, and the number of carbon atoms, excluding those of cyano substitution, for the sum of groups R¹, R², and R³ is 10, 11, or 12.

### Polynucleotides of the Invention

A novel ecdysone receptor/retinoid X receptor-based ligand inducible polypeptide coupler system of the invention may comprise an expression cassette having a polynucleotide sequence that encodes a hybrid polypeptide comprising an EcR nuclear receptor polypeptide component and an inactive signaling domain or a RXR nuclear receptor polypeptide component and an inactive signaling domain. These expression cassettes, the polynucleotides they comprise, and the hybrid polypeptides they encode are useful as components of an EcR/RXR-based ligand inducible polypeptide coupler system to modulate the activity of signaling domains within a host cell.

Thus, the present invention provides an isolated polynucleotide that encodes a hybrid polypeptide having an EcR nuclear receptor polypeptide component and an inactive signaling domain and/or a RXR nuclear receptor polypeptide component and an inactive signaling domain. The isolated polynucleotides that encode the EcR and/or RXR nuclear receptor polypeptide components of the invention comprise, but are not limited to, the polynucleotide sequences described above, including wild-type, truncated, and substitution mutation-containing EcR polypeptides described herein and/or wild-type, truncated, and chimeric RXR polypeptides described herein, including combinations thereof.

In addition, the isolated polynucleotides of the present invention can have polynucleotide sequences that encode signaling domains, including those described herein. The polynucleotide sequences of such signaling domains are readily accessible via publically available databases that are known to those of ordinary skill in the art. Such databases include, but are not limited to, GenBank (ncbi.nlm.nih.gov/genbank), UniProt (uniprot.org), and the like.

### Polypeptides of the Invention

The novel ecdysone receptor/retinoid X receptor-based ligand inducible polypeptide coupler system of the invention can comprise an expression cassette having a polynucleotide that encodes a hybrid polypeptide comprising an EcR polypeptide and/or an inactive signaling domain or a RXRpolypeptide and an inactive signaling domain. These expression cassettes, the polynucleotides they comprise, and the hybrid polypeptides they encode are useful as components of an EcR/RXR-based ligand inducible polypeptide coupler system to modulate the activity of signaling domains within a host cell.

Thus, the present invention also relates to an isolated hybrid polypeptide having an EcR polypeptide and an inactive signaling domain (e.g., signaling molecules, signaling domains, complementary protein fragments, protein subunits, and natural or engineered partial or truncated proteins) and/or a RXR polypeptide and an inactive signaling domain (e.g., signaling molecules, signaling domains, complementary protein fragments, protein subunits, and natural or engineered partial or truncated proteins) according to the invention. The EcR and/or RXR domains of the isolated polypeptides of the invention can comprise, but are not limited to, polypeptide sequences described herein, including wild-type, truncated, functional fragments, and substitution mutation-containing EcR ligand binding domains described herein and/or wild-type, truncated, functional fragments, and chimeric RXR polypeptides described herein, including combinations thereof.

In addition, the isolated hybrid polypeptides of the invention can have signaling domains (e.g., signaling molecules, signaling domains, complementary protein fragments, protein subunits, and natural or engineered partial or truncated proteins), including those described herein. The amino acid sequences of such signaling domains are readily accessible via publically available databases that are known to those of ordinary skill in the art. Such databases include, but are not limited to, GenBank (ncbi.nlm.nih.gov/genbank), UniProt (uniprot.org), and the like.

### Expression Vectors of the Invention

The novel ecdysone receptor/retinoid X receptor-based ligand inducible polypeptide coupler system of the invention comprises an expression cassette comprising a polynucleotide that encodes a hybrid polypeptide comprising an EcR ligand binding domain and an inactive signaling domain (e.g., signaling molecules, signaling domains, complementary protein fragments, protein subunits, and natural or engineered partial or truncated proteins) and/or a RXR polypeptide and an inactive signaling domain (e.g., signaling molecules, signaling domains, complementary protein fragments, protein subunits, and natural or engineered partial or truncated proteins). These expression cassettes, the polynucleotides they comprise, and the hybrid polypeptides they encode can be expressed in a host cell using any suitable expression vector. Suitable expression vectors are well known to those of ordinary skill in the art and the choice of expression vector and optimal expression conditions in view of the desired host cell can be readily determined by one of ordinary skill in the art. Exemplary expression vectors that can be employed with the invention include, but are not limited to, the expression vectors described above.

### Host Cells

As described above, the ligand inducible polypeptide coupler system of the present invention may be used to modulate protein-protein interaction, i.e., association, within a host cell. Modulation in transgenic host cells may be useful for the modulation of various proteins of interest. Thus, the invention provides an isolated host cell comprising a ligand inducible polypeptide coupler system according to the invention. The present invention also provides an isolated host cell comprising a ligand inducible polypeptide coupler system comprising one or more expression cassettes according to the invention. The invention also provides an isolated host cell comprising a polynucleotide or a polypeptide. The isolated host cell may be either a prokaryotic or a eukaryotic host cell.

In certain embodiments, the isolated host cell is a prokaryotic host cell or a eukaryotic host cell. In another specific embodiment, the isolated host cell is an invertebrate host cell or a vertebrate host cell. Such host cells may be selected from a bacterial cell, a fungal cell, a yeast cell, a nematode cell, an insect cell, a fish cell, a plant cell, an avian cell, an animal cell, and a mammalian cell. More specifically, the host cell is a yeast cell, a nematode cell, an insect cell, a plant cell, a zebrafish cell, a chicken cell, a hamster cell, a mouse cell, a rat cell, a rabbit cell, a cat cell, a dog cell, a bovine cell, a goat cell, a cow cell, a pig cell, a horse cell, a sheep cell, a simian cell, a monkey cell, a chimpanzee cell, or a human cell. Examples of host cells include, but are not limited to, fungal or yeast species such as Aspergillus, Trichoderma, Saccharomyces, Pichia, Candida, Hansenula, or bacterial species such as those in the genera Synechocystis, Synechococcus, Salmonella, Bacillus, Acinetobacter, Rhodococcus, Streptomyces, Escherichia, Pseudomonas, Methylomonas, Methylobacter, Alcaligenes, Synechocystis, Anabaena, Thiobacillus, Methanobacterium and Klebsiella, animal, and mammalian host cells.

In certain embodiments, the host cell is a yeast cell selected from the group consisting of a Saccharomyces, a Pichia, and a Candida host cell. In a specific embodiment, the host cell is a Caenorhabditis elegans nematode cell. In another specific embodiment, the host cell is a hamster cell. In another embodiment, the host cell is a murine cell. In another embodiment, the host cell is a monkey cell. In another specific embodiment, the host cell is a human cell.

In another embodiment, the host cell is a mammalian cell selected from the group consisting of a hamster cell, a mouse cell, a rat cell, a rabbit cell, a cat cell, a dog cell, a bovine cell, a goat cell, a cow cell, a pig cell, a horse cell, a sheep cell, a monkey cell, a chimpanzee cell, and a human cell. In certain embodiments the host cell is an immortalized cell, an immune cell, or a T-cell.

Host cell transformation is well known in the art and may be achieved by a variety of methods including but not limited to electroporation, viral infection, plasmid/vector transfection, non-viral vector mediated transfection, particle bombardment, and the like. Expression of desired gene products involves culturing the transformed host cells under suitable conditions and inducing expression of the transformed gene. Culture conditions and gene expression protocols in prokaryotic and eukaryotic cells are well known in the art. Cells may be harvested and the gene products isolated according to protocols specific for the gene product.

In addition, a host cell may be chosen that modulates the expression of the inserted polynucleotide, or modifies and processes the polypeptide product in the specific fashion desired.

The invention also relates to a non-human organism comprising an isolated host cell according to the invention. In certain embodiments, the non-human organism is selected from the group consisting of a bacterium, a fungus, a yeast, an animal, and a mammal. In some embodiments, the non-human organism is a yeast, a mouse, a rat, a rabbit, a cat, a dog, a bovine, a goat, a pig, a horse, a sheep, a monkey, or a chimpanzee.

In a certain embodiments, the non-human organism is a yeast selected from the group consisting of Saccharomyces, Pichia, and Candida. In another embodiment, the non-human organism is a Mus musculus mouse.

### Methods for Modulating Post-Translational Activity

Applicant's invention encompasses methods of incorporating LIPCs into polypeptides (generating heterologous polypeptides) to modulate activity of signaling domains in host cells. Specifically, Applicant's invention provides a method of inducing or inhibiting activation of signaling proteins and pathways via incorporation of LIPC components into signal activating or inhibiting polypeptides expressed in a host cell, and contacting the host cell with a ligand, to bring about the signal transduction activation or inhibition.

In one embodiment, cell signal transduction is activated by LIPC-induced dimerization of oligomerization of signaling domains (e.g., signaling molecules, signaling domains, complementary protein fragments, protein subunits, and natural or engineered partial or truncated proteins).

In another embodiment, cell signal transduction is inhibited by LIPC-induced dimerization of an inhibitory polypeptide to a cell signal transduction (activation) pathway polypeptide. In one embodiment, a component of the LIPC alone (e.g., an EcR or RxR/USP polypeptide) is the inhibitory polypeptide.

In one embodiment, LIPC polypeptides are used to modulate (i.e., activate or inhibit) intracellular protein-protein interactions. In another embodiment, LIPC polypeptides are used to modulate (i.e., activate or inhibit) extracellular protein-protein interactions. In another embodiment, LIPC polypeptides are used to modulate (i.e., activate or inhibit) transmembrane protein-protein interactions.

Genes and proteins of interest for expression and modulation of activity via LIPC in a host cell may be endogenous genes or heterologous genes. Nucleic acid or amino acid sequence information for a desired gene or protein can be located in one of many public access databases, for example, GenBank, EMBL, Swiss-Prot, and PIR, or in numerous biology-related journal publications. Thus, those of ordinary skill in the art have access to nucleic acid sequence and/or amino acid sequence information for virtually all known genes and proteins. Such information can then be used to construct the desired constructs for expression of the protein of interest (e.g., signaling domain) within the expression cassettes used in Applicant's methods described herein.

Examples of genes and proteins of interest for expression in a host cell using Applicant's methods include, but are not limited to, enzymes, reporter genes, structural proteins, transmembrane receptors, nuclear receptor, genes encoding polypeptides or signaling domains involved in a disease, a disorder, a dysfunction, a genetic defect, antibodies, targets for drug discovery, and proteomics analyses and applications, and the like.

Among the many and varied manners in which a Ligand Inducible Polypeptide Coupler (LIPC) of the present invention may be utilized and incorporated into control of or effect upon a biological cell signal transduction system, one general example is substitution of any other ligand inducible dimerization or multimerization system (such as those utilizing FK506 or rapamycin) with LIPC components of the present invention.

A specific example in which a Ligand Inducible Polypeptide Coupler (LIPC) of the present invention may be utilized and incorporated into control of a biological cell signal transduction system, is for use in generating an inducible cell “kill switch” or “suicide switch”; such as has been proposed for use in destroying genetically modified T cells (e.g., chimeric antigen receptor (CAR) T cells).

Some examples of the above-referenced sytems are reviewed and described in:
Publication number WO2015157252 (PCT/US2015/024671) “Treatment of Cancer Using Anti-CD19 Chimeric Antigen Receptor”;
Publication number WO2011146862 (PCT/US2011/037381) “Methods For Inducing Selective Apoptosis”;
Publication number WO2014164348 (PCT/US2014/022004) “Modified Caspase Polypeptides And Uses Thereof”;
Publication number WO2014151960 (PCT/US2014/026734) “Methods For Controlling T cell Proliferation”;
Publication number WO2014127261 (PCT/US2014/016527) “Chimeric Antigen Receptor And Methods of Use Therefore”;
Auslander et al., “From gene switches to mammalian designer cells: Present and future prospects”, Trends in Biotechnology, vol. 31, no. 3 pp. 155-168 (2013);
Chakravarti, et al., “Synthetic biology in cell-based cancer immunotherapy”, Trends in Biotechnology, vol. 33, issue 8, pp. 449-461 (2015);
Ciceri, et al., “Infusion of suicide-gene-engineered donor lymphocytes after family haploidentical haemopoietic stem-cell transplantation for leukaemia (the TK007 trial): A non-randomised phase I-II study”, Lancet Oncol. 10, 489-500 (2009); Medline doi:10.1016/S1470-2045(09)70074-9;
Wu, et al. “Remote control of therapeutic T cells through a small molecule-gated chimeric receptor”, 10.1126/science.aab40 77 (2015);
Vilaboa, et al.,“Gene switches for deliberate regulation of transgene rxpression: Recent advances in system development and uses”, J Genet Syndr Gene Ther 2:107. doi:10.4172/2157-7412.1000107;
Stieger, et al., “In vivo regulation using tetracycline-regulatable systems”, Adv Drug Deliv Rev 61: 527-541 (2009);
   each of the above-cited references are hereby incorporated by reference herein.

### EXAMPLE 1

### LIPC Activated Luciferase

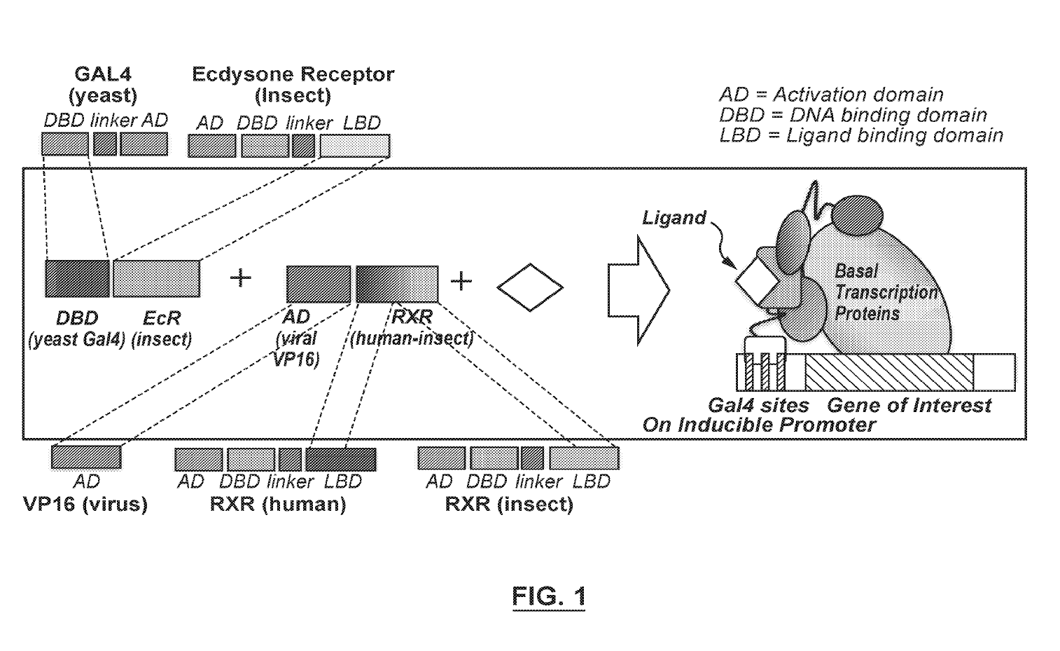

Applicant's RheoSwitch genetic switch technology drives transcription in the presence of an activating ligand. The ligand binds the EcR ligand-binding domain portion of a GAL4-EcR fusion protein, which recruits an RXR-VP16 component (see, e.g., FIG. 1). The inventors have determined that EcR and RXR domains, such as those used in the RheoSwitch® system, can act as a ligand inducible polypeptide coupler, driving association of other proteins fused to the EcR and RXR domains.

The ligand inducible polypeptide coupler operates differently than a transcriptional gene switch. Using the LIPC system, protein-protein interaction is controlled, not gene expression. Levels of activation may be regulated in a dose-dependent fashion as controlled via concentration and quantity of small molecule ligand administration.

As described herein, a split firefly luciferase system has been used to demonstate ligand-inducible EcR-RXR fusion protein association. This system represents a new method for employing protein switch components. Such a switch is fundamentally different from gene transcriptional activation switches, which are directed to controlling protein expression. Controlling protein-protein interaction, i.e., association, requires careful and specific engineering, as the molecules to be associated (e.g., dimerized or oligomerized) must have some differential function when associated and have limited, or no natural affinity for each other under the non-ligand conditions.

### Methods and Analytical Approach

A series of EcR and RXR fusions (some with a split firefly luciferase (fLuc)) proteins have been conceived and designed (see FIGS. 2-6). Split luciferase systems have been used to investigate protein-protein interactions in other cell systems (see, e.g., Luker, et al., Proc. Natl. Acad. Sci. U.S.A. 101(33): 12288-93 (2004), Paulmurugan and Gambhir, Anal. Chem. 75(5):1295-302 (2005), Fujikawa and Kato, Plant J. 52(1):185-95 (2007), and Leng, et al., PLos One 8(4):e62230 (2013), each of which is incorporated by reference herein in its entirety). The split luciferase system has an advantage over split GFP systems in that the components do not covalently bind when associated, allowing for off-rate analysis.

The fLuc protein was divided into two pieces having no intrinsic affinity for each other (such that it is inactive until brought into close association by fused protein elements) for use as a system of testing protein-protein association. HEK293 cells were transfected with the split fLuc fused to EcR and RXR domains as follows:

### Transfection

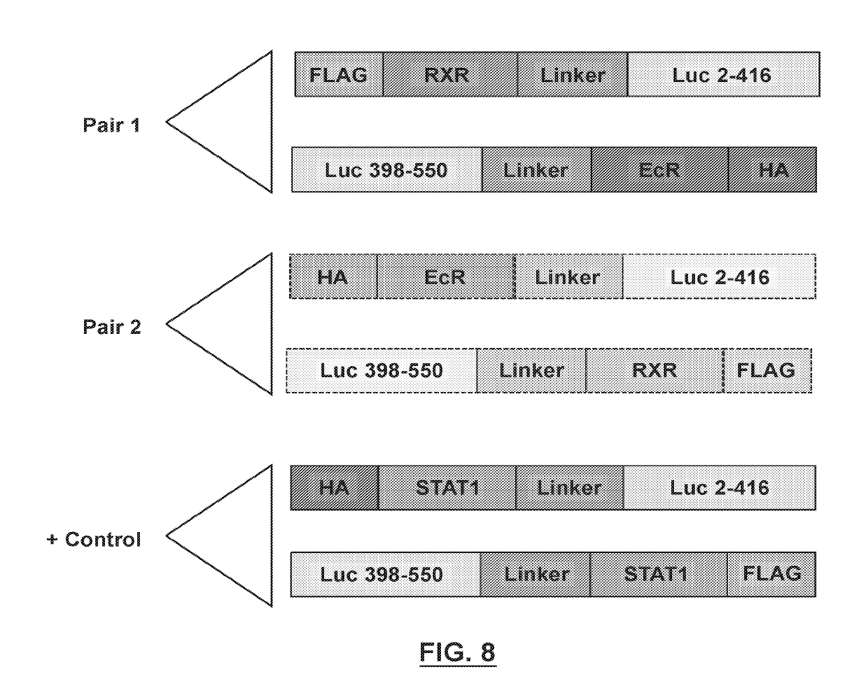

A day before transfection, 10,000 cells (293T cells) were plated into each well of a 96 well plate containing 100 μl of growth medium (Dulbecco's Modified Eagle's Medium with 10% Fetal Bovine Serum) without antibiotics. Plasmids in pairs, RxR Nluc with Cluc EcR and EcR_ Nluc with Cluc_ RxR (see FIG. 8; amino acid sequences for the constructs depicted in FIG. 8 are provided as SEQ ID NOs: 87-92, respectively. SEQ ID NOs: 91 and 92 correspond to the EcR and RXR amino sequences, respectively, employed in the constructs of FIG. 8), were transfected with Lipofectamine® 2000, according to manufacturer's specifications. Briefly, individual plasmid DNA (0.2 μg) and 0.5 μl of Lipofectamine 2000® was diluted in 25.0 μl of OptiMEM® I Reduced Serum Medium and incubated for 5 minutes at room temperature, volumes were doubled for co-transfections. Diluted plasmid DNA was combined with diluted Lipofectamine® 2000 and incubated for 20 minutes at room temperature. 50 μl of the DNA/Lipofectamine® 2000 complex was added to each well of the 96 well plate. Cells were incubated at 37° C. in a 5% CO₂ incubator for 24 hours, prior to addition of the activating ligand Veledimex.

### Bioluminescence Assay

Twenty four hours (24hrs) post-transfection, cell culture media from each well of the 96-well plate was replaced with 100 nM Veledimex activating ligand and Dimethyl sulfoxide-DMSO (negative control). Each component was diluted thousand fold in Dulbecco's Modified Eagle's Medium with 10% Fetal Bovine Serum and incubated for 6 hrs at 37° C. in a 5% CO₂ incubator. ONE-Glo™ Luciferase Assay Buffer was combined with ONE-Glo™ Luciferase Assay Substrate, which contains 5′-Fluoroluciferin (a luciferin analog). This reagent was frozen after reconstitution and stored at −20° C. until use. Luciferase ONE-Glo™ Luciferase substrate was thawed to room temperature in a water bath. The 96-well plate was removed from the incubator and equilibrated for ˜1 hr., at room temperature, plate bottom covered with Corning® 96 well microplate aluminum sealing tape, before addition of the substrate. 100 μl of the ONE-Glo™ Luciferase reagent buffer was added to each well of the 96-well plate. After 3 minutes of incubation at room temperature to ensure complete cell lysis, the 96-well plate was placed in GloMax™ 96 Microplate Luminometer to measure bioluminescence from each well.

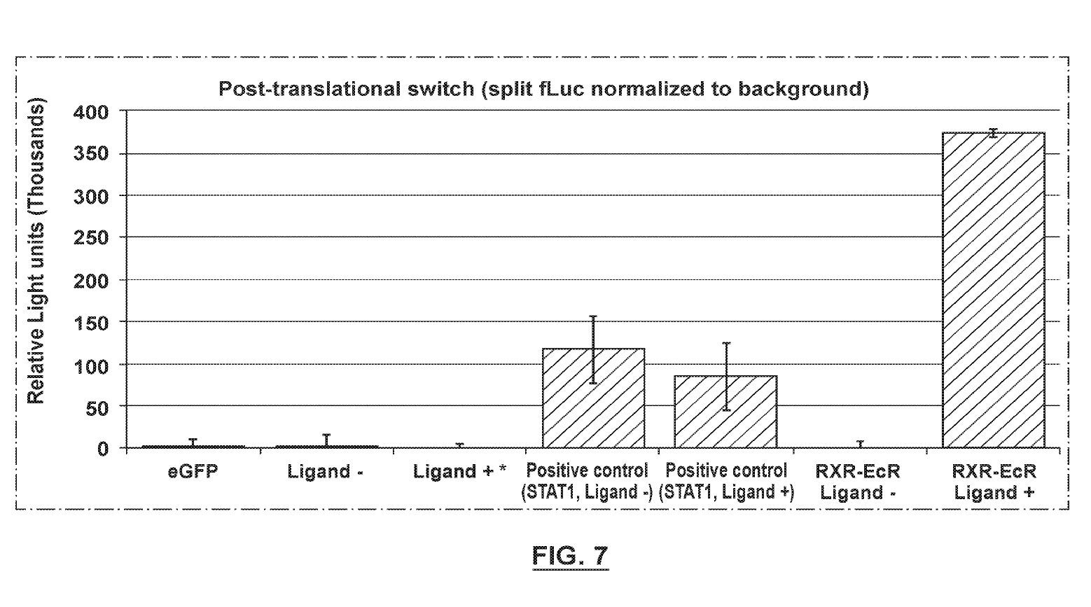

In the absence of activating ligand, only background signal was observed. fLuc signal was detected following addition of activating ligand (FIG. 7; RXR-EcR Ligand − and +, far right). The fLuc assay was performed 6 hours after addition of activating ligand. A construct using STAT1, a protein shown to homodimerize using the identical split fLuc system (see, e.g., Luker, et al., (2004)), was included for a positive control (see Table 2). Signal of the positive control appears to be unaffected by activating ligand (FIG. 7; Positive control, STAT1. Ligand − and +). As negative controls, eGFP and activating ligand alone (vehicle only) samples gave only background readings (FIG. 7; eGFP, Ligand -, and Ligand +). It should be noted that in this run the Ligand + well had a cell count slightly lower than the other wells (FIG. 7; Ligand +*). Data was normalized against mean background and reported in relative light units. Standard fLuc was run as an additional control.

Upon addition of activating ligand, a clear fLuc signal is generated using the EcR and RXR LIPC system. Only background is observed in the absence of ligand (see FIG. 7).

TABLE 2
Experimental Setup for Split Luciferase System

| | | | | | fLuc |
|---|---|---|---|---|---|
| | Group | Vector 1 | Vector 2 | Treatment | Activity |
| | −control | eGFP | −− | −− | − |
| | −control | mock | −− | −− | − |
| | −control | mock | −− | Ligand | − |
| split fLuc | +control | STAT1-fLuc | fLuc-STAT1 | −− | + |
| System | +control | STAT1-fLuc | fLuc-STAT1 | −− | + |
| | Exp | RXR-fLuc | fLuc-EcR | −− | − |
| | Exp | RXR-fLuc | fLuc-EcR | Ligand | + |
| | +control | Full fLuc | −− | −− | +++ |

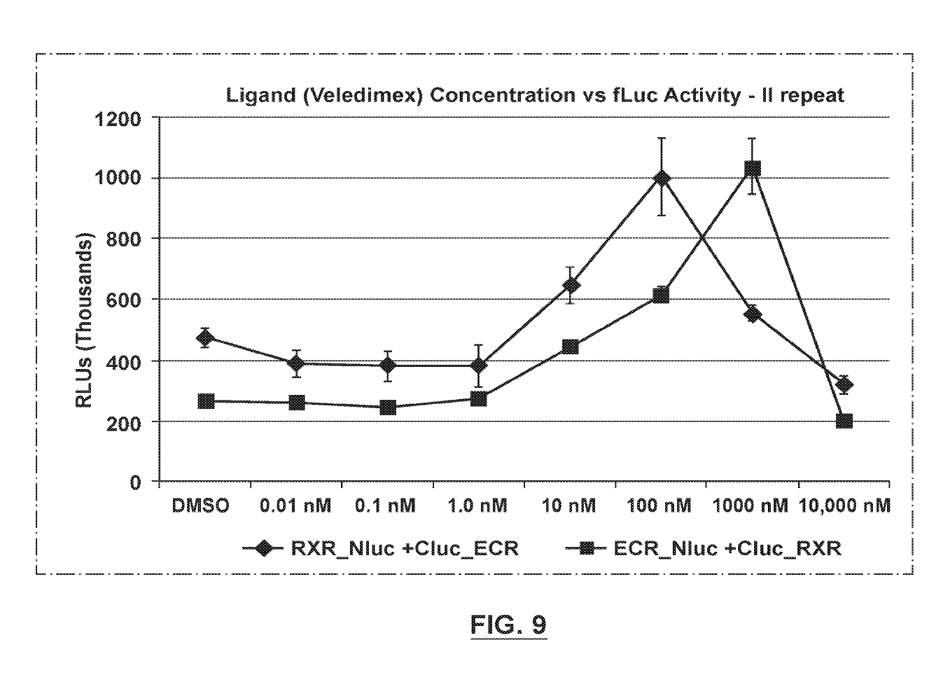

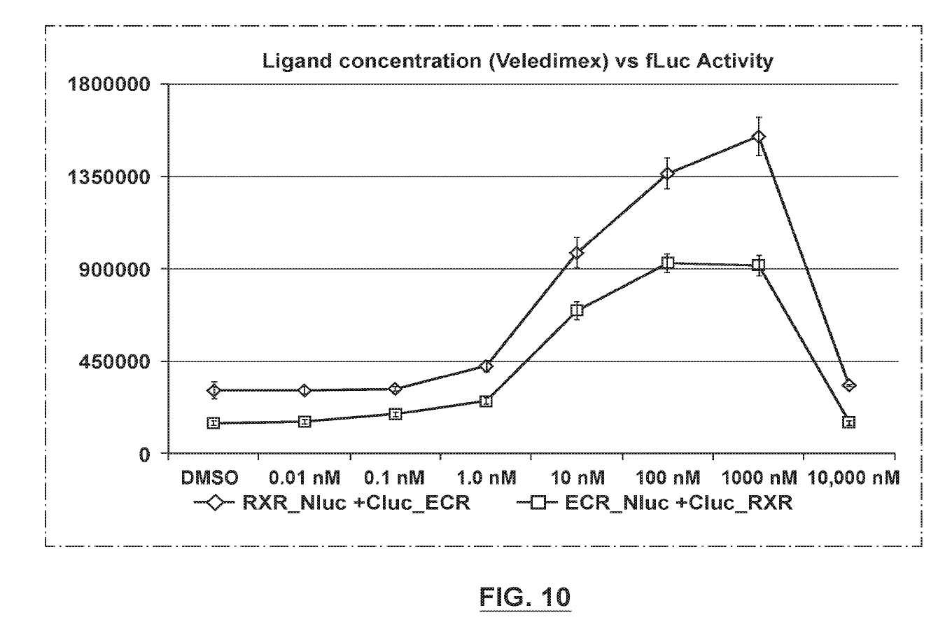

Positive signal should only be observed in complementing pairs of vectors that have been exposed to activating ligand, driving association of EcR and RXR components and restoring fLuc activity. Ligand dose response curves are shown in FIG. 9 and FIG. 10. This work serves to demonstrate EcR and RXR' s ability to drive ligand inducible polypeptide couping, i.e., ligand-mediated association or oligomerization, that can control protein-protein interactions and associations at a post-translational level.

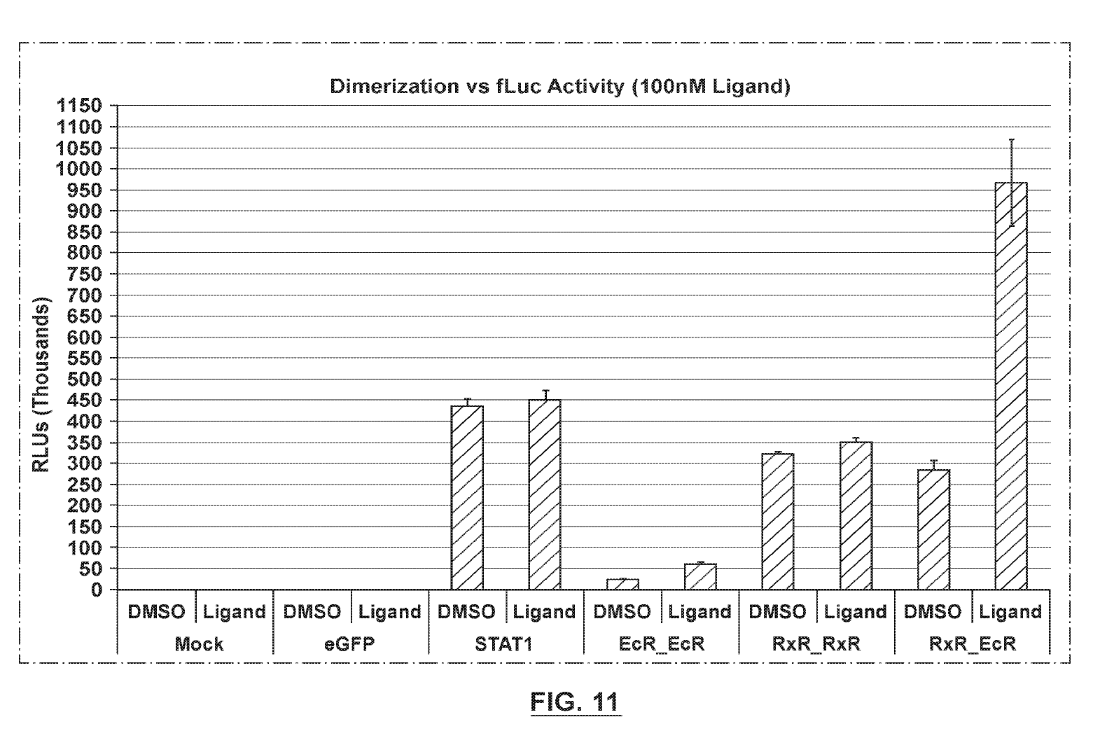

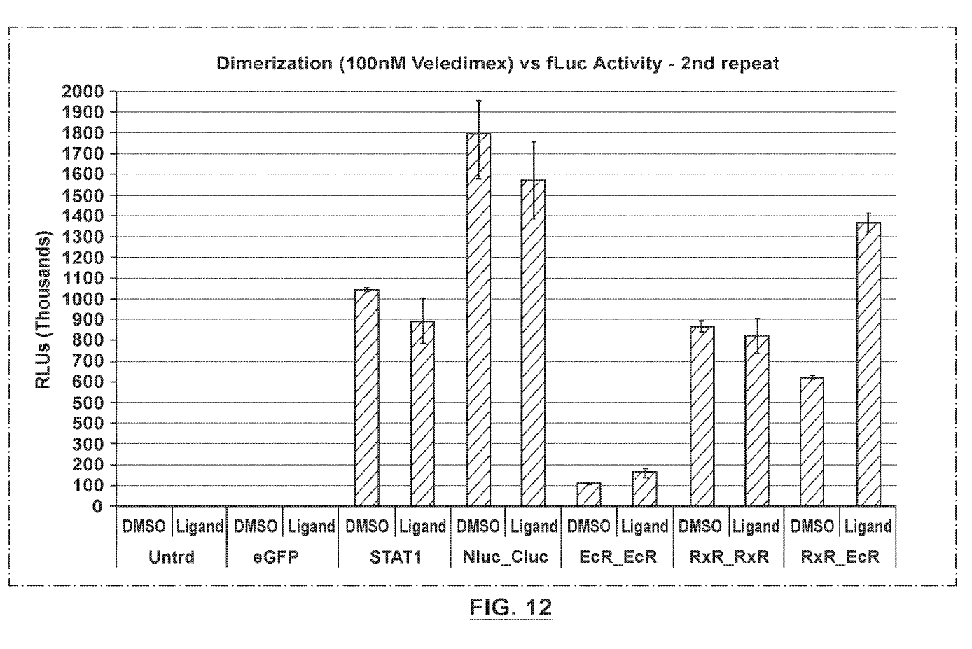

EcR dimerization induction via Veledimex ligand results are shown in FIGS. 11 and FIG. 12.

Data generated by the present system can be used to inform molecular designs for additional systems going forward. Additional uses of such a system include, but are not limited to, screening for signaling domains (e.g., signaling molecules, signaling domains, complementary protein fragments, protein subunits, and natural or engineered partial or truncated proteins) that are activated through protein-protein interaction.

Based on the experiments and results with the intracellular split fLuc reporter, new designs for LIPC systems will be undertaken. Additional configurations of EcR, RXR, and split fLuc elements will be assayed to demonstrate additional pairings. All of this information can be used to inform the generation of comparative models of the proteins that can in turn provide guidance for future designs. The current split fLuc vectors will also be tested in other important cell types for consistent activity. As the proteins are constitutively expressed in the present example, the dimerization event should be rapid when activating ligand is administered. Conversely, given that the fLuc halves have no affinity for each other and do not covalently interact, this system could also be used to examine off-rate kinetics following removal of activating ligand. Both signal onset and decay experiments are envisaged and being undertaken.

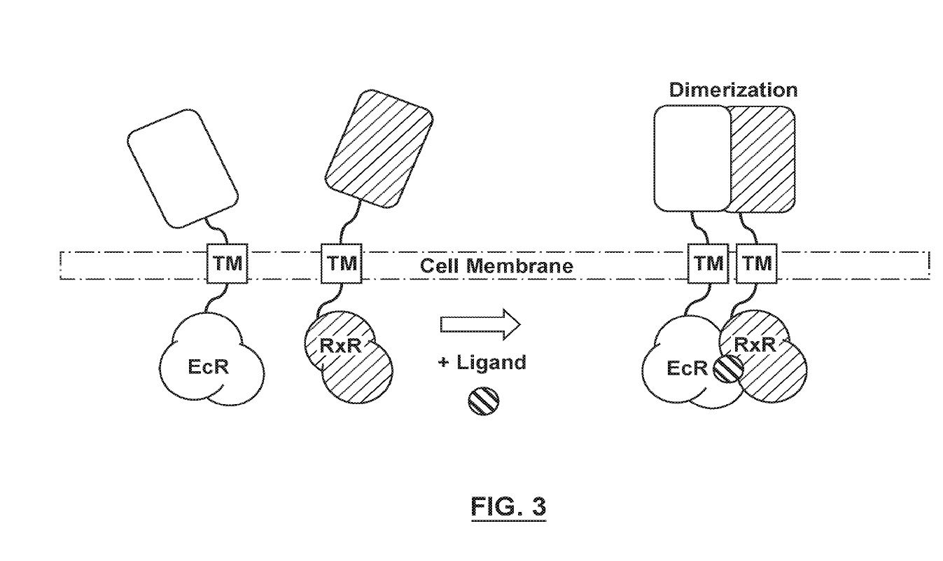

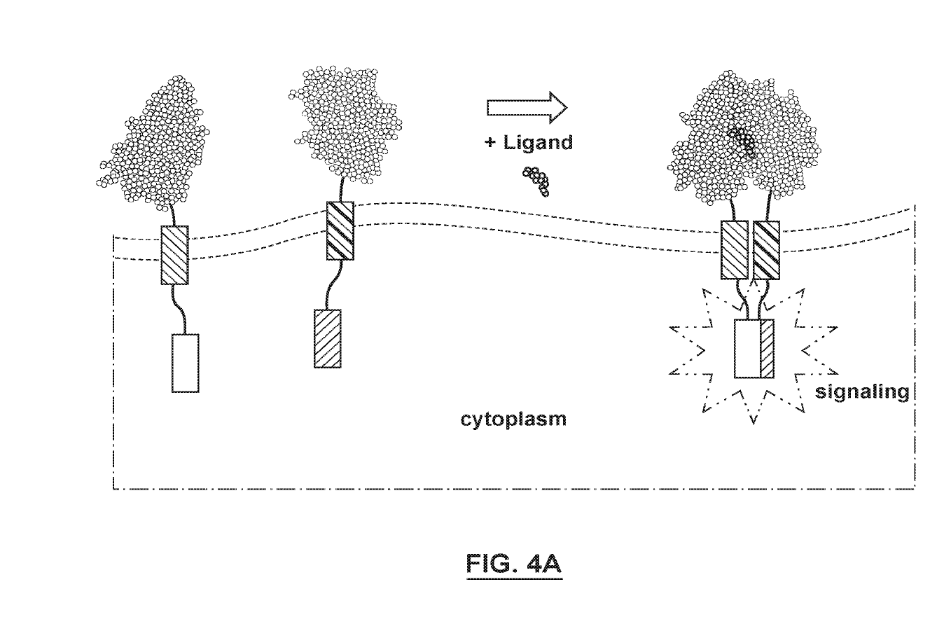

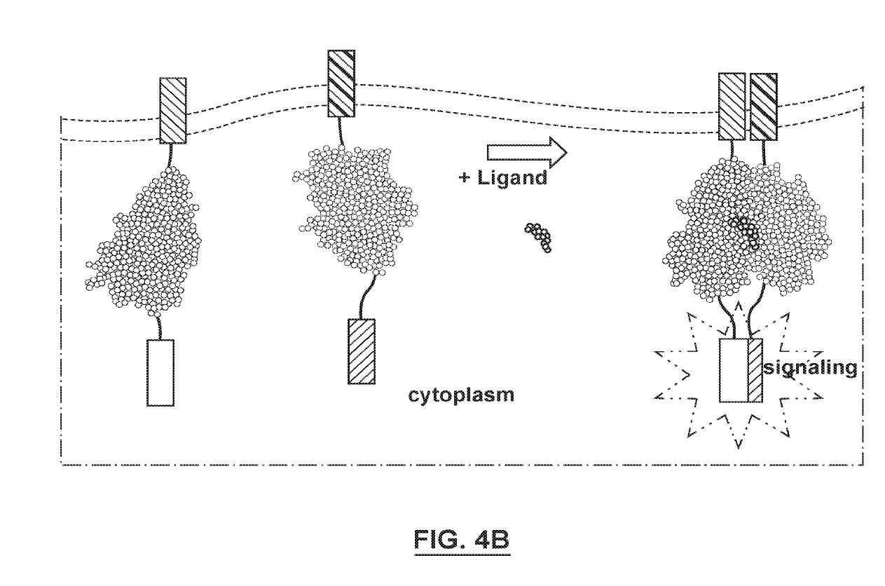

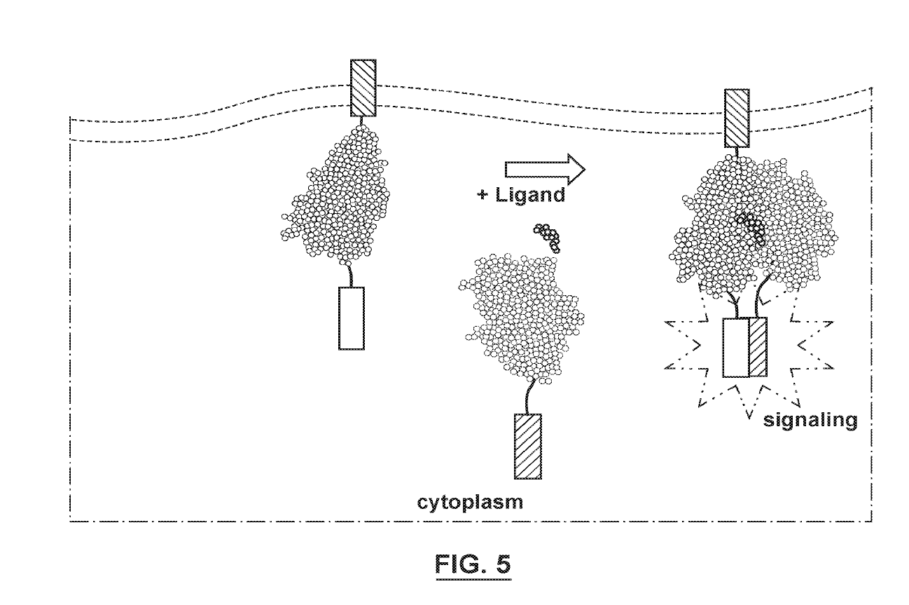

Further, additional LIPC designs are being pursued. Some of the designs are similar to those of the fLuc system above, with differences being, for example, that the molecules involved in the interaction can be single-pass type I transmembrane proteins. Initial designs and experiments will be with EcR and RXR localized intracellularly with at least portions of the fused proteins located extracellularly (see FIG. 3). Several additional configurations, however, can also be designed and tested depending on the actual assay readout. Additional designs include, but are not limited to, molecules with a transmembrane domain fused to EcR and RXR with EcR and RXR localized extracellularly and the fused proteins located intracellularly (see FIG. 4). Another configuration is where EcR and RXR components are fused to transmembrane domains yet the EcR, RXR, and fused signaling domains are all located intracellularly (see FIG. 5). Note that additional signaling domains, apart from fLuc, can be employed in the various configurations outlined above.

Further research will include experiments to understand on- and off-rates, optimal expression levels required to drive desired activation effects, and reduce (if needed) potential background (e.g., biological effects of the unpartnered proteins in the absence of ligand).

### EXAMPLE 2

### Ligand-Induced Dimerization of Nuclear Receptor Components

Experiments were performed to test if nuclear receptor domains (i.e., EcR and RxR polypeptides) could be induced to homodimerize upon addition of ligand (FIGS. 11 and 12). STAT1 was used as control polypeptide since it is reported to self dimerize independent of ligand addition. Abbreviations in the figures are:

“EcR” is Ecdysone receptor;

“EcR-EcR” means “EcR_Nluc+Cluc_EcR” which is a luciferase polypeptide split into two halves, such that an EcR polypeptide is fused to the N-terminus of a luciferase polypeptide fragment (EcR_Nluc) and another fragment of luciferase has an EcR polypeptide fused to its C-terminal end (Cluc_EcR); thereby activating luciferase (generation of bioluminescence) upon EcR homodimerization;

“RxR” is Retinoid X receptor;

“Mock” means no vector added;

“eGFP” is enhanced GFP (used as a negative control);

“RxR_EcR” means “EcR_Nluc+Cluc_RXR” which is a luciferase polypeptide split into two halves, such that an EcR polypeptide is fused to the N-terminus of a luciferase polypeptide fragment (EcR₁₃ Nluc) and another fragment of luciferase has an RxR polypeptide fused to its C-terminal end (Cluc RxR); thereby activating luciferase (generation of bioluminescence) upon EcR homodimerization;

The results (FIGS. 11 and 12) indicate that EcR domain can be induced to homo dimerize upon ligand addition. However, the difference in bioluminescence signal was relatively low, which may be due to low affinity between the EcR domains by themselves. Based on the bioluminescence output, there was a statistically significant homodimerization of EcR domains upon ligand addition. In contrast, RxR domains were, surprisingly, observed to homodimerize independent of ligand. Moreover, the strongest signal (bioluminescence) was observed via heterodimerization of RxR and EcR domains induced by the ligand. Accordingly, these results indicate a relatively strong interaction between RxR and EcR domains via heterodimerization induced by ligand. Indeed, although homodimerization of each domain was of more limited affinity, it was surprising to observe and discover the ligand-independent homodimerization of RxR domains.

Unless defined otherwise, all technical and scientific terms and any acronyms used herein have the same meanings as commonly understood by one of ordinary skill in the art in the field of this invention.

All references cited herein are incorporated by reference herein to the full extent allowed by law. The discussion of those references is intended merely to summarize the assertions made by their authors. No admission is made that any reference (or a portion of any reference) is relevant art. Applicants reserve the right to challenge the accuracy and pertinence of any cited reference.

APPENDIX I
SEQUENCES

| | |
|---|---|
| <210> SEQ ID NO: 1 | |
| <211> LENGTH: 1054 | |
| <212> TYPE: DNA | |
| <213> ORGANISM: Choristoneura fumiferana | |
| <400> SEQUENCE: 1 | |
| cctgagtgcg tagtacccga gactcagtgc gccatgaagc ggaaagagaa gaaagcacag | 60 |
| aaggagaagg acaaactgcc tgtcagcacg acgacggtgg acgaccacat gccgcccatt | 120 |
| atgcagtgtg aacctccacc tcctgaagca gcaaggattc acgaagtggt cccaaggttt | 180 |
| ctctccgaca agctgttgga gacaaaccgg cagaaaaaca tcccccagtt gacagccaac | 240 |
| cagcagttcc ttatcgccag gctcatctgg taccaggacg ggtacgagca gccttctgat | 300 |
| gaagatttga agaggattac gcagacgtgg cagcaagcgg acgatgaaaa cgaagagtct | 360 |
| gacactccct tccgccagat cacagagatg actatcctca cggtccaact tatcgtggag | 420 |
| ttcgcgaagg gattgccagg gttcgccaag atctcgcagc ctgatcaaat tacgctgctt | 480 |
| aaggcttgct caagtgaggt aatgatgctc cgagtcgcgc gacgatacga tgcggcctca | 540 |
| gacagtgttc tgttcgcgaa caaccaagcg tacactcgcg acaactaccg caaggctggc | 600 |
| atggcctacg tcatcgagga tctactgcac ttctgccggt gcatgtactc tatggcgttg | 660 |
| gacaacatcc attacgcgct gctcacggct gtcgtcatct tttctgaccg gccagggttg | 720 |
| gagcagccgc aactggtgga agaaatccag cggtactacc tgaatacgct ccgcatctat | 780 |
| atcctgaacc agctgagcgg gtcggcgcgt tcgtccgtca tatacggcaa gatcctctca | 840 |
| atcctctctg agctacgcac gctcggcatg caaaactcca acatgtgcat ctccctcaag | 900 |
| ctcaagaaca gaaagctgcc gcctttcctc gaggagatct gggatgtggc ggacatgtcg | 960 |
| cacacccaac cgccgcctat cctcgagtcc cccacgaatc tctagcccct gcgcgcacgc | 1020 |
| atcgccgatg ccgcgtccgg ccgcgctgct ctga | 1054 |
| <210> SEQ ID NO: 2 | |
| <211> LENGTH: 1288 | |
| <212> TYPE: DNA | |
| <213> ORGANISM: Choristoneura fumiferana | |
| <400> SEQUENCE: 2 | |
| aagggccctg cgccccgtca gcaagaggaa ctgtgtctgg tatgcgggga cagagcctcc | 60 |
| ggataccact acaatgcgct cacgtgtgaa gggtgtaaag ggttcttcag acggagtgtt | 120 |
| accaaaaatg cggtttatat ttgtaaattc ggtcacgctt gcgaaatgga catgtacatg | 180 |
| cgacggaaat gccaggagtg ccgcctgaag aagtgcttag ctgtaggcat gaggcctgag | 240 |
| tgcgtagtac ccgagactca gtgcgccatg aagcggaaag agaagaaagc acagaaggag | 300 |
| aaggacaaac tgcctgtcag cacgacgacg gtggacgacc acatgccgcc cattatgcag | 360 |
| tgtgaacctc cacctcctga agcagcaagg attcacgaag tggtcccaag gtttctctcc | 420 |
| gacaagctgt tggagacaaa ccggcagaaa aacatccccc agttgacagc caaccagcag | 480 |
| ttccttatcg ccaggctcat ctggtaccag gacgggtacg agcagccttc tgatgaagat | 540 |
| ttgaagagga ttacgcagac gtggcagcaa gcggacgatg aaaacgaaga gtctgacact | 600 |
| cccttccgcc agatcacaga gatgactatc ctcacggtcc aacttatcgt ggagttcgcg | 660 |
| aagggattgc cagggttcgc caagatctcg cagcctgatc aaattacgct gcttaaggct | 720 |
| tgctcaagtg aggtaatgat gctccgagtc gcgcgacgat acgatgcggc ctcagacagt | 780 |
| gttctgttcg cgaacaacca agcgtacact cgcgacaact accgcaaggc tggcatggcc | 840 |
| tacgtcatcg aggatctact gcacttctgc cggtgcatgt actctatggc gttggacaac | 900 |
| atccattacg cgctgctcac ggctgtcgtc atcttttctg accggccagg gttggagcag | 960 |
| ccgcaactgg tggaagaaat ccagcggtac tacctgaata cgctccgcat ctatatcctg | 1020 |
| aaccagctga gcgggtcggc gcgttcgtcc gtcatatacg gcaagatcct ctcaatcctc | 1080 |
| tctgagctac gcacgctcgg catgcaaaac tccaacatgt gcatctccct caagctcaag | 1140 |
| aacagaaagc tgccgccttt cctcgaggag atctgggatg tggcggacat gtcgcacacc | 1200 |
| caaccgccgc ctatcctcga gtcccccacg aatctctagc ccctgcgcgc acgcatcgcc | 1260 |
| gatgccgcgt ccggccgcgc tgctctga | 1288 |
| <210> SEQ ID NO: 3 | |
| <211> LENGTH: 1650 | |
| <212> TYPE: DNA | |
| <213> ORGANISM: Drosophila melanogaster | |
| <400> SEQUENCE: 3 | |
| cggccggaat gcgtcgtccc ggagaaccaa tgtgcgatga agcggcgcga aaagaaggcc | 60 |
| cagaaggaga aggacaaaat gaccacttcg ccgagctctc agcatggcgg caatggcagc | 120 |
| ttggcctctg gtggcggcca agactttgtt aagaaggaga ttcttgacct tatgacatgc | 180 |
| gagccgcccc agcatgccac tattccgcta ctacctgatg aaatattggc caagtgtcaa | 240 |
| gcgcgcaata taccttcctt aacgtacaat cagttggccg ttatatacaa gttaatttgg | 300 |
| taccaggatg gctatgagca gccatctgaa gaggatctca ggcgtataat gagtcaaccc | 360 |
| gatgagaacg agagccaaac ggacgtcagc tttcggcata taaccgagat aaccatactc | 420 |
| acggtccagt tgattgttga gtttgctaaa ggtctaccag cgtttacaaa gataccccag | 480 |
| gaggaccaga tcacgttact aaaggcctgc tcgtcggagg tgatgatgct gcgtatggca | 540 |
| cgacgctatg accacagctc ggactcaata ttcttcgcga ataatagatc atatacgcgg | 600 |
| gattcttaca aaatggccgg aatggctgat aacattgaag acctgctgca tttctgccgc | 660 |
| caaatgttct cgatgaaggt ggacaacgtc gaatacgcgc ttctcactgc cattgtgatc | 720 |
| ttctcggacc ggccgggcct ggagaaggcc caactagtcg aagcgatcca gagctactac | 780 |
| atcgacacgc tacgcattta tatactcaac cgccactgcg gcgactcaat gagcctcgtc | 840 |
| ttctacgcaa agctgctctc gatcctcacc gagctgcgta cgctgggcaa ccagaacgcc | 900 |
| gagatgtgtt tctcactaaa gctcaaaaac cgcaaactgc ccaagttcct cgaggagatc | 960 |
| tgggacgttc atgccatccc gccatcggtc cagtcgcacc ttcagattac ccaggaggag | 1020 |
| aacgagcgtc tcgagcgggc tgagcgtatg cgggcatcgg ttgggggcgc cattaccgcc | 1080 |
| ggcattgatt gcgactctgc ctccacttcg gcggcggcag ccgcggccca gcatcagcct | 1140 |
| cagcctcagc cccagcccca accctcctcc ctgacccaga acgattccca gcaccagaca | 1200 |
| cagccgcagc tacaacctca gctaccacct cagctgcaag gtcaactgca accccagctc | 1260 |
| caaccacagc ttcagacgca actccagcca cagattcaac cacagccaca gctccttccc | 1320 |
| gtctccgctc ccgtgcccgc ctccgtaacc gcacctggtt ccttgtccgc ggtcagtacg | 1380 |
| agcagcgaat acatgggcgg aagtgcggcc ataggaccca tcacgccggc aaccaccagc | 1440 |
| agtatcacgg ctgccgttac cgctagctcc accacatcag cggtaccgat gggcaacgga | 1500 |
| gttggagtcg gtgttggggt gggcggcaac gtcagcatgt atgcgaacgc ccagacggcg | 1560 |
| atggccttga tgggtgtagc cctgcattcg caccaagagc agcttatcgg gggagtggcg | 1620 |
| gttaagtcgg agcactcgac gactgcatag | 1650 |
| <210> SEQ ID NO: 4 | |
| <211> LENGTH: 894 | |
| <212> TYPE: DNA | |
| <213> ORGANISM: Tenebrio molitor | |
| <400> SEQUENCE: 4 | |
| aggccggaat gtgtggtacc ggaagtacag tgtgctgtta agagaaaaga gaagaaagcc | 60 |
| caaaaggaaa aagataaacc aaacagcact actaacggct caccagacgt catcaaaatt | 120 |
| gaaccagaat tgtcagattc agaaaaaaca ttgactaacg gacgcaatag gatatcacca | 180 |
| gagcaagagg agctcatact catacatcga ttggtttatt tccaaaacga atatgaacat | 240 |
| ccgtctgaag aagacgttaa acggattatc aatcagccga tagatggtga agatcagtgt | 300 |
| gagatacggt ttaggcatac cacggaaatt acgatcctga ctgtgcagct gatcgtggag | 360 |
| tttgccaagc ggttaccagg cttcgataag ctcctgcagg aagatcaaat tgctctcttg | 420 |
| aaggcatgtt caagcgaagt gatgatgttc aggatggccc gacgttacga cgtccagtcg | 480 |
| gattccatcc tcttcgtaaa caaccagcct tatccgaggg acagttacaa tttggccggt | 540 |
| atgggggaaa ccatcgaaga tctcttgcat ttttgcagaa ctatgtactc catgaaggtg | 600 |
| gataatgccg aatatgcttt actaacagcc atcgttattt tctcagagcg accgtcgttg | 660 |
| atagaaggct ggaaggtgga gaagatccaa gaaatctatt tagaggcatt gcgggcgtac | 720 |
| gtcgacaacc gaagaagccc aagccggggc acaatattcg cgaaactcct gtcagtacta | 780 |
| actgaattgc ggacgttagg caaccaaaat tcagagatgt gcatctcgtt gaaattgaaa | 840 |
| aacaaaaagt taccgccgtt cctggacgaa atctgggacg tcgacttaaa agca | 894 |
| 210> SEQ ID NO: 5 | |
| <211> LENGTH: 948 | |
| <212> TYPE: DNA | |
| <213> ORGANISM: Amblyomma americanum | |
| <400> SEQUENCE: 5 | |
| cggccggaat gtgtggtgcc ggagtaccag tgtgccatca agcgggagtc taagaagcac | 60 |
| cagaaggacc ggccaaacag cacaacgcgg gaaagtccct cggcgctgat ggcgccatct | 120 |
| tctgtgggtg gcgtgagccc caccagccag cccatgggtg gcggaggcag ctccctgggc | 180 |
| agcagcaatc acgaggagga taagaagcca gtggtgctca gcccaggagt caagcccctc | 240 |
| tcttcatctc aggaggacct catcaacaag ctagtctact accagcagga gtttgagtcg | 300 |
| ccttctgagg aagacatgaa gaaaaccacg cccttccccc tgggagacag tgaggaagac | 360 |
| aaccagcggc gattccagca cattactgag atcaccatcc tgacagtgca gctcattgtg | 420 |
| gagttctcca agcgggtccc tggctttgac acgctggcac gagaagacca gattactttg | 480 |
| ctgaaggcct gctccagtga agtgatgatg ctgagaggtg cccggaaata tgatgtgaag | 540 |
| acagattcta tagtgtttgc caataaccag ccgtacacga gggacaacta ccgcagtgcc | 600 |
| agtgtggggg actctgcaga tgccctgttc cgcttctgcc gcaagatgtg tcagctgaga | 660 |
| gtagacaacg ctgaatacgc actcctgacg gccattgtaa ttttctctga acggccatca | 720 |
| ctggtggacc cgcacaaggt ggagcgcatc caggagtact acattgagac cctgcgcatg | 780 |
| tactccgaga accaccggcc cccaggcaag aactactttg cccggctgct gtccatcttg | 840 |
| acagagctgc gcaccttggg caacatgaac gccgaaatgt gcttctcgct caaggtgcag | 900 |
| aacaagaagc tgccaccgtt cctggctgag atttgggaca tccaagag | 948 |
| <210> SEQ ID NO: 6 | |
| <211> LENGTH: 334 | |
| <212> TYPE: PRT | |
| <213> ORGANISM: Choristoneura fumiferana | |
| <400> SEQUENCE: 6 | |
| Pro Glu Cys Val Val Pro Glu Thr Gln Cys Ala Met Lys Arg Lys Glu | |
| Lys Lys Ala Gln Lys Glu Lys Asp Lys Leu Pro Val Ser Thr Thr Thr | |
| Val Asp Asp His Met Pro Pro Ile Met Gln Cys Glu Pro Pro Pro Pro | |
| Glu Ala Ala Arg Ile His Glu Val Val Pro Arg Phe Leu Ser Asp Lys | |
| Leu Leu Glu Thr Asn Arg Gln Lys Asn Ile Pro Gln Leu Thr Ala Asn | |
| Gln Gln Phe Leu Ile Ala Arg Leu Ile Trp Tyr Gln Asp Gly Tyr Glu | |
| Gln Pro Ser Asp Glu Asp Leu Lys Arg Ile Thr Gln Thr Trp Gln Gln | |
| Ala Asp Asp Glu Asn Glu Glu Ser Asp Thr Pro Phe Arg Gln Ile Thr | |
| Glu Met Thr Ile Leu Thr Val Gln Leu Ile Val Glu Phe Ala Lys Gly | |
| Leu Pro Gly Phe Ala Lys Ile Ser Gln Pro Asp Gln Ile Thr Leu Leu | |
| Lys Ala Cys Ser Ser Glu Val Met Met Leu Arg Val Ala Arg Arg Tyr | |
| Asp Ala Ala Ser Asp Ser Val Leu Phe Ala Asn Asn Gln Ala Tyr Thr | |
| Arg Asp Asn Tyr Arg Lys Ala Gly Met Ala Tyr Val Ile Glu Asp Leu | |
| Leu His Phe Cys Arg Cys Met Tyr Ser Met Ala Leu Asp Asn Ile His | |
| Tyr Ala Leu Leu Thr Ala Val Val Ile Phe Ser Asp Arg Pro Gly Leu | |
| Glu Gln Pro Gln Leu Val Glu Glu Ile Gln Arg Tyr Tyr Leu Asn Thr | |
| Leu Arg Ile Tyr Ile Leu Asn Gln Leu Ser Gly Ser Ala Arg Ser Ser | |
| Val Ile Tyr Gly Lys Ile Leu Ser Ile Leu Ser Glu Leu Arg Thr Leu | |
| Gly Met Gln Asn Ser Asn Met Cys Ile Ser Leu Lys Leu Lys Asn Arg | |
| Lys Leu Pro Pro Phe Leu Glu Glu Ile Trp Asp Val Ala Asp Met Ser | |
| His Thr Gln Pro Pro Pro Ile Leu Glu Ser Pro Thr Asn Leu | |
| <210> SEQ ID NO: 7 | |
| <211> LENGTH: 549 | |
| <212> TYPE: PRT | |
| <213> ORGANISM: Drosophila melanogaster | |
| <400> SEQUENCE: 7 | |
| Arg Pro Glu Cys Val Val Pro Glu Asn Gln Cys Ala Met Lys Arg Arg | |
| Glu Lys Lys Ala Gln Lys Glu Lys Asp Lys Met Thr Thr Ser Pro Ser | |
| Ser Gln His Gly Gly Asn Gly Ser Leu Ala Ser Gly Gly Gly Gln Asp | |
| Phe Val Lys Lys Glu Ile Leu Asp Leu Met Thr Cys Glu Pro Pro Gln | |
| His Ala Thr Ile Pro Leu Leu Pro Asp Glu Ile Leu Ala Lys Cys Gln | |
| Ala Arg Asn Ile Pro Ser Leu Thr Tyr Asn Gln Leu Ala Val Ile Tyr | |
| Lys Leu Ile Trp Tyr Gln Asp Gly Tyr Glu Gln Pro Ser Glu Glu Asp | |
| Leu Arg Arg Ile Met Ser Gln Pro Asp Glu Asn Glu Ser Gln Thr Asp | |
| Val Ser Phe Arg His Ile Thr Glu Ile Thr Ile Leu Thr Val Gln Leu | |
| Ile Val Glu Phe Ala Lys Gly Leu Pro Ala Phe Thr Lys Ile Pro Gln | |
| Glu Asp Gln Ile Thr Leu Leu Lys Ala Cys Ser Ser Glu Val Met Met | |
| Leu Arg Met Ala Arg Arg Tyr Asp His Ser Ser Asp Ser Ile Phe Phe | |
| Ala Asn Asn Arg Ser Tyr Thr Arg Asp Ser Tyr Lys Met Ala Gly Met | |
| Ala Asp Asn Ile Glu Asp Leu Leu His Phe Cys Arg Gln Met Phe Ser | |
| Met Lys Val Asp Asn Val Glu Tyr Ala Leu Leu Thr Ala Ile Val Ile | |
| Phe Ser Asp Arg Pro Gly Leu Glu Lys Ala Gln Leu Val Glu Ala Ile | |
| Gln Ser Tyr Tyr Ile Asp Thr Leu Arg Ile Tyr Ile Leu Asn Arg His | |
| Cys Gly Asp Ser Met Ser Leu Val Phe Tyr Ala Lys Leu Leu Ser Ile | |
| Leu Thr Glu Leu Arg Thr Leu Gly Asn Gln Asn Ala Glu Met Cys Phe | |
| Ser Leu Lys Leu Lys Asn Arg Lys Leu Pro Lys Phe Leu Glu Glu Ile | |
| Trp Asp Val His Ala Ile Pro Pro Ser Val Gln Ser His Leu Gln Ile | |
| Thr Gln Glu Glu Asn Glu Arg Leu Glu Arg Ala Glu Arg Met Arg Ala | |
| Ser Val Gly Gly Ala Ile Thr Ala Gly Ile Asp Cys Asp Ser Ala Ser | |
| Thr Ser Ala Ala Ala Ala Ala Ala Gln His Gln Pro Gln Pro Gln Pro | |
| Gln Pro Gln Pro Ser Ser Leu Thr Gln Asn Asp Ser Gln His Gln Thr | |
| Gln Pro Gln Leu Gln Pro Gln Leu Pro Pro Gln Leu Gln Gly Gln Leu | |
| Gln Pro Gln Leu Gln Pro Gln Leu Gln Thr Gln Leu Gln Pro Gln Ile | |
| Gln Pro Gln Pro Gln Leu Leu Pro Val Ser Ala Pro Val Pro Ala Ser | |
| Val Thr Ala Pro Gly Ser Leu Ser Ala Val Ser Thr Ser Ser Glu Tyr | |
| Met Gly Gly Ser Ala Ala Ile Gly Pro Ile Thr Pro Ala Thr Thr Ser | |
| Ser Ile Thr Ala Ala Val Thr Ala Ser Ser Thr Thr Ser Ala Val Pro | |
| Met Gly Asn Gly Val Gly Val Gly Val Gly Val Gly Gly Asn Val Ser | |
| Met Tyr Ala Asn Ala Gln Thr Ala Met Ala Leu Met Gly Val Ala Leu | |
| His Ser His Gln Glu Gln Leu Ile Gly Gly Val Ala Val Lys Ser Glu | |
| His Ser Thr Thr Ala | |
| <210> SEQ ID NO: 8 | |
| <211> LENGTH: 401 | |
| <212> TYPE: PRT | |
| <213> ORGANISM: Choristoneura fumiferana | |
| <400> SEQUENCE: 8 | |
| Cys Leu Val Cys Gly Asp Arg Ala Ser Gly Tyr His Tyr Asn Ala Leu | |
| Thr Cys Glu Gly Cys Lys Gly Phe Phe Arg Arg Ser Val Thr Lys Asn | |
| Ala Val Tyr Ile Cys Lys Phe Gly His Ala Cys Glu Met Asp Met Tyr | |
| Met Arg Arg Lys Cys Gln Glu Cys Arg Leu Lys Lys Cys Leu Ala Val | |
| Gly Met Arg Pro Glu Cys Val Val Pro Glu Thr Gln Cys Ala Met Lys | |
| Arg Lys Glu Lys Lys Ala Gln Lys Glu Lys Asp Lys Leu Pro Val Ser | |
| Thr Thr Thr Val Asp Asp His Met Pro Pro Ile Met Gln Cys Glu Pro | |
| Pro Pro Pro Glu Ala Ala Arg Ile His Glu Val Val Pro Arg Phe Leu | |
| Ser Asp Lys Leu Leu Glu Thr Asn Arg Gln Lys Asn Ile Pro Gln Leu | |
| Thr Ala Asn Gln Gln Phe Leu Ile Ala Arg Leu Ile Trp Tyr Gln Asp | |
| Gly Tyr Glu Gln Pro Ser Asp Glu Asp Leu Lys Arg Ile Thr Gln Thr | |
| Trp Gln Gln Ala Asp Asp Glu Asn Glu Glu Ser Asp Thr Pro Phe Arg | |
| Gln Ile Thr Glu Met Thr Ile Leu Thr Val Gln Leu Ile Val Glu Phe | |
| Ala Lys Gly Leu Pro Gly Phe Ala Lys Ile Ser Gln Pro Asp Gln Ile | |
| Thr Leu Leu Lys Ala Cys Ser Ser Glu Val Met Met Leu Arg Val Ala | |
| Arg Arg Tyr Asp Ala Ala Ser Asp Ser Val Leu Phe Ala Asn Asn Gln | |
| Ala Tyr Thr Arg Asp Asn Tyr Arg Lys Ala Gly Met Ala Tyr Val Ile | |
| Glu Asp Leu Leu His Phe Cys Arg Cys Met Tyr Ser Met Ala Leu Asp | |
| Asn Ile His Tyr Ala Leu Leu Thr Ala Val Val Ile Phe Ser Asp Arg | |
| Pro Gly Leu Glu Gln Pro Gln Leu Val Glu Glu Ile Gln Arg Tyr Tyr | |
| Leu Asn Thr Leu Arg Ile Tyr Ile Leu Asn Gln Leu Ser Gly Ser Ala | |
| Arg Ser Ser Val Ile Tyr Gly Lys Ile Leu Ser Ile Leu Ser Glu Leu | |
| Arg Thr Leu Gly Met Gln Asn Ser Asn Met Cys Ile Ser Leu Lys Leu | |
| Lys Asn Arg Lys Leu Pro Pro Phe Leu Glu Glu Ile Trp Asp Val Ala | |
| Asp Met Ser His Thr Gln Pro Pro Pro Ile Leu Glu Ser Pro Thr Asn | |
| Leu | |
| <210> SEQ ID NO: 9 | |
| <211> LENGTH: 298 | |
| <212> TYPE: PRT | |
| <213> ORGANISM: Tenebrio molitor | |
| <400> SEQUENCE: 9 | |
| Arg Pro Glu Cys Val Val Pro Glu Val Gln Cys Ala Val Lys Arg Lys | |
| Glu Lys Lys Ala Gln Lys Glu Lys Asp Lys Pro Asn Ser Thr Thr Asn | |
| Gly Ser Pro Asp Val Ile Lys Ile Glu Pro Glu Leu Ser Asp Ser Glu | |
| Lys Thr Leu Thr Asn Gly Arg Asn Arg Ile Ser Pro Glu Gln Glu Glu | |
| Leu Ile Leu Ile His Arg Leu Val Tyr Phe Gln Asn Glu Tyr Glu His | |
| Pro Ser Glu Glu Asp Val Lys Arg Ile Ile Asn Gln Pro Ile Asp Gly | |
| Glu Asp Gln Cys Glu Ile Arg Phe Arg His Thr Thr Glu Ile Thr Ile | |
| Leu Thr Val Gln Leu Ile Val Glu Phe Ala Lys Arg Leu Pro Gly Phe | |
| Asp Lys Leu Leu Gln Glu Asp Gln Ile Ala Leu Leu Lys Ala Cys Ser | |
| Ser Glu Val Met Met Phe Arg Met Ala Arg Arg Tyr Asp Val Gln Ser | |
| Asp Ser Ile Leu Phe Val Asn Asn Gln Pro Tyr Pro Arg Asp Ser Tyr | |
| Asn Leu Ala Gly Met Gly Glu Thr Ile Glu Asp Leu Leu His Phe Cys | |
| Arg Thr Met Tyr Ser Met Lys Val Asp Asn Ala Glu Tyr Ala Leu Leu | |
| Thr Ala Ile Val Ile Phe Ser Glu Arg Pro Ser Leu Ile Glu Gly Trp | |
| Lys Val Glu Lys Ile Gln Glu Ile Tyr Leu Glu Ala Leu Arg Ala Tyr | |
| Val Asp Asn Arg Arg Ser Pro Ser Arg Gly Thr Ile Phe Ala Lys Leu | |
| Leu Ser Val Leu Thr Glu Leu Arg Thr Leu Gly Asn Gln Asn Ser Glu | |
| Met Cys Ile Ser Leu Lys Leu Lys Asn Lys Lys Leu Pro Pro Phe Leu | |
| Asp Glu Ile Trp Asp Val Asp Leu Lys Ala | |
| <210> SEQ ID NO: 10 | |
| <211> LENGTH: 316 | |
| <212> TYPE: PRT | |
| <213> ORGANISM: Amblyomma americanum | |
| <400> SEQUENCE: 10 | |
| Arg Pro Glu Cys Val Val Pro Glu Tyr Gln Cys Ala Ile Lys Arg Glu | |
| Ser Lys Lys His Gln Lys Asp Arg Pro Asn Ser Thr Thr Arg Glu Ser | |
| Pro Ser Ala Leu Met Ala Pro Ser Ser Val Gly Gly Val Ser Pro Thr | |
| Ser Gln Pro Met Gly Gly Gly Gly Ser Ser Leu Gly Ser Ser Asn His | |
| Glu Glu Asp Lys Lys Pro Val Val Leu Ser Pro Gly Val Lys Pro Leu | |
| Ser Ser Ser Gln Glu Asp Leu Ile Asn Lys Leu Val Tyr Tyr Gln Gln | |
| Glu Phe Glu Ser Pro Ser Glu Glu Asp Met Lys Lys Thr Thr Pro Phe | |
| Pro Leu Gly Asp Ser Glu Glu Asp Asn Gln Arg Arg Phe Gln His Ile | |
| Thr Glu Ile Thr Ile Leu Thr Val Gln Leu Ile Val Glu Phe Ser Lys | |
| Arg Val Pro Gly Phe Asp Thr Leu Ala Arg Glu Asp Gln Ile Thr Leu | |
| Leu Lys Ala Cys Ser Ser Glu Val Met Met Leu Arg Gly Ala Arg Lys | |
| Tyr Asp Val Lys Thr Asp Ser Ile Val Phe Ala Asn Asn Gln Pro Tyr | |
| Thr Arg Asp Asn Tyr Arg Ser Ala Ser Val Gly Asp Ser Ala Asp Ala | |
| Leu Phe Arg Phe Cys Arg Lys Met Cys Gln Leu Arg Val Asp Asn Ala | |
| Glu Tyr Ala Leu Leu Thr Ala Ile Val Ile Phe Ser Glu Arg Pro Ser | |
| Leu Val Asp Pro His Lys Val Glu Arg Ile Gln Glu Tyr Tyr Ile Glu | |
| Thr Leu Arg Met Tyr Ser Glu Asn His Arg Pro Pro Gly Lys Asn Tyr | |
| Phe Ala Arg Leu Leu Ser Ile Leu Thr Glu Leu Arg Thr Leu Gly Asn | |
| Met Asn Ala Glu Met Cys Phe Ser Leu Lys Val Gln Asn Lys Lys Leu | |
| Pro Pro Phe Leu Ala Glu Ile Trp Asp Ile Gln Glu | |
| SEQ ID NO: 11 | |
| <211> LENGTH: 711 | |
| <212> TYPE: DNA | |
| <213> ORGANISM: Artificial Sequence | |
| <220> FEATURE: | |
| <223> OTHER INFORMATION: Chimeric RXR ligand binding domain | |
| <400> SEQUENCE: 11 | |
| gccaacgagg acatgcctgt agagaagatt ctggaagccg agcttgctgt cgagcccaag | 60 |
| actgagacat acgtggaggc aaacatgggg ctgaacccca gctcaccaaa tgaccctgtt | 120 |
| accaacatct gtcaagcagc agacaagcag ctcttcactc ttgtggagtg ggccaagagg | 180 |
| atcccacact tttctgagct gcccctagac gaccaggtca tcctgctacg ggcaggctgg | 240 |
| aacgagctgc tgatcgcctc cttctcccac cgctccatag ctgtgaaaga tgggattctc | 300 |
| ctggccaccg gcctgcacgt acaccggaac agcgctcaca gtgctggggt gggcgccatc | 360 |
| tttgacaggg tgctaacaga gctggtgtct aagatgcgtg acatgcagat ggacaagact | 420 |
| gaacttggct gcttgcgatc tgttattctt ttcaatccag aggtgagggg tttgaaatcc | 480 |
| gcccaggaag ttgaacttct acgtgaaaaa gtatatgccg ctttggaaga atatactaga | 540 |
| acaacacatc ccgatgaacc aggaagattt gcaaaacttt tgcttcgtct gccttcttta | 600 |
| cgttccatag gccttaagtg tttggagcat ttgtttttct ttcgccttat tggagatgtt | 660 |
| ccaattgata cgttcctgat ggagatgctt gaatcacctt ctgattcata a | 711 |
| <210> SEQ ID NO: 12 | |
| <211> LENGTH: 720 | |
| <212> TYPE: DNA | |
| <213> ORGANISM: Homo sapiens | |
| <400> SEQUENCE: 12 | |
| gcccccgagg agatgcctgt ggacaggatc ctggaggcag agcttgctgt ggaacagaag | 60 |
| agtgaccagg gcgttgaggg tcctggggga accgggggta gcggcagcag cccaaatgac | 120 |
| cctgtgacta acatctgtca ggcagctgac aaacagctat tcacgcttgt tgagtgggcg | 180 |
| aagaggatcc cacacttttc ctccttgcct ctggatgatc aggtcatatt gctgcgggca | 240 |
| ggctggaatg aactcctcat tgcctccttt tcacaccgat ccattgatgt tcgagatggc | 300 |
| atcctccttg ccacaggtct tcacgtgcac cgcaactcag cccattcagc aggagtagga | 360 |
| gccatctttg atcgggtgct gacagagcta gtgtccaaaa tgcgtgacat gaggatggac | 420 |
| aagacagagc ttggctgcct gagggcaatc attctgttta atccagatgc caagggcctc | 480 |
| tccaacccta gtgaggtgga ggtcctgcgg gagaaagtgt atgcatcact ggagacctac | 540 |
| tgcaaacaga agtaccctga gcagcaggga cggtttgcca agctgctgct acgtcttcct | 600 |
| gccctccggt ccattggcct taagtgtcta gagcatctgt ttttcttcaa gctcattggt | 660 |
| gacaccccca tcgacacctt cctcatggag atgcttgagg ctccccatca actggcctga | 720 |
| SEQ ID NO: 13 | |
| <211> LENGTH: 635 | |
| <212> TYPE: DNA | |
| <213> ORGANISM: Locusta migratoria | |
| <400> SEQUENCE: 13 | |
| tgcatacaga catgcctgtt gaacgcatac ttgaagctga aaaacgagtg gagtgcaaag | 60 |
| cagaaaacca agtggaatat gagctggtgg agtgggctaa acacatcccg cacttcacat | 120 |
| ccctacctct ggaggaccag gttctcctcc tcagagcagg ttggaatgaa ctgctaattg | 180 |
| cagcattttc acatcgatct gtagatgtta aagatggcat agtacttgcc actggtctca | 240 |
| cagtgcatcg aaattctgcc catcaagctg gagtcggcac aatatttgac agagttttga | 300 |
| cagaactggt agcaaagatg agagaaatga aaatggataa aactgaactt ggctgcttgc | 360 |
| gatctgttat tcttttcaat ccagaggtga ggggtttgaa atccgcccag gaagttgaac | 420 |
| ttctacgtga aaaagtatat gccgctttgg aagaatatac tagaacaaca catcccgatg | 480 |
| aaccaggaag atttgcaaaa cttttgcttc gtctgccttc tttacgttcc ataggcctta | 540 |
| agtgtttgga gcatttgttt ttctttcgcc ttattggaga tgttccaatt gatacgttcc | 600 |
| tgatggagat gcttgaatca ccttctgatt cataa | 635 |
| <210> SEQ ID NO: 14 | |
| <211> LENGTH: 236 | |
| <212> TYPE: PRT | |
| <213> ORGANISM: Artificial Sequence | |
| <220> FEATURE: | |
| <223> OTHER INFORMATION: Chimeric RXR ligand binding domain | |
| <400> SEQUENCE: 14 | |
| Ala Asn Glu Asp Met Pro Val Glu Lys Ile Leu Glu Ala Glu Leu Ala | |
| Val Glu Pro Lys Thr Glu Thr Tyr Val Glu Ala Asn Met Gly Leu Asn | |
| Pro Ser Ser Pro Asn Asp Pro Val Thr Asn Ile Cys Gln Ala Ala Asp | |
| Lys Gln Leu Phe Thr Leu Val Glu Trp Ala Lys Arg Ile Pro His Phe | |
| Ser Glu Leu Pro Leu Asp Asp Gln Val Ile Leu Leu Arg Ala Gly Trp | |
| Asn Glu Leu Leu Ile Ala Ser Phe Ser His Arg Ser Ile Ala Val Lys | |
| Asp Gly Ile Leu Leu Ala Thr Gly Leu His Val His Arg Asn Ser Ala | |
| His Ser Ala Gly Val Gly Ala Ile Phe Asp Arg Val Leu Thr Glu Leu | |
| Val Ser Lys Met Arg Asp Met Gln Met Asp Lys Thr Glu Leu Gly Cys | |
| Leu Arg Ser Val Ile Leu Phe Asn Pro Glu Val Arg Gly Leu Lys Ser | |
| Ala Gln Glu Val Glu Leu Leu Arg Glu Lys Val Tyr Ala Ala Leu Glu | |
| Glu Tyr Thr Arg Thr Thr His Pro Asp Glu Pro Gly Arg Phe Ala Lys | |
| Leu Leu Leu Arg Leu Pro Ser Leu Arg Ser Ile Gly Leu Lys Cys Leu | |
| Glu His Leu Phe Phe Phe Arg Leu Ile Gly Asp Val Pro Ile Asp Thr | |
| Phe Leu Met Glu Met Leu Glu Ser Pro Ser Asp Ser | |
| <210> SEQ ID NO: 15 | |
| <211> LENGTH: 239 | |
| <212> TYPE: PRT | |
| <213> ORGANISM: Homo sapiens | |
| <400> SEQUENCE: 15 | |
| Ala Pro Glu Glu Met Pro Val Asp Arg Ile Leu Glu Ala Glu Leu Ala | |
| Val Glu Gln Lys Ser Asp Gln Gly Val Glu Gly Pro Gly Gly Thr Gly | |
| Gly Ser Gly Ser Ser Pro Asn Asp Pro Val Thr Asn Ile Cys Gln Ala | |
| Ala Asp Lys Gln Leu Phe Thr Leu Val Glu Trp Ala Lys Arg Ile Pro | |
| His Phe Ser Ser Leu Pro Leu Asp Asp Gln Val Ile Leu Leu Arg Ala | |
| Gly Trp Asn Glu Leu Leu Ile Ala Ser Phe Ser His Arg Ser Ile Asp | |
| Val Arg Asp Gly Ile Leu Leu Ala Thr Gly Leu His Val His Arg Asn | |
| Ser Ala His Ser Ala Gly Val Gly Ala Ile Phe Asp Arg Val Leu Thr | |
| Glu Leu Val Ser Lys Met Arg Asp Met Arg Met Asp Lys Thr Glu Leu | |
| Gly Cys Leu Arg Ala Ile Ile Leu Phe Asn Pro Asp Ala Lys Gly Leu | |
| Ser Asn Pro Ser Glu Val Glu Val Leu Arg Glu Lys Val Tyr Ala Ser | |
| Leu Glu Thr Tyr Cys Lys Gln Lys Tyr Pro Glu Gln Gln Gly Arg Phe | |
| Ala Lys Leu Leu Leu Arg Leu Pro Ala Leu Arg Ser Ile Gly Leu Lys | |
| Cys Leu Glu His Leu Phe Phe Phe Lys Leu Ile Gly Asp Thr Pro Ile | |
| Asp Thr Phe Leu Met Glu Met Leu Glu Ala Pro His Gln Leu Ala | |
| <210> SEQ ID NO: 16 | |
| <211> LENGTH: 210 | |
| <212> TYPE: PRT | |
| <213> ORGANISM: Locusta migratoria | |
| <400> SEQUENCE: 16 | |
| His Thr Asp Met Pro Val Glu Arg Ile Leu Glu Ala Glu Lys Arg Val | |
| Glu Cys Lys Ala Glu Asn Gln Val Glu Tyr Glu Leu Val Glu Trp Ala | |
| Lys His Ile Pro His Phe Thr Ser Leu Pro Leu Glu Asp Gln Val Leu | |
| Leu Leu Arg Ala Gly Trp Asn Glu Leu Leu Ile Ala Ala Phe Ser His | |
| Arg Ser Val Asp Val Lys Asp Gly Ile Val Leu Ala Thr Gly Leu Thr | |
| Val His Arg Asn Ser Ala His Gln Ala Gly Val Gly Thr Ile Phe Asp | |
| Arg Val Leu Thr Glu Leu Val Ala Lys Met Arg Glu Met Lys Met Asp | |
| Lys Thr Glu Leu Gly Cys Leu Arg Ser Val Ile Leu Phe Asn Pro Glu | |
| Val Arg Gly Leu Lys Ser Ala Gln Glu Val Glu Leu Leu Arg Glu Lys | |
| Val Tyr Ala Ala Leu Glu Glu Tyr Thr Arg Thr Thr His Pro Asp Glu | |
| Pro Gly Arg Phe Ala Lys Leu Leu Leu Arg Leu Pro Ser Leu Arg Ser | |
| Ile Gly Leu Lys Cys Leu Glu His Leu Phe Phe Phe Arg Leu Ile Gly | |
| Asp Val Pro Ile Asp Thr Phe Leu Met Glu Met Leu Glu Ser Pro Ser | |
| Asp Ser | |
| <210> SEQ ID NO: 17 | |
| <211> 240 | |
| <212> PRT | |
| <213> Choristoneura fumiferana | |
| <400> SEQUENCE: 17 | |
| Leu Thr Ala Asn Gln Gln Phe Leu Ile Ala Arg Leu Ile Trp Tyr Gln | |
| Asp Gly Tyr Glu Gln Pro Ser Asp Glu Asp Leu Lys Arg Ile Thr Gln | |
| Thr Trp Gln Gln Ala Asp Asp Glu Asn Glu Glu Ser Asp Thr Pro Phe | |
| Arg Gln Ile Thr Glu Met Thr Ile Leu Thr Val Gln Leu Ile Val Glu | |
| Phe Ala Lys Gly Leu Pro Gly Phe Ala Lys Ile Ser Gln Pro Asp Gln | |
| Ile Thr Leu Leu Lys Ala Cys Ser Ser Glu Val Met Met Leu Arg Val | |
| Ala Arg Arg Tyr Asp Ala Ala Ser Asp Ser Val Leu Phe Ala Asn Asn | |
| Gln Ala Tyr Thr Arg Asp Asn Tyr Arg Lys Ala Gly Met Ala Tyr Val | |
| Ile Glu Asp Leu Leu His Phe Cys Arg Cys Met Tyr Ser Met Ala Leu | |
| Asp Asn Ile His Tyr Ala Leu Leu Thr Ala Val Val Ile Phe Ser Asp | |
| Arg Pro Gly Leu Glu Gln Pro Gln Leu Val Glu Glu Ile Gln Arg Tyr | |
| Tyr Leu Asn Thr Leu Arg Ile Tyr Ile Leu Asn Gln Leu Ser Gly Ser | |
| Ala Arg Ser Ser Val Ile Tyr Gly Lys Ile Leu Ser Ile Leu Ser Glu | |
| Leu Arg Thr Leu Gly Met Gln Asn Ser Asn Met Cys Ile Ser Leu Lys | |
| Leu Lys Asn Arg Lys Leu Pro Pro Phe Leu Glu Glu Ile Trp Asp Val | |
| <210> SEQ ID NO: 18 | |
| <211> 237 | |
| <212> PRT | |
| <213> Drosophila melanogaster | |
| <400> SEQUENCE: 18 | |
| Leu Thr Tyr Asn Gln Leu Ala Val Ile Tyr Lys Leu Ile Trp Tyr Gln | |
| Asp Gly Tyr Glu Gln Pro Ser Glu Glu Asp Leu Arg Arg Ile Met Ser | |
| Gln Pro Asp Glu Asn Glu Ser Gln Thr Asp Val Ser Phe Arg His Ile | |
| Thr Glu Ile Thr Ile Leu Thr Val Gln Leu Ile Val Glu Phe Ala Lys | |
| Gly Leu Pro Ala Phe Thr Lys Ile Pro Gln Glu Asp Gln Ile Thr Leu | |
| Leu Lys Ala Cys Ser Ser Glu Val Met Met Leu Arg Met Ala Arg Arg | |
| Tyr Asp His Ser Ser Asp Ser Ile Phe Phe Ala Asn Asn Arg Ser Tyr | |
| Thr Arg Asp Ser Tyr Lys Met Ala Gly Met Ala Asp Asn Ile Glu Asp | |
| Leu Leu His Phe Cys Arg Gln Met Phe Ser Met Lys Val Asp Asn Val | |
| Glu Tyr Ala Leu Leu Thr Ala Ile Val Ile Phe Ser Asp Arg Pro Gly | |
| Leu Glu Lys Ala Gln Leu Val Glu Ala Ile Gln Ser Tyr Tyr Ile Asp | |
| Thr Leu Arg Ile Tyr Ile Leu Asn Arg His Cys Gly Asp Ser Met Ser | |
| Leu Val Phe Tyr Ala Lys Leu Leu Ser Ile Leu Thr Glu Leu Arg Thr | |
| Leu Gly Asn Gln Asn Ala Glu Met Cys Phe Ser Leu Lys Leu Lys Asn | |
| Arg Lys Leu Pro Lys Phe Leu Glu Glu Ile Trp Asp Val | |
| <210> SEQ ID NO: 19 | |
| <211> 240 | |
| <212> PRT | |
| <213> Amblyomma americanum | |
| <400> SEQUENCE: 19 | |
| Pro Gly Val Lys Pro Leu Ser Ser Ser Gln Glu Asp Leu Ile Asn Lys | |
| Leu Val Tyr Tyr Gln Gln Glu Phe Glu Ser Pro Ser Glu Glu Asp Met | |
| Lys Lys Thr Thr Pro Phe Pro Leu Gly Asp Ser Glu Glu Asp Asn Gln | |
| Arg Arg Phe Gln His Ile Thr Glu Ile Thr Ile Leu Thr Val Gln Leu | |
| Ile Val Glu Phe Ser Lys Arg Val Pro Gly Phe Asp Thr Leu Ala Arg | |
| Glu Asp Gln Ile Thr Leu Leu Lys Ala Cys Ser Ser Glu Val Met Met | |
| Leu Arg Gly Ala Arg Lys Tyr Asp Val Lys Thr Asp Ser Ile Val Phe | |
| Ala Asn Asn Gln Pro Tyr Thr Arg Asp Asn Tyr Arg Ser Ala Ser Val | |
| Gly Asp Ser Ala Asp Ala Leu Phe Arg Phe Cys Arg Lys Met Cys Gln | |
| Leu Arg Val Asp Asn Ala Glu Tyr Ala Leu Leu Thr Ala Ile Val Ile | |
| Phe Ser Glu Arg Pro Ser Leu Val Asp Pro His Lys Val Glu Arg Ile | |
| Gln Glu Tyr Tyr Ile Glu Thr Leu Arg Met Tyr Ser Glu Asn His Arg | |
| Pro Pro Gly Lys Asn Tyr Phe Ala Arg Leu Leu Ser Ile Leu Thr Glu | |
| Leu Arg Thr Leu Gly Asn Met Asn Ala Glu Met Cys Phe Ser Leu Lys | |
| Val Gln Asn Lys Lys Leu Pro Pro Phe Leu Ala Glu Ile Trp Asp Ile | |
| <210> SEQ ID NO: 20 | |
| <211> LENGTH: 1586 | |
| <212> TYPE: DNA | |
| <213> ORGANISM: Bamecia argentifoli | |
| <400> SEQUENCE: 20 | |
| gaattcgcgg ccgctcgcaa acttccgtac ctctcacccc ctcgccagga ccccccgcca | 60 |
| accagttcac cgtcatctcc tccaatggat actcatcccc catgtcttcg ggcagctacg | 120 |
| acccttatag tcccaccaat ggaagaatag ggaaagaaga gctttcgccg gcgaatagtc | 180 |
| tgaacgggta caacgtggat agctgcgatg cgtcgcggaa gaagaaggga ggaacgggtc | 240 |
| ggcagcagga ggagctgtgt ctcgtctgcg gggaccgcgc ctccggctac cactacaacg | 300 |
| ccctcacctg cgaaggctgc aagggcttct tccgtcggag catcaccaag aatgccgtct | 360 |
| accagtgtaa atatggaaat aattgtgaaa ttgacatgta catgaggcga aaatgccaag | 420 |
| agtgtcgtct caagaagtgt ctcagcgttg gcatgaggcc agaatgtgta gttcccgaat | 480 |
| tccagtgtgc tgtgaagcga aaagagaaaa aagcgcaaaa ggacaaagat aaacctaact | 540 |
| caacgacgag ttgttctcca gatggaatca aacaagagat agatcctcaa aggctggata | 600 |
| cagattcgca gctattgtct gtaaatggag ttaaacccat tactccagag caagaagagc | 660 |
| tcatccatag gctagtttat tttcaaaatg aatatgaaca tccatcccca gaggatatca | 720 |
| aaaggatagt taatgctgca ccagaagaag aaaatgtagc tgaagaaagg tttaggcata | 780 |
| ttacagaaat tacaattctc actgtacagt taattgtgga attttctaag cgattacctg | 840 |
| gttttgacaa actaattcgt gaagatcaaa tagctttatt aaaggcatgt agtagtgaag | 900 |
| taatgatgtt tagaatggca aggaggtatg atgctgaaac agattcgata ttgtttgcaa | 960 |
| ctaaccagcc gtatacgaga gaatcataca ctgtagctgg catgggtgat actgtggagg | 1020 |
| atctgctccg attttgtcga catatgtgtg ccatgaaagt cgataacgca gaatatgctc | 1080 |
| ttctcactgc cattgtaatt ttttcagaac gaccatctct aagtgaaggc tggaaggttg | 1140 |
| agaagattca agaaatttac atagaagcat taaaagcata tgttgaaaat cgaaggaaac | 1200 |
| catatgcaac aaccattttt gctaagttac tatctgtttt aactgaacta cgaacattag | 1260 |
| ggaatatgaa ttcagaaaca tgcttctcat tgaagctgaa gaatagaaag gtgccatcct | 1320 |
| tcctcgagga gatttgggat gttgtttcat aaacagtctt acctcaattc catgttactt | 1380 |
| ttcatatttg atttatctca gcaggtggct cagtacttat cctcacatta ctgagctcac | 1440 |
| ggtatgctca tacaattata acttgtaata tcatatcggt gatgacaaat ttgttacaat | 1500 |
| attctttgtt accttaacac aatgttgatc tcataatgat gtatgaattt ttctgttttt | 1560 |
| gcaaaaaaaa aagcggccgc gaattc | 1586 |
| <210> SEQ ID NO: 21 | |
| <211> LENGTH: 1109 | |
| <212> TYPE: DNA | |
| <213> ORGANISM: Nephotetix cincticeps | |
| <400> SEQUENCE: 21 | |
| caggaggagc tctgcctgtt gtgcggagac cgagcgtcgg gataccacta caacgctctc | 60 |
| acctgcgaag gatgcaaggg cttctttcgg aggagtatca ccaaaaacgc agtgtaccag | 120 |
| tccaaatacg gcaccaattg tgaaatagac atgtatatgc ggcgcaagtg ccaggagtgc | 180 |
| cgactcaaga agtgcctcag tgtagggatg aggccagaat gtgtagtacc tgagtatcaa | 240 |
| tgtgccgtaa aaaggaaaga gaaaaaagct caaaaggaca aagataaacc tgtctcttca | 300 |
| accaatggct cgcctgaaat gagaatagac caggacaacc gttgtgtggt gttgcagagt | 360 |
| gaagacaaca ggtacaactc gagtacgccc agtttcggag tcaaacccct cagtccagaa | 420 |
| caagaggagc tcatccacag gctcgtctac ttccagaacg agtacgaaca ccctgccgag | 480 |
| gaggatctca agcggatcga gaacctcccc tgtgacgacg atgacccgtg tgatgttcgc | 540 |
| tacaaacaca ttacggagat cacaatactc acagtccagc tcatcgtgga gtttgcgaaa | 600 |
| aaactgcctg gtttcgacaa actactgaga gaggaccaga tcgtgttgct caaggcgtgt | 660 |
| tcgagcgagg tgatgatgct gcggatggcg cggaggtacg acgtccagac agactcgatc | 720 |
| ctgttcgcca acaaccagcc gtacacgcga gagtcgtaca cgatggcagg cgtgggggaa | 780 |
| gtcatcgaag atctgctgcg gttcggccga ctcatgtgct ccatgaaggt ggacaatgcc | 840 |
| gagtatgctc tgctcacggc catcgtcatc ttctccgagc ggccgaacct ggcggaagga | 900 |
| tggaaggttg agaagatcca ggagatctac ctggaggcgc tcaagtccta cgtggacaac | 960 |
| cgagtgaaac ctcgcagtcc gaccatcttc gccaaactgc tctccgttct caccgagctg | 1020 |
| cgaacactcg gcaaccagaa ctccgagatg tgcttctcgt taaactacgc aaccgcaaac | 1080 |
| atgccaccgt tcctcgaaga aatctggga | 1109 |
| <210> SEQ ID NO: 22 | |
| <211> LENGTH: 735 | |
| <212> TYPE: DNA | |
| <213> ORGANISM: Choristoneura fumiferana | |
| <400> SEQUENCE: 22 | |
| taccaggacg ggtacgagca gccttctgat gaagatttga agaggattac gcagacgtgg | 60 |
| cagcaagcgg acgatgaaaa cgaagagtct gacactccct tccgccagat cacagagatg | 120 |
| actatcctca cggtccaact tatcgtggag ttcgcgaagg gattgccagg gttcgccaag | 180 |
| atctcgcagc ctgatcaaat tacgctgctt aaggcttgct caagtgaggt aatgatgctc | 240 |
| cgagtcgcgc gacgatacga tgcggcctca gacagtgttc tgttcgcgaa caaccaagcg | 300 |
| tacactcgcg acaactaccg caaggctggc atggcctacg tcatcgagga tctactgcac | 360 |
| ttctgccggt gcatgtactc tatggcgttg gacaacatcc attacgcgct gctcacggct | 420 |
| gtcgtcatct tttctgaccg gccagggttg gagcagccgc aactggtgga agaaatccag | 480 |
| cggtactacc tgaatacgct ccgcatctat atcctgaacc agctgagcgg gtcggcgcgt | 540 |
| tcgtccgtca tatacggcaa gatcctctca atcctctctg agctacgcac gctcggcatg | 600 |
| caaaactcca acatgtgcat ctccctcaag ctcaagaaca gaaagctgcc gcctttcctc | 660 |
| gaggagatct gggatgtggc ggacatgtcg cacacccaac cgccgcctat cctcgagtcc | 720 |
| cccacgaatc tctag | 735 |
| <210> SEQ ID NO: 23 | |
| <211> LENGTH: 1338 | |
| <212> TYPE: DNA | |
| <213> ORGANISM: Drosophila melanogaster | |
| <400> SEQUENCE: 23 | |
| tatgagcagc catctgaaga ggatctcagg cgtataatga gtcaacccga tgagaacgag | 60 |
| agccaaacgg acgtcagctt tcggcatata accgagataa ccatactcac ggtccagttg | 120 |
| attgttgagt ttgctaaagg tctaccagcg tttacaaaga taccccagga ggaccagatc | 180 |
| acgttactaa aggcctgctc gtcggaggtg atgatgctgc gtatggcacg acgctatgac | 240 |
| cacagctcgg actcaatatt cttcgcgaat aatagatcat atacgcggga ttcttacaaa | 300 |
| atggccggaa tggctgataa cattgaagac ctgctgcatt tctgccgcca aatgttctcg | 360 |
| atgaaggtgg acaacgtcga atacgcgctt ctcactgcca ttgtgatctt ctcggaccgg | 420 |
| ccgggcctgg agaaggccca actagtcgaa gcgatccaga gctactacat cgacacgcta | 480 |
| cgcatttata tactcaaccg ccactgcggc gactcaatga gcctcgtctt ctacgcaaag | 540 |
| ctgctctcga tcctcaccga gctgcgtacg ctgggcaacc agaacgccga gatgtgtttc | 600 |
| tcactaaagc tcaaaaaccg caaactgccc aagttcctcg aggagatctg ggacgttcat | 660 |
| gccatcccgc catcggtcca gtcgcacctt cagattaccc aggaggagaa cgagcgtctc | 720 |
| gagcgggctg agcgtatgcg ggcatcggtt gggggcgcca ttaccgccgg cattgattgc | 780 |
| gactctgcct ccacttcggc ggcggcagcc gcggcccagc atcagcctca gcctcagccc | 840 |
| cagccccaac cctcctccct gacccagaac gattcccagc accagacaca gccgcagcta | 900 |
| caacctcagc taccacctca gctgcaaggt caactgcaac cccagctcca accacagctt | 960 |
| cagacgcaac tccagccaca gattcaacca cagccacagc tccttcccgt ctccgctccc | 1020 |
| gtgcccgcct ccgtaaccgc acctggttcc ttgtccgcgg tcagtacgag cagcgaatac | 1080 |
| atgggcggaa gtgcggccat aggacccatc acgccggcaa ccaccagcag tatcacggct | 1140 |
| gccgttaccg ctagctccac cacatcagcg gtaccgatgg gcaacggagt tggagtcggt | 1200 |
| gttggggtgg gcggcaacgt cagcatgtat gcgaacgccc agacggcgat ggccttgatg | 1260 |
| ggtgtagccc tgcattcgca ccaagagcag cttatcgggg gagtggcggt taagtcggag | 1320 |
| cactcgacga ctgcatag | 1338 |
| <210> SEQ ID NO: 24 | |
| <211> LENGTH: 960 | |
| <212> TYPE: DNA | |
| <213> ORGANISM: Choristoneura fumiferana | |
| <400> SEQUENCE: 24 | |
| cctgagtgcg tagtacccga gactcagtgc gccatgaagc ggaaagagaa gaaagcacag | 60 |
| aaggagaagg acaaactgcc tgtcagcacg acgacggtgg acgaccacat gccgcccatt | 120 |
| atgcagtgtg aacctccacc tcctgaagca gcaaggattc acgaagtggt cccaaggttt | 180 |
| ctctccgaca agctgttgga gacaaaccgg cagaaaaaca tcccccagtt gacagccaac | 240 |
| cagcagttcc ttatcgccag gctcatctgg taccaggacg ggtacgagca gccttctgat | 300 |
| gaagatttga agaggattac gcagacgtgg cagcaagcgg acgatgaaaa cgaagagtct | 360 |
| gacactccct tccgccagat cacagagatg actatcctca cggtccaact tatcgtggag | 420 |
| ttcgcgaagg gattgccagg gttcgccaag atctcgcagc ctgatcaaat tacgctgctt | 480 |
| aaggcttgct caagtgaggt aatgatgctc cgagtcgcgc gacgatacga tgcggcctca | 540 |
| gacagtgttc tgttcgcgaa caaccaagcg tacactcgcg acaactaccg caaggctggc | 600 |
| atggcctacg tcatcgagga tctactgcac ttctgccggt gcatgtactc tatggcgttg | 660 |
| gacaacatcc attacgcgct gctcacggct gtcgtcatct tttctgaccg gccagggttg | 720 |
| gagcagccgc aactggtgga agaaatccag cggtactacc tgaatacgct ccgcatctat | 780 |
| atcctgaacc agctgagcgg gtcggcgcgt tcgtccgtca tatacggcaa gatcctctca | 840 |
| atcctctctg agctacgcac gctcggcatg caaaactcca acatgtgcat ctccctcaag | 900 |
| ctcaagaaca gaaagctgcc gcctttcctc gaggagatct gggatgtggc ggacatgtcg | 960 |
| <210> SEQ ID NO: 25 | |
| <211> LENGTH: 969 | |
| <212> TYPE: DNA | |
| <213> ORGANISM: Drosophila melanogaster | |
| <400> SEQUENCE: 25 | |
| cggccggaat gcgtcgtccc ggagaaccaa tgtgcgatga agcggcgcga aaagaaggcc | 60 |
| cagaaggaga aggacaaaat gaccacttcg ccgagctctc agcatggcgg caatggcagc | 120 |
| ttggcctctg gtggcggcca agactttgtt aagaaggaga ttcttgacct tatgacatgc | 180 |
| gagccgcccc agcatgccac tattccgcta ctacctgatg aaatattggc caagtgtcaa | 240 |
| gcgcgcaata taccttcctt aacgtacaat cagttggccg ttatatacaa gttaatttgg | 300 |
| taccaggatg gctatgagca gccatctgaa gaggatctca ggcgtataat gagtcaaccc | 360 |
| gatgagaacg agagccaaac ggacgtcagc tttcggcata taaccgagat aaccatactc | 420 |
| acggtccagt tgattgttga gtttgctaaa ggtctaccag cgtttacaaa gataccccag | 480 |
| gaggaccaga tcacgttact aaaggcctgc tcgtcggagg tgatgatgct gcgtatggca | 540 |
| cgacgctatg accacagctc ggactcaata ttcttcgcga ataatagatc atatacgcgg | 600 |
| gattcttaca aaatggccgg aatggctgat aacattgaag acctgctgca tttctgccgc | 660 |
| caaatgttct cgatgaaggt ggacaacgtc gaatacgcgc ttctcactgc cattgtgatc | 720 |
| ttctcggacc ggccgggcct ggagaaggcc caactagtcg aagcgatcca gagctactac | 780 |
| atcgacacgc tacgcattta tatactcaac cgccactgcg gcgactcaat gagcctcgtc | 840 |
| ttctacgcaa agctgctctc gatcctcacc gagctgcgta cgctgggcaa ccagaacgcc | 900 |
| gagatgtgtt tctcactaaa gctcaaaaac cgcaaactgc ccaagttcct cgaggagatc | 960 |
| tgggacgtt | 969 |
| <210> SEQ ID NO: 26 | |
| <211> LENGTH: 244 | |
| <212> TYPE: PRT | |
| <213> ORGANISM: Choristoneura fumiferana | |
| <400> SEQUENCE: 26 | |
| Tyr Gln Asp Gly Tyr Glu Gln Pro Ser Asp Glu Asp Leu Lys Arg Ile | |
| Thr Gln Thr Trp Gln Gln Ala Asp Asp Glu Asn Glu Glu Ser Asp Thr | |
| Pro Phe Arg Gln Ile Thr Glu Met Thr Ile Leu Thr Val Gln Leu Ile | |
| Val Glu Phe Ala Lys Gly Leu Pro Gly Phe Ala Lys Ile Ser Gln Pro | |
| Asp Gln Ile Thr Leu Leu Lys Ala Cys Ser Ser Glu Val Met Met Leu | |
| Arg Val Ala Arg Arg Tyr Asp Ala Ala Ser Asp Ser Val Leu Phe Ala | |
| Asn Asn Gln Ala Tyr Thr Arg Asp Asn Tyr Arg Lys Ala Gly Met Ala | |
| Tyr Val Ile Glu Asp Leu Leu His Phe Cys Arg Cys Met Tyr Ser Met | |
| Ala Leu Asp Asn Ile His Tyr Ala Leu Leu Thr Ala Val Val Ile Phe | |
| Ser Asp Arg Pro Gly Leu Glu Gln Pro Gln Leu Val Glu Glu Ile Gln | |
| Arg Tyr Tyr Leu Asn Thr Leu Arg Ile Tyr Ile Leu Asn Gln Leu Ser | |
| Gly Ser Ala Arg Ser Ser Val Ile Tyr Gly Lys Ile Leu Ser Ile Leu | |
| Ser Glu Leu Arg Thr Leu Gly Met Gln Asn Ser Asn Met Cys Ile Ser | |
| Leu Lys Leu Lys Asn Arg Lys Leu Pro Pro Phe Leu Glu Glu Ile Trp | |
| Asp Val Ala Asp Met Ser His Thr Gln Pro Pro Pro Ile Leu Glu Ser | |
| Pro Thr Asn Leu | |
| <210> SEQ ID NO: 27 | |
| <211> LENGTH: 445 | |
| <212> TYPE: PRT | |
| <213> ORGANISM: Drosophila melanogaster | |
| <400> SEQUENCE: 27 | |
| Tyr Glu Gln Pro Ser Glu Glu Asp Leu Arg Arg Ile Met Ser Gln Pro | |
| Asp Glu Asn Glu Ser Gln Thr Asp Val Ser Phe Arg His Ile Thr Glu | |
| Ile Thr Ile Leu Thr Val Gln Leu Ile Val Glu Phe Ala Lys Gly Leu | |
| Pro Ala Phe Thr Lys Ile Pro Gln Glu Asp Gln Ile Thr Leu Leu Lys | |
| Ala Cys Ser Ser Glu Val Met Met Leu Arg Met Ala Arg Arg Tyr Asp | |
| His Ser Ser Asp Ser Ile Phe Phe Ala Asn Asn Arg Ser Tyr Thr Arg | |
| Asp Ser Tyr Lys Met Ala Gly Met Ala Asp Asn Ile Glu Asp Leu Leu | |
| His Phe Cys Arg Gln Met Phe Ser Met Lys Val Asp Asn Val Glu Tyr | |
| Ala Leu Leu Thr Ala Ile Val Ile Phe Ser Asp Arg Pro Gly Leu Glu | |
| Lys Ala Gln Leu Val Glu Ala Ile Gln Ser Tyr Tyr Ile Asp Thr Leu | |
| Arg Ile Tyr Ile Leu Asn Arg His Cys Gly Asp Ser Met Ser Leu Val | |
| Phe Tyr Ala Lys Leu Leu Ser Ile Leu Thr Glu Leu Arg Thr Leu Gly | |
| Asn Gln Asn Ala Glu Met Cys Phe Ser Leu Lys Leu Lys Asn Arg Lys | |
| Leu Pro Lys Phe Leu Glu Glu Ile Trp Asp Val His Ala Ile Pro Pro | |
| Ser Val Gln Ser His Leu Gln Ile Thr Gln Glu Glu Asn Glu Arg Leu | |
| Glu Arg Ala Glu Arg Met Arg Ala Ser Val Gly Gly Ala Ile Thr Ala | |
| Gly Ile Asp Cys Asp Ser Ala Ser Thr Ser Ala Ala Ala Ala Ala Ala | |
| Gln His Gln Pro Gln Pro Gln Pro Gln Pro Gln Pro Ser Ser Leu Thr | |
| Gln Asn Asp Ser Gln His Gln Thr Gln Pro Gln Leu Gln Pro Gln Leu | |
| Pro Pro Gln Leu Gln Gly Gln Leu Gln Pro Gln Leu Gln Pro Gln Leu | |
| Gln Thr Gln Leu Gln Pro Gln Ile Gln Pro Gln Pro Gln Leu Leu Pro | |
| Val Ser Ala Pro Val Pro Ala Ser Val Thr Ala Pro Gly Ser Leu Ser | |
| Ala Val Ser Thr Ser Ser Glu Tyr Met Gly Gly Ser Ala Ala Ile Gly | |
| Pro Ile Thr Pro Ala Thr Thr Ser Ser Ile Thr Ala Ala Val Thr Ala | |
| Ser Ser Thr Thr Ser Ala Val Pro Met Gly Asn Gly Val Gly Val Gly | |
| Val Gly Val Gly Gly Asn Val Ser Met Tyr Ala Asn Ala Gln Thr Ala | |
| Met Ala Leu Met Gly Val Ala Leu His Ser His Gln Glu Gln Leu Ile | |
| Gly Gly Val Ala Val Lys Ser Glu His Ser Thr Thr Ala | |
| <210> SEQ ID NO: 28 | |
| <211> LENGTH: 320 | |
| <212> TYPE: PRT | |
| <213> ORGANISM: Choristoneura fumiferana | |
| <400> SEQUENCE: 28 | |
| Pro Glu Cys Val Val Pro Glu Thr Gln Cys Ala Met Lys Arg Lys Glu | |
| Lys Lys Ala Gln Lys Glu Lys Asp Lys Leu Pro Val Ser Thr Thr Thr | |
| Val Asp Asp His Met Pro Pro Ile Met Gln Cys Glu Pro Pro Pro Pro | |
| Glu Ala Ala Arg Ile His Glu Val Val Pro Arg Phe Leu Ser Asp Lys | |
| Leu Leu Glu Thr Asn Arg Gln Lys Asn Ile Pro Gln Leu Thr Ala Asn | |
| Gln Gln Phe Leu Ile Ala Arg Leu Ile Trp Tyr Gln Asp Gly Tyr Glu | |
| Gln Pro Ser Asp Glu Asp Leu Lys Arg Ile Thr Gln Thr Trp Gln Gln | |
| Ala Asp Asp Glu Asn Glu Glu Ser Asp Thr Pro Phe Arg Gln Ile Thr | |
| Glu Met Thr Ile Leu Thr Val Gln Leu Ile Val Glu Phe Ala Lys Gly | |
| Leu Pro Gly Phe Ala Lys Ile Ser Gln Pro Asp Gln Ile Thr Leu Leu | |
| Lys Ala Cys Ser Ser Glu Val Met Met Leu Arg Val Ala Arg Arg Tyr | |
| Asp Ala Ala Ser Asp Ser Val Leu Phe Ala Asn Asn Gln Ala Tyr Thr | |
| Arg Asp Asn Tyr Arg Lys Ala Gly Met Ala Tyr Val Ile Glu Asp Leu | |
| Leu His Phe Cys Arg Cys Met Tyr Ser Met Ala Leu Asp Asn Ile His | |
| Tyr Ala Leu Leu Thr Ala Val Val Ile Phe Ser Asp Arg Pro Gly Leu | |
| Glu Gln Pro Gln Leu Val Glu Glu Ile Gln Arg Tyr Tyr Leu Asn Thr | |
| Leu Arg Ile Tyr Ile Leu Asn Gln Leu Ser Gly Ser Ala Arg Ser Ser | |
| Val Ile Tyr Gly Lys Ile Leu Ser Ile Leu Ser Glu Leu Arg Thr Leu | |
| Gly Met Gln Asn Ser Asn Met Cys Ile Ser Leu Lys Leu Lys Asn Arg | |
| Lys Leu Pro Pro Phe Leu Glu Glu Ile Trp Asp Val Ala Asp Met Ser | |
| <210> SEQ ID NO: 29 | |
| <211> LENGTH: 323 | |
| <212> TYPE: PRT | |
| <213> ORGANISM: Drosophila melanogaster | |
| <400> SEQUENCE: 29 | |
| Arg Pro Glu Cys Val Val Pro Glu Asn Gln Cys Ala Met Lys Arg Arg | |
| Glu Lys Lys Ala Gln Lys Glu Lys Asp Lys Met Thr Thr Ser Pro Ser | |
| Ser Gln His Gly Gly Asn Gly Ser Leu Ala Ser Gly Gly Gly Gln Asp | |
| Phe Val Lys Lys Glu Ile Leu Asp Leu Met Thr Cys Glu Pro Pro Gln | |
| His Ala Thr Ile Pro Leu Leu Pro Asp Glu Ile Leu Ala Lys Cys Gln | |
| Ala Arg Asn Ile Pro Ser Leu Thr Tyr Asn Gln Leu Ala Val Ile Tyr | |
| Lys Leu Ile Trp Tyr Gln Asp Gly Tyr Glu Gln Pro Ser Glu Glu Asp | |
| Leu Arg Arg Ile Met Ser Gln Pro Asp Glu Asn Glu Ser Gln Thr Asp | |
| Val Ser Phe Arg His Ile Thr Glu Ile Thr Ile Leu Thr Val Gln Leu | |
| Ile Val Glu Phe Ala Lys Gly Leu Pro Ala Phe Thr Lys Ile Pro Gln | |
| Glu Asp Gln Ile Thr Leu Leu Lys Ala Cys Ser Ser Glu Val Met Met | |
| Leu Arg Met Ala Arg Arg Tyr Asp His Ser Ser Asp Ser Ile Phe Phe | |
| Ala Asn Asn Arg Ser Tyr Thr Arg Asp Ser Tyr Lys Met Ala Gly Met | |
| Ala Asp Asn Ile Glu Asp Leu Leu His Phe Cys Arg Gln Met Phe Ser | |
| Met Lys Val Asp Asn Val Glu Tyr Ala Leu Leu Thr Ala Ile Val Ile | |
| Phe Ser Asp Arg Pro Gly Leu Glu Lys Ala Gln Leu Val Glu Ala Ile | |
| Gln Ser Tyr Tyr Ile Asp Thr Leu Arg Ile Tyr Ile Leu Asn Arg His | |
| Cys Gly Asp Ser Met Ser Leu Val Phe Tyr Ala Lys Leu Leu Ser Ile | |
| Leu Thr Glu Leu Arg Thr Leu Gly Asn Gln Asn Ala Glu Met Cys Phe | |
| Ser Leu Lys Leu Lys Asn Arg Lys Leu Pro Lys Phe Leu Glu Glu Ile | |
| Trp Asp Val | |
| <210> SEQ ID NO: 30 | |
| <211> LENGTH: 987 | |
| <212> TYPE: DNA | |
| <213> ORGANISM: Artificial Sequence | |
| <221> NAME/KEY: misc_feature | |
| <400> SEQUENCE: 30 | |
| tgtgctatct gtggggaccg ctcctcaggc aaacactatg gggtatacag ttgtgagggc | 60 |
| tgcaagggct tcttcaagag gacagtacgc aaagacctga cctacacctg ccgagacaac | 120 |
| aaggactgcc tgatcgacaa gagacagcgg aaccggtgtc agtactgccg ctaccagaag | 180 |
| tgcctggcca tgggcatgaa gcgggaagct gtgcaggagg agcggcagcg gggcaaggac | 240 |
| cggaatgaga acgaggtgga gtccaccagc agtgccaacg aggacatgcc tgtagagaag | 300 |
| attctggaag ccgagcttgc tgtcgagccc aagactgaga catacgtgga ggcaaacatg | 360 |
| gggctgaacc ccagctcacc aaatgaccct gttaccaaca tctgtcaagc agcagacaag | 420 |
| cagctcttca ctcttgtgga gtgggccaag aggatcccac acttttctga gctgccccta | 480 |
| gacgaccagg tcatcctgct acgggcaggc tggaacgagc tgctgatcgc ctccttctcc | 540 |
| caccgctcca tagctgtgaa agatgggatt ctcctggcca ccggcctgca cgtacaccgg | 600 |
| aacagcgctc acagtgctgg ggtgggcgcc atctttgaca gggtgctaac agagctggtg | 660 |
| tctaagatgc gtgacatgca gatggacaag acggagctgg gctgcctgcg agccattgtc | 720 |
| ctgttcaacc ctgactctaa ggggctctca aaccctgctg aggtggaggc gttgagggag | 780 |
| aaggtgtatg cgtcactaga agcgtactgc aaacacaagt accctgagca gccgggcagg | 840 |
| tttgccaagc tgctgctccg cctgcctgca ctgcgttcca tcgggctcaa gtgcctggag | 900 |
| cacctgttct tcttcaagct catcggggac acgcccatcg acaccttcct catggagatg | 960 |
| ctggaggcac cacatcaagc cacctag | 987 |
| <210> SEQ ID NO: 31 | |
| <211> LENGTH: 789 | |
| <212> TYPE: DNA | |
| <213> ORGANISM: Artificial Sequence | |
| <221> NAME/KEY: misc_feature | |
| <400> SEQUENCE: 31 | |
| aagcgggaag ctgtgcagga ggagcggcag cggggcaagg accggaatga gaacgaggtg | 60 |
| gagtccacca gcagtgccaa cgaggacatg cctgtagaga agattctgga agccgagctt | 120 |
| gctgtcgagc ccaagactga gacatacgtg gaggcaaaca tggggctgaa ccccagctca | 180 |
| ccaaatgacc ctgttaccaa catctgtcaa gcagcagaca agcagctctt cactcttgtg | 240 |
| gagtgggcca agaggatccc acacttttct gagctgcccc tagacgacca ggtcatcctg | 300 |
| ctacgggcag gctggaacga gctgctgatc gcctccttct cccaccgctc catagctgtg | 360 |
| aaagatggga ttctcctggc caccggcctg cacgtacacc ggaacagcgc tcacagtgct | 420 |
| ggggtgggcg ccatctttga cagggtgcta acagagctgg tgtctaagat gcgtgacatg | 480 |
| cagatggaca agacggagct gggctgcctg cgagccattg tcctgttcaa ccctgactct | 540 |
| aaggggctct caaaccctgc tgaggtggag gcgttgaggg agaaggtgta tgcgtcacta | 600 |
| gaagcgtact gcaaacacaa gtaccctgag cagccgggca ggtttgccaa gctgctgctc | 660 |
| cgcctgcctg cactgcgttc catcgggctc aagtgcctgg agcacctgtt cttcttcaag | 720 |
| ctcatcgggg acacgcccat cgacaccttc ctcatggaga tgctggaggc accacatcaa | 780 |
| gccacctag | 789 |
| <210> SEQ ID NO: 32 | |
| <211> LENGTH: 714 | |
| <212> TYPE: DNA | |
| <213> ORGANISM: Artificial Sequence | |
| <221> NAME/KEY: misc_feature | |
| <400> SEQUENCE: 32 | |
| gccaacgagg acatgcctgt agagaagatt ctggaagccg agcttgctgt cgagcccaag | 60 |
| actgagacat acgtggaggc aaacatgggg ctgaacccca gctcaccaaa tgaccctgtt | 120 |
| accaacatct gtcaagcagc agacaagcag ctcttcactc ttgtggagtg ggccaagagg | 180 |
| atcccacact tttctgagct gcccctagac gaccaggtca tcctgctacg ggcaggctgg | 240 |
| aacgagctgc tgatcgcctc cttctcccac cgctccatag ctgtgaaaga tgggattctc | 300 |
| ctggccaccg gcctgcacgt acaccggaac agcgctcaca gtgctggggt gggcgccatc | 360 |
| tttgacaggg tgctaacaga gctggtgtct aagatgcgtg acatgcagat ggacaagacg | 420 |
| gagctgggct gcctgcgagc cattgtcctg ttcaaccctg actctaaggg gctctcaaac | 480 |
| cctgctgagg tggaggcgtt gagggagaag gtgtatgcgt cactagaagc gtactgcaaa | 540 |
| cacaagtacc ctgagcagcc gggcaggttt gccaagctgc tgctccgcct gcctgcactg | 600 |
| cgttccatcg ggctcaagtg cctggagcac ctgttcttct tcaagctcat cggggacacg | 660 |
| cccatcgaca ccttcctcat ggagatgctg gaggcaccac atcaagccac ctag | 714 |
| <210> SEQ ID NO: 33 | |
| <211> LENGTH: 536 | |
| <212> TYPE: DNA | |
| <213> ORGANISM: Artificial Sequence | |
| <221> NAME/KEY: misc_feature | |
| <400> SEQUENCE: 33 | |
| ggatcccaca cttttctgag ctgcccctag acgaccaggt catcctgcta cgggcaggct | 60 |
| ggaacgagct gctgatcgcc tccttctccc accgctccat agctgtgaaa gatgggattc | 120 |
| tcctggccac cggcctgcac gtacaccgga acagcgctca cagtgctggg gtgggcgcca | 180 |
| tctttgacag ggtgctaaca gagctggtgt ctaagatgcg tgacatgcag atggacaaga | 240 |
| cggagctggg ctgcctgcga gccattgtcc tgttcaaccc tgactctaag gggctctcaa | 300 |
| accctgctga ggtggaggcg ttgagggaga aggtgtatgc gtcactagaa gcgtactgca | 360 |
| aacacaagta ccctgagcag ccgggcaggt ttgccaagct gctgctccgc ctgcctgcac | 420 |
| tgcgttccat cgggctcaag tgcctggagc acctgttctt cttcaagctc atcggggaca | 480 |
| cgcccatcga caccttcctc atggagatgc tggaggcacc acatcaagcc acctag | 536 |
| <210> SEQ ID NO: 34 | |
| <211> LENGTH: 672 | |
| <212> TYPE: DNA | |
| <213> ORGANISM: Artificial Sequence | |
| <221> NAME/KEY: misc_feature | |
| <400> SEQUENCE: 34 | |
| gccaacgagg acatgcctgt agagaagatt ctggaagccg agcttgctgt cgagcccaag | 60 |
| actgagacat acgtggaggc aaacatgggg ctgaacccca gctcaccaaa tgaccctgtt | 120 |
| accaacatct gtcaagcagc agacaagcag ctcttcactc ttgtggagtg ggccaagagg | 180 |
| atcccacact tttctgagct gcccctagac gaccaggtca tcctgctacg ggcaggctgg | 240 |
| aacgagctgc tgatcgcctc cttctcccac cgctccatag ctgtgaaaga tgggattctc | 300 |
| ctggccaccg gcctgcacgt acaccggaac agcgctcaca gtgctggggt gggcgccatc | 360 |
| tttgacaggg tgctaacaga gctggtgtct aagatgcgtg acatgcagat ggacaagacg | 420 |
| gagctgggct gcctgcgagc cattgtcctg ttcaaccctg actctaaggg gctctcaaac | 480 |
| cctgctgagg tggaggcgtt gagggagaag gtgtatgcgt cactagaagc gtactgcaaa | 540 |
| cacaagtacc ctgagcagcc gggcaggttt gccaagctgc tgctccgcct gcctgcactg | 600 |
| cgttccatcg ggctcaagtg cctggagcac ctgttcttct tcaagctcat cggggacacg | 660 |
| cccatcgaca cc | 672 |
| <210> SEQ ID NO: 35 | |
| <211> LENGTH: 1123 | |
| <212> TYPE: DNA | |
| <213> ORGANISM: Artificial Sequence | |
| <220> FEATURE: | |
| <221> NAME/KEY: misc_feature | |
| <223> OTHER INFORMATION: Novel Sequence | |
| <400> SEQUENCE: 35 | |
| tgcgccatct gcggggaccg ctcctcaggc aagcactatg gagtgtacag ctgcgagggg | 60 |
| tgcaagggct tcttcaagcg gacggtgcgc aaggacctga cctacacctg ccgcgacaac | 120 |
| aaggactgcc tgattgacaa gcggcagcgg aaccggtgcc agtactgccg ctaccagaag | 180 |
| tgcctggcca tgggcatgaa gcgggaagcc gtgcaggagg agcggcagcg tggcaaggac | 240 |
| cggaacgaga atgaggtgga gtcgaccagc agcgccaacg aggacatgcc ggtggagagg | 300 |
| atcctggagg ctgagctggc cgtggagccc aagaccgaga cctacgtgga ggcaaacatg | 360 |
| gggctgaacc ccagctcgcc gaacgaccct gtcaccaaca tttgccaagc agccgacaaa | 420 |
| cagcttttca ccctggtgga gtgggccaag cggatcccac acttctcaga gctgcccctg | 480 |
| gacgaccagg tcatcctgct gcgggcaggc tggaatgagc tgctcatcgc ctccttctcc | 540 |
| caccgctcca tcgccgtgaa ggacgggatc ctcctggcca ccgggctgca cgtccaccgg | 600 |
| aacagcgccc acagcgcagg ggtgggcgcc atctttgaca gggtgctgac ggagcttgtg | 660 |
| tccaagatgc gggacatgca gatggacaag acggagctgg gctgcctgcg cgccatcgtc | 720 |
| ctctttaacc ctgactccaa ggggctctcg aacccggccg aggtggaggc gctgagggag | 780 |
| aaggtctatg cgtccttgga ggcctactgc aagcacaagt acccagagca gccgggaagg | 840 |
| ttcgctaagc tcttgctccg cctgccggct ctgcgctcca tcgggctcaa atgcctggaa | 900 |
| catctcttct tcttcaagct catcggggac acacccattg acaccttcct tatggagatg | 960 |
| ctggaggcgc cgcaccaaat gacttaggcc tgcgggccca tcctttgtgc ccacccgttc | 1020 |
| tggccaccct gcctggacgc cagctgttct tctcagcctg agccctgtcc ctgcccttct | 1080 |
| ctgcctggcc tgtttggact ttggggcaca gcctgtcact gct | 1123 |
| <210> SEQ ID NO: 36 | |
| <211> LENGTH: 925 | |
| <212> TYPE: DNA | |
| <213> ORGANISM: Artificial Sequence | |
| <220> FEATURE: | |
| <221> NAME/KEY: misc_feature | |
| <223> OTHER INFORMATION: Novel Sequence | |
| <400> SEQUENCE: 36 | |
| aagcgggaag ccgtgcagga ggagcggcag cgtggcaagg accggaacga gaatgaggtg | 60 |
| gagtcgacca gcagcgccaa cgaggacatg ccggtggaga ggatcctgga ggctgagctg | 120 |
| gccgtggagc ccaagaccga gacctacgtg gaggcaaaca tggggctgaa ccccagctcg | 180 |
| ccgaacgacc ctgtcaccaa catttgccaa gcagccgaca aacagctttt caccctggtg | 240 |
| gagtgggcca agcggatccc acacttctca gagctgcccc tggacgacca ggtcatcctg | 300 |
| ctgcgggcag gctggaatga gctgctcatc gcctccttct cccaccgctc catcgccgtg | 360 |
| aaggacggga tcctcctggc caccgggctg cacgtccacc ggaacagcgc ccacagcgca | 420 |
| ggggtgggcg ccatctttga cagggtgctg acggagcttg tgtccaagat gcgggacatg | 480 |
| cagatggaca agacggagct gggctgcctg cgcgccatcg tcctctttaa ccctgactcc | 540 |
| aaggggctct cgaacccggc cgaggtggag gcgctgaggg agaaggtcta tgcgtccttg | 600 |
| gaggcctact gcaagcacaa gtacccagag cagccgggaa ggttcgctaa gctcttgctc | 660 |
| cgcctgccgg ctctgcgctc catcgggctc aaatgcctgg aacatctctt cttcttcaag | 720 |
| ctcatcgggg acacacccat tgacaccttc cttatggaga tgctggaggc gccgcaccaa | 780 |
| atgacttagg cctgcgggcc catcctttgt gcccacccgt tctggccacc ctgcctggac | 840 |
| gccagctgtt cttctcagcc tgagccctgt ccctgccctt ctctgcctgg cctgtttgga | 900 |
| ctttggggca cagcctgtca ctgct | 925 |
| <210> SEQ ID NO: 37 | |
| <211> LENGTH: 850 | |
| <212> TYPE: DNA | |
| <213> ORGANISM: Artificial Sequence | |
| <220> FEATURE: | |
| <221> NAME/KEY: misc_feature | |
| <223> OTHER INFORMATION: Novel Sequence | |
| <400> SEQUENCE: 37 | |
| gccaacgagg acatgccggt ggagaggatc ctggaggctg agctggccgt ggagcccaag | 60 |
| accgagacct acgtggaggc aaacatgggg ctgaacccca gctcgccgaa cgaccctgtc | 120 |
| accaacattt gccaagcagc cgacaaacag cttttcaccc tggtggagtg ggccaagcgg | 180 |
| atcccacact tctcagagct gcccctggac gaccaggtca tcctgctgcg ggcaggctgg | 240 |
| aatgagctgc tcatcgcctc cttctcccac cgctccatcg ccgtgaagga cgggatcctc | 300 |
| ctggccaccg ggctgcacgt ccaccggaac agcgcccaca gcgcaggggt gggcgccatc | 360 |
| tttgacaggg tgctgacgga gcttgtgtcc aagatgcggg acatgcagat ggacaagacg | 420 |
| gagctgggct gcctgcgcgc catcgtcctc tttaaccctg actccaaggg gctctcgaac | 480 |
| ccggccgagg tggaggcgct gagggagaag gtctatgcgt ccttggaggc ctactgcaag | 540 |
| cacaagtacc cagagcagcc gggaaggttc gctaagctct tgctccgcct gccggctctg | 600 |
| cgctccatcg ggctcaaatg cctggaacat ctcttcttct tcaagctcat cggggacaca | 660 |
| cccattgaca ccttccttat ggagatgctg gaggcgccgc accaaatgac ttaggcctgc | 720 |
| gggcccatcc tttgtgccca cccgttctgg ccaccctgcc tggacgccag ctgttcttct | 780 |
| cagcctgagc cctgtccctg cccttctctg cctggcctgt ttggactttg gggcacagcc | 840 |
| tgtcactgct | 850 |
| <210> SEQ ID NO: 38 | |
| <211> LENGTH: 670 | |
| <212> TYPE: DNA | |
| <213> ORGANISM: Artificial Sequence | |
| <221> NAME/KEY: misc_feature | |
| <400> SEQUENCE: 38 | |
| atcccacact tctcagagct gcccctggac gaccaggtca tcctgctgcg ggcaggctgg | 60 |
| aatgagctgc tcatcgcctc cttctcccac cgctccatcg ccgtgaagga cgggatcctc | 120 |
| ctggccaccg ggctgcacgt ccaccggaac agcgcccaca gcgcaggggt gggcgccatc | 180 |
| tttgacaggg tgctgacgga gcttgtgtcc aagatgcggg acatgcagat ggacaagacg | 240 |
| gagctgggct gcctgcgcgc catcgtcctc tttaaccctg actccaaggg gctctcgaac | 300 |
| ccggccgagg tggaggcgct gagggagaag gtctatgcgt ccttggaggc ctactgcaag | 360 |
| cacaagtacc cagagcagcc gggaaggttc gctaagctct tgctccgcct gccggctctg | 420 |
| cgctccatcg ggctcaaatg cctggaacat ctcttcttct tcaagctcat cggggacaca | 480 |
| cccattgaca ccttccttat ggagatgctg gaggcgccgc accaaatgac ttaggcctgc | 540 |
| gggcccatcc tttgtgccca cccgttctgg ccaccctgcc tggacgccag ctgttcttct | 600 |
| cagcctgagc cctgtccctg cccttctctg cctggcctgt ttggactttg gggcacagcc | 660 |
| tgtcactgct | 670 |
| <210> SEQ ID NO: 39 | |
| <211> LENGTH: 672 | |
| <212> TYPE: DNA | |
| <213> ORGANISM: Artificial Sequence | |
| <221> NAME/KEY: misc_feature | |
| <400> SEQUENCE: 39 | |
| gccaacgagg acatgccggt ggagaggatc ctggaggctg agctggccgt ggagcccaag | 60 |
| accgagacct acgtggaggc aaacatgggg ctgaacccca gctcgccgaa cgaccctgtc | 120 |
| accaacattt gccaagcagc cgacaaacag cttttcaccc tggtggagtg ggccaagcgg | 180 |
| atcccacact tctcagagct gcccctggac gaccaggtca tcctgctgcg ggcaggctgg | 240 |
| aatgagctgc tcatcgcctc cttctcccac cgctccatcg ccgtgaagga cgggatcctc | 300 |
| ctggccaccg ggctgcacgt ccaccggaac agcgcccaca gcgcaggggt gggcgccatc | 360 |
| tttgacaggg tgctgacgga gcttgtgtcc aagatgcggg acatgcagat ggacaagacg | 420 |
| gagctgggct gcctgcgcgc catcgtcctc tttaaccctg actccaaggg gctctcgaac | 480 |
| ccggccgagg tggaggcgct gagggagaag gtctatgcgt ccttggaggc ctactgcaag | 540 |
| cacaagtacc cagagcagcc gggaaggttc gctaagctct tgctccgcct gccggctctg | 600 |
| cgctccatcg ggctcaaatg cctggaacat ctcttcttct tcaagctcat cggggacaca | 660 |
| cccattgaca cc | 672 |
| <210> SEQ ID NO: 40 | |
| <211> LENGTH: 328 | |
| <212> TYPE: PRT | |
| <213> ORGANISM: Artificial Sequence | |
| <221> NAME/KEY: misc_feature | |
| <400> SEQUENCE: 40 | |
| Cys Ala Ile Cys Gly Asp Arg Ser Ser Gly Lys His Tyr Gly Val Tyr | |
| Ser Cys Glu Gly Cys Lys Gly Phe Phe Lys Arg Thr Val Arg Lys Asp | |
| Leu Thr Tyr Thr Cys Arg Asp Asn Lys Asp Cys Leu Ile Asp Lys Arg | |
| Gln Arg Asn Arg Cys Gln Tyr Cys Arg Tyr Gln Lys Cys Leu Ala Met | |
| Gly Met Lys Arg Glu Ala Val Gln Glu Glu Arg Gln Arg Gly Lys Asp | |
| Arg Asn Glu Asn Glu Val Glu Ser Thr Ser Ser Ala Asn Glu Asp Met | |
| Pro Val Glu Lys Ile Leu Glu Ala Glu Leu Ala Val Glu Pro Lys Thr | |
| Glu Thr Tyr Val Glu Ala Asn Met Gly Leu Asn Pro Ser Ser Pro Asn | |
| Asp Pro Val Thr Asn Ile Cys Gln Ala Ala Asp Lys Gln Leu Phe Thr | |
| Leu Val Glu Trp Ala Lys Arg Ile Pro His Phe Ser Glu Leu Pro Leu | |
| Asp Asp Gln Val Ile Leu Leu Arg Ala Gly Trp Asn Glu Leu Leu Ile | |
| Ala Ser Phe Ser His Arg Ser Ile Ala Val Lys Asp Gly Ile Leu Leu | |
| Ala Thr Gly Leu His Val His Arg Asn Ser Ala His Ser Ala Gly Val | |
| Gly Ala Ile Phe Asp Arg Val Leu Thr Glu Leu Val Ser Lys Met Arg | |
| Asp Met Gln Met Asp Lys Thr Glu Leu Gly Cys Leu Arg Ala Ile Val | |
| Leu Phe Asn Pro Asp Ser Lys Gly Leu Ser Asn Pro Ala Glu Val Glu | |
| Ala Leu Arg Glu Lys Val Tyr Ala Ser Leu Glu Ala Tyr Cys Lys His | |
| Lys Tyr Pro Glu Gln Pro Gly Arg Phe Ala Lys Leu Leu Leu Arg Leu | |
| Pro Ala Leu Arg Ser Ile Gly Leu Lys Cys Leu Glu His Leu Phe Phe | |
| Phe Lys Leu Ile Gly Asp Thr Pro Ile Asp Thr Phe Leu Met Glu Met | |
| Leu Glu Ala Pro His Gln Ala Thr | |
| 325 | |
| <210> SEQ ID NO: 41 | |
| <211> LENGTH: 262 | |
| <212> TYPE: PRT | |
| <213> ORGANISM: Artificial Sequence | |
| <221> NAME/KEY: misc_feature | |
| <400> SEQUENCE: 41 | |
| Lys Arg Glu Ala Val Gln Glu Glu Arg Gln Arg Gly Lys Asp Arg Asn | |
| Glu Asn Glu Val Glu Ser Thr Ser Ser Ala Asn Glu Asp Met Pro Val | |
| Glu Lys Ile Leu Glu Ala Glu Leu Ala Val Glu Pro Lys Thr Glu Thr | |
| Tyr Val Glu Ala Asn Met Gly Leu Asn Pro Ser Ser Pro Asn Asp Pro | |
| Val Thr Asn Ile Cys Gln Ala Ala Asp Lys Gln Leu Phe Thr Leu Val | |
| Glu Trp Ala Lys Arg Ile Pro His Phe Ser Glu Leu Pro Leu Asp Asp | |
| Gln Val Ile Leu Leu Arg Ala Gly Trp Asn Glu Leu Leu Ile Ala Ser | |
| Phe Ser His Arg Ser Ile Ala Val Lys Asp Gly Ile Leu Leu Ala Thr | |
| Gly Leu His Val His Arg Asn Ser Ala His Ser Ala Gly Val Gly Ala | |
| Ile Phe Asp Arg Val Leu Thr Glu Leu Val Ser Lys Met Arg Asp Met | |
| Gln Met Asp Lys Thr Glu Leu Gly Cys Leu Arg Ala Ile Val Leu Phe | |
| Asn Pro Asp Ser Lys Gly Leu Ser Asn Pro Ala Glu Val Glu Ala Leu | |
| Arg Glu Lys Val Tyr Ala Ser Leu Glu Ala Tyr Cys Lys His Lys Tyr | |
| Pro Glu Gln Pro Gly Arg Phe Ala Lys Leu Leu Leu Arg Leu Pro Ala | |
| Leu Arg Ser Ile Gly Leu Lys Cys Leu Glu His Leu Phe Phe Phe Lys | |
| Leu Ile Gly Asp Thr Pro Ile Asp Thr Phe Leu Met Glu Met Leu Glu | |
| Ala Pro His Gln Ala Thr | |
| 260 | |
| <210> SEQ ID NO: 42 | |
| <211> LENGTH: 237 | |
| <212> TYPE: PRT | |
| <213> ORGANISM: Artificial Sequence | |
| <221> NAME/KEY: misc_feature | |
| <400> SEQUENCE: 42 | |
| Ala Asn Glu Asp Met Pro Val Glu Lys Ile Leu Glu Ala Glu Leu Ala | |
| Val Glu Pro Lys Thr Glu Thr Tyr Val Glu Ala Asn Met Gly Leu Asn | |
| Pro Ser Ser Pro Asn Asp Pro Val Thr Asn Ile Cys Gln Ala Ala Asp | |
| Lys Gln Leu Phe Thr Leu Val Glu Trp Ala Lys Arg Ile Pro His Phe | |
| Ser Glu Leu Pro Leu Asp Asp Gln Val Ile Leu Leu Arg Ala Gly Trp | |
| Asn Glu Leu Leu Ile Ala Ser Phe Ser His Arg Ser Ile Ala Val Lys | |
| Asp Gly Ile Leu Leu Ala Thr Gly Leu His Val His Arg Asn Ser Ala | |
| His Ser Ala Gly Val Gly Ala Ile Phe Asp Arg Val Leu Thr Glu Leu | |
| Val Ser Lys Met Arg Asp Met Gln Met Asp Lys Thr Glu Leu Gly Cys | |
| Leu Arg Ala Ile Val Leu Phe Asn Pro Asp Ser Lys Gly Leu Ser Asn | |
| Pro Ala Glu Val Glu Ala Leu Arg Glu Lys Val Tyr Ala Ser Leu Glu | |
| Ala Tyr Cys Lys His Lys Tyr Pro Glu Gln Pro Gly Arg Phe Ala Lys | |
| Leu Leu Leu Arg Leu Pro Ala Leu Arg Ser Ile Gly Leu Lys Cys Leu | |
| Glu His Leu Phe Phe Phe Lys Leu Ile Gly Asp Thr Pro Ile Asp Thr | |
| Phe Leu Met Glu Met Leu Glu Ala Pro His Gln Ala Thr | |
| <210> SEQ ID NO: 43 | |
| <211> LENGTH: 177 | |
| <212> TYPE: PRT | |
| <213> ORGANISM: Artificial Sequence | |
| <221> NAME/KEY: misc_feature | |
| <400> SEQUENCE: 43 | |
| Ile Pro His Phe Ser Glu Leu Pro Leu Asp Asp Gln Val Ile Leu Leu | |
| Arg Ala Gly Trp Asn Glu Leu Leu Ile Ala Ser Phe Ser His Arg Ser | |
| Ile Ala Val Lys Asp Gly Ile Leu Leu Ala Thr Gly Leu His Val His | |
| Arg Asn Ser Ala His Ser Ala Gly Val Gly Ala Ile Phe Asp Arg Val | |
| Leu Thr Glu Leu Val Ser Lys Met Arg Asp Met Gln Met Asp Lys Thr | |
| Glu Leu Gly Cys Leu Arg Ala Ile Val Leu Phe Asn Pro Asp Ser Lys | |
| Gly Leu Ser Asn Pro Ala Glu Val Glu Ala Leu Arg Glu Lys Val Tyr | |
| Ala Ser Leu Glu Ala Tyr Cys Lys His Lys Tyr Pro Glu Gln Pro Gly | |
| Arg Phe Ala Lys Leu Leu Leu Arg Leu Pro Ala Leu Arg Ser Ile Gly | |
| Leu Lys Cys Leu Glu His Leu Phe Phe Phe Lys Leu Ile Gly Asp Thr | |
| Pro Ile Asp Thr Phe Leu Met Glu Met Leu Glu Ala Pro His Gln Ala | |
| Thr | |
| <210> SEQ ID NO: 44 | |
| <211> LENGTH: 224 | |
| <212> TYPE: PRT | |
| <213> ORGANISM: Artificial Sequence | |
| <221> NAME/KEY: misc_feature | |
| <400> SEQUENCE: 44 | |
| Ala Asn Glu Asp Met Pro Val Glu Lys Ile Leu Glu Ala Glu Leu Ala | |
| Val Glu Pro Lys Thr Glu Thr Tyr Val Glu Ala Asn Met Gly Leu Asn | |
| Pro Ser Ser Pro Asn Asp Pro Val Thr Asn Ile Cys Gln Ala Ala Asp | |
| Lys Gln Leu Phe Thr Leu Val Glu Trp Ala Lys Arg Ile Pro His Phe | |
| Ser Glu Leu Pro Leu Asp Asp Gln Val Ile Leu Leu Arg Ala Gly Trp | |
| Asn Glu Leu Leu Ile Ala Ser Phe Ser His Arg Ser Ile Ala Val Lys | |
| Asp Gly Ile Leu Leu Ala Thr Gly Leu His Val His Arg Asn Ser Ala | |
| His Ser Ala Gly Val Gly Ala Ile Phe Asp Arg Val Leu Thr Glu Leu | |
| Val Ser Lys Met Arg Asp Met Gln Met Asp Lys Thr Glu Leu Gly Cys | |
| Leu Arg Ala Ile Val Leu Phe Asn Pro Asp Ser Lys Gly Leu Ser Asn | |
| Pro Ala Glu Val Glu Ala Leu Arg Glu Lys Val Tyr Ala Ser Leu Glu | |
| Ala Tyr Cys Lys His Lys Tyr Pro Glu Gln Pro Gly Arg Phe Ala Lys | |
| Leu Leu Leu Arg Leu Pro Ala Leu Arg Ser Ile Gly Leu Lys Cys Leu | |
| Glu His Leu Phe Phe Phe Lys Leu Ile Gly Asp Thr Pro Ile Asp Thr | |
| <210> SEQ ID NO: 45 | |
| <211> LENGTH: 328 | |
| <212> TYPE: PRT | |
| <213> ORGANISM: Artificial Sequence | |
| <221> NAME/KEY: misc_feature | |
| <400> SEQUENCE: 45 | |
| Cys Ala Ile Cys Gly Asp Arg Ser Ser Gly Lys His Tyr Gly Val Tyr | |
| Ser Cys Glu Gly Cys Lys Gly Phe Phe Lys Arg Thr Val Arg Lys Asp | |
| Leu Thr Tyr Thr Cys Arg Asp Asn Lys Asp Cys Leu Ile Asp Lys Arg | |
| Gln Arg Asn Arg Cys Gln Tyr Cys Arg Tyr Gln Lys Cys Leu Ala Met | |
| Gly Met Lys Arg Glu Ala Val Gln Glu Glu Arg Gln Arg Gly Lys Asp | |
| Arg Asn Glu Asn Glu Val Glu Ser Thr Ser Ser Ala Asn Glu Asp Met | |
| Pro Val Glu Arg Ile Leu Glu Ala Glu Leu Ala Val Glu Pro Lys Thr | |
| Glu Thr Tyr Val Glu Ala Asn Met Gly Leu Asn Pro Ser Ser Pro Asn | |
| Asp Pro Val Thr Asn Ile Cys Gln Ala Ala Asp Lys Gln Leu Phe Thr | |
| Leu Val Glu Trp Ala Lys Arg Ile Pro His Phe Ser Glu Leu Pro Leu | |
| Asp Asp Gln Val Ile Leu Leu Arg Ala Gly Trp Asn Glu Leu Leu Ile | |
| Ala Ser Phe Ser His Arg Ser Ile Ala Val Lys Asp Gly Ile Leu Leu | |
| Ala Thr Gly Leu His Val His Arg Asn Ser Ala His Ser Ala Gly Val | |
| Gly Ala Ile Phe Asp Arg Val Leu Thr Glu Leu Val Ser Lys Met Arg | |
| Asp Met Gln Met Asp Lys Thr Glu Leu Gly Cys Leu Arg Ala Ile Val | |
| Leu Phe Asn Pro Asp Ser Lys Gly Leu Ser Asn Pro Ala Glu Val Glu | |
| Ala Leu Arg Glu Lys Val Tyr Ala Ser Leu Glu Ala Tyr Cys Lys His | |
| Lys Tyr Pro Glu Gln Pro Gly Arg Phe Ala Lys Leu Leu Leu Arg Leu | |
| Pro Ala Leu Arg Ser Ile Gly Leu Lys Cys Leu Glu His Leu Phe Phe | |
| Phe Lys Leu Ile Gly Asp Thr Pro Ile Asp Thr Phe Leu Met Glu Met | |
| Leu Glu Ala Pro His Gln Met Thr | |
| <210> SEQ ID NO: 46 | |
| <211> LENGTH: 262 | |
| <212> TYPE: PRT | |
| <213> ORGANISM: Artificial Sequence | |
| <221> NAME/KEY: misc_feature | |
| <400> SEQUENCE: 46 | |
| Lys Arg Glu Ala Val Gln Glu Glu Arg Gln Arg Gly Lys Asp Arg Asn | |
| Glu Asn Glu Val Glu Ser Thr Ser Ser Ala Asn Glu Asp Met Pro Val | |
| Glu Arg Ile Leu Glu Ala Glu Leu Ala Val Glu Pro Lys Thr Glu Thr | |
| Tyr Val Glu Ala Asn Met Gly Leu Asn Pro Ser Ser Pro Asn Asp Pro | |
| Val Thr Asn Ile Cys Gln Ala Ala Asp Lys Gln Leu Phe Thr Leu Val | |
| Glu Trp Ala Lys Arg Ile Pro His Phe Ser Glu Leu Pro Leu Asp Asp | |
| Gln Val Ile Leu Leu Arg Ala Gly Trp Asn Glu Leu Leu Ile Ala Ser | |
| Phe Ser His Arg Ser Ile Ala Val Lys Asp Gly Ile Leu Leu Ala Thr | |
| Gly Leu His Val His Arg Asn Ser Ala His Ser Ala Gly Val Gly Ala | |
| Ile Phe Asp Arg Val Leu Thr Glu Leu Val Ser Lys Met Arg Asp Met | |
| Gln Met Asp Lys Thr Glu Leu Gly Cys Leu Arg Ala Ile Val Leu Phe | |
| Asn Pro Asp Ser Lys Gly Leu Ser Asn Pro Ala Glu Val Glu Ala Leu | |
| Arg Glu Lys Val Tyr Ala Ser Leu Glu Ala Tyr Cys Lys His Lys Tyr | |
| Pro Glu Gln Pro Gly Arg Phe Ala Lys Leu Leu Leu Arg Leu Pro Ala | |
| Leu Arg Ser Ile Gly Leu Lys Cys Leu Glu His Leu Phe Phe Phe Lys | |
| Leu Ile Gly Asp Thr Pro Ile Asp Thr Phe Leu Met Glu Met Leu Glu | |
| Ala Pro His Gln Met Thr | |
| <210> SEQ ID NO: 47 | |
| <211> LENGTH: 237 | |
| <212> TYPE: PRT | |
| <213> ORGANISM: Artificial Sequence | |
| <221> NAME/KEY: misc_feature | |
| <400> SEQUENCE: 47 | |
| Ala Asn Glu Asp Met Pro Val Glu Arg Ile Leu Glu Ala Glu Leu Ala | |
| Val Glu Pro Lys Thr Glu Thr Tyr Val Glu Ala Asn Met Gly Leu Asn | |
| Pro Ser Ser Pro Asn Asp Pro Val Thr Asn Ile Cys Gln Ala Ala Asp | |
| Lys Gln Leu Phe Thr Leu Val Glu Trp Ala Lys Arg Ile Pro His Phe | |
| Ser Glu Leu Pro Leu Asp Asp Gln Val Ile Leu Leu Arg Ala Gly Trp | |
| Asn Glu Leu Leu Ile Ala Ser Phe Ser His Arg Ser Ile Ala Val Lys | |
| Asp Gly Ile Leu Leu Ala Thr Gly Leu His Val His Arg Asn Ser Ala | |
| His Ser Ala Gly Val Gly Ala Ile Phe Asp Arg Val Leu Thr Glu Leu | |
| Val Ser Lys Met Arg Asp Met Gln Met Asp Lys Thr Glu Leu Gly Cys | |
| Leu Arg Ala Ile Val Leu Phe Asn Pro Asp Ser Lys Gly Leu Ser Asn | |
| Pro Ala Glu Val Glu Ala Leu Arg Glu Lys Val Tyr Ala Ser Leu Glu | |
| Ala Tyr Cys Lys His Lys Tyr Pro Glu Gln Pro Gly Arg Phe Ala Lys | |
| Leu Leu Leu Arg Leu Pro Ala Leu Arg Ser Ile Gly Leu Lys Cys Leu | |
| Glu His Leu Phe Phe Phe Lys Leu Ile Gly Asp Thr Pro Ile Asp Thr | |
| Phe Leu Met Glu Met Leu Glu Ala Pro His Gln Met Thr | |
| <210> SEQ ID NO: 48 | |
| <211> LENGTH: 177 | |
| <212> TYPE: PRT | |
| <213> ORGANISM: Artificial Sequence | |
| <<221> NAME/KEY: misc_feature | |
| <400> SEQUENCE: 48 | |
| Ile Pro His Phe Ser Glu Leu Pro Leu Asp Asp Gln Val Ile Leu Leu | |
| Arg Ala Gly Trp Asn Glu Leu Leu Ile Ala Ser Phe Ser His Arg Ser | |
| Ile Ala Val Lys Asp Gly Ile Leu Leu Ala Thr Gly Leu His Val His | |
| Arg Asn Ser Ala His Ser Ala Gly Val Gly Ala Ile Phe Asp Arg Val | |
| Leu Thr Glu Leu Val Ser Lys Met Arg Asp Met Gln Met Asp Lys Thr | |
| Glu Leu Gly Cys Leu Arg Ala Ile Val Leu Phe Asn Pro Asp Ser Lys | |
| Gly Leu Ser Asn Pro Ala Glu Val Glu Ala Leu Arg Glu Lys Val Tyr | |
| Ala Ser Leu Glu Ala Tyr Cys Lys His Lys Tyr Pro Glu Gln Pro Gly | |
| Arg Phe Ala Lys Leu Leu Leu Arg Leu Pro Ala Leu Arg Ser Ile Gly | |
| Leu Lys Cys Leu Glu His Leu Phe Phe Phe Lys Leu Ile Gly Asp Thr | |
| Pro Ile Asp Thr Phe Leu Met Glu Met Leu Glu Ala Pro His Gln Met | |
| Thr | |
| <210> SEQ ID NO: 49 | |
| <211> LENGTH: 224 | |
| <212> TYPE: PRT | |
| <213> ORGANISM: Artificial Sequence | |
| <221> NAME/KEY: misc_feature | |
| <400> SEQUENCE: 49 | |
| Ala Asn Glu Asp Met Pro Val Glu Arg Ile Leu Glu Ala Glu Leu Ala | |
| Val Glu Pro Lys Thr Glu Thr Tyr Val Glu Ala Asn Met Gly Leu Asn | |
| Pro Ser Ser Pro Asn Asp Pro Val Thr Asn Ile Cys Gln Ala Ala Asp | |
| Lys Gln Leu Phe Thr Leu Val Glu Trp Ala Lys Arg Ile Pro His Phe | |
| Ser Glu Leu Pro Leu Asp Asp Gln Val Ile Leu Leu Arg Ala Gly Trp | |
| Asn Glu Leu Leu Ile Ala Ser Phe Ser His Arg Ser Ile Ala Val Lys | |
| Asp Gly Ile Leu Leu Ala Thr Gly Leu His Val His Arg Asn Ser Ala | |
| His Ser Ala Gly Val Gly Ala Ile Phe Asp Arg Val Leu Thr Glu Leu | |
| Val Ser Lys Met Arg Asp Met Gln Met Asp Lys Thr Glu Leu Gly Cys | |
| Leu Arg Ala Ile Val Leu Phe Asn Pro Asp Ser Lys Gly Leu Ser Asn | |
| Pro Ala Glu Val Glu Ala Leu Arg Glu Lys Val Tyr Ala Ser Leu Glu | |
| Ala Tyr Cys Lys His Lys Tyr Pro Glu Gln Pro Gly Arg Phe Ala Lys | |
| Leu Leu Leu Arg Leu Pro Ala Leu Arg Ser Ile Gly Leu Lys Cys Leu | |
| Glu His Leu Phe Phe Phe Lys Leu Ile Gly Asp Thr Pro Ile Asp Thr | |
| <210> SEQ ID NO: 50 | |
| <211> LENGTH: 635 | |
| <212> TYPE: DNA | |
| <213> ORGANISM: Locusta migratoria | |
| <400> SEQUENCE: 50 | |
| tgcatacaga catgcctgtt gaacgcatac ttgaagctga aaaacgagtg gagtgcaaag | 60 |
| cagaaaacca agtggaatat gagctggtgg agtgggctaa acacatcccg cacttcacat | 120 |
| ccctacctct ggaggaccag gttctcctcc tcagagcagg ttggaatgaa ctgctaattg | 180 |
| cagcattttc acatcgatct gtagatgtta aagatggcat agtacttgcc actggtctca | 240 |
| cagtgcatcg aaattctgcc catcaagctg gagtcggcac aatatttgac agagttttga | 300 |
| cagaactggt agcaaagatg agagaaatga aaatggataa aactgaactt ggctgcttgc | 360 |
| gatctgttat tcttttcaat ccagaggtga ggggtttgaa atccgcccag gaagttgaac | 420 |
| ttctacgtga aaaagtatat gccgctttgg aagaatatac tagaacaaca catcccgatg | 480 |
| aaccaggaag atttgcaaaa cttttgcttc gtctgccttc tttacgttcc ataggcctta | 540 |
| agtgtttgga gcatttgttt ttctttcgcc ttattggaga tgttccaatt gatacgttcc | 600 |
| tgatggagat gcttgaatca ccttctgatt cataa | 635 |
| <210> SEQ ID NO: 51 | |
| <211> LENGTH: 687 | |
| <212> TYPE: DNA | |
| <213> ORGANISM: Amblyomma americanum | |
| <400> SEQUENCE: 51 | |
| cctcctgaga tgcctctgga gcgcatactg gaggcagagc tgcgggttga gtcacagacg | 60 |
| gggaccctct cggaaagcgc acagcagcag gatccagtga gcagcatctg ccaagctgca | 120 |
| gaccgacagc tgcaccagct agttcaatgg gccaagcaca ttccacattt tgaagagctt | 180 |
| ccccttgagg accgcatggt gttgctcaag gctggctgga acgagctgct cattgctgct | 240 |
| ttctcccacc gttctgttga cgtgcgtgat ggcattgtgc tcgctacagg tcttgtggtg | 300 |
| cagcggcata gtgctcatgg ggctggcgtt ggggccatat ttgatagggt tctcactgaa | 360 |
| ctggtagcaa agatgcgtga gatgaagatg gaccgcactg agcttggatg cctgcttgct | 420 |
| gtggtacttt ttaatcctga ggccaagggg ctgcggacct gcccaagtgg aggccctgag | 480 |
| ggagaaagtg tatctgcctt ggaagagcac tgccggcagc agtacccaga ccagcctggg | 540 |
| cgctttgcca agctgctgct gcggttgcca gctctgcgca gtattggcct caagtgcctc | 600 |
| gaacatctct ttttcttcaa gctcatcggg gacacgccca tcgacaactt tcttctttcc | 660 |
| atgctggagg ccccctctga cccctaa | 687 |
| <210> SEQ ID NO: 52 | |
| <211> LENGTH: 693 | |
| <212> TYPE: DNA | |
| <213> ORGANISM: Amblyomma americanum | |
| <400> SEQUENCE: 52 | |
| tctccggaca tgccactcga acgcattctc gaagccgaga tgcgcgtcga gcagccggca | 60 |
| ccgtccgttt tggcgcagac ggccgcatcg ggccgcgacc ccgtcaacag catgtgccag | 120 |
| gctgccccgc cacttcacga gctcgtacag tgggcccggc gaattccgca cttcgaagag | 180 |
| cttcccatcg aggatcgcac cgcgctgctc aaagccggct ggaacgaact gcttattgcc | 240 |
| gccttttcgc accgttctgt ggcggtgcgc gacggcatcg ttctggccac cgggctggtg | 300 |
| gtgcagcggc acagcgcaca cggcgcaggc gttggcgaca tcttcgaccg cgtactagcc | 360 |
| gagctggtgg ccaagatgcg cgacatgaag atggacaaaa cggagctcgg ctgcctgcgc | 420 |
| gccgtggtgc tcttcaatcc agacgccaag ggtctccgaa acgccaccag agtagaggcg | 480 |
| ctccgcgaga aggtgtatgc ggcgctggag gagcactgcc gtcggcacca cccggaccaa | 540 |
| ccgggtcgct tcggcaagct gctgctgcgg ctgcctgcct tgcgcagcat cgggctcaaa | 600 |
| tgcctcgagc atctgttctt cttcaagctc atcggagaca ctcccataga cagcttcctg | 660 |
| ctcaacatgc tggaggcacc ggcagacccc tag | 693 |
| <210> SEQ ID NO: 53 | |
| <211> LENGTH: 801 | |
| <212> TYPE: DNA | |
| <213> ORGANISM: Celuca pugilator | |
| <400> SEQUENCE: 53 | |
| tcagacatgc caattgccag catacgggag gcagagctca gcgtggatcc catagatgag | 60 |
| cagccgctgg accaaggggt gaggcttcag gttccactcg cacctcctga tagtgaaaag | 120 |
| tgtagcttta ctttaccttt tcatcccgtc agtgaagtat cctgtgctaa ccctctgcag | 180 |
| gatgtggtga gcaacatatg ccaggcagct gacagacatc tggtgcagct ggtggagtgg | 240 |
| gccaagcaca tcccacactt cacagacctt cccatagagg accaagtggt attactcaaa | 300 |
| gccgggtgga acgagttgct tattgcctca ttctcacacc gtagcatggg cgtggaggat | 360 |
| ggcatcgtgc tggccacagg gctcgtgatc cacagaagta gtgctcacca ggctggagtg | 420 |
| ggtgccatat ttgatcgtgt cctctctgag ctggtggcca agatgaagga gatgaagatt | 480 |
| gacaagacag agctgggctg ccttcgctcc atcgtcctgt tcaacccaga tgccaaagga | 540 |
| ctaaactgcg tcaatgatgt ggagatcttg cgtgagaagg tgtatgctgc cctggaggag | 600 |
| tacacacgaa ccacttaccc tgatgaacct ggacgctttg ccaagttgct tctgcgactt | 660 |
| cctgcactca ggtctatagg cctgaagtgt cttgagtacc tcttcctgtt taagctgatt | 720 |
| ggagacactc ccctggacag ctacttgatg aagatgctcg tagacaaccc aaatacaagc | 780 |
| gtcactcccc ccaccagcta g | 801 |
| <210> SEQ ID NO: 54 | |
| <211> LENGTH: 690 | |
| <212> TYPE: DNA | |
| <213> ORGANISM: Tenebrio molitor | |
| <400> SEQUENCE: 54 | |
| gccgagatgc ccctcgacag gataatcgag gcggagaaac ggatagaatg cacacccgct | 60 |
| ggtggctctg gtggtgtcgg agagcaacac gacggggtga acaacatctg tcaagccact | 120 |
| aacaagcagc tgttccaact ggtgcaatgg gctaagctca tacctcactt tacctcgttg | 180 |
| ccgatgtcgg accaggtgct tttattgagg gcaggatgga atgaattgct catcgccgca | 240 |
| ttctcgcaca gatctataca ggcgcaggat gccatcgttc tagccacggg gttgacagtt | 300 |
| aacaaaacgt cggcgcacgc cgtgggcgtg ggcaacatct acgaccgcgt cctctccgag | 360 |
| ctggtgaaca agatgaaaga gatgaagatg gacaagacgg agctgggctg cttgagagcc | 420 |
| atcatcctct acaaccccac gtgtcgcggc atcaagtccg tgcaggaagt ggagatgctg | 480 |
| cgtgagaaaa tttacggcgt gctggaagag tacaccagga ccacccaccc gaacgagccc | 540 |
| ggcaggttcg ccaaactgct tctgcgcctc ccggccctca ggtccatcgg gttgaaatgt | 600 |
| tccgaacacc tctttttctt caagctgatc ggtgatgttc caatagacac gttcctgatg | 660 |
| gagatgctgg agtctccggc ggacgcttag | 690 |
| <210> SEQ ID NO: 55 | |
| <211> LENGTH: 681 | |
| <212> TYPE: DNA | |
| <213> ORGANISM: Apis mellifera | |
| <400> SEQUENCE: 55 | |
| cattcggaca tgccgatcga gcgtatcctg gaggccgaga agagagtcga atgtaagatg | 60 |
| gagcaacagg gaaattacga gaatgcagtg tcgcacattt gcaacgccac gaacaaacag | 120 |
| ctgttccagc tggtagcatg ggcgaaacac atcccgcatt ttacctcgtt gccactggag | 180 |
| gatcaggtac ttctgctcag ggccggttgg aacgagttgc tgatagcctc cttttcccac | 240 |
| cgttccatcg acgtgaagga cggtatcgtg ctggcgacgg ggatcaccgt gcatcggaac | 300 |
| tcggcgcagc aggccggcgt gggcacgata ttcgaccgtg tcctctcgga gcttgtctcg | 360 |
| aaaatgcgtg aaatgaagat ggacaggaca gagcttggct gtctcagatc tataatactc | 420 |
| ttcaatcccg aggttcgagg actgaaatcc atccaggaag tgaccctgct ccgtgagaag | 480 |
| atctacggcg ccctggaggg ttattgccgc gtagcttggc ccgacgacgc tggaagattc | 540 |
| gcgaaattac ttctacgcct gcccgccatc cgctcgatcg gattaaagtg cctcgagtac | 600 |
| ctgttcttct tcaaaatgat cggtgacgta ccgatcgacg attttctcgt ggagatgtta | 660 |
| gaatcgcgat cagatcctta g | 681 |
| <210> SEQ ID NO: 56 | |
| <211> LENGTH: 210 | |
| <212> TYPE: PRT | |
| <213> ORGANISM: Locusta migratoria | |
| <400> SEQUENCE: 56 | |
| His Thr Asp Met Pro Val Glu Arg Ile Leu Glu Ala Glu Lys Arg Val | |
| Glu Cys Lys Ala Glu Asn Gln Val Glu Tyr Glu Leu Val Glu Trp Ala | |
| Lys His Ile Pro His Phe Thr Ser Leu Pro Leu Glu Asp Gln Val Leu | |
| Leu Leu Arg Ala Gly Trp Asn Glu Leu Leu Ile Ala Ala Phe Ser His | |
| Arg Ser Val Asp Val Lys Asp Gly Ile Val Leu Ala Thr Gly Leu Thr | |
| Val His Arg Asn Ser Ala His Gln Ala Gly Val Gly Thr Ile Phe Asp | |
| Arg Val Leu Thr Glu Leu Val Ala Lys Met Arg Glu Met Lys Met Asp | |
| Lys Thr Glu Leu Gly Cys Leu Arg Ser Val Ile Leu Phe Asn Pro Glu | |
| Val Arg Gly Leu Lys Ser Ala Gln Glu Val Glu Leu Leu Arg Glu Lys | |
| Val Tyr Ala Ala Leu Glu Glu Tyr Thr Arg Thr Thr His Pro Asp Glu | |
| Pro Gly Arg Phe Ala Lys Leu Leu Leu Arg Leu Pro Ser Leu Arg Ser | |
| Ile Gly Leu Lys Cys Leu Glu His Leu Phe Phe Phe Arg Leu Ile Gly | |
| Asp Val Pro Ile Asp Thr Phe Leu Met Glu Met Leu Glu Ser Pro Ser | |
| Asp Ser | |
| <210> SEQ ID NO: 57 | |
| <211> LENGTH: 228 | |
| <212> TYPE: PRT | |
| <213> ORGANISM: Amblyomma americanum | |
| <400> SEQUENCE: 57 | |
| Pro Pro Glu Met Pro Leu Glu Arg Ile Leu Glu Ala Glu Leu Arg Val | |
| Glu Ser Gln Thr Gly Thr Leu Ser Glu Ser Ala Gln Gln Gln Asp Pro | |
| Val Ser Ser Ile Cys Gln Ala Ala Asp Arg Gln Leu His Gln Leu Val | |
| Gln Trp Ala Lys His Ile Pro His Phe Glu Glu Leu Pro Leu Glu Asp | |
| Arg Met Val Leu Leu Lys Ala Gly Trp Asn Glu Leu Leu Ile Ala Ala | |
| Phe Ser His Arg Ser Val Asp Val Arg Asp Gly Ile Val Leu Ala Thr | |
| Gly Leu Val Val Gln Arg His Ser Ala His Gly Ala Gly Val Gly Ala | |
| Ile Phe Asp Arg Val Leu Thr Glu Leu Val Ala Lys Met Arg Glu Met | |
| Lys Met Asp Arg Thr Glu Leu Gly Cys Leu Leu Ala Val Val Leu Phe | |
| Asn Pro Glu Ala Lys Gly Leu Arg Thr Cys Pro Ser Gly Gly Pro Glu | |
| Gly Glu Ser Val Ser Ala Leu Glu Glu His Cys Arg Gln Gln Tyr Pro | |
| Asp Gln Pro Gly Arg Phe Ala Lys Leu Leu Leu Arg Leu Pro Ala Leu | |
| Arg Ser Ile Gly Leu Lys Cys Leu Glu His Leu Phe Phe Phe Lys Leu | |
| Ile Gly Asp Thr Pro Ile Asp Asn Phe Leu Leu Ser Met Leu Glu Ala | |
| Pro Ser Asp Pro | |
| <210> SEQ ID NO: 58 | |
| <211> LENGTH: 230 | |
| <212> TYPE: PRT | |
| <213> ORGANISM: Amblyomma americanum | |
| <400> SEQUENCE: 58 | |
| Ser Pro Asp Met Pro Leu Glu Arg Ile Leu Glu Ala Glu Met Arg Val | |
| Glu Gln Pro Ala Pro Ser Val Leu Ala Gln Thr Ala Ala Ser Gly Arg | |
| Asp Pro Val Asn Ser Met Cys Gln Ala Ala Pro Pro Leu His Glu Leu | |
| Val Gln Trp Ala Arg Arg Ile Pro His Phe Glu Glu Leu Pro Ile Glu | |
| Asp Arg Thr Ala Leu Leu Lys Ala Gly Trp Asn Glu Leu Leu Ile Ala | |
| Ala Phe Ser His Arg Ser Val Ala Val Arg Asp Gly Ile Val Leu Ala | |
| Thr Gly Leu Val Val Gln Arg His Ser Ala His Gly Ala Gly Val Gly | |
| Asp Ile Phe Asp Arg Val Leu Ala Glu Leu Val Ala Lys Met Arg Asp | |
| Met Lys Met Asp Lys Thr Glu Leu Gly Cys Leu Arg Ala Val Val Leu | |
| Phe Asn Pro Asp Ala Lys Gly Leu Arg Asn Ala Thr Arg Val Glu Ala | |
| Leu Arg Glu Lys Val Tyr Ala Ala Leu Glu Glu His Cys Arg Arg His | |
| His Pro Asp Gln Pro Gly Arg Phe Gly Lys Leu Leu Leu Arg Leu Pro | |
| Ala Leu Arg Ser Ile Gly Leu Lys Cys Leu Glu His Leu Phe Phe Phe | |
| Lys Leu Ile Gly Asp Thr Pro Ile Asp Ser Phe Leu Leu Asn Met Leu | |
| Glu Ala Pro Ala Asp Pro | |
| <210> SEQ ID NO: 59 | |
| <211> LENGTH: 266 | |
| <212> TYPE: PRT | |
| <213> ORGANISM: Celuca pugilator | |
| <400> SEQUENCE: 59 | |
| Ser Asp Met Pro Ile Ala Ser Ile Arg Glu Ala Glu Leu Ser Val Asp | |
| Pro Ile Asp Glu Gln Pro Leu Asp Gln Gly Val Arg Leu Gln Val Pro | |
| Leu Ala Pro Pro Asp Ser Glu Lys Cys Ser Phe Thr Leu Pro Phe His | |
| Pro Val Ser Glu Val Ser Cys Ala Asn Pro Leu Gln Asp Val Val Ser | |
| Asn Ile Cys Gln Ala Ala Asp Arg His Leu Val Gln Leu Val Glu Trp | |
| Ala Lys His Ile Pro His Phe Thr Asp Leu Pro Ile Glu Asp Gln Val | |
| Val Leu Leu Lys Ala Gly Trp Asn Glu Leu Leu Ile Ala Ser Phe Ser | |
| His Arg Ser Met Gly Val Glu Asp Gly Ile Val Leu Ala Thr Gly Leu | |
| Val Ile His Arg Ser Ser Ala His Gln Ala Gly Val Gly Ala Ile Phe | |
| Asp Arg Val Leu Ser Glu Leu Val Ala Lys Met Lys Glu Met Lys Ile | |
| Asp Lys Thr Glu Leu Gly Cys Leu Arg Ser Ile Val Leu Phe Asn Pro | |
| Asp Ala Lys Gly Leu Asn Cys Val Asn Asp Val Glu Ile Leu Arg Glu | |
| Lys Val Tyr Ala Ala Leu Glu Glu Tyr Thr Arg Thr Thr Tyr Pro Asp | |
| Glu Pro Gly Arg Phe Ala Lys Leu Leu Leu Arg Leu Pro Ala Leu Arg | |
| Ser Ile Gly Leu Lys Cys Leu Glu Tyr Leu Phe Leu Phe Lys Leu Ile | |
| Gly Asp Thr Pro Leu Asp Ser Tyr Leu Met Lys Met Leu Val Asp Asn | |
| Pro Asn Thr Ser Val Thr Pro Pro Thr Ser | |
| <210> SEQ ID NO: 60 | |
| <211> LENGTH: 229 | |
| <212> TYPE: PRT | |
| <213> ORGANISM: Tenebrio molitor | |
| <400> SEQUENCE: 60 | |
| Ala Glu Met Pro Leu Asp Arg Ile Ile Glu Ala Glu Lys Arg Ile Glu | |
| Cys Thr Pro Ala Gly Gly Ser Gly Gly Val Gly Glu Gln His Asp Gly | |
| Val Asn Asn Ile Cys Gln Ala Thr Asn Lys Gln Leu Phe Gln Leu Val | |
| Gln Trp Ala Lys Leu Ile Pro His Phe Thr Ser Leu Pro Met Ser Asp | |
| Gln Val Leu Leu Leu Arg Ala Gly Trp Asn Glu Leu Leu Ile Ala Ala | |
| Phe Ser His Arg Ser Ile Gln Ala Gln Asp Ala Ile Val Leu Ala Thr | |
| Gly Leu Thr Val Asn Lys Thr Ser Ala His Ala Val Gly Val Gly Asn | |
| Ile Tyr Asp Arg Val Leu Ser Glu Leu Val Asn Lys Met Lys Glu Met | |
| Lys Met Asp Lys Thr Glu Leu Gly Cys Leu Arg Ala Ile Ile Leu Tyr | |
| Asn Pro Thr Cys Arg Gly Ile Lys Ser Val Gln Glu Val Glu Met Leu | |
| Arg Glu Lys Ile Tyr Gly Val Leu Glu Glu Tyr Thr Arg Thr Thr His | |
| Pro Asn Glu Pro Gly Arg Phe Ala Lys Leu Leu Leu Arg Leu Pro Ala | |
| Leu Arg Ser Ile Gly Leu Lys Cys Ser Glu His Leu Phe Phe Phe Lys | |
| Leu Ile Gly Asp Val Pro Ile Asp Thr Phe Leu Met Glu Met Leu Glu | |
| Ser Pro Ala Asp Ala | |
| <210> SEQ ID NO: 61 | |
| <211> LENGTH: 226 | |
| <212> TYPE: PRT | |
| <213> ORGANISM: Apis mellifera | |
| <400> SEQUENCE: 61 | |
| His Ser Asp Met Pro Ile Glu Arg Ile Leu Glu Ala Glu Lys Arg Val | |
| Glu Cys Lys Met Glu Gln Gln Gly Asn Tyr Glu Asn Ala Val Ser His | |
| Ile Cys Asn Ala Thr Asn Lys Gln Leu Phe Gln Leu Val Ala Trp Ala | |
| Lys His Ile Pro His Phe Thr Ser Leu Pro Leu Glu Asp Gln Val Leu | |
| Leu Leu Arg Ala Gly Trp Asn Glu Leu Leu Ile Ala Ser Phe Ser His | |
| Arg Ser Ile Asp Val Lys Asp Gly Ile Val Leu Ala Thr Gly Ile Thr | |
| Val His Arg Asn Ser Ala Gln Gln Ala Gly Val Gly Thr Ile Phe Asp | |
| Arg Val Leu Ser Glu Leu Val Ser Lys Met Arg Glu Met Lys Met Asp | |
| Arg Thr Glu Leu Gly Cys Leu Arg Ser Ile Ile Leu Phe Asn Pro Glu | |
| Val Arg Gly Leu Lys Ser Ile Gln Glu Val Thr Leu Leu Arg Glu Lys | |
| Ile Tyr Gly Ala Leu Glu Gly Tyr Cys Arg Val Ala Trp Pro Asp Asp | |
| Ala Gly Arg Phe Ala Lys Leu Leu Leu Arg Leu Pro Ala Ile Arg Ser | |
| Ile Gly Leu Lys Cys Leu Glu Tyr Leu Phe Phe Phe Lys Met Ile Gly | |
| Asp Val Pro Ile Asp Asp Phe Leu Val Glu Met Leu Glu Ser Arg Ser | |
| Asp Pro | |
| <210> SEQ ID NO: 62 | |
| <211> LENGTH: 714 | |
| <212> TYPE: DNA | |
| <213> ORGANISM: Mus musculus | |
| <400> SEQUENCE: 62 | |
| gccaacgagg acatgcctgt agagaagatt ctggaagccg agcttgctgt cgagcccaag | 60 |
| actgagacat acgtggaggc aaacatgggg ctgaacccca gctcaccaaa tgaccctgtt | 120 |
| accaacatct gtcaagcagc agacaagcag ctcttcactc ttgtggagtg ggccaagagg | 180 |
| atcccacact tttctgagct gcccctagac gaccaggtca tcctgctacg ggcaggctgg | 240 |
| aacgagctgc tgatcgcctc cttctcccac cgctccatag ctgtgaaaga tgggattctc | 300 |
| ctggccaccg gcctgcacgt acaccggaac agcgctcaca gtgctggggt gggcgccatc | 360 |
| tttgacaggg tgctaacaga gctggtgtct aagatgcgtg acatgcagat ggacaagacg | 420 |
| gagctgggct gcctgcgagc cattgtcctg ttcaaccctg actctaaggg gctctcaaac | 480 |
| cctgctgagg tggaggcgtt gagggagaag gtgtatgcgt cactagaagc gtactgcaaa | 540 |
| cacaagtacc ctgagcagcc gggcaggttt gccaagctgc tgctccgcct gcctgcactg | 600 |
| cgttccatcg ggctcaagtg cctggagcac ctgttcttct tcaagctcat cggggacacg | 660 |
| cccatcgaca ccttcctcat ggagatgctg gaggcaccac atcaagccac ctag | 714 |
| <210> SEQ ID NO: 63 | |
| <211> LENGTH: 720 | |
| <212> TYPE: DNA | |
| <213> ORGANISM: Mus musculus | |
| <400> SEQUENCE: 63 | |
| gcccctgagg agatgcctgt ggacaggatc ctggaggcag agcttgctgt ggagcagaag | 60 |
| agtgaccaag gcgttgaggg tcctggggcc accgggggtg gtggcagcag cccaaatgac | 120 |
| ccagtgacta acatctgcca ggcagctgac aaacagctgt tcacactcgt tgagtgggca | 180 |
| aagaggatcc cgcacttctc ctccctacct ctggacgatc aggtcatact gctgcgggca | 240 |
| ggctggaacg agctcctcat tgcgtccttc tcccatcggt ccattgatgt ccgagatggc | 300 |
| atcctcctgg ccacgggtct tcatgtgcac agaaactcag cccattccgc aggcgtggga | 360 |
| gccatctttg atcgggtgct gacagagcta gtgtccaaaa tgcgtgacat gaggatggac | 420 |
| aagacagagc ttggctgcct gcgggcaatc atcatgttta atccagacgc caagggcctc | 480 |
| tccaaccctg gagaggtgga gatccttcgg gagaaggtgt acgcctcact ggagacctat | 540 |
| tgcaagcaga agtaccctga gcagcagggc cggtttgcca agctgctgtt acgtcttcct | 600 |
| gccctccgct ccatcggcct caagtgtctg gagcacctgt tcttcttcaa gctcattggc | 660 |
| gacaccccca ttgacacctt cctcatggag atgcttgagg ctccccacca gctagcctga | 720 |
| <210> SEQ ID NO: 64 | |
| <211> LENGTH: 705 | |
| <212> TYPE: DNA | |
| <213> ORGANISM: Mus musculus | |
| <400> SEQUENCE: 64 | |
| agccacgaag acatgcccgt ggagaggatt ctagaagccg aacttgctgt ggaaccaaag | 60 |
| acagaatcct acggtgacat gaacgtggag aactcaacaa atgaccctgt taccaacata | 120 |
| tgccatgctg cagataagca acttttcacc ctcgttgagt gggccaaacg catcccccac | 180 |
| ttctcagatc tcaccttgga ggaccaggtc attctactcc gggcagggtg gaatgaactg | 240 |
| ctcattgcct ccttctccca ccgctcggtt tccgtccagg atggcatcct gctggccacg | 300 |
| ggcctccacg tgcacaggag cagcgctcac agccggggag tcggctccat cttcgacaga | 360 |
| gtccttacag agttggtgtc caagatgaaa gacatgcaga tggataagtc agagctgggg | 420 |
| tgcctacggg ccatcgtgct gtttaaccca gatgccaagg gtttatccaa cccctctgag | 480 |
| gtggagactc ttcgagagaa ggtttatgcc accctggagg cctataccaa gcagaagtat | 540 |
| ccggaacagc caggcaggtt tgccaagctt ctgctgcgtc tccctgctct gcgctccatc | 600 |
| ggcttgaaat gcctggaaca cctcttcttc ttcaagctca ttggagacac tcccatcgac | 660 |
| agcttcctca tggagatgtt ggagacccca ctgcagatca cctga | 705 |
| <210> SEQ ID NO: 65 | |
| <211> LENGTH: 850 | |
| <212> TYPE: DNA | |
| <213> ORGANISM: Homo sapiens | |
| <400> SEQUENCE: 65 | |
| gccaacgagg acatgccggt ggagaggatc ctggaggctg agctggccgt ggagcccaag | 60 |
| accgagacct acgtggaggc aaacatgggg ctgaacccca gctcgccgaa cgaccctgtc | 120 |
| accaacattt gccaagcagc cgacaaacag cttttcaccc tggtggagtg ggccaagcgg | 180 |
| atcccacact tctcagagct gcccctggac gaccaggtca tcctgctgcg ggcaggctgg | 240 |
| aatgagctgc tcatcgcctc cttctcccac cgctccatcg ccgtgaagga cgggatcctc | 300 |
| ctggccaccg ggctgcacgt ccaccggaac agcgcccaca gcgcaggggt gggcgccatc | 360 |
| tttgacaggg tgctgacgga gcttgtgtcc aagatgcggg acatgcagat ggacaagacg | 420 |
| gagctgggct gcctgcgcgc catcgtcctc tttaaccctg actccaaggg gctctcgaac | 480 |
| ccggccgagg tggaggcgct gagggagaag gtctatgcgt ccttggaggc ctactgcaag | 540 |
| cacaagtacc cagagcagcc gggaaggttc gctaagctct tgctccgcct gccggctctg | 600 |
| cgctccatcg ggctcaaatg cctggaacat ctcttcttct tcaagctcat cggggacaca | 660 |
| cccattgaca ccttccttat ggagatgctg gaggcgccgc accaaatgac ttaggcctgc | 720 |
| gggcccatcc tttgtgccca cccgttctgg ccaccctgcc tggacgccag ctgttcttct | 780 |
| cagcctgagc cctgtccctg cccttctctg cctggcctgt ttggactttg gggcacagcc | 840 |
| tgtcactgct | 850 |
| <210> SEQ ID NO: 66 | |
| <211> LENGTH: 720 | |
| <212> TYPE: DNA | |
| <213> ORGANISM: Homo sapiens | |
| <400> SEQUENCE: 66 | |
| gcccccgagg agatgcctgt ggacaggatc ctggaggcag agcttgctgt ggaacagaag | 60 |
| agtgaccagg gcgttgaggg tcctggggga accgggggta gcggcagcag cccaaatgac | 120 |
| cctgtgacta acatctgtca ggcagctgac aaacagctat tcacgcttgt tgagtgggcg | 180 |
| aagaggatcc cacacttttc ctccttgcct ctggatgatc aggtcatatt gctgcgggca | 240 |
| ggctggaatg aactcctcat tgcctccttt tcacaccgat ccattgatgt tcgagatggc | 300 |
| atcctccttg ccacaggtct tcacgtgcac cgcaactcag cccattcagc aggagtagga | 360 |
| gccatctttg atcgggtgct gacagagcta gtgtccaaaa tgcgtgacat gaggatggac | 420 |
| aagacagagc ttggctgcct gagggcaatc attctgttta atccagatgc caagggcctc | 480 |
| tccaacccta gtgaggtgga ggtcctgcgg gagaaagtgt atgcatcact ggagacctac | 540 |
| tgcaaacaga agtaccctga gcagcaggga cggtttgcca agctgctgct acgtcttcct | 600 |
| gccctccggt ccattggcct taagtgtcta gagcatctgt ttttcttcaa gctcattggt | 660 |
| gacaccccca tcgacacctt cctcatggag atgcttgagg ctccccatca actggcctga | 720 |
| <210> SEQ ID NO: 67 | |
| <211> LENGTH: 705 | |
| <212> TYPE: DNA | |
| <213> ORGANISM: Homo sapiens | |
| <400> SEQUENCE: 67 | |
| ggtcatgaag acatgcctgt ggagaggatt ctagaagctg aacttgctgt tgaaccaaag | 60 |
| acagaatcct atggtgacat gaatatggag aactcgacaa atgaccctgt taccaacata | 120 |
| tgtcatgctg ctgacaagca gcttttcacc ctcgttgaat gggccaagcg tattccccac | 180 |
| ttctctgacc tcaccttgga ggaccaggtc attttgcttc gggcagggtg gaatgaattg | 240 |
| ctgattgcct ctttctccca ccgctcagtt tccgtgcagg atggcatcct tctggccacg | 300 |
| ggtttacatg tccaccggag cagtgcccac agtgctgggg tcggctccat ctttgacaga | 360 |
| gttctaactg agctggtttc caaaatgaaa gacatgcaga tggacaagtc ggaactggga | 420 |
| tgcctgcgag ccattgtact ctttaaccca gatgccaagg gcctgtccaa cccctctgag | 480 |
| gtggagactc tgcgagagaa ggtttatgcc acccttgagg cctacaccaa gcagaagtat | 540 |
| ccggaacagc caggcaggtt tgccaagctg ctgctgcgcc tcccagctct gcgttccatt | 600 |
| ggcttgaaat gcctggagca cctcttcttc ttcaagctca tcggggacac ccccattgac | 660 |
| accttcctca tggagatgtt ggagaccccg ctgcagatca cctga | 705 |
| <210> SEQ ID NO: 68 | |
| <211> LENGTH: 237 | |
| <212> TYPE: PRT | |
| <213> ORGANISM: Mus musculus | |
| <400> SEQUENCE: 68 | |
| Ala Asn Glu Asp Met Pro Val Glu Lys Ile Leu Glu Ala Glu Leu Ala | |
| Val Glu Pro Lys Thr Glu Thr Tyr Val Glu Ala Asn Met Gly Leu Asn | |
| Pro Ser Ser Pro Asn Asp Pro Val Thr Asn Ile Cys Gln Ala Ala Asp | |
| Lys Gln Leu Phe Thr Leu Val Glu Trp Ala Lys Arg Ile Pro His Phe | |
| Ser Glu Leu Pro Leu Asp Asp Gln Val Ile Leu Leu Arg Ala Gly Trp | |
| Asn Glu Leu Leu Ile Ala Ser Phe Ser His Arg Ser Ile Ala Val Lys | |
| Asp Gly Ile Leu Leu Ala Thr Gly Leu His Val His Arg Asn Ser Ala | |
| His Ser Ala Gly Val Gly Ala Ile Phe Asp Arg Val Leu Thr Glu Leu | |
| Val Ser Lys Met Arg Asp Met Gln Met Asp Lys Thr Glu Leu Gly Cys | |
| Leu Arg Ala Ile Val Leu Phe Asn Pro Asp Ser Lys Gly Leu Ser Asn | |
| Pro Ala Glu Val Glu Ala Leu Arg Glu Lys Val Tyr Ala Ser Leu Glu | |
| Ala Tyr Cys Lys His Lys Tyr Pro Glu Gln Pro Gly Arg Phe Ala Lys | |
| Leu Leu Leu Arg Leu Pro Ala Leu Arg Ser Ile Gly Leu Lys Cys Leu | |
| Glu His Leu Phe Phe Phe Lys Leu Ile Gly Asp Thr Pro Ile Asp Thr | |
| Phe Leu Met Glu Met Leu Glu Ala Pro His Gln Ala Thr | |
| <210> SEQ ID NO: 69 | |
| <211> LENGTH: 239 | |
| <212> TYPE: PRT | |
| <213> ORGANISM: Mus musculus | |
| <400> SEQUENCE: 69 | |
| Ala Pro Glu Glu Met Pro Val Asp Arg Ile Leu Glu Ala Glu Leu Ala | |
| Val Glu Gln Lys Ser Asp Gln Gly Val Glu Gly Pro Gly Ala Thr Gly | |
| Gly Gly Gly Ser Ser Pro Asn Asp Pro Val Thr Asn Ile Cys Gln Ala | |
| Ala Asp Lys Gln Leu Phe Thr Leu Val Glu Trp Ala Lys Arg Ile Pro | |
| His Phe Ser Ser Leu Pro Leu Asp Asp Gln Val Ile Leu Leu Arg Ala | |
| Gly Trp Asn Glu Leu Leu Ile Ala Ser Phe Ser His Arg Ser Ile Asp | |
| Val Arg Asp Gly Ile Leu Leu Ala Thr Gly Leu His Val His Arg Asn | |
| Ser Ala His Ser Ala Gly Val Gly Ala Ile Phe Asp Arg Val Leu Thr | |
| Glu Leu Val Ser Lys Met Arg Asp Met Arg Met Asp Lys Thr Glu Leu | |
| Gly Cys Leu Arg Ala Ile Ile Met Phe Asn Pro Asp Ala Lys Gly Leu | |
| Ser Asn Pro Gly Glu Val Glu Ile Leu Arg Glu Lys Val Tyr Ala Ser | |
| Leu Glu Thr Tyr Cys Lys Gln Lys Tyr Pro Glu Gln Gln Gly Arg Phe | |
| Ala Lys Leu Leu Leu Arg Leu Pro Ala Leu Arg Ser Ile Gly Leu Lys | |
| Cys Leu Glu His Leu Phe Phe Phe Lys Leu Ile Gly Asp Thr Pro Ile | |
| Asp Thr Phe Leu Met Glu Met Leu Glu Ala Pro His Gln Leu Ala | |
| <210> SEQ ID NO: 70 | |
| <211> LENGTH: 234 | |
| <212> TYPE: PRT | |
| <213> ORGANISM: Mus musculus | |
| <400> SEQUENCE: 70 | |
| Ser His Glu Asp Met Pro Val Glu Arg Ile Leu Glu Ala Glu Leu Ala | |
| Val Glu Pro Lys Thr Glu Ser Tyr Gly Asp Met Asn Val Glu Asn Ser | |
| Thr Asn Asp Pro Val Thr Asn Ile Cys His Ala Ala Asp Lys Gln Leu | |
| Phe Thr Leu Val Glu Trp Ala Lys Arg Ile Pro His Phe Ser Asp Leu | |
| Thr Leu Glu Asp Gln Val Ile Leu Leu Arg Ala Gly Trp Asn Glu Leu | |
| Leu Ile Ala Ser Phe Ser His Arg Ser Val Ser Val Gln Asp Gly Ile | |
| Leu Leu Ala Thr Gly Leu His Val His Arg Ser Ser Ala His Ser Arg | |
| Gly Val Gly Ser Ile Phe Asp Arg Val Leu Thr Glu Leu Val Ser Lys | |
| Met Lys Asp Met Gln Met Asp Lys Ser Glu Leu Gly Cys Leu Arg Ala | |
| Ile Val Leu Phe Asn Pro Asp Ala Lys Gly Leu Ser Asn Pro Ser Glu | |
| Val Glu Thr Leu Arg Glu Lys Val Tyr Ala Thr Leu Glu Ala Tyr Thr | |
| Lys Gln Lys Tyr Pro Glu Gln Pro Gly Arg Phe Ala Lys Leu Leu Leu | |
| Arg Leu Pro Ala Leu Arg Ser Ile Gly Leu Lys Cys Leu Glu His Leu | |
| Phe Phe Phe Lys Leu Ile Gly Asp Thr Pro Ile Asp Ser Phe Leu Met | |
| Glu Met Leu Glu Thr Pro Leu Gln Ile Thr | |
| <210> SEQ ID NO: 71 | |
| <211> LENGTH: 237 | |
| <212> TYPE: PRT | |
| <213> ORGANISM: Homo sapiens | |
| <400> SEQUENCE: 71 | |
| Ala Asn Glu Asp Met Pro Val Glu Arg Ile Leu Glu Ala Glu Leu Ala | |
| Val Glu Pro Lys Thr Glu Thr Tyr Val Glu Ala Asn Met Gly Leu Asn | |
| Pro Ser Ser Pro Asn Asp Pro Val Thr Asn Ile Cys Gln Ala Ala Asp | |
| Lys Gln Leu Phe Thr Leu Val Glu Trp Ala Lys Arg Ile Pro His Phe | |
| Ser Glu Leu Pro Leu Asp Asp Gln Val Ile Leu Leu Arg Ala Gly Trp | |
| Asn Glu Leu Leu Ile Ala Ser Phe Ser His Arg Ser Ile Ala Val Lys | |
| Asp Gly Ile Leu Leu Ala Thr Gly Leu His Val His Arg Asn Ser Ala | |
| His Ser Ala Gly Val Gly Ala Ile Phe Asp Arg Val Leu Thr Glu Leu | |
| Val Ser Lys Met Arg Asp Met Gln Met Asp Lys Thr Glu Leu Gly Cys | |
| Leu Arg Ala Ile Val Leu Phe Asn Pro Asp Ser Lys Gly Leu Ser Asn | |
| Pro Ala Glu Val Glu Ala Leu Arg Glu Lys Val Tyr Ala Ser Leu Glu | |
| Ala Tyr Cys Lys His Lys Tyr Pro Glu Gln Pro Gly Arg Phe Ala Lys | |
| Leu Leu Leu Arg Leu Pro Ala Leu Arg Ser Ile Gly Leu Lys Cys Leu | |
| Glu His Leu Phe Phe Phe Lys Leu Ile Gly Asp Thr Pro Ile Asp Thr | |
| Phe Leu Met Glu Met Leu Glu Ala Pro His Gln Met Thr | |
| <210> SEQ ID NO: 72 | |
| <211> LENGTH: 239 | |
| <212> TYPE: PRT | |
| <213> ORGANISM: Homo sapiens | |
| <400> SEQUENCE: 72 | |
| Ala Pro Glu Glu Met Pro Val Asp Arg Ile Leu Glu Ala Glu Leu Ala | |
| Val Glu Gln Lys Ser Asp Gln Gly Val Glu Gly Pro Gly Gly Thr Gly | |
| Gly Ser Gly Ser Ser Pro Asn Asp Pro Val Thr Asn Ile Cys Gln Ala | |
| Ala Asp Lys Gln Leu Phe Thr Leu Val Glu Trp Ala Lys Arg Ile Pro | |
| His Phe Ser Ser Leu Pro Leu Asp Asp Gln Val Ile Leu Leu Arg Ala | |
| Gly Trp Asn Glu Leu Leu Ile Ala Ser Phe Ser His Arg Ser Ile Asp | |
| Val Arg Asp Gly Ile Leu Leu Ala Thr Gly Leu His Val His Arg Asn | |
| Ser Ala His Ser Ala Gly Val Gly Ala Ile Phe Asp Arg Val Leu Thr | |
| Glu Leu Val Ser Lys Met Arg Asp Met Arg Met Asp Lys Thr Glu Leu | |
| Gly Cys Leu Arg Ala Ile Ile Leu Phe Asn Pro Asp Ala Lys Gly Leu | |
| Ser Asn Pro Ser Glu Val Glu Val Leu Arg Glu Lys Val Tyr Ala Ser | |
| Leu Glu Thr Tyr Cys Lys Gln Lys Tyr Pro Glu Gln Gln Gly Arg Phe | |
| Ala Lys Leu Leu Leu Arg Leu Pro Ala Leu Arg Ser Ile Gly Leu Lys | |
| Cys Leu Glu His Leu Phe Phe Phe Lys Leu Ile Gly Asp Thr Pro Ile | |
| Asp Thr Phe Leu Met Glu Met Leu Glu Ala Pro His Gln Leu Ala | |
| <210> SEQ ID NO: 73 | |
| <211> LENGTH: 234 | |
| <212> TYPE: PRT | |
| <213> ORGANISM: Homo sapiens | |
| <400> SEQUENCE: 73 | |
| Gly His Glu Asp Met Pro Val Glu Arg Ile Leu Glu Ala Glu Leu Ala | |
| Val Glu Pro Lys Thr Glu Ser Tyr Gly Asp Met Asn Met Glu Asn Ser | |
| Thr Asn Asp Pro Val Thr Asn Ile Cys His Ala Ala Asp Lys Gln Leu | |
| Phe Thr Leu Val Glu Trp Ala Lys Arg Ile Pro His Phe Ser Asp Leu | |
| Thr Leu Glu Asp Gln Val Ile Leu Leu Arg Ala Gly Trp Asn Glu Leu | |
| Leu Ile Ala Ser Phe Ser His Arg Ser Val Ser Val Gln Asp Gly Ile | |
| Leu Leu Ala Thr Gly Leu His Val His Arg Ser Ser Ala His Ser Ala | |
| Gly Val Gly Ser Ile Phe Asp Arg Val Leu Thr Glu Leu Val Ser Lys | |
| Met Lys Asp Met Gln Met Asp Lys Ser Glu Leu Gly Cys Leu Arg Ala | |
| Ile Val Leu Phe Asn Pro Asp Ala Lys Gly Leu Ser Asn Pro Ser Glu | |
| Val Glu Thr Leu Arg Glu Lys Val Tyr Ala Thr Leu Glu Ala Tyr Thr | |
| Lys Gln Lys Tyr Pro Glu Gln Pro Gly Arg Phe Ala Lys Leu Leu Leu | |
| Arg Leu Pro Ala Leu Arg Ser Ile Gly Leu Lys Cys Leu Glu His Leu | |
| Phe Phe Phe Lys Leu Ile Gly Asp Thr Pro Ile Asp Thr Phe Leu Met | |
| Glu Met Leu Glu Thr Pro Leu Gln Ile Thr | |
| <210> SEQ ID NO: 74 | |
| <211> LENGTH: 516 | |
| <212> TYPE: DNA | |
| <213> ORGANISM: Locusta migratoria | |
| <400> SEQUENCE: 74 | |
| atccctacct ctggaggacc aggttctcct cctcagagca ggttggaatg aactgctaat | 60 |
| tgcagcattt tcacatcgat ctgtagatgt taaagatggc atagtacttg ccactggtct | 120 |
| cacagtgcat cgaaattctg cccatcaagc tggagtcggc acaatatttg acagagtttt | 180 |
| gacagaactg gtagcaaaga tgagagaaat gaaaatggat aaaactgaac ttggctgctt | 240 |
| gcgatctgtt attcttttca atccagaggt gaggggtttg aaatccgccc aggaagttga | 300 |
| acttctacgt gaaaaagtat atgccgcttt ggaagaatat actagaacaa cacatcccga | 360 |
| tgaaccagga agatttgcaa aacttttgct tcgtctgcct tctttacgtt ccataggcct | 420 |
| taagtgtttg gagcatttgt tttctttcgc cttattggag atgttccaat tgatacgttc | 480 |
| ctgatggaga tgcttgaatc accttctgat tcataa | 516 |
| <210> SEQ ID NO: 75 | |
| <211> LENGTH: 528 | |
| <212> TYPE: DNA | |
| <213> ORGANISM: Amblyomma americanum | |
| <400> SEQUENCE: 75 | |
| attccacatt ttgaagagct tccccttgag gaccgcatgg tgttgctcaa ggctggctgg | 60 |
| aacgagctgc tcattgctgc tttctcccac cgttctgttg acgtgcgtga tggcattgtg | 120 |
| ctcgctacag gtcttgtggt gcagcggcat agtgctcatg gggctggcgt tggggccata | 180 |
| tttgataggg ttctcactga actggtagca aagatgcgtg agatgaagat ggaccgcact | 240 |
| gagcttggat gcctgcttgc tgtggtactt tttaatcctg aggccaaggg gctgcggacc | 300 |
| tgcccaagtg gaggccctga gggagaaagt gtatctgcct tggaagagca ctgccggcag | 360 |
| cagtacccag accagcctgg gcgctttgcc aagctgctgc tgcggttgcc agctctgcgc | 420 |
| agtattggcc tcaagtgcct cgaacatctc tttttcttca agctcatcgg ggacacgccc | 480 |
| atcgacaact ttcttctttc catgctggag gccccctctg acccctaa | 528 |
| <210> SEQ ID NO: 76 | |
| <211> LENGTH: 531 | |
| <212> TYPE: DNA | |
| <213> ORGANISM: Amblyomma americanum | |
| <400> SEQUENCE: 76 | |
| attccgcact tcgaagagct tcccatcgag gatcgcaccg cgctgctcaa agccggctgg | 60 |
| aacgaactgc ttattgccgc cttttcgcac cgttctgtgg cggtgcgcga cggcatcgtt | 120 |
| ctggccaccg ggctggtggt gcagcggcac agcgcacacg gcgcaggcgt tggcgacatc | 180 |
| ttcgaccgcg tactagccga gctggtggcc aagatgcgcg acatgaagat ggacaaaacg | 240 |
| gagctcggct gcctgcgcgc cgtggtgctc ttcaatccag acgccaaggg tctccgaaac | 300 |
| gccaccagag tagaggcgct ccgcgagaag gtgtatgcgg cgctggagga gcactgccgt | 360 |
| cggcaccacc cggaccaacc gggtcgcttc ggcaagctgc tgctgcggct gcctgccttg | 420 |
| cgcagcatcg ggctcaaatg cctcgagcat ctgttcttct tcaagctcat cggagacact | 480 |
| cccatagaca gcttcctgct caacatgctg gaggcaccgg cagaccccta g | 531 |
| <210> SEQ ID NO: 77 | |
| <211> LENGTH: 552 | |
| <212> TYPE: DNA | |
| <213> ORGANISM: Celuca pugilator | |
| <400> SEQUENCE: 77 | |
| atcccacact tcacagacct tcccatagag gaccaagtgg tattactcaa agccgggtgg | 60 |
| aacgagttgc ttattgcctc attctcacac cgtagcatgg gcgtggagga tggcatcgtg | 120 |
| ctggccacag ggctcgtgat ccacagaagt agtgctcacc aggctggagt gggtgccata | 180 |
| tttgatcgtg tcctctctga gctggtggcc aagatgaagg agatgaagat tgacaagaca | 240 |
| gagctgggct gccttcgctc catcgtcctg ttcaacccag atgccaaagg actaaactgc | 300 |
| gtcaatgatg tggagatctt gcgtgagaag gtgtatgctg ccctggagga gtacacacga | 360 |
| accacttacc ctgatgaacc tggacgcttt gccaagttgc ttctgcgact tcctgcactc | 420 |
| aggtctatag gcctgaagtg tcttgagtac ctcttcctgt ttaagctgat tggagacact | 480 |
| cccctggaca gctacttgat gaagatgctc gtagacaacc caaatacaag cgtcactccc | 540 |
| cccaccagct ag | 552 |
| <210> SEQ ID NO: 78 | |
| <211> LENGTH: 531 | |
| <212> TYPE: DNA | |
| <213> ORGANISM: Tenebrio molitor | |
| <400> SEQUENCE: 78 | |
| atacctcact ttacctcgtt gccgatgtcg gaccaggtgc ttttattgag ggcaggatgg | 60 |
| aatgaattgc tcatcgccgc attctcgcac agatctatac aggcgcagga tgccatcgtt | 120 |
| ctagccacgg ggttgacagt taacaaaacg tcggcgcacg ccgtgggcgt gggcaacatc | 180 |
| tacgaccgcg tcctctccga gctggtgaac aagatgaaag agatgaagat ggacaagacg | 240 |
| gagctgggct gcttgagagc catcatcctc tacaacccca cgtgtcgcgg catcaagtcc | 300 |
| gtgcaggaag tggagatgct gcgtgagaaa atttacggcg tgctggaaga gtacaccagg | 360 |
| accacccacc cgaacgagcc cggcaggttc gccaaactgc ttctgcgcct cccggccctc | 420 |
| aggtccatcg ggttgaaatg ttccgaacac ctctttttct tcaagctgat cggtgatgtt | 480 |
| ccaatagaca cgttcctgat ggagatgctg gagtctccgg cggacgctta g | 531 |
| <210> SEQ ID NO: 79 | |
| <211> LENGTH: 531 | |
| <212> TYPE: DNA | |
| <213> ORGANISM: Apis mellifera | |
| <400> SEQUENCE: 79 | |
| atcccgcatt ttacctcgtt gccactggag gatcaggtac ttctgctcag ggccggttgg | 60 |
| aacgagttgc tgatagcctc cttttcccac cgttccatcg acgtgaagga cggtatcgtg | 120 |
| ctggcgacgg ggatcaccgt gcatcggaac tcggcgcagc aggccggcgt gggcacgata | 180 |
| ttcgaccgtg tcctctcgga gcttgtctcg aaaatgcgtg aaatgaagat ggacaggaca | 240 |
| gagcttggct gtctcagatc tataatactc ttcaatcccg aggttcgagg actgaaatcc | 300 |
| atccaggaag tgaccctgct ccgtgagaag atctacggcg ccctggaggg ttattgccgc | 360 |
| gtagcttggc ccgacgacgc tggaagattc gcgaaattac ttctacgcct gcccgccatc | 420 |
| cgctcgatcg gattaaagtg cctcgagtac ctgttcttct tcaaaatgat cggtgacgta | 480 |
| ccgatcgacg attttctcgt ggagatgtta gaatcgcgat cagatcctta g | 531 |
| <210> SEQ ID NO: 80 | |
| <211> LENGTH: 176 | |
| <212> TYPE: PRT | |
| <213> ORGANISM: Locusta migratoria | |
| <400> SEQUENCE: 80 | |
| Ile Pro His Phe Thr Ser Leu Pro Leu Glu Asp Gln Val Leu Leu Leu | |
| Arg Ala Gly Trp Asn Glu Leu Leu Ile Ala Ala Phe Ser His Arg Ser | |
| Val Asp Val Lys Asp Gly Ile Val Leu Ala Thr Gly Leu Thr Val His | |
| Arg Asn Ser Ala His Gln Ala Gly Val Gly Thr Ile Phe Asp Arg Val | |
| Leu Thr Glu Leu Val Ala Lys Met Arg Glu Met Lys Met Asp Lys Thr | |
| Glu Leu Gly Cys Leu Arg Ser Val Ile Leu Phe Asn Pro Glu Val Arg | |
| Gly Leu Lys Ser Ala Gln Glu Val Glu Leu Leu Arg Glu Lys Val Tyr | |
| Ala Ala Leu Glu Glu Tyr Thr Arg Thr Thr His Pro Asp Glu Pro Gly | |
| Arg Phe Ala Lys Leu Leu Leu Arg Leu Pro Ser Leu Arg Ser Ile Gly | |
| Leu Lys Cys Leu Glu His Leu Phe Phe Phe Arg Leu Ile Gly Asp Val | |
| Pro Ile Asp Thr Phe Leu Met Glu Met Leu Glu Ser Pro Ser Asp Ser | |
| <210> SEQ ID NO: 81 | |
| <211> LENGTH: 175 | |
| <212> TYPE: PRT | |
| <213> ORGANISM: Amblyomma americanum | |
| <400> SEQUENCE: 81 | |
| Ile Pro His Phe Glu Glu Leu Pro Leu Glu Asp Arg Met Val Leu Leu | |
| Lys Ala Gly Trp Asn Glu Leu Leu Ile Ala Ala Phe Ser His Arg Ser | |
| Val Asp Val Arg Asp Gly Ile Val Leu Ala Thr Gly Leu Val Val Gln | |
| Arg His Ser Ala His Gly Ala Gly Val Gly Ala Ile Phe Asp Arg Val | |
| Leu Thr Glu Leu Val Ala Lys Met Arg Glu Met Lys Met Asp Arg Thr | |
| Glu Leu Gly Cys Leu Leu Ala Val Val Leu Phe Asn Pro Glu Ala Lys | |
| Gly Leu Arg Thr Cys Pro Ser Gly Gly Pro Glu Gly Glu Ser Val Ser | |
| Ala Leu Glu Glu His Cys Arg Gln Gln Tyr Pro Asp Gln Pro Gly Arg | |
| Phe Ala Lys Leu Leu Leu Arg Leu Pro Ala Leu Arg Ser Ile Gly Leu | |
| Lys Cys Leu Glu His Leu Phe Phe Phe Lys Leu Ile Gly Asp Thr Pro | |
| Ile Asp Asn Phe Leu Leu Ser Met Leu Glu Ala Pro Ser Asp Pro | |
| <210> SEQ ID NO: 82 | |
| <211> LENGTH: 176 | |
| <212> TYPE: PRT | |
| <213> ORGANISM: Amblyomma americanum | |
| <400> SEQUENCE: 82 | |
| Ile Pro His Phe Glu Glu Leu Pro Ile Glu Asp Arg Thr Ala Leu Leu | |
| Lys Ala Gly Trp Asn Glu Leu Leu Ile Ala Ala Phe Ser His Arg Ser | |
| Val Ala Val Arg Asp Gly Ile Val Leu Ala Thr Gly Leu Val Val Gln | |
| Arg His Ser Ala His Gly Ala Gly Val Gly Asp Ile Phe Asp Arg Val | |
| Leu Ala Glu Leu Val Ala Lys Met Arg Asp Met Lys Met Asp Lys Thr | |
| Glu Leu Gly Cys Leu Arg Ala Val Val Leu Phe Asn Pro Asp Ala Lys | |
| Gly Leu Arg Asn Ala Thr Arg Val Glu Ala Leu Arg Glu Lys Val Tyr | |
| Ala Ala Leu Glu Glu His Cys Arg Arg His His Pro Asp Gln Pro Gly | |
| Arg Phe Gly Lys Leu Leu Leu Arg Leu Pro Ala Leu Arg Ser Ile Gly | |
| Leu Lys Cys Leu Glu His Leu Phe Phe Phe Lys Leu Ile Gly Asp Thr | |
| Pro Ile Asp Ser Phe Leu Leu Asn Met Leu Glu Ala Pro Ala Asp Pro | |
| <210> SEQ ID NO: 83 | |
| <211> LENGTH: 183 | |
| <212> TYPE: PRT | |
| <213> ORGANISM: Celuca pugilator | |
| <400> SEQUENCE: 83 | |
| Ile Pro His Phe Thr Asp Leu Pro Ile Glu Asp Gln Val Val Leu Leu | |
| Lys Ala Gly Trp Asn Glu Leu Leu Ile Ala Ser Phe Ser His Arg Ser | |
| Met Gly Val Glu Asp Gly Ile Val Leu Ala Thr Gly Leu Val Ile His | |
| Arg Ser Ser Ala His Gln Ala Gly Val Gly Ala Ile Phe Asp Arg Val | |
| Leu Ser Glu Leu Val Ala Lys Met Lys Glu Met Lys Ile Asp Lys Thr | |
| Glu Leu Gly Cys Leu Arg Ser Ile Val Leu Phe Asn Pro Asp Ala Lys | |
| Gly Leu Asn Cys Val Asn Asp Val Glu Ile Leu Arg Glu Lys Val Tyr | |
| Ala Ala Leu Glu Glu Tyr Thr Arg Thr Thr Tyr Pro Asp Glu Pro Gly | |
| Arg Phe Ala Lys Leu Leu Leu Arg Leu Pro Ala Leu Arg Ser Ile Gly | |
| Leu Lys Cys Leu Glu Tyr Leu Phe Leu Phe Lys Leu Ile Gly Asp Thr | |
| Pro Leu Asp Ser Tyr Leu Met Lys Met Leu Val Asp Asn Pro Asn Thr | |
| Ser Val Thr Pro Pro Thr Ser | |
| <210> SEQ ID NO: 84 | |
| <211> LENGTH: 176 | |
| <212> TYPE: PRT | |
| <213> ORGANISM: Tenebrio molitor | |
| <400> SEQUENCE: 84 | |
| Ile Pro His Phe Thr Ser Leu Pro Met Ser Asp Gln Val Leu Leu Leu | |
| Arg Ala Gly Trp Asn Glu Leu Leu Ile Ala Ala Phe Ser His Arg Ser | |
| Ile Gln Ala Gln Asp Ala Ile Val Leu Ala Thr Gly Leu Thr Val Asn | |
| Lys Thr Ser Ala His Ala Val Gly Val Gly Asn Ile Tyr Asp Arg Val | |
| Leu Ser Glu Leu Val Asn Lys Met Lys Glu Met Lys Met Asp Lys Thr | |
| Glu Leu Gly Cys Leu Arg Ala Ile Ile Leu Tyr Asn Pro Thr Cys Arg | |
| Gly Ile Lys Ser Val Gln Glu Val Glu Met Leu Arg Glu Lys Ile Tyr | |
| Gly Val Leu Glu Glu Tyr Thr Arg Thr Thr His Pro Asn Glu Pro Gly | |
| Arg Phe Ala Lys Leu Leu Leu Arg Leu Pro Ala Leu Arg Ser Ile Gly | |
| Leu Lys Cys Ser Glu His Leu Phe Phe Phe Lys Leu Ile Gly Asp Val | |
| Pro Ile Asp Thr Phe Leu Met Glu Met Leu Glu Ser Pro Ala Asp Ala | |
| <210> SEQ ID NO: 85 | |
| <211> LENGTH: 176 | |
| <212> TYPE: PRT | |
| <213> ORGANISM: Apis mellifera | |
| <400> SEQUENCE: 85 | |
| Ile Pro His Phe Thr Ser Leu Pro Leu Glu Asp Gln Val Leu Leu Leu | |
| Arg Ala Gly Trp Asn Glu Leu Leu Ile Ala Ser Phe Ser His Arg Ser | |
| Ile Asp Val Lys Asp Gly Ile Val Leu Ala Thr Gly Ile Thr Val His | |
| Arg Asn Ser Ala Gln Gln Ala Gly Val Gly Thr Ile Phe Asp Arg Val | |
| Leu Ser Glu Leu Val Ser Lys Met Arg Glu Met Lys Met Asp Arg Thr | |
| Glu Leu Gly Cys Leu Arg Ser Ile Ile Leu Phe Asn Pro Glu Val Arg | |
| Gly Leu Lys Ser Ile Gln Glu Val Thr Leu Leu Arg Glu Lys Ile Tyr | |
| Gly Ala Leu Glu Gly Tyr Cys Arg Val Ala Trp Pro Asp Asp Ala Gly | |
| Arg Phe Ala Lys Leu Leu Leu Arg Leu Pro Ala Ile Arg Ser Ile Gly | |
| Leu Lys Cys Leu Glu Tyr Leu Phe Phe Phe Lys Met Ile Gly Asp Val | |
| Pro Ile Asp Asp Phe Leu Val Glu Met Leu Glu Ser Arg Ser Asp Pro | |
| <210> SEQ ID NO: 86 | |
| <211> LENGTH: 259 | |
| <212> TYPE: PRT | |
| <213> ORGANISM: Choristoneura fumiferana | |
| <400> SEQUENCE: 86 | |
| Leu Thr Ala Asn Gln Gln Phe Leu Ile Ala Arg Leu Ile Trp Tyr Gln | |
| Asp Gly Tyr Glu Gln Pro ser Asp Glu Asp Leu Lys Arg Ile Thr Gln | |
| Thr Trp Gln Gln Ala Asp Asp Glu Asn Glu Glu ser Asp Thr Pro Phe | |
| Arg Gln Ile Thr Glu Met Thr Ile Leu Thr Val Gln Leu Ile Val Glu | |
| Phe Ala Lys Gly Leu Pro Gly Phe Ala Lys Ile ser Gln Pro Asp Gln | |
| Ile Thr Leu Leu Lys Ala cys ser ser Glu Val Met Met Leu Arg Val | |
| Ala Arg Arg Tyr Asp Ala Ala ser Asp ser Val Leu Phe Ala Asn Asn | |
| Gln Ala Tyr Thr Arg Asp Asn Tyr Arg Lys Ala Gly Met Ala Tyr Val | |
| Ile Glu Asp Leu Leu His Phe cys Arg cys Met Tyr ser Met Ala Leu | |
| Asp Asn Ile His Tyr Ala Leu Leu Thr Ala val val Ile Phe ser Asp | |
| Arg Pro Gly Leu Glu Gln Pro Gln Leu val Glu Glu Ile Gln Arg Tyr | |
| Tyr Leu Asn Thr Leu Arg Ile Tyr Ile Leu Asn Gln Leu ser Gly ser | |
| Ala Arg ser ser Val Ile Tyr Gly Lys Ile Leu ser Ile Leu ser Glu | |
| Leu Arg Thr Leu Gly Met Gln Asn ser Asn Met cys Ile Ser Leu Lys | |
| Leu Lys Asn Arg Lys Leu Pro Pro Phe Leu Glu Glu Ile Trp Asp val | |
| Ala Asp Met ser His Thr Gln Pro Pro Pro Ile Leu Glu ser Pro Thr | |
| Asn Leu Gly | |
| <210> SEQ ID NO: 87 | |
| <211> LENGTH: 674 | |
| <212> TYPE: PRT | |
| <213> ORGANISM: Artificial | |
| <400> SEQUENCE: 87 | |
| Met Asp Tyr Lys Asp Asp Asp Asp Lys Glu Met Pro Val Asp Arg Ile | |
| Leu Glu Ala Glu Leu Ala Val Glu Gln Lys Ser Asp Gln Gly Val Glu | |
| Gly Pro Gly Gly Thr Gly Gly Ser Gly Ser Ser Pro Asn Asp Pro Val | |
| Thr Asn Ile Cys Gln Ala Ala Asp Lys Gln Leu Phe Thr Leu Val Glu | |
| Trp Ala Lys Arg Ile Pro His Phe Ser Ser Leu Pro Leu Asp Asp Gln | |
| Val Ile Leu Leu Arg Ala Gly Trp Asn Glu Leu Leu Ile Ala Ser Phe | |
| Ser His Arg Ser Ile Asp Val Arg Asp Gly Ile Leu Leu Ala Thr Gly | |
| Leu His Val His Arg Asn Ser Ala His Ser Ala Gly Val Gly Ala Ile | |
| Phe Asp Arg Val Leu Thr Glu Leu Val Ser Lys Met Arg Asp Met Arg | |
| Met Asp Lys Thr Glu Leu Gly Cys Leu Arg Ala Ile Ile Leu Phe Asn | |
| Pro Glu Val Arg Gly Leu Lys Ser Ala Gln Glu Val Glu Leu Leu Arg | |
| Glu Lys Val Tyr Ala Ala Leu Glu Glu Tyr Thr Arg Thr Thr His Pro | |
| Asp Glu Pro Gly Arg Phe Ala Lys Leu Leu Leu Arg Leu Pro Ser Leu | |
| Arg Ser Ile Gly Leu Lys Cys Leu Glu His Leu Phe Phe Phe Arg Leu | |
| Ile Gly Asp Val Pro Ile Asp Thr Phe Leu Met Glu Met Leu Glu Ser | |
| Pro Ser Asp Ser Gln Ile Ser Tyr Ala Ser Arg Gly Gly Gly Ser Ser | |
| Gly Gly Gly Glu Asp Ala Lys Asn Ile Lys Lys Gly Pro Ala Pro Phe | |
| Tyr Pro Leu Glu Asp Gly Thr Ala Gly Glu Gln Leu His Lys Ala Met | |
| Lys Arg Tyr Ala Leu Val Pro Gly Thr Ile Ala Phe Thr Asp Ala His | |
| Ile Glu Val Asn Ile Thr Tyr Ala Glu Tyr Phe Glu Met Ser Val Arg | |
| Leu Ala Glu Ala Met Lys Arg Tyr Gly Leu Asn Thr Asn His Arg Ile | |
| Val Val Cys Ser Glu Asn Ser Leu Gln Phe Phe Met Pro Val Leu Gly | |
| Ala Leu Phe Ile Gly Val Ala Val Ala Pro Ala Asn Asp Ile Tyr Asn | |
| Glu Arg Glu Leu Leu Asn Ser Met Asn Ile Ser Gln Pro Thr Val Val | |
| Phe Val Ser Lys Lys Gly Leu Gln Lys Ile Leu Asn Val Gln Lys Lys | |
| Leu Pro Ile Ile Gln Lys Ile Ile Ile Met Asp Ser Lys Thr Asp Tyr | |
| Gln Gly Phe Gln Ser Met Tyr Thr Phe Val Thr Ser His Leu Pro Pro | |
| Gly Phe Asn Glu Tyr Asp Phe Val Pro Glu Ser Phe Asp Arg Asp Lys | |
| Thr Ile Ala Leu Ile Met Asn Ser Ser Gly Ser Thr Gly Leu Pro Lys | |
| Gly Val Ala Leu Pro His Arg Thr Ala Cys Val Arg Phe Ser His Ala | |
| Arg Asp Pro Ile Phe Gly Asn Gln Ile Ile Pro Asp Thr Ala Ile Leu | |
| Ser Val Val Pro Phe His His Gly Phe Gly Met Phe Thr Thr Leu Gly | |
| Tyr Leu Ile Cys Gly Phe Arg Val Val Leu Met Tyr Arg Phe Glu Glu | |
| Glu Leu Phe Leu Arg Ser Leu Gln Asp Tyr Lys Ile Gln Ser Ala Leu | |
| Leu Val Pro Thr Leu Phe Ser Phe Phe Ala Lys Ser Thr Leu Ile Asp | |
| Lys Tyr Asp Leu Ser Asn Leu His Glu Ile Ala Ser Gly Gly Ala Pro | |
| Leu Ser Lys Glu Val Gly Glu Ala Val Ala Lys Arg Phe His Leu Pro | |
| Gly Ile Arg Gln Gly Tyr Gly Leu Thr Glu Thr Thr Ser Ala Ile Leu | |
| Ile Thr Pro Glu Gly Asp Asp Lys Pro Gly Ala Val Gly Lys Val Val | |
| Pro Phe Phe Glu Ala Lys Val Val Asp Leu Asp Thr Gly Lys Thr Leu | |
| Gly Val Asn Gln Arg Gly Glu Leu Cys Val Arg Gly Pro Met Ile Met | |
| Ser Gly Tyr Val Asn Asn Pro Glu Ala Thr Asn Ala Leu Ile Asp Lys | |
| Asp Gly | |
| <210> SEQ ID NO: 88 | |
| <211> LENGTH: 463 | |
| <212> TYPE: PRT | |
| <213> ORGANISM: Artificial | |
| <400> SEQUENCE: 88 | |
| Gln Val Ala Pro Ala Glu Leu Glu Ser Ile Leu Leu Gln His Pro Asn | |
| Ile Phe Asp Ala Gly Val Ala Gly Leu Pro Asp Asp Asp Ala Gly Glu | |
| Leu Pro Ala Ala Val Val Val Leu Glu His Gly Lys Thr Met Thr Glu | |
| Lys Glu Ile Val Asp Tyr Val Ala Ser Gln Val Thr Thr Ala Lys Lys | |
| Leu Arg Gly Gly Val Val Phe Val Asp Glu Val Pro Lys Gly Leu Thr | |
| Gly Lys Leu Asp Ala Arg Lys Ile Arg Glu Ile Leu Ile Lys Ala Lys | |
| Lys Gly Gly Lys Ser Lys Leu Gly Gly Gly Ser Ser Gly Gly Gly Gln | |
| Ile Ser Tyr Ala Ser Arg Gly Arg Pro Glu Cys Val Val Pro Glu Thr | |
| Gln Cys Ala Met Lys Arg Lys Glu Lys Lys Ala Gln Lys Glu Lys Asp | |
| Lys Leu Pro Val Ser Thr Thr Thr Val Asp Asp His Met Pro Pro Ile | |
| Met Gln Cys Glu Pro Pro Pro Pro Glu Ala Ala Arg Ile His Glu Val | |
| Val Pro Arg Phe Leu Ser Asp Lys Leu Leu Val Thr Asn Arg Gln Lys | |
| Asn Ile Pro Gln Leu Thr Ala Asn Gln Gln Phe Leu Ile Ala Arg Leu | |
| Ile Trp Tyr Gln Asp Gly Tyr Glu Gln Pro Ser Asp Glu Asp Leu Lys | |
| Arg Ile Thr Gln Thr Trp Gln Gln Ala Asp Asp Glu Asn Glu Glu Ser | |
| Asp Thr Pro Phe Arg Gln Ile Thr Glu Met Thr Ile Leu Thr Val Gln | |
| Leu Ile Val Glu Phe Ala Lys Gly Leu Pro Gly Phe Ala Lys Ile Ser | |
| Gln Pro Asp Gln Ile Thr Leu Leu Lys Ala Cys Ser Ser Glu Val Met | |
| Met Leu Arg Val Ala Arg Arg Tyr Asp Ala Ala Ser Asp Ser Ile Leu | |
| Phe Ala Asn Asn Gln Ala Tyr Thr Arg Asp Asn Tyr Arg Lys Ala Gly | |
| Met Ala Glu Val Ile Glu Asp Leu Leu His Phe Cys Arg Cys Met Tyr | |
| Ser Met Ala Leu Asp Asn Ile His Tyr Ala Leu Leu Thr Ala Val Val | |
| Ile Phe Ser Asp Arg Pro Gly Leu Glu Gln Pro Gln Leu Val Glu Glu | |
| Ile Gln Arg Tyr Tyr Leu Asn Thr Leu Arg Ile Tyr Ile Leu Asn Gln | |
| Leu Ser Gly Ser Ala Arg Ser Ser Val Ile Tyr Gly Lys Ile Leu Ser | |
| Ile Leu Ser Glu Leu Arg Thr Leu Gly Met Gln Asn Ser Asn Met Cys | |
| Ile Ser Leu Lys Leu Lys Asn Arg Lys Leu Pro Pro Phe Leu Glu Glu | |
| Ile Trp Asp Val Ala Asp Met Ser His Thr Gln Pro Pro Pro Ile Leu | |
| Glu Ser Pro Thr Asn Leu Tyr Pro Tyr Asp Val Pro Asp Tyr Ala | |
| <210> SEQ ID NO: 89 | |
| <211> LENGTH: 675 | |
| <212> TYPE: PRT | |
| <213> ORGANISM: Artificial | |
| <400> SEQUENCE: 89 | |
| Trp Tyr Gln Asp Gly Tyr Glu Gln Pro Ser Asp Glu Asp Leu Lys Arg | |
| Ile Thr Gln Thr Trp Gln Gln Ala Asp Asp Glu Asn Glu Glu Ser Asp | |
| Thr Pro Phe Arg Gln Ile Thr Glu Met Thr Ile Leu Thr Val Gln Leu | |
| Ile Val Glu Phe Ala Lys Gly Leu Pro Gly Phe Ala Lys Ile Ser Gln | |
| Pro Asp Gln Ile Thr Leu Leu Lys Ala Cys Ser Ser Glu Val Met Met | |
| Leu Arg Val Ala Arg Arg Tyr Asp Ala Ala Ser Asp Ser Ile Leu Phe | |
| Ala Asn Asn Gln Ala Tyr Thr Arg Asp Asn Tyr Arg Lys Ala Gly Met | |
| Ala Glu Val Ile Glu Asp Leu Leu His Phe Cys Arg Cys Met Tyr Ser | |
| Met Ala Leu Asp Asn Ile His Tyr Ala Leu Leu Thr Ala Val Val Ile | |
| Phe Ser Asp Arg Pro Gly Leu Glu Gln Pro Gln Leu Val Glu Glu Ile | |
| Gln Arg Tyr Tyr Leu Asn Thr Leu Arg Ile Tyr Ile Leu Asn Gln Leu | |
| Ser Gly Ser Ala Arg Ser Ser Val Ile Tyr Gly Lys Ile Leu Ser Ile | |
| Leu Ser Glu Leu Arg Thr Leu Gly Met Gln Asn Ser Asn Met Cys Ile | |
| Ser Leu Lys Leu Lys Asn Arg Lys Leu Pro Pro Phe Leu Glu Glu Ile | |
| Trp Asp Val Ala Asp Met Ser His Thr Gln Pro Pro Pro Ile Leu Glu | |
| Ser Pro Thr Asn Leu Gln Ile Ser Tyr Ala Ser Arg Gly Gly Gly Ser | |
| Ser Gly Gly Gly Glu Asp Ala Lys Asn Ile Lys Lys Gly Pro Ala Pro | |
| Phe Tyr Pro Leu Glu Asp Gly Thr Ala Gly Glu Gln Leu His Lys Ala | |
| Met Lys Arg Tyr Ala Leu Val Pro Gly Thr Ile Ala Phe Thr Asp Ala | |
| His Ile Glu Val Asn Ile Thr Tyr Ala Glu Tyr Phe Glu Met Ser Val | |
| Arg Leu Ala Glu Ala Met Lys Arg Tyr Gly Leu Asn Thr Asn His Arg | |
| Ile Val Val Cys Ser Glu Asn Ser Leu Gln Phe Phe Met Pro Val Leu | |
| Gly Ala Leu Phe Ile Gly Val Ala Val Ala Pro Ala Asn Asp Ile Tyr | |
| Asn Glu Arg Glu Leu Leu Asn Ser Met Asn Ile Ser Gln Pro Thr Val | |
| Val Phe Val Ser Lys Lys Gly Leu Gln Lys Ile Leu Asn Val Gln Lys | |
| Lys Leu Pro Ile Ile Gln Lys Ile Ile Ile Met Asp Ser Lys Thr Asp | |
| Tyr Gln Gly Phe Gln Ser Met Tyr Thr Phe Val Thr Ser His Leu Pro | |
| Pro Gly Phe Asn Glu Tyr Asp Phe Val Pro Glu Ser Phe Asp Arg Asp | |
| Lys Thr Ile Ala Leu Ile Met Asn Ser Ser Gly Ser Thr Gly Leu Pro | |
| Lys Gly Val Ala Leu Pro His Arg Thr Ala Cys Val Arg Phe Ser His | |
| Ala Arg Asp Pro Ile Phe Gly Asn Gln Ile Ile Pro Asp Thr Ala Ile | |
| Leu Ser Val Val Pro Phe His His Gly Phe Gly Met Phe Thr Thr Leu | |
| Gly Tyr Leu Ile Cys Gly Phe Arg Val Val Leu Met Tyr Arg Phe Glu | |
| Glu Glu Leu Phe Leu Arg Ser Leu Gln Asp Tyr Lys Ile Gln Ser Ala | |
| Leu Leu Val Pro Thr Leu Phe Ser Phe Phe Ala Lys Ser Thr Leu Ile | |
| Asp Lys Tyr Asp Leu Ser Asn Leu His Glu Ile Ala Ser Gly Gly Ala | |
| Pro Leu Ser Lys Glu Val Gly Glu Ala Val Ala Lys Arg Phe His Leu | |
| Pro Gly Ile Arg Gln Gly Tyr Gly Leu Thr Glu Thr Thr Ser Ala Ile | |
| Leu Ile Thr Pro Glu Gly Asp Asp Lys Pro Gly Ala Val Gly Lys Val | |
| Val Pro Phe Phe Glu Ala Lys Val Val Asp Leu Asp Thr Gly Lys Thr | |
| Leu Gly Val Asn Gln Arg Gly Glu Leu Cys Val Arg Gly Pro Met Ile | |
| Met Ser Gly Tyr Val Asn Asn Pro Glu Ala Thr Asn Ala Leu Ile Asp | |
| Lys Asp Gly | |
| <210> SEQ ID NO: 90 | |
| <211> LENGTH: 412 | |
| <212> TYPE: PRT | |
| <213> ORGANISM: Artificial | |
| <400> SEQUENCE: 90 | |
| Met Ser Gly Tyr Val Asn Asn Pro Glu Ala Thr Asn Ala Leu Ile Asp | |
| Lys Asp Gly Trp Leu His Ser Gly Asp Ile Ala Tyr Trp Asp Glu Asp | |
| Glu His Phe Phe Ile Val Asp Arg Leu Lys Ser Leu Ile Lys Tyr Lys | |
| Gly Tyr Gln Val Ala Pro Ala Glu Leu Glu Ser Ile Leu Leu Gln His | |
| Pro Asn Ile Phe Asp Ala Gly Val Ala Gly Leu Pro Asp Asp Asp Ala | |
| Gly Glu Leu Pro Ala Ala Val Val Val Leu Glu His Gly Lys Thr Met | |
| Thr Glu Lys Glu Ile Val Asp Tyr Val Ala Ser Gln Val Thr Thr Ala | |
| Lys Lys Leu Arg Gly Gly Val Val Phe Val Asp Glu Val Pro Lys Gly | |
| Leu Thr Gly Lys Leu Asp Ala Arg Lys Ile Arg Glu Ile Leu Ile Lys | |
| Ala Lys Lys Gly Gly Lys Ser Lys Leu Gly Gly Gly Ser Ser Gly Gly | |
| Gly Gln Ile Ser Tyr Ala Ser Arg Gly Glu Met Pro Val Asp Arg Ile | |
| Leu Glu Ala Glu Leu Ala Val Glu Gln Lys Ser Asp Gln Gly Val Glu | |
| Gly Pro Gly Gly Thr Gly Gly Ser Gly Ser Ser Pro Asn Asp Pro Val | |
| Thr Asn Ile Cys Gln Ala Ala Asp Lys Gln Leu Phe Thr Leu Val Glu | |
| Trp Ala Lys Arg Ile Pro His Phe Ser Ser Leu Pro Leu Asp Asp Gln | |
| Val Ile Leu Leu Arg Ala Gly Trp Asn Glu Leu Leu Ile Ala Ser Phe | |
| Ser His Arg Ser Ile Asp Val Arg Asp Gly Ile Leu Leu Ala Thr Gly | |
| Leu His Val His Arg Asn Ser Ala His Ser Ala Gly Val Gly Ala Ile | |
| Phe Asp Arg Val Leu Thr Glu Leu Val Ser Lys Met Arg Asp Met Arg | |
| Met Asp Lys Thr Glu Leu Gly Cys Leu Arg Ala Ile Ile Leu Phe Asn | |
| Pro Glu Val Arg Gly Leu Lys Ser Ala Gln Glu Val Glu Leu Leu Arg | |
| Glu Lys Val Tyr Ala Ala Leu Glu Glu Tyr Thr Arg Thr Thr His Pro | |
| Asp Glu Pro Gly Arg Phe Ala Lys Leu Leu Leu Arg Leu Pro Ser Leu | |
| Arg Ser Ile Gly Leu Lys Cys Leu Glu His Leu Phe Phe Phe Arg Leu | |
| Ile Gly Asp Val Pro Ile Asp Thr Phe Leu Met Glu Met Leu Glu Ser | |
| Pro Ser Asp Ser Asp Tyr Lys Asp Asp Asp Asp Lys | |
| <210> SEQ ID NO: 91 | |
| <211> LENGTH: 1189 | |
| <212> TYPE: PRT | |
| <213> ORGANISM: Artificial | |
| <400> SEQUENCE: 91 | |
| Met Tyr Pro Tyr Asp Val Pro Asp Tyr Ala Ser Gln Trp Tyr Glu Leu | |
| Gln Gln Leu Asp Ser Lys Phe Leu Glu Gln Val His Gln Leu Tyr Asp | |
| Asp Ser Phe Pro Met Glu Ile Arg Gln Tyr Leu Ala Gln Trp Leu Glu | |
| Lys Gln Asp Trp Glu His Ala Ala Asn Asp Val Ser Phe Ala Thr Ile | |
| Arg Phe His Asp Leu Leu Ser Gln Leu Asp Asp Gln Tyr Ser Arg Phe | |
| Ser Leu Glu Asn Asn Phe Leu Leu Gln His Asn Ile Arg Lys Ser Lys | |
| Arg Asn Leu Gln Asp Asn Phe Gln Glu Asp Pro Ile Gln Met Ser Met | |
| Ile Ile Tyr Ser Cys Leu Lys Glu Glu Arg Lys Ile Leu Glu Asn Ala | |
| Gln Arg Phe Asn Gln Ala Gln Ser Gly Asn Ile Gln Ser Thr Val Met | |
| Leu Asp Lys Gln Lys Glu Leu Asp Ser Lys Val Arg Asn Val Lys Asp | |
| Lys Val Met Cys Ile Glu His Glu Ile Lys Ser Leu Glu Asp Leu Gln | |
| Asp Glu Tyr Asp Phe Lys Cys Lys Thr Leu Gln Asn Arg Glu His Glu | |
| Thr Asn Gly Val Ala Lys Ser Asp Gln Lys Gln Glu Gln Leu Leu Leu | |
| Lys Lys Met Tyr Leu Met Leu Asp Asn Lys Arg Lys Glu Val Val His | |
| Lys Ile Ile Glu Leu Leu Asn Val Thr Glu Leu Thr Gln Asn Ala Leu | |
| Ile Asn Asp Glu Leu Val Glu Trp Lys Arg Arg Gln Gln Ser Ala Cys | |
| Ile Gly Gly Pro Pro Asn Ala Cys Leu Asp Gln Leu Gln Asn Trp Phe | |
| Thr Ile Val Ala Glu Ser Leu Gln Gln Val Arg Gln Gln Leu Lys Lys | |
| Leu Glu Glu Leu Glu Gln Lys Tyr Thr Tyr Glu His Asp Pro Ile Thr | |
| Lys Asn Lys Gln Val Leu Trp Asp Arg Thr Phe Ser Leu Phe Gln Gln | |
| Leu Ile Gln Ser Ser Phe Val Val Glu Arg Gln Pro Cys Met Pro Thr | |
| His Pro Gln Arg Pro Leu Val Leu Lys Thr Gly Val Gln Phe Thr Val | |
| Lys Leu Arg Leu Leu Val Lys Leu Gln Glu Leu Asn Tyr Asn Leu Lys | |
| Val Lys Val Leu Phe Asp Lys Asp Val Asn Glu Arg Asn Thr Val Lys | |
| Gly Phe Arg Lys Phe Asn Ile Leu Gly Thr His Thr Lys Val Met Asn | |
| Met Glu Glu Ser Thr Asn Gly Ser Leu Ala Ala Glu Phe Arg His Leu | |
| Gln Leu Lys Glu Gln Lys Asn Ala Gly Thr Arg Thr Asn Glu Gly Pro | |
| Leu Ile Val Thr Glu Glu Leu His Ser Leu Ser Phe Glu Thr Gln Leu | |
| Cys Gln Pro Gly Leu Val Ile Asp Leu Glu Thr Thr Ser Leu Pro Val | |
| Val Val Ile Ser Asn Val Ser Gln Leu Pro Ser Gly Trp Ala Ser Ile | |
| Leu Trp Tyr Asn Met Leu Val Ala Glu Pro Arg Asn Leu Ser Phe Phe | |
| Leu Thr Pro Pro Cys Ala Arg Trp Ala Gln Leu Ser Glu Val Leu Ser | |
| Trp Gln Phe Ser Ser Val Thr Lys Arg Gly Leu Asn Val Asp Gln Leu | |
| Asn Met Leu Gly Glu Lys Leu Leu Gly Pro Asn Ala Ser Pro Asp Gly | |
| Leu Ile Pro Trp Thr Arg Phe Cys Lys Glu Asn Ile Asn Asp Lys Asn | |
| Phe Pro Phe Trp Leu Trp Ile Glu Ser Ile Leu Glu Leu Ile Lys Lys | |
| His Leu Leu Pro Leu Trp Asn Asp Gly Cys Ile Met Gly Phe Ile Ser | |
| Lys Glu Arg Glu Arg Ala Leu Leu Lys Asp Gln Gln Pro Gly Thr Phe | |
| Leu Leu Arg Phe Ser Glu Ser Ser Arg Glu Gly Ala Ile Thr Phe Thr | |
| Trp Val Glu Arg Ser Gln Asn Gly Gly Glu Pro Asp Phe His Ala Val | |
| Glu Pro Tyr Thr Lys Lys Glu Leu Ser Ala Val Thr Phe Pro Asp Ile | |
| Ile Arg Asn Tyr Lys Val Met Ala Ala Glu Asn Ile Pro Glu Asn Pro | |
| Leu Lys Tyr Leu Tyr Pro Asn Ile Asp Lys Asp His Ala Phe Gly Lys | |
| Tyr Tyr Ser Arg Pro Lys Glu Ala Pro Glu Pro Met Glu Leu Asp Gly | |
| Pro Lys Gly Thr Gly Tyr Ile Lys Thr Glu Leu Ile Ser Val Ser Glu | |
| Val His Pro Ser Arg Leu Gln Thr Thr Asp Asn Leu Leu Pro Met Ser | |
| Pro Glu Glu Phe Asp Glu Val Ser Arg Ile Val Gly Ser Val Glu Phe | |
| Asp Ser Met Met Asn Thr Val Gln Ile Ser Tyr Ala Ser Arg Gly Gly | |
| Gly Ser Ser Gly Gly Gly Glu Asp Ala Lys Asn Ile Lys Lys Gly Pro | |
| Ala Pro Phe Tyr Pro Leu Glu Asp Gly Thr Ala Gly Glu Gln Leu His | |
| Lys Ala Met Lys Arg Tyr Ala Leu Val Pro Gly Thr Ile Ala Phe Thr | |
| Asp Ala His Ile Glu Val Asn Ile Thr Tyr Ala Glu Tyr Phe Glu Met | |
| Ser Val Arg Leu Ala Glu Ala Met Lys Arg Tyr Gly Leu Asn Thr Asn | |
| His Arg Ile Val Val Cys Ser Glu Asn Ser Leu Gln Phe Phe Met Pro | |
| Val Leu Gly Ala Leu Phe Ile Gly Val Ala Val Ala Pro Ala Asn Asp | |
| Ile Tyr Asn Glu Arg Glu Leu Leu Asn Ser Met Asn Ile Ser Gln Pro | |
| Thr Val Val Phe Val Ser Lys Lys Gly Leu Gln Lys Ile Leu Asn Val | |
| Gln Lys Lys Leu Pro Ile Ile Gln Lys Ile Ile Ile Met Asp Ser Lys | |
| Thr Asp Tyr Gln Gly Phe Gln Ser Met Tyr Thr Phe Val Thr Ser His | |
| Leu Pro Pro Gly Phe Asn Glu Tyr Asp Phe Val Pro Glu Ser Phe Asp | |
| Arg Asp Lys Thr Ile Ala Leu Ile Met Asn Ser Ser Gly Ser Thr Gly | |
| Leu Pro Lys Gly Val Ala Leu Pro His Arg Thr Ala Cys Val Arg Phe | |
| Ser His Ala Arg Asp Pro Ile Phe Gly Asn Gln Ile Ile Pro Asp Thr | |
| Ala Ile Leu Ser Val Val Pro Phe His His Gly Phe Gly Met Phe | |
| Thr Thr Leu Gly Tyr Leu Ile Cys Gly Phe Arg Val Val Leu Met | |
| Tyr Arg Phe Glu Glu Glu Leu Phe Leu Arg Ser Leu Gln Asp Tyr | |
| Lys Ile Gln Ser Ala Leu Leu Val Pro Thr Leu Phe Ser Phe Phe | |
| Ala Lys Ser Thr Leu Ile Asp Lys Tyr Asp Leu Ser Asn Leu His | |
| Glu Ile Ala Ser Gly Gly Ala Pro Leu Ser Lys Glu Val Gly Glu | |
| Ala Val Ala Lys Arg Phe His Leu Pro Gly Ile Arg Gln Gly Tyr | |
| Gly Leu Thr Glu Thr Thr Ser Ala Ile Leu Ile Thr Pro Glu Gly | |
| Asp Asp Lys Pro Gly Ala Val Gly Lys Val Val Pro Phe Phe Glu | |
| Ala Lys Val Val Asp Leu Asp Thr Gly Lys Thr Leu Gly Val Asn | |
| Gln Arg Gly Glu Leu Cys Val Arg Gly Pro Met Ile Met Ser Gly | |
| Tyr Val Asn Asn Pro Glu Ala Thr Asn Ala Leu Ile Asp Lys Asp | |
| Gly | |
| <210> SEQ ID NO: 92 | |
| <211> LENGTH: 926 | |
| <212> TYPE: PRT | |
| <213> ORGANISM: Artificial | |
| <400> SEQUENCE: 92 | |
| Met Ser Gly Tyr Val Asn Asn Pro Glu Ala Thr Asn Ala Leu Ile Asp | |
| Lys Asp Gly Trp Leu His Ser Gly Asp Ile Ala Tyr Trp Asp Glu Asp | |
| Glu His Phe Phe Ile Val Asp Arg Leu Lys Ser Leu Ile Lys Tyr Lys | |
| Gly Tyr Gln Val Ala Pro Ala Glu Leu Glu Ser Ile Leu Leu Gln His | |
| Pro Asn Ile Phe Asp Ala Gly Val Ala Gly Leu Pro Asp Asp Asp Ala | |
| Gly Glu Leu Pro Ala Ala Val Val Val Leu Glu His Gly Lys Thr Met | |
| Thr Glu Lys Glu Ile Val Asp Tyr Val Ala Ser Gln Val Thr Thr Ala | |
| Lys Lys Leu Arg Gly Gly Val Val Phe Val Asp Glu Val Pro Lys Gly | |
| Leu Thr Gly Lys Leu Asp Ala Arg Lys Ile Arg Glu Ile Leu Ile Lys | |
| Ala Lys Lys Gly Gly Lys Ser Lys Leu Gly Gly Gly Ser Ser Gly Gly | |
| Gly Gln Ile Ser Tyr Ala Ser Arg Gly Ser Gln Trp Tyr Glu Leu Gln | |
| Gln Leu Asp Ser Lys Phe Leu Glu Gln Val His Gln Leu Tyr Asp Asp | |
| Ser Phe Pro Met Glu Ile Arg Gln Tyr Leu Ala Gln Trp Leu Glu Lys | |
| Gln Asp Trp Glu His Ala Ala Asn Asp Val Ser Phe Ala Thr Ile Arg | |
| Phe His Asp Leu Leu Ser Gln Leu Asp Asp Gln Tyr Ser Arg Phe Ser | |
| Leu Glu Asn Asn Phe Leu Leu Gln His Asn Ile Arg Lys Ser Lys Arg | |
| Asn Leu Gln Asp Asn Phe Gln Glu Asp Pro Ile Gln Met Ser Met Ile | |
| Ile Tyr Ser Cys Leu Lys Glu Glu Arg Lys Ile Leu Glu Asn Ala Gln | |
| Arg Phe Asn Gln Ala Gln Ser Gly Asn Ile Gln Ser Thr Val Met Leu | |
| Asp Lys Gln Lys Glu Leu Asp Ser Lys Val Arg Asn Val Lys Asp Lys | |
| Val Met Cys Ile Glu His Glu Ile Lys Ser Leu Glu Asp Leu Gln Asp | |
| Glu Tyr Asp Phe Lys Cys Lys Thr Leu Gln Asn Arg Glu His Glu Thr | |
| Asn Gly Val Ala Lys Ser Asp Gln Lys Gln Glu Gln Leu Leu Leu Lys | |
| Lys Met Tyr Leu Met Leu Asp Asn Lys Arg Lys Glu Val Val His Lys | |
| Ile Ile Glu Leu Leu Asn Val Thr Glu Leu Thr Gln Asn Ala Leu Ile | |
| Asn Asp Glu Leu Val Glu Trp Lys Arg Arg Gln Gln Ser Ala Cys Ile | |
| Gly Gly Pro Pro Asn Ala Cys Leu Asp Gln Leu Gln Asn Trp Phe Thr | |
| Ile Val Ala Glu Ser Leu Gln Gln Val Arg Gln Gln Leu Lys Lys Leu | |
| Glu Glu Leu Glu Gln Lys Tyr Thr Tyr Glu His Asp Pro Ile Thr Lys | |
| Asn Lys Gln Val Leu Trp Asp Arg Thr Phe Ser Leu Phe Gln Gln Leu | |
| Ile Gln Ser Ser Phe Val Val Glu Arg Gln Pro Cys Met Pro Thr His | |
| Pro Gln Arg Pro Leu Val Leu Lys Thr Gly Val Gln Phe Thr Val Lys | |
| Leu Arg Leu Leu Val Lys Leu Gln Glu Leu Asn Tyr Asn Leu Lys Val | |
| Lys Val Leu Phe Asp Lys Asp Val Asn Glu Arg Asn Thr Val Lys Gly | |
| Phe Arg Lys Phe Asn Ile Leu Gly Thr His Thr Lys Val Met Asn Met | |
| Glu Glu Ser Thr Asn Gly Ser Leu Ala Ala Glu Phe Arg His Leu Gln | |
| Leu Lys Glu Gln Lys Asn Ala Gly Thr Arg Thr Asn Glu Gly Pro Leu | |
| Ile Val Thr Glu Glu Leu His Ser Leu Ser Phe Glu Thr Gln Leu Cys | |
| Gln Pro Gly Leu Val Ile Asp Leu Glu Thr Thr Ser Leu Pro Val Val | |
| Val Ile Ser Asn Val Ser Gln Leu Pro Ser Gly Trp Ala Ser Ile Leu | |
| Trp Tyr Asn Met Leu Val Ala Glu Pro Arg Asn Leu Ser Phe Phe Leu | |
| Thr Pro Pro Cys Ala Arg Trp Ala Gln Leu Ser Glu Val Leu Ser Trp | |
| Gln Phe Ser Ser Val Thr Lys Arg Gly Leu Asn Val Asp Gln Leu Asn | |
| Met Leu Gly Glu Lys Leu Leu Gly Pro Asn Ala Ser Pro Asp Gly Leu | |
| Ile Pro Trp Thr Arg Phe Cys Lys Glu Asn Ile Asn Asp Lys Asn Phe | |
| Pro Phe Trp Leu Trp Ile Glu Ser Ile Leu Glu Leu Ile Lys Lys His | |
| Leu Leu Pro Leu Trp Asn Asp Gly Cys Ile Met Gly Phe Ile Ser Lys | |
| Glu Arg Glu Arg Ala Leu Leu Lys Asp Gln Gln Pro Gly Thr Phe Leu | |
| Leu Arg Phe Ser Glu Ser Ser Arg Glu Gly Ala Ile Thr Phe Thr Trp | |
| Val Glu Arg Ser Gln Asn Gly Gly Glu Pro Asp Phe His Ala Val Glu | |
| Pro Tyr Thr Lys Lys Glu Leu Ser Ala Val Thr Phe Pro Asp Ile Ile | |
| Arg Asn Tyr Lys Val Met Ala Ala Glu Asn Ile Pro Glu Asn Pro Leu | |
| Lys Tyr Leu Tyr Pro Asn Ile Asp Lys Asp His Ala Phe Gly Lys Tyr | |
| Tyr Ser Arg Pro Lys Glu Ala Pro Glu Pro Met Glu Leu Asp Gly Pro | |
| Lys Gly Thr Gly Tyr Ile Lys Thr Glu Leu Ile Ser Val Ser Glu Val | |
| His Pro Ser Arg Leu Gln Thr Thr Asp Asn Leu Leu Pro Met Ser Pro | |
| Glu Glu Phe Asp Glu Val Ser Arg Ile Val Gly Ser Val Glu Phe Asp | |
| Ser Met Met Asn Thr Val Asp Tyr Lys Asp Asp Asp Asp Lys | |
| <210> SEQ ID NO: 93 | |
| <211> LENGTH: 335 | |
| <212> TYPE: PRT | |
| <213> ORGANISM: Artificial | |
| <400> SEQUENCE: 93 | |
| <223> artificial | |
| Arg Pro Glu Cys Val Val Pro Glu Thr Gln Cys Ala Met Lys Arg Lys | |
| Glu Lys Lys Ala Gln Lys Glu Lys Asp Lys Leu Pro Val Ser Thr Thr | |
| Thr Val Asp Asp His Met Pro Pro Ile Met Gln Cys Glu Pro Pro Pro | |
| Pro Glu Ala Ala Arg Ile His Glu Val Val Pro Arg Phe Leu Ser Asp | |
| Lys Leu Leu Val Thr Asn Arg Gln Lys Asn Ile Pro Gln Leu Thr Ala | |
| Asn Gln Gln Phe Leu Ile Ala Arg Leu Ile Trp Tyr Gln Asp Gly Tyr | |
| Glu Gln Pro Ser Asp Glu Asp Leu Lys Arg Ile Thr Gln Thr Trp Gln | |
| Gln Ala Asp Asp Glu Asn Glu Glu Ser Asp Thr Pro Phe Arg Gln Ile | |
| Thr Glu Met Thr Ile Leu Thr Val Gln Leu Ile Val Glu Phe Ala Lys | |
| Gly Leu Pro Gly Phe Ala Lys Ile Ser Gln Pro Asp Gln Ile Thr Leu | |
| Leu Lys Ala Cys Ser Ser Glu Val Met Met Leu Arg Val Ala Arg Arg | |
| Tyr Asp Ala Ala Ser Asp Ser Ile Leu Phe Ala Asn Asn Gln Ala Tyr | |
| Thr Arg Asp Asn Tyr Arg Lys Ala Gly Met Ala Glu Val Ile Glu Asp | |
| Leu Leu His Phe Cys Arg Cys Met Tyr Ser Met Ala Leu Asp Asn Ile | |
| His Tyr Ala Leu Leu Thr Ala Val Val Ile Phe Ser Asp Arg Pro Gly | |
| Leu Glu Gln Pro Gln Leu Val Glu Glu Ile Gln Arg Tyr Tyr Leu Asn | |
| Thr Leu Arg Ile Tyr Ile Leu Asn Gln Leu Ser Gly Ser Ala Arg Ser | |
| Ser Val Ile Tyr Gly Lys Ile Leu Ser Ile Leu Ser Glu Leu Arg Thr | |
| Leu Gly Met Gln Asn Ser Asn Met Cys Ile Ser Leu Lys Leu Lys Asn | |
| Arg Lys Leu Pro Pro Phe Leu Glu Glu Ile Trp Asp Val Ala Asp Met | |
| Ser His Thr Gln Pro Pro Pro Ile Leu Glu Ser Pro Thr Asn Leu | |
| <210> SEQ ID NO: 94 | |
| <211> LENGTH: 235 | |
| <212> TYPE: PRT | |
| <213> ORGANISM: Artificial | |
| <400> SEQUENCE: 94 | |
| Glu Met Pro Val Asp Arg Ile Leu Glu Ala Glu Leu Ala Val Glu Gln | |
| Lys Ser Asp Gln Gly Val Glu Gly Pro Gly Gly Thr Gly Gly Ser Gly | |
| Ser Ser Pro Asn Asp Pro Val Thr Asn Ile Cys Gln Ala Ala Asp Lys | |
| Gln Leu Phe Thr Leu Val Glu Trp Ala Lys Arg Ile Pro His Phe Ser | |
| Ser Leu Pro Leu Asp Asp Gln Val Ile Leu Leu Arg Ala Gly Trp Asn | |
| Glu Leu Leu Ile Ala Ser Phe Ser His Arg Ser Ile Asp Val Arg Asp | |
| Gly Ile Leu Leu Ala Thr Gly Leu His Val His Arg Asn Ser Ala His | |
| Ser Ala Gly Val Gly Ala Ile Phe Asp Arg Val Leu Thr Glu Leu Val | |
| Ser Lys Met Arg Asp Met Arg Met Asp Lys Thr Glu Leu Gly Cys Leu | |
| Arg Ala Ile Ile Leu Phe Asn Pro Glu Val Arg Gly Leu Lys Ser Ala | |
| Gln Glu Val Glu Leu Leu Arg Glu Lys Val Tyr Ala Ala Leu Glu Glu | |
| Tyr Thr Arg Thr Thr His Pro Asp Glu Pro Gly Arg Phe Ala Lys Leu | |
| Leu Leu Arg Leu Pro Ser Leu Arg Ser Ile Gly Leu Lys Cys Leu Glu | |
| His Leu Phe Phe Phe Arg Leu Ile Gly Asp Val Pro Ile Asp Thr Phe | |
| Leu Met Glu Met Leu Glu Ser Pro Ser Asp Ser | |

What is claimed is:

1. Two polypeptides comprising a first non-naturally occurring polypeptide comprising a fragment or domain of a nuclear receptor protein and a second non-naturally occurring polypeptide comprising a different fragment or domain of a nuclear receptor protein, wherein the first polypeptide is capable of binding an activating ligand, wherein the second polypeptide is capable of associating with the first polypeptide in the presence of the activating ligand, wherein each of the first and second polypeptides further comprise heterologous amino acids or polypeptide sequences such that activating ligand induced association of the first and second polypeptides results in an activated functional, biological or cell signal transduction condition.

2. The first and second polypeptide of claim 1, wherein one or both nuclear receptor protein fragments or domains comprise an arthropod nuclear receptor amino acid sequence.

3. The first and second polypeptide of claim 1 or 2, wherein one or both nuclear receptor protein fragments or domains comprise a Group H nuclear receptor amino acid sequence.

4. The first and second polypeptide of any one of claims 1 to 3, wherein the nuclear receptor amino acid sequence of the first polypeptide comprises an ecdysone receptor (EcR) ligand binding domain, polypeptide fragment, or substitution mutant thereof.

5. The first and second polypeptide of any one of claims 1 to 4, wherein the second polypeptide nuclear receptor protein fragment or domain comprises a mammalian nuclear receptor amino acid sequence.

6. The first and second polypeptide of claim 5, wherein the mammalian nuclear receptor protein fragment or domain comprises a RXR nuclear receptor polypeptide fragment, or substitution mutant thereof.

7. The first and second polypeptide of any one of claims 1 to 6, wherein the second polypeptide nuclear receptor protein fragment or domain comprises a chimera of invertebrate and mammalian nuclear receptor amino acid sequences, or substitution mutants thereof.

8. The first and second polypeptide of claim 7, wherein the second polypeptide nuclear receptor protein fragment or domain comprises a chimera of invertebrate USP (RXR homologue) and mammalian RXR nuclear receptor amino acid sequences, or substitution mutants thereof.

9. A ligand inducible polypeptide coupling (LIPC) system comprising:
a) A first non-naturally occurring polypeptide comprising a fragment or domain of an arthropod nuclear receptor protein, and
b) A second non-naturally occurring polypeptide comprising a fragment or domain of an arthropod and/or mammalian nuclear receptor protein,
wherein the first and second polypeptides comprise additional heterologous sequences capable of producing an activated functional, biological or cell signal transduction condition following contact with an activating ligand.

10. The LIPC system of claim 9, wherein one or both nuclear receptor protein fragments or domains comprise a Group H nuclear receptor amino acid sequence.

11. The LIPC system of claim 9 or 10, wherein the first polypeptide comprises an ecdysone receptor (EcR) ligand binding domain, polypeptide fragment, or substitution mutant thereof.

12. The LIPC system of any one of claims 9 to 11, wherein the second polypeptide comprises a mammalian nuclear receptor amino acid sequence.

13. The LIPC system of claim 12, wherein the second polypeptide comprises a RXR nuclear receptor polypeptide fragment, or substitution mutant thereof.

14. The LIPC system of any one of claims 9 to 13, wherein the second polypeptide comprises a chimera of invertebrate and mammalian nuclear receptor amino acid sequences, or substitution mutants thereof.

15. The LIPC system of claim 14, wherein the second polypeptide comprises a chimera of invertebrate USP (RXR homologue) and mammalian RXR nuclear receptor amino acid sequences, or substitution mutants thereof.

16. The first and second polypeptides in any one of claims 1 to 8, or the LIPC system of any one of claims 9-15, wherein at least one of the nuclear receptor protein fragments are derived from an ecdysone receptor polypeptide selected from the group consisting of a spruce budworm Choristoneura fumiferana EcR (“CfEcR”) LBD, a beetle Tenebrio molitor EcR (“TmEcR”) LBD, a Manduca sexta EcR (“MsEcR”) LBD, a Heliothies virescens EcR (“HvEcR”) LBD, a midge Chironomus tentans EcR (“CfEcR”) LBD, a silk moth Bombyx mori EcR (“BmEcR”) LBD, a fruit fly Drosophila melanogaster EcR (“DmEcR”) LBD, a mosquito Aedes aegypti EcR (“AaEcR”) LBD, a blowfly Lucilia capitata EcR (“LcEcR”) LBD, a blowfly Lucilia cuprina EcR (“LucEcR”) LBD, a Mediterranean fruit fly Ceratitis capitata EcR (“CcEcR”) LBD, a locust Locusta migratoria EcR (“LmEcR”) LBD, an aphid Myzus persicae EcR (“MpEcR”) LBD, a fiddler crab Celuca pugilator EcR (“CpEcR”) LBD, a whitefly Bamecia argentifoli EcR (BaEcR) LBD, a leafhopper Nephotetix cincticeps EcR (NcEcR) LBD, and an ixodid tick Amblyomma americanum EcR (“AmaEcR”) LBD.

17. The first and second polypeptides in any one of claims 1 to 8, or the LIPC system of any one of claims 9-15, wherein at least one of the nuclear receptor protein fragments are derived from an ecdysone receptor polypeptide encoded by a polynucleotide comprising a nucleic acid sequence of SEQ ID NO: 1 (CfEcR-DEF), SEQ ID NO: 2 (CfEcR-CDEF), SEQ ID NO: 3 (DmEcR-DEF), SEQ ID NO: 4 (TmEcR-DEF) SEQ ID NO: 5 (AmaEcR-DEF), or a polynucleotide encoding a functional variant that is substantially identical thereto.

18. The first and second polypeptides or the LIPC system of claims 16-17, wherein at least one of the ecdysone receptor polypeptides comprises a polypeptide sequence of SEQ ID NO: 6 (CfEcR-DEF), SEQ ID NO: 7 (DmEcR-DEF), SEQ ID NO: 8 (CfEcR-CDEF), SEQ ID NO: 9 (TmEcR-DEF), SEQ ID NO: 10 (AmaEcR-DEF), or a polypeptide sequence substantially identical thereto.

19. The first and second polypeptides or the LIPC system of any one of claims 16-18, wherein the ecdysone receptor polypeptide sequence comprises about or at least 1, 2, 3, 4, 5, 6, 7, 8, 9, 10, 11, 12, 13, 14, 15, 16, 17, 18, 19, 20, 21, 22, 23, 24, or substitution mutations relative to the corresponding wild-type ecdysone receptor polypeptide.

20. The first and second polypeptides or the LIPC system of any one of claims 16-19, wherein the ecdysone receptor polypeptide is encoded by a polynucleotide comprising a codon mutation that results in a substitution of an amino acid residue, wherein the amino acid residue is at a position equivalent to or analogous to a) amino acid residue 20, 21, 48, 51, 52, 55, 58, 59, 61, 62, 92, 93, 95, 96, 107, 109, 110, 120, 123, 125, 175, 218, 219, 223, 230, 234, or 238 of SEQ ID NO: 17, b) amino acid residues 95 and 110 of SEQ ID NO: 17, c) amino acid residues 218 and 219 of SEQ ID NO: 17, d) amino acid residues 107 and 175 of SEQ ID NO: 17, e) amino acid residues 127 and 175 of SEQ ID NO: 17, f) amino acid residues 107 and 127 of SEQ ID NO: 17, g) amino acid residues 107, 127 and 175 of SEQ ID NO: 17, h) amino acid residues 52, 107 and 175 of SEQ ID NO: 17, i) amino acid residues 96, 107 and 175 of SEQ ID NO: 17, j) amino acid residues 107, 110 and 175 of SEQ ID NO: 17, k) amino acid residue 107, 121, 213, or 217 of SEQ ID NO: 18, or 1) amino acid residue 91 or 105 of SEQ ID NO: 19.

21. The first and second polypeptides or the LIPC system of any one of claims 16-20, wherein the substitution mutation is selected from the group consisting of a) E20A, Q21A, F48A, I51A, T52A, T52V, T52I, T52L, T55A, T58A, V59A, L61A, I62A, M92A, M93A, R95A, V96A, V96T, V96D, V96M, V1071, F109A, A110P, A110S, A110M, A110L, Y120A, A123F, M125A, R175E, M218A, C219A, L223A, L230A, L234A, W238A, R95A/A110P, M218A/C219A, V107I/R175E, Y127E/R175E, V107I/Y127E, V107I/Y127E/R175E, T52V/V107I/R175E, V96A/V107I/R175E, T52A/V107I/R175E, V96T/V107I/R175E, or V107I/A110P/R175E substitution mutation of SEQ ID NO: 17, b) A107P, G121R, G121L, N213A, C217A, or C217S substitution mutation of SEQ ID NO: 18, and c) G91A or A105P substitution mutation of SEQ ID NO: 19.

22. The first and second polypeptides or the LIPC system of any one of claims 16-21, wherein the retinoid X receptor polypeptide comprises a polypeptide selected from the group consisting of a vertebrate retinoid X receptor polypeptide, an invertebrate retinoid X receptor polypeptide (USP), and a chimeric retinoid X polypeptide comprising polypeptide fragments from a vertebrate and invertebrate RXR.

23. The first and second polypeptides or the LIPC system of claim 22, wherein the chimeric retinoid X receptor polypeptide comprises at least two different retinoid X receptor polypeptide fragments selected from the group consisting of a vertebrate species retinoid X receptor polypeptide fragment, an invertebrate species retinoid X receptor polypeptide fragment, and a non-Dipteran/non-Lepidopteran invertebrate species retinoid X receptor polypeptide fragment.

24. The first and second polypeptides or the LIPC system of claim 23, wherein the chimeric retinoid X receptor polypeptide comprises a retinoid X receptor polypeptide comprising at least one retinoid X receptor polypeptide fragment selected from the group consisting of an EF-domain helix 1, an EF-domain helix 2, an EF-domain helix 3, an EF-domain helix 4, an EF-domain helix 5, an EF-domain helix 6, an EF-domain helix 7, an EF-domain helix 8, an EF-domain helix 9, an EF-domain helix 10, an EF-domain helix 11, an EF-domain helix 12, an F-domain, and an EF-domain β-pleated sheet, wherein the retinoid X receptor polypeptide fragment is from a different species retinoid X receptor polypeptide or a different isoform retinoid X receptor polypeptide than the second retinoid X receptor polypeptide fragment.

25. The first and second polypeptides or the LIPC system of claim 22, wherein the chimeric retinoid X receptor polypeptide is encoded by a polynucleotide comprising a nucleic acid sequence of a) SEQ ID NO: 11, b) nucleotides 1-348 of SEQ ID NO: 12 and nucleotides 268-630 of SEQ ID NO: 13, c) nucleotides 1-408 of SEQ ID NO: 12 and nucleotides 337-630 of SEQ ID NO: 13, d) nucleotides 1465 of SEQ ID NO: 12 and nucleotides 403-630 of SEQ ID NO: 13, e) nucleotides 1-555 of SEQ ID NO: 12 and nucleotides 490-630 of SEQ ID NO: 13, f) nucleotides 1-624 of SEQ ID NO: 12 and nucleotides 547-630 of SEQ ID NO: 13, g) nucleotides 1-645 of SEQ ID NO: 12 and nucleotides 601-630 of SEQ ID NO: 13, and h) nucleotides 1-717 of SEQ ID NO: 12, nucleotides 613-630 of SEQ ID NO: 13, or a polynucleotide encoding a functional variant that is substantially identical thereto.

26. The first and second polypeptides or the LIPC system of claim 22, wherein the chimeric retinoid X polypeptide comprises a polypeptide sequence of a) SEQ ID NO: 14, b) amino acids 1-116 of SEQ ID NO: 15 and amino acids 90-210 of SEQ ID NO: 16, c) amino acids 1-136 of SEQ ID NO: 15 and amino acids 113-210 of SEQ ID NO: 16, d) amino acids 1-155 of SEQ ID NO: 15 and amino acids 135-210 of SEQ ID NO: 16, e) amino acids 1-185 of SEQ ID NO: 15 and amino acids 164-210 of SEQ ID NO: 16, f) amino acids 1-208 of SEQ ID NO: 15 and amino acids 183-210 of SEQ ID NO: 16, g) amino acids 1-215 of SEQ ID NO: 15 and amino acids 201-210 of SEQ ID NO: 16, and h) amino acids 1-239 of SEQ ID NO: 15, amino acids 205-210 of SEQ ID NO: 16, or a polypeptide sequence substantially identical thereto.

27. The first and second polypeptides or the LIPC system of any one of claims 1-26, wherein one or both additional heterologous sequences comprise a transmembrane domain.

28. The first and second polypeptides or the LIPC system of claim 27, wherein at least one of the transmembrane domains is a single-pass type I transmembrane domain.

29. An isolated polynucleotide comprising a polynucleotide sequence that encodes the first or second polypeptides in any one of claims 1 to 28.

30. A first polynucleotide comprising a nucleotide sequence encoding the first polypeptide and a second polynucleotide comprising a nucleotide sequence encoding the second polypeptide in any one of claims 1 to 28.

31. A vector comprising one of the polynucleotides of claim 29 or 30.

32. A vector comprising both of the polynucleotides of claim 29 or 30.

33. The vector of claim 31 or 32, wherein said vector is an expression vector.

34. A host cell comprising the vector of any one of claims 31 to 33.

35. The host cell of claim 34, wherein the host cell is a mammalian T-cell.

36. The host cell of claim 34, wherein the host cell is a human T-cell.

37. A method of inducing cell signal transduction comprising introducing the first and second polypeptides or the LIPC system of any one claims 1-28, the polynucleotides of claim 29 or 30, or the vector of any one of claims 31 to 33 into a host cell and contacting the host cell with an activating ligand.

38. The first and second polypeptides or the LIPC system of any one claims 1-28, the polynucleotides of claim 29 or 30, the vector of any one of claims 31 to 33, or the method of any one of claims 34 to 36, wherein the activating ligand is
c) a compound of the formula: 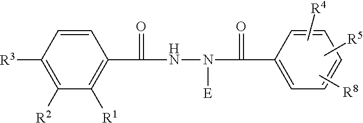
wherein:
E is a (C₄-C₆)alkyl containing a tertiary carbon or a cyano(C₃-C₅)alkyl containing a tertiary carbon; R¹ is H, Me, Et, i-Pr, F, formyl, CF₃, CHF₂, CHCl₂, CH₂F, CH₂Cl, CH₂OH, CH₂OMe, CH₂CN, CN, C≡CH, 1-propynyl, 2-propynyl, vinyl, OH, OMe, OEt, cyclopropyl, CF₂CF₃, CH═CHCN, allyl, azido, SCN, or SCHF₂;
R² is H, Me, Et, n-Pr, i-Pr, formyl, CF₃, CHF₂, CHCl₂, CH₂F, CH₂Cl, CH₂OH, CH₂OMe, CH₂CN, CN, C≡CH, 1-propynyl, 2-propynyl, vinyl, Ac, F, Cl, OH, OMe, OEt, O-n-Pr, OAc, NMe₂, NEt₂, SMe, SEt, SOCF₃, OCF₂CF₂H, COEt, cyclopropyl, CF₂CF₃, CH═CHCN, allyl, azido, OCF₃, OCHF₂, O-i-Pr, SCN, SCHF₂, SOMe, NH—CN, or joined with R³ and the phenyl carbons to which R² and R³ are attached to form an ethylenedioxy, a dihydrofuryl ring with the oxygen adjacent to a phenyl carbon, or a dihydropyryl ring with the oxygen adjacent to a phenyl carbon;
R³ is H, Et, or joined with R² and the phenyl carbons to which R² and R³ are attached to form an ethylenedioxy, a dihydrofuryl ring with the oxygen adjacent to a phenyl carbon, or a dihydropyryl ring with the oxygen adjacent to a phenyl carbon;
R⁴, R⁵, and R⁶ are independently H, Me, Et, F, Cl, Br, formyl, CF₃, CHF₂, CHCl₂, CH₂F, CH₂Cl, CH₂OH, CN, C≡CH, 1-propynyl, 2-propynyl, vinyl, OMe, OEt, SMe, or Set; or
d) an ecdysone, 20-hydroxyecdysone, ponasterone A, muristerone A, an oxysterol, a 22(R) hydroxycholesterol, 24(S) hydroxycholesterol, 25-epoxycholesterol, T0901317, 5-alpha-6-alpha-epoxycholesterol-3-sulfate, 7-ketocholesterol-3-sulfate, farnesol, a bile acid, a 1,1-biphosphonate ester, or a Juvenile hormone III.

39. The first and second polypeptides or the LIPC system of any one claims 1-28, the polynucleotides of claim 29 or 30, the vector of any one of claims 31 to 33, or the method of any one of claims 34 to 36, wherein the activating ligand is a compound of the formula: 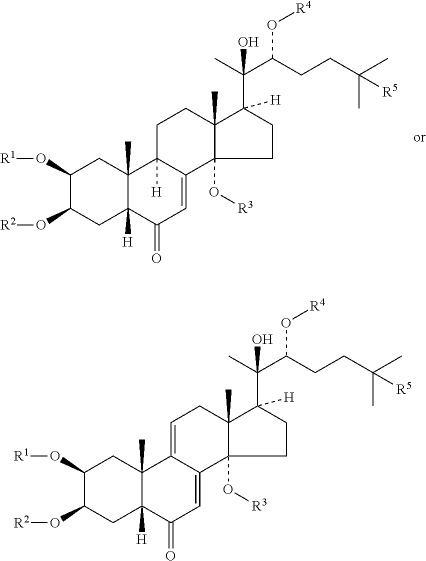
wherein R¹, R², R³, and R⁴ are: a) H, (C₁-C₆)alkyl; (C₁-C₆)haloalkyl; (C₁-C₆)cyanoalkyl; (C₁-C₆)hydroxyalkyl; (C₁-C₄)alkoxy(C₁-C₆)alkyl; (C₂-C₆)alkenyl optionally substituted with halo, cyano, hydroxyl, or (C₁-C₄)alkyl; (C₂-C₆)alkynyl optionally substituted with halo, cyano, hydroxyl, or (C₁-C₄)alkyl; (C₃-C₅)cycloalkyl optionally substituted with halo, cyano, hydroxyl, or (C₁-C₄)alkyl; or b) unsubstituted or substituted benzyl wherein the substituents are independently 1 to 5 H, halo, nitro, cyano, hydroxyl, (C₁-C₆)alkyl, or (Ci-C₆)alkoxy; and
R⁵ is H; OH; F; Cl; or (C₁-C₆)alkoxy;
provided that: when R¹, R², R³, and R⁴ are isopropyl, then R⁵ is not hydroxyl;
when R⁵ is H, hydroxyl, methoxy, or fluoro, then at least one of R¹, R², R³, and R⁴ is not H;
when only one of R¹, R², R³, and R⁴ is methyl, and R⁵ is H or hydroxyl, then the remainder of R¹, R², R³, and R⁴ are not H;
when both R⁴ and one of R¹, R², and R³ are methyl, then R⁵ is neither H nor hydroxyl;
when R¹, R², R³, and R⁴ are all methyl, then R⁵ is not hydroxyl;
when R¹, R², and R³ are all H and R⁵ is hydroxyl, then R⁴ is not ethyl, n-propyl, n-butyl, allyl, or benzyl.

40. The first and second polypeptides or the LIPC system in any one claims 1-28, the polynucleotides of claim 29 or 30, the vector of any one of claims 31 to 33, or the method of any one of claims 34 to 36, wherein the activating ligand is a compound of the formula: 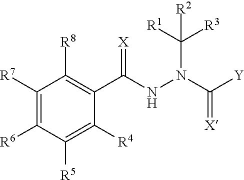
wherein X and X′ are independently 0 or S;
Y is:
(a) substituted or unsubstituted phenyl wherein the substitutents are independently 1-5H, (C₁-C₄)alkyl, (C₁-C₄)alkoxy, (C₂-C₄)alkenyl, halo (F, Cl, Br, I), (C₁-C₄)haloalkyl, hydroxy, amino, cyano, or nitro; or
(b) substituted or unsubstituted 2-pyridyl, 3-pyridyl, or 4-pyridyl, wherein the substitutents are independently 1-4H, (C₁-C₄)alkyl, (C₁-C₄)alkoxy, (C₂-C₄)alkenyl, halo (F, Cl, Br, I), (C₁-C₄)haloalkyl, hydroxy, amino, cyano, or nitro;
R¹ and R² are independently: H; cyano; cyano-substituted or unsubstituted (C₁-C₇) branched or straight-chain alkyl; cyano-substituted or unsubstituted (C₂-C₇) branched or straight-chain alkenyl; cyano-substituted or unsubstituted (C₃-C₇) branched or straight-chain alkenylalkyl; or together the valences of R¹ and R² form a (C₁-C₇) cyano-substituted or unsubstituted alkylidene group (R^{a}R^{b}C═) wherein the sum of non-substituent carbons in R^{a} and R^{b} is 0-6;
R³ is H, methyl, ethyl, n-propyl, isopropyl, or cyano;
R⁴, R⁷, and R⁸ are independently: H, (C₁-C₄)alkyl, (C₁-C₄)alkoxy, (C₂-C₄)alkenyl, halo (F, Cl, Br, I), (C₁-C₄)haloalkyl, hydroxy, amino, cyano, or nitro; and
R⁵ and R⁶ are independently: H, (C₁-C₄)alkyl, (C₂-C₄)alkenyl, (C₃-C₄)alkenylalkyl, halo (F, Cl, Br, I), C₁-C₄ haloalkyl, (C₁-C₄)alkoxy, hydroxy, amino, cyano, nitro, or together as a linkage of the type (—OCHR⁹CHR¹⁰O—) form a ring with the phenyl carbons to which they are attached;
wherein R⁹ and R¹⁰ are independently: H, halo, (C₁-C₃)alkyl, (C₂-C₃)alkenyl, (C₁-C₃)alkoxy(C₁-C₃)alkyl, benzoyloxy(C₁-C₃)alkyl, hydroxy(C₁-C₃)alkyl, halo(C₁-C₃)alkyl, formyl, formyl(C₁-C₃)alkyl, cyano, cyano(C₁-C₃)alkyl, carboxy, carboxy(C₁-C₃)alkyl, (C₁-C₃)alkoxycarbonyl(C₁-C₃)alkyl, (C₁-C₃)alkylcarbonyl(C₁-C₃)alkyl, (C₁-C₃)alkanoyloxy(C₁-C₃)alkyl, amino(C₁-C₃)alkyl, (C₁-C₃)alkylamino(C₁-C₃)alkyl (—(CH₂)ₙR^{c}R^{e}), oximo (—CH═NOH), oximo(C₁-C₃)alkyl, (C₁-C₃)alkoximo (—C═NOR^{d}), alkoximo(C₁-C₃)alkyl, (C₁-C₃)carboxamido (—C(O)NR^{e}R^{f}), (C₁-C₃)carboxamido(C₁-C₃)alkyl, C₁-C₃)semicarbazido (—C═NNHC(O)NR^{e}R^{f}), semicarbazido(C₁-C₃)alkyl, aminocarbonyloxy (—OC(O)NHR^{g}), aminocarbonyloxy(C₁-C₃)alkyl, pentafluorophenyloxycarbonyl, pentafluorophenyloxycarbonyl(C₁-C₃)alkyl, p-toluenesulfonyl oxy(C₁-C₃)alkyl, arylsulfonyl oxy(C₁-C₃)alkyl, (C₁-C₃)thio(C₁-C₃)alkyl, (C₁-C₃)alkylsulfoxido(C₁-C₃)alkyl, (C₁-C₃)alkylsulfonyl(C₁-C₃)alkyl, or (C₁-C₅)trisubstituted-siloxy(C₁-C₃)alkyl (—(CH₂),SiOR^{d}R^{e}R^{g}); wherein n=1-3, R^{c} and R^{d} represent straight or branched hydrocarbon chains of the indicated length, R^{e}, R^{f} represent H or straight or branched hydrocarbon chains of the indicated length, R^{g} represents (C₁-C₃)alkyl or aryl optionally substituted with halo or (C₁-C₃)alkyl, and R^{c}, R^{d}, R^{e}, R^{f}, and R^{g} are independent of one another; provided that
i) when R⁹ and R¹⁰ are both H, or
ii) when either R⁹ or R¹⁰ are halo, (C₁-C₃)alkyl, (C₁-C₃)alkoxy(C₁-C₃)alkyl, or benzoyloxy(C₁-C₃)alkyl, or
iii) when R⁵ and R⁶ do not together form a linkage of the type (—OCHR⁹CHR¹⁰O—),
then the number of carbon atoms, excluding those of cyano substitution, for either or both of groups R¹ or R² is greater than 4, and the number of carbon atoms, excluding those of cyano substitution, for the sum of groups R¹, R², and R³ is 10, 11, or 12.

41. A method of measuring ligand-induced cell signal transduction comprising:
a) introducing the first and second polypeptides or the LIPC system of any one claims 1-28, the polynucleotides of claim 29 or 30, or the vector of any one of claims 31 to 33 into a host cell;
b) contacting the host cell with an activating ligand; and,
c) quantitating the absolute or relative amount of ligand-induced biological activity or polypeptide oligomerization.
